(19) United States
(12) Patent Application Publication
(10) Pub. No.: US 2022/0127247 A1
(43) Pub. Date: Apr. 28, 2022

(54) N-(PYRIDIN-2-YL)PYRIDINE-SULFONAMIDE DERIVATIVES AND THEIR USE IN THE TREATMENT OF DISEASE

(71) Applicant: NOVARTIS AG, Basel (CH)

(72) Inventors: Mihai AZIMIOARA, San Diego, CA (US); Bei CHEN, San Diego, CA (US); Robert EPPLE, Solana Beach, CA (US); James Paul LAJINESS, San Diego, CA (US); Casey Jacob Nelson MATHISON, San Diego, CA (US); Juliet NABAKKA, San Diego, CA (US); Victor Ivanovich NIKULIN, Carlsbad, CA (US); Sejal PATEL, Lexington, MA (US); Dean Paul PHILLIPS, San Marcos, CA (US); Paul Vincent RUCKER, Carlsbad, CA (US); Andrew VALIERE, Descanso, CA (US); Baogen WU, San Diego, CA (US); Shanshan YAN, San Diego, CA (US); Xuefeng ZHU, San Diego, CA (US)

(21) Appl. No.: 17/428,353

(22) PCT Filed: Feb. 4, 2020
(86) PCT No.: PCT/IB2020/050884

§ 371 Date: Aug. 4, 2021

Related U.S. Application Data
(60) Provisional application No. 62/801,717, filed on Feb. 6, 2019.

(51) Int. Cl. C07D 401/12; C07D 401/14; A61K 45/06; A61K 31/444; A61K 31/4545; A61K 31/506; A61K 31/496; C07D 405/14

(52) U.S. Cl. CPC C07D 401/12; C07D 401/14; A61K 45/06; C07D 405/14; A61K 31/4545; A61K 31/506; A61K 31/496; A61K 31/444

(57) ABSTRACT

The invention relates to heterocyclic compounds of the formula (I) in which all of the variables are as defined in the specification; capable of modulating the activity of CFTR. The invention further provides a method for manufacturing compounds of the invention, and its therapeutic uses. The invention further provides methods to their preparation, to their medical use, in particular to their use in the treatment and management of diseases or disorders including Cystic fibrosis and related disorders.

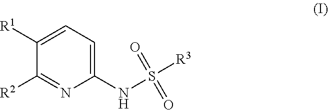

### FIELD OF THE INVENTION

The present invention relates to compounds and pharmaceutically acceptable salts thereof, which comprise an N-(pyridin-2-yl)pyridine-sulfonamide moiety. The present invention further relates to the use of such compounds in the treatment of respiratory diseases. The present invention further relates to the use of such compounds in the treatment of pancreatitis. The present invention further relates to pharmaceutical compositions comprising such compounds, a pharmaceutically acceptable carrier and optionally at least one additional therapeutic agent. The present invention further relates to combinations comprising such compounds and at least one additional therapeutic agent. The present invention further relates to the use of such pharmaceutical compositions and combinations in the treatment of respiratory diseases. The present invention further relates to the use of such pharmaceutical compositions and combinations in the treatment of pancreatitis.

### BACKGROUND OF THE INVENTION

Cystic fibrosis (CF) is an autosomal genetic disease that affects approximately 30,000 people in the United States and approximately 70,000 people worldwide. Approximately 1,000 new cases of CF are diagnosed each year. Most patients are diagnosed with CF by the age of two, and more than half of the CF population is 18 years in age or older. Despite progress in the treatment of CF, there is no cure.

Cystic fibrosis (CF) is caused by loss-of-function mutations in the CF transmembrane conductance regulator (CFTR) protein, a cAMP-regulated chloride channel expressed primarily at the apical plasma membrane of secretory epithelia in the airways, pancreas, intestine, and other tissues. CFTR is a large, multidomain glycoprotein consisting of two membrane-spanning domains, two nucleotide-binding domains (NBD1 and NBD2) that bind and hydrolyze ATP, and a regulatory (R) domain that gates the channel by phosphorylation. Nearly 2000 mutations in the CFTR gene have been identified that produce the loss-of-function phenotype by impairing its translation, cellular processing, and/or chloride channel gating. The F508del mutation, which is present in at least one allele in ˜90% of CF patients, impairs CFTR folding, stability at the endoplasmic reticulum and plasma membrane, and chloride channel gating (Dalemans et al. 1991; Denning et al. 1992; Lukacs et al. 1993; Du et al. 2005). Other mutations primarily alter channel gating (e.g., G551 D), conductance (e.g., R117H), or translation (e.g., G542X) (Welsh and Smith 1993). The fundamental premise of CFTR corrector and potentiator therapy for CF is that correction of the underlying defects in the cellular processing and chloride channel function of CF-causing mutant CFTR alleles will be of clinical benefit. Correctors are principally targeted at F508del cellular misprocessing, whereas potentiators are intended to restore cAMP-dependent chloride channel activity to mutant CFTRs at the cell surface. In contrast to current therapies, such as antibiotics, anti-inflammatory agents, mucolytics, nebulized hypertonic saline, and pancreatic enzyme replacement, which treat CF disease manifestations, correctors and potentiators correct the underlying CFTR anion channel defect.

### SUMMARY OF THE INVENTION

There remains a need for new treatments and therapies for cystic fibrosis and related disorders, including asthma, COPD, chronic bronchitis and emphysema. In addition, there remains a need for new treatments and therapies for pancreatitis. The invention provides compounds of formula (I), and sub-formulae thereof, pharmaceutically acceptable salts thereof, pharmaceutical compositions thereof and combinations thereof, wherein the compounds formula (I), and sub-formulae thereof, are CFTR correctors. The invention further provides methods of treating, preventing, or ameliorating cystic fibrosis and related disorders, where the method comprises administering to a subject in need thereof an effective amount of a CFTR corrector of the present invention, either in combination with a CFTR potentiator (dual combination) or in combination with a CFTR potentiator and a different CFTR corrector (triple combination). Various embodiments of the present invention are described herein.

In one aspect of the present invention are compounds having the structure of formula (I), or a pharmaceutically acceptable salt thereof:

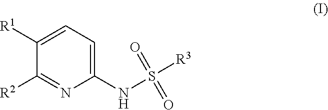

wherein R¹, R² and R³ are as defined herein.

Another aspect of the present invention are compounds having the structure of formula (Ia), or a pharmaceutically acceptable salt thereof:

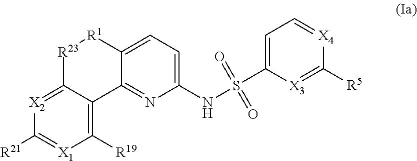

wherein: X₁, X₂, X₃, X₄, R¹, R⁵, R¹⁹, R²¹ and R²³ and L₂ are as defined herein.

In another aspect, the invention provides a pharmaceutical compositions comprising a therapeutically effective amount of a compound of the present invention, or a pharmaceutically acceptable salt thereof, and a pharmaceutically acceptable carrier.

In another aspect, the invention provides a pharmaceutical compositions comprising a compound of the present invention, or a pharmaceutically acceptable salt thereof, and a pharmaceutically acceptable carrier.

In another aspect, the invention provides a method for treating a Cystic Fibrosis Transmembrane Conductance Regulator (CFTR) mediated disease in a subject comprising administering to the subject therapeutically effective amount of a compound of the present invention, or a pharmaceutically acceptable salt thereof.

In another aspect, the invention provides a method for treating a Cystic Fibrosis Transmembrane Conductance Regulator (CFTR) mediated disease in a subject comprising administering to the subject a compound of the present invention, or a pharmaceutically acceptable salt thereof.

In another aspect, the invention provides the use of a compound of the present invention, or a pharmaceutically acceptable salt thereof, in the manufacture of a medicament for the treatment of a Cystic Fibrosis Transmembrane Conductance Regulator (CFTR) mediated disease.

In another aspect, the invention provides the use of a compound of the present invention, or a pharmaceutically acceptable salt thereof, for the treatment of a Cystic Fibrosis Transmembrane Conductance Regulator (CFTR) mediated disease.

In another aspect, the invention provides a compound of the present invention, or a pharmaceutically acceptable salt thereof, for use in the treatment of a Cystic Fibrosis Transmembrane Conductance Regulator (CFTR) mediated disease.

In another aspect, the invention provides a method for treating a Cystic Fibrosis Transmembrane Conductance Regulator (CFTR) mediated disease in a subject comprising administering to the subject a pharmaceutical composition comprising a therapeutically effective amount of a compound of the present invention, or a pharmaceutically acceptable salt thereof.

In another aspect, the invention provides a method for treating a Cystic Fibrosis Transmembrane Conductance Regulator (CFTR) mediated disease in a subject comprising administering to the subject a pharmaceutical composition comprising a compound of the present invention, or a pharmaceutically acceptable salt thereof.

In another aspect, the invention provides the use of a pharmaceutical composition comprising a compound of the present invention, or a pharmaceutically acceptable salt thereof, for the treatment of a Cystic Fibrosis Transmembrane Conductance Regulator (CFTR) mediated disease.

In another aspect, the invention provides a pharmaceutical composition comprising a compound of the present invention, or a pharmaceutically acceptable salt thereof, for use in the treatment of a Cystic Fibrosis Transmembrane Conductance Regulator (CFTR) mediated disease.

In another aspect, the invention provides a pharmaceutical combination comprising a therapeutically effective amount of a compound of the present invention, or a pharmaceutically acceptable salt thereof, and one or more additional therapeutic agents and optionally further comprising a pharmaceutically acceptable carrier.

In another aspect, the invention provides a pharmaceutical combination comprising a compound of the present invention, or a pharmaceutically acceptable salt thereof, and one or more additional therapeutic agents and optionally further comprising a pharmaceutically acceptable carrier.

In another aspect, the invention provides the use of a pharmaceutical combination of the present invention in the treatment of a Cystic Fibrosis Transmembrane Conductance Regulator (CFTR) mediated disease.

### DETAILED DESCRIPTION OF THE INVENTION

Various enumerated embodiments of the present invention are described herein. It will be recognized that features specified in each embodiment may be combined with other specified features to provide further embodiments of the present invention.

### Definitions

The term “alkyl,” as used herein, refers to a saturated branched or straight chain hydrocarbon. In certain embodiments an alkyl group is a “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” or “C₁-C₁₀alkyl”, wherein the terms “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” and “C₁-C₁₀alkyl”, as used herein, refer to an alkyl group containing at least 1, and at most 3, 4, 5, 6, 7, 8, 9 or 10 carbon atoms, respectively. Non-limiting examples of alkyl groups include methyl, ethyl, n-propyl, isopropyl, n-butyl, isobutyl, sec-butyl, tert-butyl, n-pentyl, isopentyl, hexyl, heptyl, octyl, nonyl, decyl and the like. In certain embodiments such alkyl groups are optionally substituted.

The term “alkoxy”, as used herein, refers to —O-alkyl or -alkyl-O—, wherein the “alkyl” group is as as defined herein. In certain embodiments an alkoxy group is a “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” or “C₁-C₁₀alkoxy”, wherein the terms “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” and “C₁-C₁₀alkoxy”, as used herein refer to —O—C₁-C₃alkyl, —O—C₁-C₄alkyl, —O—C₁-C₅alkyl, —O—C₁-C₆alkyl, —O—C₁-C₇alkyl, —O—C₁-C₈alkyl, —O—C₁-C₉alkyl or —O—C₁-C₁₀alkyl, respectively. Non-limiting examples of “alkoxy” groups include methoxy, ethoxy, n-propoxy, isopropoxy, n-butoxy, isobutoxy, sec-butoxy, tert-butoxy, n-pentoxy, isopentoxy, hexoxy, heptoxy, octoxy, nonoxy, decoxy and the like. In certain embodiments such alkoxy groups are optionally substituted.

The term “aryl,” as used herein, refers to an aromatic monocyclic ring system having 6 carbon atoms as ring members, an aromatic fused bicyclic ring system having 9-10 carbon atoms as ring members, or an aromatic fused tricyclic ring systems having 14 carbon atoms as ring members. Non-limiting examples of an aryl group, as used herein, include phenyl, naphthalenyl, fluorenyl, indenyl, azulenyl, anthracenyl, phenanthrenyl and the like. In certain embodiments such aryl groups are optionally substituted. In preferred embodiments an aryl group is a phenyl.

The term “C₃-C₈cycloalkyl” as used herein, refers to a saturated, monocyclic hydrocarbon ring system having 3 to 8 carbon atoms as ring members. Non-limiting examples of such “C₃-C₈cycloalkyl” groups include cyclopropyl, cyclobutyl, cyclopentyl, cyclohexyl, cycloheptyl and cyclooctyl groups. In certain embodiments such cycloalkyl groups are optionally substituted.

The term “deuterium-substituted C₁₋C₆alkyl”, as used herein, refers to the respective “C₁-C₆alkyl”, as defined herein, wherein at least one of the hydrogen atoms of the “C₁-C₆alkyl” is replaced by a deuterium atom. The deuterium-substituted C₁-C₆alkyl group can be monodeuterated, wherein one hydrogen atom of the “C₁-C₆alkyl” is replaced by one deuterium atom. The deuterium-substituted C₁₋C₆alkyl group can be dideuterated, wherein two hydrogen atoms of the “C₁-C₆alkyl” are each replaced by a deuterium atom. The deuterium-substituted C₁₋C₆alkyl groups can be trideuterated, wherein three hydrogen atoms of the “C₁-C₆alkyl” are each replaced by a deuterium atom. Furthermore, the deuterium-substituted C₁₋C₆alkyl group can be polydeuterated, wherein four or more hydrogen atoms of the “C₁-C₆alkyl” are each replaced by a deuterium atom. Non-limiting examples of a “deuterium-substituted C₁₋C₆alkyl” groups include —CH₂D, —CHD₂, —CD₃, —CH₂CH₂D, —CH₂CHD₂, —CH₂CD₃ and —CD₂CD₃.

The terms “halo-substituted C₁₋C₆alkyl” and “C₁-C₆haloalkyl” are used interchangeably herein and as used herein, refer to the respective “C₁-C₆alkyl”, as defined herein, wherein at least one of the hydrogen atoms of the “C₁-C₆alkyl” is replaced by a halo atom. The halo-substituted C₁₋C₆alkyl or C₁-C₆haloalkyl groups can be monoC₁-C₆haloalkyl, wherein such C₁-C₆haloalkyl groups have one iodo, one bromo, one chloro or one fluoro. Additionally, the C₁-C₆haloalkyl groups can be diC₁-C₆haloalkyl wherein such C₁-C₆haloalkyl groups can have two halo atoms independently selected from iodo, bromo, chloro or fluoro. Furthermore, the C₁-C₆haloalkyl groups can be polyC₁-C₆haloalkyl wherein such C₁-C₆haloalkyl groups can have two or more of the same halo atoms or a combination of two or more different halo atoms. Such polyC₁-C₆haloalkyl can be perhaloC₁-C₆haloalkyl where all the hydrogen atoms of the respective C₁-C₆alkyl have been replaced with halo atoms and the halo atoms can be the same or a combination of different halo atoms. Non-limiting examples of “halo-substituted C₁₋C₆alkyl” and “C₁-C₆haloalkyl” groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, trifluoroethyl, difluoropropyl, dichloroethyl and dichloropropyl.

The terms “halo-substituted C₁₋C₆alkoxy” and “C₁-C₆haloalkoxy” are used interchangeably herein and as used herein, refer to the respective “C₁-C₆alkoxy”, as defined herein, wherein at least one of the hydrogen atoms of the “C₁-C₆alkyl” of the “C₁-C₆haloalkoxy” is replaced by a halo atom. The halo-substituted C₁₋C₆alkoxy or C₁-C₆haloalkoxy groups can be monoC₁-C₆haloalkoxy, wherein such C₁-C₆haloalkoxy groups have one iodo, one bromo, one chloro or one fluoro. Additionally, the C₁-C₆haloalkoxy groups can be diC₁-C₆haloalkoxy wherein such C₁-C₆haloalkoxy groups can have two halo atoms independently selected from iodo, bromo, chloro or fluoro. Furthermore, the C₁-C₆haloalkoxy groups can be polyC₁-C₆haloalkoxy wherein such C₁-C₆haloalkoxy groups can have two or more of the same halo atoms or a combination of two or more different halo atoms. Such polyC₁-C₆haloalkoxy can be perhaloC₁-C₆haloalkoxy where all the hydrogen atoms of the respective C₁-C₆alkoxy have been replaced with halo atoms and the halo atoms can be the same or a combination of different halo atoms. Non-limiting examples of “halo-substituted C₁. C₆alkoxy” and “C₁-C₆haloalkoxy” groups include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, trifluoroethoxy, difluoropropoxy, dichloroethoxy and dichloropropoxy.

The terms “halo” or “halogen” as used herein, refer to fluoro, chloro, bromo and iodo.

The term “heteroaryl,” as used herein, refers to i) an aromatic, 5-6 membered monocyclic ring system wherein 1 to 4 ring members are independently selected from the heteroatoms N, O and S, ii) an aromatic, 9-10 membered fused bicyclic ring system wherein 1 to 4 ring members are independently selected from the heteroatoms N, O and S and, iii) an aromatic, 14 membered fused tricyclic ring system wherein 1 to 4 ring members are independently selected from the heteroatoms N, O and S. Non-limiting examples of heteroaryl groups, as used herein, include benzofuranyl, benzofurazanyl, benzoxazolyl, benzopyranyl, benzthiazolyl, benzothienyl, benzazepinyl, benzimidazolyl, benzothiopyranyl, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridyl, pyridazinyl, pyrazinyl, pyrimidinyl, quinoxalinyl, quinolinyl, quinazolinyl, thiazolyl, thiadiazolyl, thienyl, triazinyl, triazolyl and tetrazolyl. In certain embodiments such heteroaryl groups are optionally substituted. In preferred embodiments a heteroaryl group is a pyridyl.

The term “heteroatoms” as used herein, refers to nitrogen (N), oxygen (O) or sulfur (S) atoms.

The term “heterocycloalkyl,” as used herein refers to i) a monocyclic ring structure having 4 to 6 ring members, wherein one to two of the ring members are independently selected from N, NH, NR³⁶, O or —S—, wherein R³⁶ is C₁-C₆alkyl and ii) a fused bicyclic ring structure having 8 to 10 ring members, wherein one to two of the ring members are independently selected from N, NH, NR³⁶, O or —S—, wherein R³⁶ is C₁-C₆alkyl. Non-limiting examples of 4-6 membered heterocycloalkyl groups, as used herein, include azetadinyl, azetadin-1-yl, azetadin-2-yl, azetadin-3-yl, oxetanyl, oxetan-2-yl, oxetan-3-yl, oxetan-4-yl, thietanyl, thietan-2-yl, thietan-3-yl, thietan-4-yl, pyrrolidinyl, pyrrolidin-1-yl, pyrrolidin-2-yl, pyrrolidin-3-yl, pyrrolidin-4-yl, pyrrolidin-5-yl, tetrahydrofuranyl, tetrahydrofuran-2-yl, tetrahydrofuran-3-yl, tetrahydrofuran-4-yl, tetrahydrofuran-5-yl, tetrahydrothienyl, tetrahydrothien-2-yl, tetrahydrothien-3-yl, tetrahydrothien-4-yl, tetrahydrothien-5-yl, piperidinyl, piperidin-1-yl, piperidin-2-yl, piperidin-3-yl, piperidin-4-yl, piperidin-5-yl, piperidin-6-yl, tetrahydropyranyl, tetrahydropyran-2-yl, tetrahydropyran-3-yl, tetrahydropyran-4-yl, tetrahydropyran-5-yl, tetrahydropyran-6-yl, tetrahydrothiopyranyl, tetrahydrothiopyran-2-yl, tetrahydrothiopyran-3-yl, tetrahydrothiopyran-4-yl, tetrahydrothiopyran-5-yl, tetrahydrothiopyran-6-yl, piperazinyl, piperazin-1-yl, piperazin-2-yl, piperazin-3-yl, piperazin-4-yl, piperazin-5-yl, piperazin-6-yl, morpholinyl, morpholin-2-yl, morpholin-3-yl, morpholin-4-yl, morpholin-5-yl, morpholin-6-yl, thiomorpholinyl, thiomorpholin-2-yl, thiomorpholin-3-yl, thiomorpholin-4-yl, thiomorpholin-5-yl, thiomorpholin-6-yl, oxathianyl, oxathian-2-yl, oxathian-3-yl, oxathian-5-yl, oxathian-6-yl, dithianyl, dithian-2-yl, dithian-3-yl, dithian-5-yl, dithian-6-yl, dioxolanyl, dioxolan-2-yl, dioxolan-4-yl, dioxolan-5-yl, thioxanyl, thioxan-2-yl, thioxan-3-yl, thioxan-4-yl, thioxan-5-yl, dithiolanyl, dithiolan-2-yl, dithiolan-4-yl, dithiolan-5-yl, pyrazolidinyl, pyrazolidin-1-yl, pyrazolidin-2-yl, pyrazolidin-3-yl, pyrazolidin-4-yl, pyrazolidin-5-yl, 2-azabicyclo[4.2.0]octanyl, octahydro-1H-cyclopenta[b]pyridine and decahydroquinoline. In certain embodiments such heterocycloalkyl groups are optionally substituted.

The term “optionally substituted,” as used herein, means that the referenced group may or may not be substituted with one or more additional group(s) individually and independently selected from alkyl, alkenyl, alkynyl, cycloalkyl, aryl, heteroaryl, heterocycloalkyl, hydroxyl, alkoxy, mercaptyl, cyano, halo, carbonyl, thiocarbonyl, isocyanato, thiocyanato, isothiocyanato, nitro, perhaloalkyl, perfluoroalkyl, and amino, including mono- and di-substituted amino groups, and the protected derivatives thereof. Non-limiting examples of optional substituents include, halo, —CN, ═O, ═N—OH, ═N—OR, ═N—R, —OR, —C(O)R, —C(O)OR, —OC(O)R, —OC(O)OR, —C(O)NHR, —C(O)NR₂, —OC(O)NHR, —OC(O)NR₂, —SR—, —S(O)R, —S(O)₂R, —NHR, —N(R)₂, —NHC(O)R, —NRC(O)R, —NHC(O)OR, —NRC(O)OR, S(O)₂NHR, —S(O)₂N(R)₂, —NHS(O)₂NR₂, —NRS(O)₂NR₂, —NHS(O)₂R, —NRS(O)₂R, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy, where each R is independently selected from H, halo, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy. The placement and number of such substituent groups is done in accordance with the well-understood valence limitations of each group, for example ═O is a suitable substituent for an alkyl group but not for an aryl group.

As used herein, “CFTR” stands for cystic fibrosis transmembrane conductance regulator.

As used herein, “mutations” can refer to mutations in the CFTR gene or the CFTR protein. A “CFTR mutation” refers to a mutation in the CFTR gene, and a “CFTR mutation” refers to a mutation in the CFTR protein. A genetic defect or mutation, or a change in the nucleotides in a gene in general results in a mutation in the CFTR protein translated from that gene.

As used herein, a “F508del mutation” or “F508del” is a specific mutation within the CFTR protein. The mutation is a deletion of the three nucleotides that comprise the codon for amino acid phenylalanine at position 508, resulting in CFTR protein that lacks this phenylalanine residue.

The term “CFTR gating mutation” as used herein means a CFTR mutation that results in the production of a CFTR protein for which the predominant defect is a low channel open probability compared to normal CFTR (Van Goor, F., Hadida S. and Grootenhuis P., “Pharmacological Rescue of Mutant CFTR function for the Treatment of Cystic Fibrosis”, Top. Med. Chem. 3: 91-120 (2008)). Gating mutations include, but are not limited to, G551D, G178R, S549N, S549R, G551S, G970R, G1244E, S1251N, S1255P, and G1349D.

As used herein, a patient who is “homozygous” for a particular mutation, e.g. F508del, has the same mutation on each allele.

As used herein, a patient who is “heterozygous” for a particular mutation, e.g. F508del, has this mutation on one allele, and a different mutation on the other allele.

As used herein, the term “modulator” refers to a compound that increases the activity of a biological compound such as a protein. For example, a CFTR modulator is a compound that increases the activity of CFTR. The increase in activity resulting from a CFTR modulator may be through a corrector mechanism or a potentiator mechanism as described below.

As used herein, the term “CFTR corrector” refers to a compound that increases the amount of functional CFTR protein at the cell surface, resulting in enhanced ion transport.

As used herein, the term “CFTR potentiator” refers to a compound that increases the channel activity of CFTR protein located at the cell surface, resulting in enhanced ion transport.

As used herein, the term “modulating” as used herein means increasing or decreasing by a measurable amount.

As used herein, the term “inducing,” as in inducing CFTR activity, refers to increasing CFTR activity, whether by the corrector, potentiator, or other mechanism.

As used herein “asthma” includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.) Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

The terms “combination” or “pharmaceutical combination,” as used herein, refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein the term “co-administer” refers to the presence of two active agents in the blood of an individual. Active agents that are co-administered can be concurrently or sequentially delivered.

The terms “composition” or “pharmaceutical composition,” as used herein, refers to a compound of the present invention, or a pharmaceutically acceptable salt thereof, together with at least one pharmaceutically acceptable carrier, in a form suitable for oral or parenteral administration.

A “patient,” “subject” or “individual” are used interchangeably and refer to either a human or non-human animal. The term includes mammals such as humans. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The phrase “pharmaceutically acceptable” indicates that the substance, composition or dosage form must be compatible chemically and/or toxicologically, with the other ingredients comprising a formulation, and/or the mammal being treated therewith.

The term “a subject in need of such treatment”, refers to a subject which would benefit biologically, medically or in quality of life from such treatment.

The term “therapeutically effective amount,” as used herein, refers to an amount of a compound of the present invention that will ameliorate symptoms, alleviate conditions, slow or delay disease progression, prevent a disease, or elicit the biological or medical response of a subject, for example, increasing the amount of functional CFTR protein at the cell surface, resulting in enhanced ion transport or increasing the channel activity of CFTR protein located at the cell surface, resulting in enhanced ion transport.

As used herein, the term “treat”, “treating” or “treatment” of any disease or disorder, refers to the management and care of a patient for the purpose of combating the disease, condition, or disorder and includes the administration of a compound of the present invention to prevent the onset of the symptoms or complications, alleviating the symptoms or complications, or eliminating the disease, condition or disorder.

In addition, the terms “treatment,” “treating,” as used herein, generally mean the improvement of CF or its symptoms or lessening the severity of CF or its symptoms in a subject. “Treatment,” as used herein, includes, but is not limited to, the following: (i) to ameliorating the disease or disorder (i.e., slowing or arresting or reducing the development of the disease or at least one of the clinical symptoms thereof); (ii) to alleviating or ameliorating at least one physical parameter including those which may not be discernible by the patient; or (iii) to preventing or delaying the onset or development or progression of the disease or disorder. (iiii) increased growth of the subject, increased weight gain, reduction of mucus in the lungs, improved pancreatic and/or liver function, reduced cases of chest infections, and/or reduced instances of coughing or shortness of breath. Improvements in or lessening the severity of any of these conditions can be readily assessed according to standard methods and techniques known in the art.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

The compound names provided herein were obtained using ChemDraw Ultra version 14.0 (CambridgeSoft®) or JChem version 17.2.1300.1489 (ChemAxon).

As used herein, the term “a,” “an,” “the” and similar terms used in the context of the present invention (especially in the context of the claims) are to be construed to cover both the singular and plural unless otherwise indicated herein or clearly contradicted by the context.

### Compounds of the Invention

The invention therefore provides a compound having the structure of formula (I):

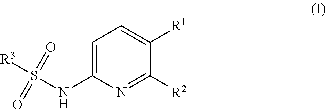

wherein:
R¹ is H, C₁₋C₆alkyl, halo, halo-substituted C₁₋C₆alkyl, deuterium substituted C₁₋C₆alkyl, C₁₋C₆alkoxy or halo-substituted C₁₋C₆alkoxy;
R² is selected from:
   a) a phenyl substituted with 1 to 2 R⁴ groups;
   b) an unsubstituted 5 to 6 membered heteroaryl having 1 to 3 heteroatoms independently selected from N, O and S,
   and
   c) a 5 to 6 membered heteroaryl having 1 to 3 heteroatoms independently selected from N, O and S, wherein the heteroaryl is substituted with 1 to 3 R⁴ groups;
R³ is a pyridin-2-yl or a pyridin-4-yl, wherein the pyridin-2-yl or a pyridin-4-yl is substituted with an R⁵ group;
each R⁴ is independently selected from D, C₁₋C₆alkyl, phenyl, phenoxy, halo, C₁₋C₆alkoxy, C₃₋C₈cycloalkyl, C₂₋C₆alkenyl, halo-substituted C₁₋C₆alkyl, deuterium substituted C₁-C₆alkyl, halo-substituted C₁₋C₆alkoxy and a 5 to 6 membered heteroaryl having 1 to 3 heteroatoms independently selected from N, O and S;
R⁵ is selected from
   a) —NR⁶R⁷;
   b) —OR¹¹;
   c) —S(CR⁸R⁹)ₙC(═O)OR¹⁰,
   d) an unsubstituted 6 membered heterocycloalkyl having 1 to 2 ring members independently selected from N, O and S;
   and
   e) a 5 to 6 membered monocyclic heterocycloalkyl having 1 to 2 N heteroatoms as ring members, wherein the heterocycloalkyl is substituted with 1 to 2 R¹² groups;
R⁶ is H, —C₁₋C₆alkyl, halo-substituted C₁₋C₆alkyl, C₃₋C₈cycloalkyl, —(CR⁸R⁹)ₙOR¹⁴ or —(CR⁸R⁹)ₘR¹⁶;
R⁷ is H, —C₁₋C₆alkyl, —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, —(CR¹⁴R¹⁵)ₘC(═O)OR¹⁰, —CHR¹²R¹⁸, a monocyclic C₃₋C₈cycloalkyl substituted with 1 to 2 R¹² groups, a bicyclic C₇₋C₁₀cycloalkenyl substituted with 1 to 2 R¹² groups or a bicyclic C₇₋C₁₀cycloalkyl substituted with 1 to 2 R¹² groups;
each R⁸ is independently selected from H, D, deuterium substituted C₁₋C₆alkyl and C₁₋C₆alkyl;
each R⁹ is independently selected from H, D, deuterium substituted C₁₋C₆alkyl and C₁₋C₆alkyl;
each R¹⁰ is independently selected from H, and C₁₋C₆alkyl;
R¹¹ is —(CR⁸R⁹)ₙC(═O)OR¹³ or a C₃₋C₈cycloalkyl substituted with 1 to 2 R¹² groups,
each R¹² is independently selected from C₁₋C₆alkyl, —OH, —(CR⁸R⁹)ₙC(═O)OR¹³, —C(═O)NH(CR¹⁴R¹⁵)ₘC(═O)OR¹³, —C(═O)OR¹³, (CR¹⁴R¹⁵)ₘC(═O)OR¹³, —O(CR⁸R⁹)ₙC(═O)OR¹³, benzoic acid and tetrazolyl;
each R¹³ is independently selected from H, and C₁₋C₆alkyl;
each R¹⁴ is independently selected from H, D, deuterium substituted C₁₋₆alkyl and C₁₋₆alkyl;
each R¹⁵ is independently selected from H, D, deuterium substituted C₁₋C₆alkyl and C₁₋C₆alkyl;
or R¹⁴ and R¹⁵ together with the carbon in CR¹⁴R¹⁵ form a C₃₋C₈cycloalkyl;
R¹⁶ is a C₃₋C₈cycloalkyl;
R¹⁷ is
   a) a C₃₋C₈cycloalkyl substituted with 1 to 2 R¹² groups,
   or
   b) a 5 to 6 membered monocyclic heterocycloalkyl having 1 to 2 ring members independently selected from N, O and S substituted with 1 to 2 R¹² groups;
R¹⁸ is adamantanyl;
each m is independently selected from 1, 2, 3, 4, 5, 6, 7, 8, 9 and 10;
and
each n is independently selected from 1, 2, 3, 4, 5, 6, 7, 8, 9 and 10.

Unless specified otherwise, the term “compound of the invention”, “compounds of the invention”, “compound of the present invention” or “compounds of the present invention” refers to a compound or compounds of formula (I), subformulae thereof (such as formula (Ia), formula (Ib), formula (Ic), formula (Id), formula (Ie), formula (If and formula (Ig) and exemplified compounds, and salts thereof, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates).

Certain aspects and examples of the compounds of the present invention are provided in the following listing of additional, enumerated embodiments. It will be recognized that features specified in each embodiment may be combined with other specified features to provide further embodiments of the present invention.

### Embodiment 1

The compound of formula (I), or a pharmaceutically acceptable salt thereof, wherein R² is selected from a phenyl substituted with 1 to 2 R⁴ groups, an unsubstituted 5 to 6 membered heteroaryl having 1 to 3 heteroatoms independently selected from N, O and S, and a 5 to 6 membered heteroaryl having 1 to 3 heteroatoms independently selected from N, O and S substituted with 1 to 3 R⁴ groups.

### Embodiment 2

The compound of formula (I), or a pharmaceutically acceptable salt thereof, wherein R² is a phenyl substituted with 1 to 2 R⁴ groups, or R² is a 5 to 6 membered heteroaryl having 1 to 3 heteroatoms independently selected from N, O and S substituted with 1 to 3 R⁴ groups.

### Embodiment 3

The compound of formula (I), or a pharmaceutically acceptable salt thereof, wherein R² is a phenyl substituted with 1 to 2 R⁴ groups.

### Embodiment 4

The compound of formula (I), or a pharmaceutically acceptable salt thereof, wherein R² is a 5 to 6 membered heteroaryl having 1 to 3 heteroatoms independently selected from N, O and S substituted with 1 to 3 R⁴ groups.

### Embodiment 5

The compound of formula (I), or a pharmaceutically acceptable salt thereof, or any one of embodiments 1 to 4, wherein, each R⁴ is independently selected from D, C₁₋₆ alkyl, phenyl, phenoxy, halo, C₁-C₆alkoxy, C₃-C₈cycloalkyl, C₂-C₆alkenyl, halo-substituted C₁₋₆ alkyl, deuterium substituted C₁₋₆alkyl, halo-substituted C₁₋₆alkoxy and a 5 to 6 membered heteroaryl having 1 to 3 heteroatoms independently selected from N, O and S

### Embodiment 6

The compound of formula (I), or a pharmaceutically acceptable salt thereof, or any one of embodiments 1 to 5, wherein each R⁴ is independently selected from D, C₁₋₆ alkyl, phenyl, phenoxy, halo, C₁₋₆alkoxy, C₃₋₈cycloalkyl, C₂₋₆alkenyl, and a 5 to 6 membered heteroaryl having 1 to 3 heteroatoms independently selected from N, O and S.

### Embodiment 7

The compound of formula (I), or a pharmaceutically acceptable salt thereof, or any one of embodiments 1 to 6, wherein each R⁴ is independently selected from D, C₁₋₆ alkyl, halo, C₁₋₆alkoxy and C₂₋₆alkenyl.

### Embodiment 8

The compound of formula (I), or a pharmaceutically acceptable salt thereof, or any one of embodiments 1 to 6, wherein each R⁴ is independently selected from phenyl, phenoxy, C₃₋₈cycloalkyl and a 5 to 6 membered heteroaryl having 1 to 3 heteroatoms independently selected from N, O and S.

### Embodiment 9

The compound of formula (I), or a pharmaceutically acceptable salt thereof, or any one of embodiments 1 to 6, wherein each R⁴ is independently selected from D, methyl, ethyl, propyl, isopropyl, butyl, tert-butyl, CD₃, phenyl, phenoxy, Cl, F, methoxy, cyclopropyl, cyclobutyl, ethenyl, pyrimidinyl and pyridyl.

### Embodiment 10

The compound of formula (I), or a pharmaceutically acceptable salt thereof, or any one of embodiments 1 to 6, wherein each R⁴ is independently selected from methyl, ethyl, isopropyl, tert-butyl, phenyl, phenoxy, Cl, methoxy, cyclopropyl, cyclobutyl, ethenyl, pyrimidinyl and pyridyl.

### Embodiment 11

The compound of formula (I), or a pharmaceutically acceptable salt thereof, or any one of embodiments 1 to 6, wherein each R⁴ is independently selected from methyl, ethyl, isopropyl, tert-butyl, F and ethenyl.

### Embodiment 12

The compound of formula (I), or a pharmaceutically acceptable salt thereof, or any one of embodiments 1 to 6, wherein each R⁴ is independently selected from methyl, ethyl, isopropyl, tert-butyl and ethenyl.

### Embodiment 13

The compound of formula (I), or a pharmaceutically acceptable salt thereof, or any one of embodiments 1 to 6, wherein each R⁴ is independently selected from phenyl, phenoxy, cyclopropyl, cyclobutyl, pyrimidinyl and pyridyl.

### Embodiment 14

The compound of formula (I), or a pharmaceutically acceptable salt thereof, or any one of embodiments 1 to 6, wherein each R⁴ is independently selected from cyclopropyl and cyclobutyl.

### Embodiment 15

The compound of formula (I), or a pharmaceutically acceptable salt thereof, or any one of embodiments 1 to 6, wherein each R⁴ is independently selected from phenyl, phenoxy, pyrimidinyl and pyridyl.

### Embodiment 16

The compound of formula (I), or a pharmaceutically acceptable salt thereof, or any one of embodiments 1 to 3, wherein R² is

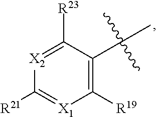

where
X₁ is CR²⁰ or N; X₂ is CR²² or N;
R¹⁹ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆ alkoxy or a 5 to 6 membered heteroaryl having 1 to 3 heteroatoms independently selected from N, O and S;
R²⁰ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆ alkoxy or a 5 to 6 membered heteroaryl having 1 to 3 heteroatoms independently selected from N, O and S;
R²¹ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆ alkoxy or a 5 to 6 membered heteroaryl having 1 to 3 heteroatoms independently selected from N, O and S;
R²² is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆ alkoxy or a 5 to 6 membered heteroaryl having 1 to 3 heteroatoms independently selected from N, O and S; and
R²³ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆ alkoxy or a 5 to 6 membered heteroaryl having 1 to 3 heteroatoms independently selected from N, O and S.

### Embodiment 17

The compound of formula (I), or a pharmaceutically acceptable salt thereof, or of embodiments 16, wherein R² is

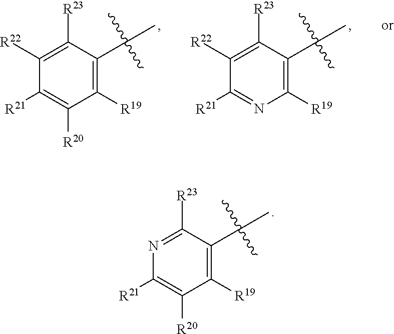

### Embodiment 18

The compound of formula (I), or a pharmaceutically acceptable salt thereof, or of embodiments 16, wherein R² is

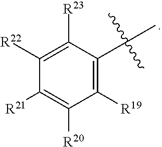

### Embodiment 19

The compound of formula (I), or a pharmaceutically acceptable salt thereof, or of embodiments 16, wherein R² is

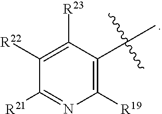

### Embodiment 20

The compound of formula (I), or a pharmaceutically acceptable salt thereof, or of embodiments 16, wherein R² is

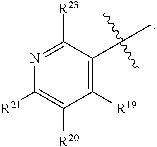

### Embodiment 21

The compound of formula (I), or a pharmaceutically acceptable salt thereof, wherein R² is an unsubstituted 5 to 6 membered heteroaryl having 1 to 3 heteroatoms independently selected from N, O and S.

### Embodiment 22

The compound of formula (I), or a pharmaceutically acceptable salt thereof, or any one of embodiments 1 to 21, wherein R³ is a pyridin-2-yl or a pyridin-4-yl, wherein the pyridin-2-yl or a pyridin-4-yl is substituted with an R⁵ group.

### Embodiment 23

The compound of formula (I), or a pharmaceutically acceptable salt thereof, or any one of embodiments 1 to 21, wherein R³ is a pyridin-2-yl substituted with an R⁵ group.

### Embodiment 24

The compound of formula (I), or a pharmaceutically acceptable salt thereof, or any one of embodiments 1 to 21, wherein R³ is a pyridin-4-yl substituted with an R⁵ group.

### Embodiment 25

The compound of formula (I), or a pharmaceutically acceptable salt thereof, or any one of embodiments 1 to 21, wherein R³ is

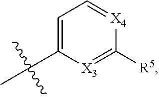

where X₃ is CH and X₄ is N, or X₃ is N and X₄ is CH.

### Embodiment 26

The compound of formula (I), or a pharmaceutically acceptable salt thereof, or of embodiment 25, wherein R³ is

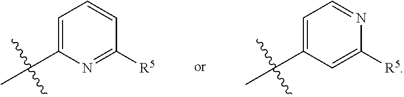

### Embodiment 27

The compound of formula (I), or a pharmaceutically acceptable salt thereof, or of embodiment 25, wherein R³ is

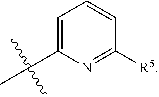

### Embodiment 28

The compound of formula (I), or a pharmaceutically acceptable salt thereof, or of embodiment 25, wherein R³ is

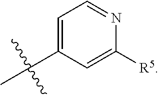

### Embodiment 29

The compound of formula (I), or a pharmaceutically acceptable salt thereof, having the structure of formula (Ia), or a pharmaceutically acceptable salt thereof,

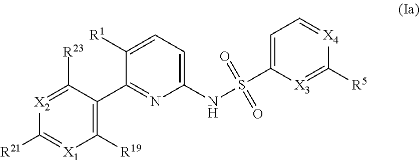

wherein:
X₁ is CR²⁰ or N; X₂ is CR²² or N; X₃ is CH and X₄ is N, or X₃ is N and X₄ is CH;
R¹ is H, C₁₋₆ alkyl, halo, halo-substituted C₁₋₆alkyl, deuterium substituted C₁₋₆alkyl, C₁₋₆ alkoxy or halo-substituted C₁₋₆alkoxy;
R⁵ is selected from
   a) —NR⁶R⁷;
   b) —OR¹¹;
   c) —S(CR⁸R⁹)ₙC(═O)OR¹⁰,
   d) an unsubstituted 6 membered heterocycloalkyl having 1 to 2 ring members independently selected from N, O and S;
   and
   e) a 5 to 6 membered monocyclic heterocycloalkyl having 1 to 2 N heteroatoms as ring members, wherein the heterocycloalkyl is substituted with 1 to 2 R¹² groups;
R⁶ is H, —C₁₋₆alkyl, halo-substituted C₁₋₆alkyl, C₃₋₈cycloalkyl, —(CR⁸R⁹)ₙOR¹⁴, or —(CR⁸R⁹)ₘR¹⁶;
R⁷ is H, —C₁₋₆alkyl, —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, —(CR¹⁴R¹⁵)ₘC(═O)OR¹⁰, —CHR¹²R¹⁸, a monocyclic C₃₋₈cycloalkyl substituted with 1 to 2 R¹² groups, a bicyclic C₇₋₁₀cycloalkenyl substituted with 1 to 2 R¹² groups or a bicyclic C₇₋₁₀cycloalkyl substituted with 1 to 2 R¹² groups;
each R⁸ is independently selected from H, D, deuterium substituted C₁₋₆alkyl and C₁₋₆alkyl;
each R⁹ is independently selected from H, D, deuterium substituted C₁₋₆alkyl and C₁₋₆alkyl;
each R¹⁰ is independently selected from H, and C₁₋₆alkyl;
R¹¹ is —(CR⁸R⁹)ₙC(═O)OR¹³;
each R¹² is independently selected from C₁₋₆alkyl, —OH, —(CR⁸R⁹)ₙC(═O)OR¹³, —C(═O)NH(CR¹⁴R¹⁵)ₘC(═O)OR¹³, —C(═O)OR¹³, —(CR¹⁴R¹⁵)ₘC(═O)OR¹³, —O(CR⁸R⁹)ₙC(═O)OR¹³, benzoic acid and tetrazolyl;
each R¹³ is independently selected from H, and C₁₋₆alkyl;
each R¹⁴ is independently selected from H, D, deuterium substituted C₁₋₆alkyl and C₁₋₆alkyl;
each R¹⁵ is independently selected from H, D, deuterium substituted C₁₋₆alkyl and C₁₋₆alkyl;
or R¹⁴ and R¹⁵ together with the carbon in CR¹⁴R¹⁵ form a C₃₋₈cycloalkyl;
R¹⁶ is a C₃₋₈cycloalkyl;
R¹⁷ is
   a) a C₃₋₈cycloalkyl substituted with 1 to 2 R¹² groups,
   or
   b) a 5 to 6 membered monocyclic heterocycloalkyl having 1 to 2 ring members independently selected from N, O and S substituted with 1 to 2 R¹² groups;
R¹⁸ is adamantanyl;
R¹⁹ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S;
R²⁰ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆ alkoxy or a 5 to 6 membered heteroaryl having 1 to 3 heteroatoms independently selected from N, O and S;
R²¹ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S;
R²² is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S;
R²³ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S;
each m is independently selected from 1, 2, 3, 4, 5, 6, 7, 8, 9 and 10;
and
each n is independently selected from 1, 2, 3, 4, 5, 6, 7, 8, 9 and 10.

### Embodiment 30

The compound of formula (I) or formula (Ia), or a pharmaceutically acceptable salt thereof wherein:
R¹ is Cl, F or CF₃;
R⁵ is selected from
   a) —NR⁶R⁷;
   b) —OR¹¹;
   c) —S(CH₂)₂C(═O)OH,
   d) a piperidinyl substituted with 1 to 2 R¹² groups;
   e) a piperazinyl is substituted with 1 to 2 R¹² groups;
   and
   f) a pyrrolidinyl substituted with 1 to 2 R¹² groups;
R⁶ is H, methyl, ethyl, propyl, isopropyl, butyl, isobutyl, 3,3,3-trifluoropropyl, cyclopropyl, cyclobutyl, —(CH₂)ₙOR¹⁰, or —CH₂R¹⁶;
R⁷ is —CH₂C(═O)OH, —(CH₂)₂C(═O)OH, —(CH₂)₃C(═O)OH, —(CH₂)₄C(═O)OH, —CH₂R¹⁷, —CH₂(CR¹⁴R¹⁵)C(═O)OH, —(CR¹⁴R¹⁵)₂C(═O)OH, —CHR¹²R¹⁸, a cyclohexyl substituted with 1 to 2 R¹² groups, a bicyclo[2.2.1]heptenyl substituted with 1 to 2 R¹² groups, or a bicyclo[2.2.1]heptanyl substituted with 1 to 2 R¹² groups;
R¹⁰ is H or methyl;
R¹¹ is —(CH₂)₂C(═O)OR¹³;
each R¹² is independently selected from methyl, —OH, —C(═O)OR¹³, —CH₂C(═O)OR¹³, —(CH₂)₂C(═O)OR¹³, —(CH₂)₃C(═O)OR¹³, —(CR¹⁴R¹⁵)C(═O)OR¹³, —(CR¹⁴R¹⁵)₂C(═O)OR¹³, —OCH₂C(═O)OR¹³, —CH₂C(═O)OR¹³, —C(═O)NH(CR¹⁴R¹⁵)C(═O)OR¹³, benzoic acid and tetrazolyl;
each R¹³ is independently selected from H, methyl and ethyl;
each R¹⁴ is independently selected from H, methyl and ethyl;
each R¹⁵ is independently selected from H, methyl and ethyl;
or R¹⁴ and R¹⁵ together with the carbon in CR¹⁴R¹⁵ form a cyclopropyl, a cyclobutyl or a cyclopentyl;
R¹⁶ is a cyclopropyl;
R¹⁷ is
   a) a cyclobutyl substituted with 1 to 2 R¹² groups;
   b) a cyclopentyl substituted with 1 to 2 R¹² groups;
   or
   c) a tetrahydro-2H-pyranyl substituted with 1 to 2 R¹² groups;
R¹⁸ is adamantanyl;
R¹⁹ is independently selected from methyl, ethyl, isopropyl, tert-butyl, phenyl, phenoxy, Cl, F, cyclopropyl, cyclobutyl, ethenyl, a pyrimidinyl and a pyridyl;
R²⁰ is H, Cl, F or methoxy;
R²¹ is H;
R²² is H, Cl or F;
and
R²³ is H or methyl.

### Embodiment 31

The compound of formula (Ia), or a pharmaceutically acceptable salt thereof, or any one of embodiments 16, 29 or 30, wherein X₁ is CR²⁰.

### Embodiment 32

The compound of formula (Ia), or a pharmaceutically acceptable salt thereof, or any one of embodiments 16, 29 or 30, wherein X₁ is N.

### Embodiment 33

The compound of formula (Ia), or a pharmaceutically acceptable salt thereof, or any one of embodiments 16, 29, 30 or 31, wherein X₂ is CR²⁰.

### Embodiment 34

The compound of formula (Ia), or a pharmaceutically acceptable salt thereof, or any one of embodiments 16, 29, 30 or 31, wherein X₂ is N.

### Embodiment 35

The compound of formula (Ia), or a pharmaceutically acceptable salt thereof, having the structure of formula (Ib), or a pharmaceutically acceptable salt thereof,

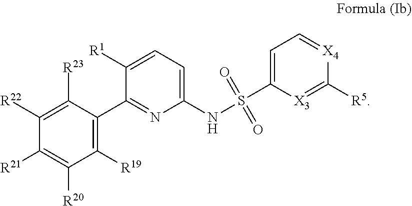

### Embodiment 36

The compound of formula (Ia), or a pharmaceutically acceptable salt thereof, having the structure of formula (Ic), or a pharmaceutically acceptable salt thereof,

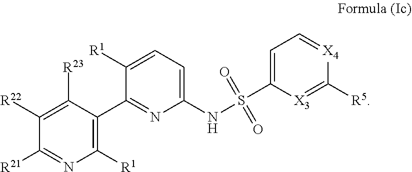

### Embodiment 37

The compound of formula (Ia), or a pharmaceutically acceptable salt thereof, having the structure of formula (Id), or a pharmaceutically acceptable salt thereof,

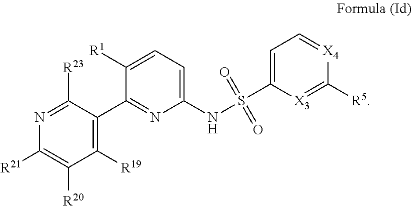

### Embodiment 38

The compound of formula (Ia), or a pharmaceutically acceptable salt thereof, or any one of embodiments 25 or 29 to 37, wherein X₃ is CH and X₄ is N.

### Embodiment 39

The compound of formula (Ia), or a pharmaceutically acceptable salt thereof, or any one of embodiments 25 or 29 to 37, wherein X₃ is N and X₄ is CH.

### Embodiment 40

The compound of formula (Ia), or a pharmaceutically acceptable salt thereof, having the structure of formula (Ie), or a pharmaceutically acceptable salt thereof,

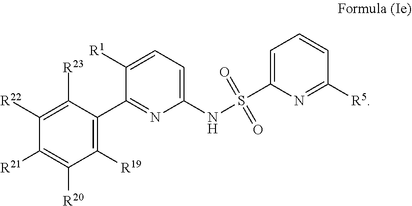

### Embodiment 41

The compound of formula (Ia), or a pharmaceutically acceptable salt thereof, having the structure of formula (If), or a pharmaceutically acceptable salt thereof,

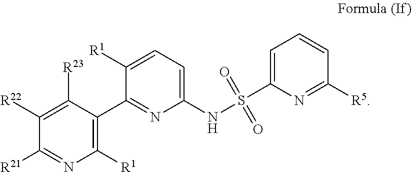

### Embodiment 42

The compound of formula (Ia), or a pharmaceutically acceptable salt thereof, having the structure of formula (Ig), or a pharmaceutically acceptable salt thereof,

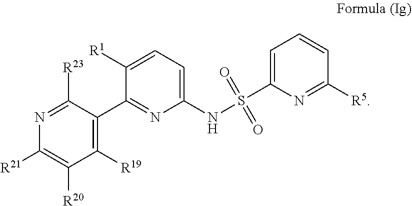

### Embodiment 43

The compound of formula (I), or a pharmaceutically acceptable salt thereof, or any one of embodiments 1 to 42, wherein R¹ is H, C₁₋₆ alkyl or deuterium substituted C₁₋₆ alkyl.

### Embodiment 44

The compound of formula (I), or a pharmaceutically acceptable salt thereof, or any one of embodiments 1 to 42, wherein R¹ is halo or halo-substituted C₁₋₆alkyl.

### Embodiment 45

The compound of formula (I), or a pharmaceutically acceptable salt thereof, or any one of embodiments 1 to 42, wherein R¹ is Cl, F or CF₃.

### Embodiment 46

The compound of formula (I), or a pharmaceutically acceptable salt thereof, or any one of embodiments 1 to 42, wherein R¹ is Cl or CF₃.

### Embodiment 47

The compound of formula (I), or a pharmaceutically acceptable salt thereof, or any one of embodiments 1 to 42, wherein R¹ is halo.

### Embodiment 48

The compound of formula (I), or a pharmaceutically acceptable salt thereof, or any one of embodiments 1 to 42, wherein R¹ is Cl or F.

### Embodiment 49

The compound of formula (I), or a pharmaceutically acceptable salt thereof, or any one of embodiments 1 to 42, wherein R¹ is Cl.

### Embodiment 50

The compound of formula (I), or a pharmaceutically acceptable salt thereof, or any one of embodiments 1 to 42, wherein R¹ is halo-substituted C₁₋₆alkyl.

### Embodiment 51

The compound of formula (I), or a pharmaceutically acceptable salt thereof, or any one of embodiments 1 to 42, wherein R¹ is CF₃.

### Embodiment 52

The compound of formula (I), or a pharmaceutically acceptable salt thereof, or any one of embodiments 1 to 42, wherein R¹ is H, C₁₋₆alkoxy or halo-substituted C₁₋₆ alkoxy.

### Embodiment 53

The compound of formula (I), or a pharmaceutically acceptable salt thereof, or any one of embodiments 1 to 52, wherein R⁵ is selected from
a) —NR⁶R⁷;
b) —OR¹¹;
c) —S(CR⁸R⁹)ₙC(═O)OR¹⁰,
d) an unsubstituted 6 membered heterocycloalkyl having 1 to 2 ring members independently selected from N, O and S;
and
e) a 5 to 6 membered monocyclic heterocycloalkyl having 1 to 2 N heteroatoms as ring members, wherein the heterocycloalkyl is substituted with 1 to 2 R¹² groups.

### Embodiment 54

The compound of formula (I), or a pharmaceutically acceptable salt thereof, or any one of embodiments 1 to 52, wherein R⁵ is —NR⁶R⁷.

### Embodiment 55

The compound of formula (I), or a pharmaceutically acceptable salt thereof, or any one of embodiments 1 to 52, wherein R⁵ is —OR¹¹.

### Embodiment 56

The compound of formula (I), or a pharmaceutically acceptable salt thereof, or any one of embodiments 1 to 52, wherein R⁵ is —S(CR⁸R⁹)ₙC(═O)OR¹⁰.

### Embodiment 57

The compound of formula (I), or a pharmaceutically acceptable salt thereof, or any one of embodiments 1 to 52, wherein R⁵ is a 5 to 6 membered monocyclic heterocycloalkyl having 1 to 2 N heteroatoms as ring members, wherein the heterocycloalkyl is substituted with 1 to 2 R¹² groups.

### Embodiment 58

The compound of formula (I), or a pharmaceutically acceptable salt thereof, or any one of embodiments 1 to 52, wherein R⁵ is a piperidinyl substituted with 1 to 2 R¹² groups.

### Embodiment 59

The compound of formula (I), or a pharmaceutically acceptable salt thereof, or any one of embodiments 1 to 52, wherein R⁵ is a piperazinyl is substituted with 1 to 2 R¹² groups.

### Embodiment 60

The compound of formula (I), or a pharmaceutically acceptable salt thereof, or any one of embodiments 1 to 52, wherein R⁵ is a pyrrolidinyl substituted with 1 to 2 R¹² groups.

### Embodiment 61

The compound of formula (I), or a pharmaceutically acceptable salt thereof, or any one of embodiments 1 to 54, wherein R⁶ is H, —C₁₋₆alkyl, halo-substituted C₁₋₆alkyl, C₃₋₈cycloalkyl, —(CR⁸R⁹)ₙOR¹⁴, or —(CR⁸R⁹)ₘR¹⁶.

### Embodiment 62

The compound of formula (I), or a pharmaceutically acceptable salt thereof, or any one of embodiments 1 to 54, wherein R⁶ is H, methyl, ethyl, propyl, isopropyl, butyl, isobutyl, 3,3,3-trifluoropropyl, cyclopropyl, cyclobutyl, —(CH₂)ₙOR¹⁰, or —CH₂R¹⁶.

### Embodiment 63

The compound of formula (I), or a pharmaceutically acceptable salt thereof, or any one of embodiments 1 to 54, wherein R⁶ is H.

### Embodiment 64

The compound of formula (I), or a pharmaceutically acceptable salt thereof, or any one of embodiments 1 to 54, wherein R⁶ is —C₁₋₆alkyl.

### Embodiment 65

The compound of formula (I), or a pharmaceutically acceptable salt thereof, or any one of embodiments 1 to 54, wherein R⁶ is methyl, ethyl, propyl, isopropyl, butyl or isobutyl.

### Embodiment 66

The compound of formula (I), or a pharmaceutically acceptable salt thereof, or any one of embodiments 1 to 54, wherein R⁶ is halo-substituted C₁₋₆alkyl.

### Embodiment 67

The compound of formula (I), or a pharmaceutically acceptable salt thereof, or any one of embodiments 1 to 54, wherein R⁶ is 3,3,3-trifluoropropyl.

### Embodiment 68

The compound of formula (I), or a pharmaceutically acceptable salt thereof, or any one of embodiments 1 to 54, wherein R⁶ is C₃₋₈cycloalkyl.

### Embodiment 69

The compound of formula (I), or a pharmaceutically acceptable salt thereof, or any one of embodiments 1 to 54, wherein R⁶ is cyclopropyl or cyclobutyl.

### Embodiment 70

The compound of formula (I), or a pharmaceutically acceptable salt thereof, or any one of embodiments 1 to 54, wherein R⁶ is —(CR⁸R⁹)ₙOR¹⁴.

### Embodiment 71

The compound of formula (I), or a pharmaceutically acceptable salt thereof, or any one of embodiments 1 to 54, wherein R⁶ is —(CR⁸R⁹)ₘR¹⁶.

### Embodiment 72

The compound of formula (I), or a pharmaceutically acceptable salt thereof, or any one of embodiments 1 to 54 or embodiments 61 to 71, wherein R⁷ is H, —C₁₋₆alkyl, —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, —(CR¹⁴R¹⁵)ₘC(═O)OR¹⁰, —CHR¹²R¹⁸, a monocyclic C₃₋₈cycloalkyl substituted with 1 to 2 R¹² groups, a bicyclic C₇₋₁₀cycloalkenyl substituted with 1 to 2 R¹² groups or a bicyclic C₇₋₁₀cycloalkyl substituted with 1 to 2 R¹² groups.

### Embodiment 73

The compound of formula (I), or a pharmaceutically acceptable salt thereof, or any one of embodiments 1 to 54 or embodiments 61 to 71, wherein R⁷ is H, —C₁₋₆alkyl, —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, —(CR¹⁴R¹⁵)ₘC(═O)OR¹⁰, —CHR¹²R¹⁸, a cyclohexyl substituted with 1 to 2 R¹² groups, a bicyclo[2.2.1]heptenyl substituted with 1 to 2 R¹² groups, or a bicyclo[2.2.1]heptanyl substituted with 1 to 2 R¹² groups.

### Embodiment 74

The compound of formula (I), or a pharmaceutically acceptable salt thereof, or any one of embodiments 1 to 54 or 61 to 71, wherein R⁷ is —(CR¹⁴R¹⁵)ₙR¹⁷.

### Embodiment 75

The compound of formula (I), or a pharmaceutically acceptable salt thereof, or any one of embodiments 1 to 54 or 61 to 71, wherein R⁷ is —(CR⁸R⁹)ₙC(═O)OR¹⁰.

### Embodiment 76

The compound of formula (I), or a pharmaceutically acceptable salt thereof, or any one of embodiments 1 to 54 or 61 to 71, wherein R⁷ is —(CR⁸R⁹)ₙ(CR¹⁴R¹⁵)ₘC(═O)OR¹⁰.

### Embodiment 77

The compound of formula (I), or a pharmaceutically acceptable salt thereof, or any one of embodiments 1 to 54 or 61 to 71, wherein R⁷ is —(CR¹⁴R¹⁵)ₘC(═O)OR¹⁰.

### Embodiment 78

The compound of formula (I), or a pharmaceutically acceptable salt thereof, or any one of embodiments 1 to 54 or 61 to 71, wherein R⁷—CHR¹²R¹⁸.

### Embodiment 79

The compound of formula (I), or a pharmaceutically acceptable salt thereof, or any one of embodiments 1 to 54 or 61 to 71, wherein R⁷ is a monocyclic C₃₋₈cycloalkyl substituted with 1 to 2 R¹² groups.

### Embodiment 80

The compound of formula (I), or a pharmaceutically acceptable salt thereof, or any one of embodiments 1 to 54 or 61 to 71, wherein R⁷ is a cyclohexyl substituted with 1 to 2 R¹² groups.

### Embodiment 81

The compound of formula (I), or a pharmaceutically acceptable salt thereof, or any one of embodiments 1 to 54 or 61 to 71, wherein R⁷ is a bicyclic C₇₋₁₀cycloalkenyl substituted with 1 to 2 R¹² groups.

### Embodiment 82

The compound of formula (I), or a pharmaceutically acceptable salt thereof, or any one of embodiments 1 to 54 or 61 to 71, wherein R⁷ is a bicyclo[2.2.1]heptenyl substituted with 1 to 2 R¹² groups.

### Embodiment 83

The compound of formula (I), or a pharmaceutically acceptable salt thereof, or any one of embodiments 1 to 54 or 61 to 71, wherein R⁷ is a bicyclic C₇₋₁₀cycloalkyl substituted with 1 to 2 R¹² groups.

### Embodiment 84

The compound of formula (I), or a pharmaceutically acceptable salt thereof, or any one of embodiments 1 to 54 or 61 to 71, wherein R⁷ is a bicyclo[2.2.1]heptanyl substituted with 1 to 2 R¹² groups.

### Embodiment 85

The compound of formula (I), or a pharmaceutically acceptable salt thereof, or any one of embodiments 1 to 54 or 72 to 74, wherein R¹⁷ is a C₃₋₈cycloalkyl substituted with 1 to 2 R¹² groups or a 5 to 6 membered monocyclic heterocycloalkyl having 1 to 2 ring members independently selected from N, O and S substituted with 1 to 2 R¹² groups.

### Embodiment 86

The compound of formula (I), or a pharmaceutically acceptable salt thereof, or any one of embodiments 1 to 54 or 72 to 74, wherein R¹⁷ is a C₃₋₈cycloalkyl substituted with 1 to 2 R¹² groups.

### Embodiment 87

The compound of formula (I), or a pharmaceutically acceptable salt thereof, or any one of embodiments 1 to 54 or 72 to 74, wherein R¹⁷ is a cyclobutyl substituted with 1 to 2 R¹² groups.

### Embodiment 88

The compound of formula (I), or a pharmaceutically acceptable salt thereof, or any one of embodiments 1 to 54 or 72 to 74, wherein R¹⁷ is a cyclopentyl substituted with 1 to 2 R¹² groups.

### Embodiment 89

The compound of formula (I), or a pharmaceutically acceptable salt thereof, or any one of embodiments 1 to 54 or 72 to 74, wherein R¹⁷ is a 5 to 6 membered monocyclic heterocycloalkyl having 1 to 2 ring members independently selected from N, O and S substituted with 1 to 2 R¹² groups.

### Embodiment 90

The compound of formula (I), or a pharmaceutically acceptable salt thereof, or any one of embodiments 1 to 54 or 72 to 74, wherein R¹⁷ is a tetrahydro-2H-pyranyl substituted with 1 to 2 R¹² groups.

### Embodiment 91

The compound of formula (I), or a pharmaceutically acceptable salt thereof, or any one of embodiments 1 to 73 or 78 to 90, wherein each R¹² is independently selected from C₁₋₆alkyl, —OH, —(CR⁸R⁹)ₙC(═O)OR¹³, —C(═O)NH(CR¹⁴R¹⁵)ₘC(═O)OR¹³, —C(═O)OR¹³, —(CR¹⁴R¹⁵)ₘC(═O)OR¹³, —O(CR⁸R⁹)ₙC(═O)OR¹³, benzoic acid and tetrazolyl.

### Embodiment 92

The compound of formula (I), or a pharmaceutically acceptable salt thereof, or any one of embodiments 1 to 73 or 78 to 90, wherein each R¹² is independently selected from C₁₋₆alkyl, —OH, —(CR⁸R⁹)ₙC(═O)OR¹³, —(CR¹⁴R¹⁵)ₘC(═O)OR¹³, and —C(═O)OR¹³.

### Embodiment 93

The compound of formula (I), or a pharmaceutically acceptable salt thereof, or any one of embodiments 1 to 73 or 78 to 90, wherein each R¹² is independently selected from methyl, —OH, —(CR⁸R⁹)ₙC(═O)OR¹³, —(CR¹⁴R¹⁵)ₘC(═O)OR¹³, and —C(═O)OR¹³.

### Embodiment 94

The compound of formula (I), or a pharmaceutically acceptable salt thereof, or any one of embodiments 1 to 73 or 78 to 90, wherein each R¹² is independently selected from C₁₋₆alkyl, —(CR⁸R⁹)ₙC(═O)OR¹³ and —C(═O)OR¹³.

### Embodiment 95

The compound of formula (I), or a pharmaceutically acceptable salt thereof, or any one of embodiments 1 to 73 or 78 to 90, wherein each R¹² is independently selected from methyl, —(CR⁸R⁹)ₙC(═O)OR¹³ and —C(═O)OR¹³.

### Embodiment 96

The compound of formula (I), or a pharmaceutically acceptable salt thereof, or any one of embodiments 1 to 73 or 78 to 90, wherein each R¹² is independently selected from —C(═O)NH(CR¹⁴R¹⁵)ₘC(═O)OR¹³, —O(CR⁸R⁹)ₙC(═O)OR¹³, benzoic acid and tetrazolyl.

### Embodiment 97

The compound of formula (I), or a pharmaceutically acceptable salt thereof, or any one of embodiments 1 to 53 or 55, wherein R¹¹ is —(CR⁸R⁹)ₙC(═O)OR¹³.

### Embodiment 98

The compound of formula (I), or a pharmaceutically acceptable salt thereof, or any one of embodiments 1 to 53, 56, 61, 70 to 73, 75, 76 or 91 to 97, wherein each R⁸ is independently selected from H, D, deuterium substituted C₁₋₆alkyl and C₁₋₆alkyl.

### Embodiment 99

The compound of formula (I), or a pharmaceutically acceptable salt thereof, or any one of embodiments 1 to 53, 56, 61, 70 to 73, 75, 76 or 91 to 97, wherein each R⁸ is H.

### Embodiment 100

The compound of formula (I), or a pharmaceutically acceptable salt thereof, or any one of embodiments 1 to 53, 56, 61, 70 to 73, 75, 76 or 91 to 97, wherein each R⁸ is D.

### Embodiment 101

The compound of formula (I), or a pharmaceutically acceptable salt thereof, or any one of embodiments 1 to 53, 56, 61, 70 to 73, 75, 76 or 91 to 97, wherein each R⁸ is deuterium substituted C₁₋₆alkyl.

### Embodiment 102

The compound of formula (I), or a pharmaceutically acceptable salt thereof, or any one of embodiments 1 to 53, 56, 61, 70 to 73, 75, 76 or 91 to 97, wherein each R⁸ is C₁₋₆alkyl.

### Embodiment 103

The compound of formula (I), or a pharmaceutically acceptable salt thereof, or any one of embodiments 1 to 53, 56, 61, 70 to 73, 75, 76 or 91 to 102, wherein each R⁹ is independently selected from H, D, deuterium substituted C₁₋₆alkyl and C₁₋₆alkyl.

### Embodiment 104

The compound of formula (I), or a pharmaceutically acceptable salt thereof, or any one of embodiments 1 to 53, 56, 61, 70 to 73, 75, 76 or 91 to 102, wherein each R⁹ is H.

### Embodiment 105

The compound of formula (I), or a pharmaceutically acceptable salt thereof, or any one of embodiments 1 to 53, 56, 61, 70 to 73, 75, 76 or 91 to 102, wherein each R⁹ is D.

### Embodiment 106

The compound of formula (I), or a pharmaceutically acceptable salt thereof, or any one of embodiments 1 to 53, 56, 61, 70 to 73, 75, 76 or 91 to 102, wherein each R⁹ is deuterium substituted C₁₋₆alkyl.

### Embodiment 107

The compound of formula (I), or a pharmaceutically acceptable salt thereof, or any one of embodiments 1 to 53, 56, 61, 70 to 73, 75, 76 or 91 to 102, wherein each R⁹ is C₁₋₆alkyl.

### Embodiment 108

The compound of formula (I), or a pharmaceutically acceptable salt thereof, or any one of embodiments 1 to 53, 56, 61, 70, 72, 73, 75 to 77, wherein each R¹⁰ is independently selected from H, and C₁₋₆alkyl.

### Embodiment 109

The compound of formula (I), or a pharmaceutically acceptable salt thereof, or any one of embodiments 1 to 53, 56, 61, 70, 72, 73, 75 to 77, wherein each R¹⁰ is H.

### Embodiment 110

The compound of formula (I), or a pharmaceutically acceptable salt thereof, or any one of embodiments 1 to 53, 56, 61, 70, 72, 73, 75 to 77, wherein each R¹⁰ is C₁₋₆alkyl.

### Embodiment 111

The compound of formula (I), or a pharmaceutically acceptable salt thereof, or any one of embodiments 1 to 53, 56, 61, 70, 72, 73, 75 to 77, wherein each R¹⁰ is methyl.

### Embodiment 112

The compound of formula (I), or a pharmaceutically acceptable salt thereof, or any one of embodiments 1 to 53, 55, 57 to 60, 72, 73 or 78 to 97, wherein each R¹³ is independently selected from H and C₁₋₆alkyl.

### Embodiment 113

The compound of formula (I), or a pharmaceutically acceptable salt thereof, or any one of embodiments 1 to 53, 55, 57 to 60, 72, 73 or 78 to 97, wherein each R¹³ is H.

### Embodiment 114

The compound of formula (I), or a pharmaceutically acceptable salt thereof, or any one of embodiments 1 to 53, 55, 57 to 60, 72, 73 or 78 to 97, wherein each R¹³ is C₁₋₆alkyl.

### Embodiment 115

The compound of formula (I), or a pharmaceutically acceptable salt thereof, or any one of embodiments 1 to 53, 55, 57 to 60, 72, 73 or 78 to 97, wherein each R¹³ is methyl or ethyl.

### Embodiment 116

The compound of formula (I), or a pharmaceutically acceptable salt thereof, or any one of embodiments 1 to 53, 57 to 60, 72 to 93 or 96, wherein each R¹⁴ is independently selected from H, D, deuterium substituted C₁₋₆alkyl and C₁₋₆alkyl.

### Embodiment 117

The compound of formula (I), or a pharmaceutically acceptable salt thereof, or any one of embodiments 1 to 53, 57 to 60, 72 to 93 or 96, wherein each R¹⁴ is H.

### Embodiment 118

The compound of formula (I), or a pharmaceutically acceptable salt thereof, or any one of embodiments 1 to 53, 57 to 60, 72 to 93 or 96, wherein each R¹⁴ is C₁₋₆alkyl.

### Embodiment 119

The compound of formula (I), or a pharmaceutically acceptable salt thereof, or any one of embodiments 1 to 53, 57 to 60, 72 to 93 or 96, wherein each R¹⁴ is methyl or ethyl.

### Embodiment 120

The compound of formula (I), or a pharmaceutically acceptable salt thereof, or any one of embodiments 1 to 53, 57 to 60, 72 to 93, 96 or 116-119, wherein each R¹⁵ is independently selected from H, D, deuterium substituted C₁₋₆alkyl and C₁₋₆alkyl.

### Embodiment 121

The compound of formula (I), or a pharmaceutically acceptable salt thereof, or any one of embodiments 1 to 53, 57 to 60, 72 to 93, 96 or 116-119, wherein each R¹⁵ is H.

### Embodiment 122

The compound of formula (I), or a pharmaceutically acceptable salt thereof, or any one of embodiments 1 to 53, 57 to 60, 72 to 93, 96 or 116-119, wherein each R¹⁵ is C₁₋₆alkyl.

### Embodiment 123

The compound of formula (I), or a pharmaceutically acceptable salt thereof, or any one of embodiments 1 to 53, 57 to 60, 72 to 93, 96 or 116-119, wherein each R¹⁵ is methyl or ethyl.

### Embodiment 124

The compound of formula (I), or a pharmaceutically acceptable salt thereof, or any one of embodiments 1 to 53, 57 to 60, 72 to 93, or 96, wherein R¹⁴ and R¹⁵ together with the carbon in CR¹⁴R¹⁵ form a C₃₋₈cycloalkyl.

### Embodiment 125

The compound of formula (I), or a pharmaceutically acceptable salt thereof, or any one of embodiments 1 to 53, 57 to 60, 72 to 93, or 96, wherein R¹⁴ and R¹⁵ together with the carbon in CR¹⁴R¹⁵ form a cyclopropyl, a cyclobutyl or a cyclopentyl.

### Embodiment 126

The compound of formula (I), or a pharmaceutically acceptable salt thereof, or any one of embodiments 1 to 54, 61, 62 or 71 wherein R¹⁶ is a C₃₋₈cycloalkyl.

### Embodiment 127

The compound of formula (I), or a pharmaceutically acceptable salt thereof, or any one of embodiments 1 to 54, 61, 62 or 71, wherein R¹⁶ is a cyclopropyl.

### Embodiment 128

The compound of formula (I), or a pharmaceutically acceptable salt thereof, or any one of embodiments 1 to 54, 72, 73 or 77, wherein R¹⁸ is adamantanyl.

### Embodiment 129

The compound of formula (I), or a pharmaceutically acceptable salt thereof, or any one of embodiments 16 to 128, wherein R¹⁹ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S.

### Embodiment 130

The compound of formula (I), or a pharmaceutically acceptable salt thereof, or any one of embodiments 16 to 128, wherein R¹⁹ is H, C₁₋₆alkyl, phenyl, phenoxy, halo, C₁₋₆alkoxy, C₃₋₈cycloalkyl, C₂₋₆alkenyl, or a 5 to 6 membered heteroaryl having 1 to 3 heteroatoms independently selected from N, O and S.

### Embodiment 131

The compound of formula (I), or a pharmaceutically acceptable salt thereof, or any one of embodiments 16 to 128, wherein R¹⁹ is D, methyl, ethyl, propyl, isopropyl, butyl, tert-butyl, CD₃, phenyl, phenoxy, Cl, F, methoxy, cyclopropyl, cyclobutyl, ethenyl, pyrimidinyl or pyridyl.

### Embodiment 132

The compound of formula (I), or a pharmaceutically acceptable salt thereof, or any one of embodiments 16 to 128, wherein R¹⁹ is methyl, ethyl, isopropyl, tert-butyl, phenyl, phenoxy, Cl, methoxy, cyclopropyl, cyclobutyl, ethenyl, pyrimidinyl or pyridyl.

### Embodiment 133

The compound of formula (I), or a pharmaceutically acceptable salt thereof, or any one of embodiments 16 to 128, wherein R¹⁹ is methyl, ethyl, isopropyl, tert-butyl or ethenyl.

### Embodiment 134

The compound of formula (I), or a pharmaceutically acceptable salt thereof, or any one of embodiments 16 to 128, wherein R¹⁹ is phenyl, phenoxy, cyclopropyl, cyclobutyl, pyrimidinyl or pyridyl.

### Embodiment 135

The compound of formula (I), or a pharmaceutically acceptable salt thereof, or any one of embodiments 16 to 128, wherein R¹⁹ is cyclopropyl or cyclobutyl.

### Embodiment 136

The compound of formula (I), or a pharmaceutically acceptable salt thereof, or any one of embodiments 16 to 128, wherein R¹⁹ is phenyl, phenoxy, pyrimidinyl and pyridyl.

### Embodiment 137

The compound of formula (I), or a pharmaceutically acceptable salt thereof, or any one of embodiments 16 to 128, wherein R¹⁹ is Cl.

### Embodiment 138

The compound of formula (I), or a pharmaceutically acceptable salt thereof, or any one of embodiments 16 to 138, wherein R²⁰ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S.

### Embodiment 139

The compound of formula (I), or a pharmaceutically acceptable salt thereof, or any one of embodiments 16 to 138, wherein R²⁰ is H, C₁₋₆alkyl, phenyl, phenoxy, halo, C₁₋₆alkoxy, C₃₋₈cycloalkyl, C₂₋₆alkenyl, or a 5 to 6 membered heteroaryl having 1 to 3 heteroatoms independently selected from N, O and S.

### Embodiment 140

The compound of formula (I), or a pharmaceutically acceptable salt thereof, or any one of embodiments 16 to 138, wherein R²⁰ is H, halo or C₁₋₆alkoxy.

### Embodiment 141

The compound of formula (I), or a pharmaceutically acceptable salt thereof, or any one of embodiments 16 to 138, wherein R²⁰ is H, D, methyl, ethyl, propyl, isopropyl, butyl, tert-butyl, CD₃, phenyl, phenoxy, Cl, F, methoxy, cyclopropyl, cyclobutyl, ethenyl, pyrimidinyl and pyridyl.

### Embodiment 142

The compound of formula (I), or a pharmaceutically acceptable salt thereof, or any one of embodiments 16 to 138, wherein R²⁰ is H, Cl, F or methoxy.

### Embodiment 143

The compound of formula (I), or a pharmaceutically acceptable salt thereof, or any one of embodiments 16 to 138, wherein R²⁰ is H.

### Embodiment 144

The compound of formula (I), or a pharmaceutically acceptable salt thereof, or any one of embodiments 16 to 138, wherein R²⁰ is F.

### Embodiment 145

The compound of formula (I), or a pharmaceutically acceptable salt thereof, or any one of embodiments 16 to 138, wherein R²⁰ is methoxy.

### Embodiment 146

The compound of formula (I), or a pharmaceutically acceptable salt thereof, or any one of embodiments 16 to 145, wherein R²¹ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S.

### Embodiment 147

The compound of formula (I), or a pharmaceutically acceptable salt thereof, or any one of embodiments 16 to 145, wherein R²¹ is H.

### Embodiment 148

The compound of formula (I), or a pharmaceutically acceptable salt thereof, or any one of embodiments 16 to 147, wherein R²² is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S.

### Embodiment 149

The compound of formula (I), or a pharmaceutically acceptable salt thereof, or any one of embodiments 16 to 147, wherein R²² is H, C₁₋₆alkyl, phenyl, phenoxy, halo, C₁₋₆alkoxy, C₃₋₈cycloalkyl, C₂₋₆alkenyl, or a 5 to 6 membered heteroaryl having 1 to 3 heteroatoms independently selected from N, O and S.

### Embodiment 150

The compound of formula (I), or a pharmaceutically acceptable salt thereof, or any one of embodiments 16 to 147, wherein R²² is H or halo.

### Embodiment 151

The compound of formula (I), or a pharmaceutically acceptable salt thereof, or any one of embodiments 16 to 147, wherein R²² is H, D, methyl, ethyl, propyl, isopropyl, butyl, tert-butyl, CD₃, phenyl, phenoxy, Cl, F, methoxy, cyclopropyl, cyclobutyl, ethenyl, pyrimidinyl and pyridyl.

### Embodiment 152

The compound of formula (I), or a pharmaceutically acceptable salt thereof, or any one of embodiments 14 to 144, wherein R²² is H, Cl, or F.

### Embodiment 153

The compound of formula (I), or a pharmaceutically acceptable salt thereof, or any one of embodiments 16 to 147, wherein R²² is H.

### Embodiment 154

The compound of formula (I), or a pharmaceutically acceptable salt thereof, or any one of embodiments 16 to 147, wherein R²² is F.

### Embodiment 155

The compound of formula (I), or a pharmaceutically acceptable salt thereof, or any one of embodiments 16 to 154, wherein R²³ is H, C₁₋₆alkyl, phenyl, phenoxy, halo, C₁₋₆alkoxy, C₃₋₈cycloalkyl, C₂₋₆alkenyl, halo-substituted C₁₋₆alkyl, D, deuterium substituted C₁₋₆alkyl, halo-substituted C₁₋₆alkoxy or a 5 to 6 membered heteroaryl having 1 to 3 heteroatoms independently selected from N, O and S.

### Embodiment 156

The compound of formula (I), or a pharmaceutically acceptable salt thereof, or any one of embodiments 16 to 154, wherein R²³ is H, C₁₋₆alkyl, phenyl, phenoxy, halo, C₁₋₆alkoxy, C₃₋₈cycloalkyl, C₂₋₆alkenyl, or a 5 to 6 membered heteroaryl having 1 to 3 heteroatoms independently selected from N, O and S.

### Embodiment 157

The compound of formula (I), or a pharmaceutically acceptable salt thereof, or any one of embodiments 16 to 154, wherein R²³ is H or C₁₋₆alkyl.

### Embodiment 158

The compound of formula (I), or a pharmaceutically acceptable salt thereof, or any one of embodiments 16 to 154, wherein R²³ is H, D, methyl, ethyl, propyl, isopropyl, butyl, tert-butyl, CD₃, phenyl, phenoxy, Cl, F, methoxy, cyclopropyl, cyclobutyl, ethenyl, pyrimidinyl and pyridyl.

### Embodiment 159

The compound of formula (I), or a pharmaceutically acceptable salt thereof, or any one of embodiments 16 to 154, wherein R²³ is H or methyl.

### Embodiment 160

The compound of formula (I), or a pharmaceutically acceptable salt thereof, or any one of embodiments 16 to 154, wherein R²³ is H.

### Embodiment 161

The compound of formula (I), or a pharmaceutically acceptable salt thereof, or any one of embodiments 16 to 154, wherein R²³ is methyl.

### Embodiment 162

The compound of formula (I), or a pharmaceutically acceptable salt thereof, or any one of embodiments 1 to 161, wherein each m is independently selected from 1, 2, 3, 4, 5, 6, 7, 8, 9 and 10.

### Embodiment 163

The compound of formula (I), or a pharmaceutically acceptable salt thereof, or any one of embodiments 1 to 161, wherein each m is independently selected from 1, 2, 3, 4, and 5.

### Embodiment 164

The compound of formula (I), or a pharmaceutically acceptable salt thereof, or any one of embodiments 1 to 161, wherein each m is independently selected from 1, 2 and 3.

### Embodiment 165

The compound of formula (I), or a pharmaceutically acceptable salt thereof, or any one of embodiments 1 to 165, wherein m is 1.

### Embodiment 166

The compound of formula (I), or a pharmaceutically acceptable salt thereof, or any one of embodiments 1 to 165, wherein each n is independently selected from 1, 2, 3, 4, 5, 6, 7, 8, 9 and 10.

### Embodiment 167

The compound of formula (I), or a pharmaceutically acceptable salt thereof, or any one of embodiments 1 to 165, wherein each n is independently selected from 1, 2, 3 and 4.

### Embodiment 168

The compound of formula (I), or a pharmaceutically acceptable salt thereof, or any one of embodiments 1 to 165, wherein each n is independently selected from 1, 2 and 3.

### Embodiment 169

The compound of formula (I), or a pharmaceutically acceptable salt thereof, or any one of embodiments 1 to 165, wherein each n is independently selected from 1 and 2.

### Embodiment 170

The compound of formula (I), or a pharmaceutically acceptable salt thereof, or any one of embodiments 1 to 165, wherein n is 1.

### Embodiment 171

The compound of formula (I), or a pharmaceutically acceptable salt thereof, or any one of embodiments 1 to 52 wherein R⁵ is an unsubstituted 6 membered heterocycloalkyl having 1 to 2 ring members independently selected from N, O and S.

### Embodiment 172

The compound of of formula (I) or any one of Embodiments 1 to 171 selected from
3-((6-(N-(5-chloro-6-(2,6-dimethylphenyl)pyridin-2-yl)sulfamoyl)pyridin-2-yl)amino)propanoic acid;
3-((4-(N-(5-chloro-6-(2,6-dimethylphenyl)pyridin-2-yl)sulfamoyl)pyridin-2-yl)amino)propanoic acid;
4-((6-(N-(5-chloro-6-(2,6-dimethylphenyl)pyridin-2-yl)sulfamoyl)pyridin-2-yl)amino)butanoic acid;
5-((6-(N-(5-chloro-6-(2,6-dimethylphenyl)pyridin-2-yl)sulfamoyl)pyridin-2-yl)amino)pentanoic acid;
2-(1-(6-(N-(5-chloro-6-(2,6-dimethylphenyl)pyridin-2-yl)sulfamoyl)pyridin-2-yl)piperidin-4-yl)acetic acid;
1-(6-(N-(6-([1,1′-biphenyl]-2-yl)-5-chloropyridin-2-yl)sulfamoyl)pyridin-2-yl)-4-methylpiperidine-4-carboxylic acid;
1-(6-(N-(6-([1,1′-biphenyl]-2-yl)-5-(trifluoromethyl)pyridin-2-yl)sulfamoyl)pyridin-2-yl)-4-methylpiperidine-4-carboxylic acid;
4-(4-(6-(N-(6-(2-chloro-5-methoxyphenyl)-5-(trifluoromethyl)pyridin-2-yl)sulfamoyl)pyridin-2-yl)piperazin-1-yl)benzoic acid:
2-(1-(6-(N-(6-(5-fluoro-2-methylphenyl)-5-(trifluoromethyl)pyridin-2-yl)sulfamoyl)pyridin-2-yl)-4-methylpiperidin-4-yl)acetic acid;
2-(4-methyl-1-(6-(N-(6-(o-tolyl)-5-(trifluoromethyl)pyridin-2-yl)sulfamoyl)pyridin-2-yl)piperidin-4-yl)acetic acid;
2,2-dimethyl-3-((6-(N-(6-(o-tolyl)-5-(trifluoromethyl)pyridin-2-yl)sulfamoyl)pyridin-2-yl)amino)propanoic acid;
2-(1-(6-(N-(6-(2-ethylphenyl)-5-(trifluoromethyl)pyridin-2-yl)sulfamoyl)pyridin-2-yl)-4-methylpiperidin-4-yl)acetic acid;
2-(1-(6-(N-(6-([1,1′-biphenyl]-2-yl)-5-(trifluoromethyl)pyridin-2-yl)sulfamoyl)pyridin-2-yl)-4-methylpiperidin-4-yl)acetic acid;
2-methyl-2-(1-(6-(N-(6-(o-tolyl)-5-(trifluoromethyl)pyridin-2-yl)sulfamoyl)pyridin-2-yl)piperidin-4-yl)propanoic acid;
2-(4-hydroxy-1-(6-(N-(6-(o-tolyl)-5-(trifluoromethyl)pyridin-2-yl)sulfamoyl)pyridin-2-yl)piperidin-4-yl)-2-methylpropanoic acid;
1-(((6-(N-(6-(o-tolyl)-5-(trifluoromethyl)pyridin-2-yl)sulfamoyl)pyridin-2-yl)amino)methyl)cyclopropane-1-carboxylic acid;
1-(((6-(N-(6-(o-tolyl)-5-(trifluoromethyl)pyridin-2-yl)sulfamoyl)pyridin-2-yl)amino)methyl)cyclobutane-1-carboxylic acid;
2-(1-(6-(N-(6-(3-fluoro-2-methylphenyl)-5-(trifluoromethyl)pyridin-2-yl)sulfamoyl)pyridin-2-yl)-4-methylpiperidin-4-yl)acetic acid;
2-(1-(6-(N-(6-(o-tolyl)-5-(trifluoromethyl)pyridin-2-yl)sulfamoyl)pyridin-2-yl)piperidin-4-yl)acetic acid;
2-(1-(6-(N-(6-(2-ethylphenyl)-5-(trifluoromethyl)pyridin-2-yl)sulfamoyl)pyridin-2-yl)piperidin-4-yl)acetic acid;
2-(1-(6-(N-(6-([1,1′-biphenyl]-2-yl)-5-(trifluoromethyl)pyridin-2-yl)sulfamoyl)pyridin-2-yl)piperidin-4-yl)acetic acid;
2-(1-(6-(N-(6-(2-cyclobutylphenyl)-5-(trifluoromethyl)pyridin-2-yl)sulfamoyl)pyridin-2-yl)-4-methylpiperidin-4-yl)acetic acid;
2-(1-(6-(N-(6-(2-isopropylphenyl)-5-(trifluoromethyl)pyridin-2-yl)sulfamoyl)pyridin-2-yl)-4-methylpiperidin-4-yl)acetic acid;
2-(1-(6-(N-(6-(2-(tert-butyl)phenyl)-5-(trifluoromethyl)pyridin-2-yl)sulfamoyl)pyridin-2-yl)-4-methylpiperidin-4-yl)acetic acid;
2-(1-(6-(N-(6-(2-cyclobutylphenyl)-5-(trifluoromethyl)pyridin-2-yl)sulfamoyl)pyridin-2-yl)piperidin-4-yl)acetic acid;
4-methyl-1-(6-(N-(6-(2-(pyrimidin-2-yl)phenyl)-5-(trifluoromethyl)pyridin-2-yl)sulfamoyl)pyridin-2-yl)piperidine-4-carboxylic acid;
4-methyl-1-(6-(N-(6-(2-(pyridin-4-yl)phenyl)-5-(trifluoromethyl)pyridin-2-yl)sulfamoyl)pyridin-2-yl)piperidine-4-carboxylic acid;
4-methyl-1-(6-(N-(6-(2-phenoxyphenyl)-5-(trifluoromethyl)pyridin-2-yl)sulfamoyl)pyridin-2-yl)piperidine-4-carboxylic acid;
1-(6-(N-(6-([1,1′-biphenyl]-2-yl)-5-chloropyridin-2-yl)sulfamoyl)pyridin-2-yl)-3-methylpiperidine-3-carboxylic acid;
1-(6-(N-(4′-isopropyl-3-(trifluoromethyl)-[2,3′-bipyridin]-6-yl)sulfamoyl)pyridin-2-yl)-3-methylpiperidine-3-carboxylic acid;
2-((1-(6-(N-(6-(2-isopropylphenyl)-5-(trifluoromethyl)pyridin-2-yl)sulfamoyl)pyridin-2-yl)piperidin-4-yl)ₙxy)acetic acid;
2-(1-(6-(N-(6-(o-tolyl)-5-(trifluoromethyl)pyridin-2-yl)sulfamoyl)pyridin-2-yl)pyrrolidin-3-yl)acetic acid;
2-(1-(6-(N-(6-(2-cyclopropylphenyl)-5-(trifluoromethyl)pyridin-2-yl)sulfamoyl)pyridin-2-yl)pyrrolidin-3-yl)acetic acid;
3-(methyl(6-(N-(6-(o-tolyl)-5-(trifluoromethyl)pyridin-2-yl)sulfamoyl)pyridin-2-yl)amino)propanoic acid;
3-((6-(N-(6-(o-tolyl)-5-(trifluoromethyl)pyridin-2-yl)sulfamoyl)pyridin-2-yl)amino)propanoic acid;
4-((6-(N-(6-(o-tolyl)-5-(trifluoromethyl)pyridin-2-yl)sulfamoyl)pyridin-2-yl)amino)butanoic acid;
4-(methyl(6-(N-(6-(o-tolyl)-5-(trifluoromethyl)pyridin-2-yl)sulfamoyl)pyridin-2-yl)amino)butanoic acid;
3-((6-(N-(6-(2-cyclopropylphenyl)-5-(trifluoromethyl)pyridin-2-yl)sulfamoyl)pyridin-2-yl)(methyl)amino)propanoic acid;
3-((6-(N-(6-(2-cyclopropylphenyl)-5-(trifluoromethyl)pyridin-2-yl)sulfamoyl)pyridin-2-yl)amino)propanoic acid;
4-((6-(N-(6-(2-cyclopropylphenyl)-5-(trifluoromethyl)pyridin-2-yl)sulfamoyl)pyridin-2-yl)amino)butanoic acid;
4-((6-(N-(6-(2-cyclopropylphenyl)-5-(trifluoromethyl)pyridin-2-yl)sulfamoyl)pyridin-2-yl)(methyl)amino)butanoic acid;
5-((6-(N-(6-(2-cyclopropylphenyl)-5-(trifluoromethyl)pyridin-2-yl)sulfamoyl)pyridin-2-yl)amino)pentanoic acid;
5-((6-(N-(6-(o-tolyl)-5-(trifluoromethyl)pyridin-2-yl)sulfamoyl)pyridin-2-yl)amino)pentanoic acid;
2-(1-(6-(N-(6-(o-tolyl)-5-(trifluoromethyl)pyridin-2-yl)sulfamoyl)pyridin-2-yl)piperidin-3-yl)acetic acid;
3-((6-(N-(6-(2-isopropylphenyl)-5-(trifluoromethyl)pyridin-2-yl)sulfamoyl)pyridin-2-yl)(methyl)amino)propanoic acid;
3-((6-(N-(6-(5-fluoro-2-methylphenyl)-5-(trifluoromethyl)pyridin-2-yl)sulfamoyl)pyridin-2-yl)(methyl)amino)-2,2-dimethylpropanoic acid;
2,2-dimethyl-3-(methyl(6-(N-(6-(o-tolyl)-5-(trifluoromethyl)pyridin-2-yl)sulfamoyl)pyridin-2-yl)amino)propanoic acid;
3-((6-(N-(6-(3-fluoro-2-methylphenyl)-5-(trifluoromethyl)pyridin-2-yl)sulfamoyl)pyridin-2-yl)(methyl)amino)-2,2-dimethylpropanoic acid;
3-((6-(N-(6-(5-fluoro-2-methylphenyl)-5-(trifluoromethyl)pyridin-2-yl)sulfamoyl)pyridin-2-yl)(methyl)amino)propanoic acid;
3-((6-(N-(6-(3-fluoro-2-methylphenyl)-5-(trifluoromethyl)pyridin-2-yl)sulfamoyl)pyridin-2-yl)(methyl)amino)propanoic acid;
2-(1-(6-(N-(6-(3-fluoro-2-methylphenyl)-5-(trifluoromethyl)pyridin-2-yl)sulfamoyl)pyridin-2-yl)-3-methylpiperidin-3-yl)acetic acid;
2-(3-methyl-1-(6-(N-(6-(o-tolyl)-5-(trifluoromethyl)pyridin-2-yl)sulfamoyl)pyridin-2-yl)piperidin-3-yl)acetic acid;
2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid;
4-(ethyl(6-(N-(6-(2-isopropylphenyl)-5-(trifluoromethyl)pyridin-2-yl)sulfamoyl)pyridin-2-yl)amino)butanoic acid;
3-(ethyl(6-(N-(6-(o-tolyl)-5-(trifluoromethyl)pyridin-2-yl)sulfamoyl)pyridin-2-yl)amino)propanoic acid;
3-(ethyl(6-(N-(6-(2-ethylphenyl)-5-(trifluoromethyl)pyridin-2-yl)sulfamoyl)pyridin-2-yl)amino)propanoic acid;
4-((6-(N-(6-(2-isopropylphenyl)-5-(trifluoromethyl)pyridin-2-yl)sulfamoyl)pyridin-2-yl)(methyl)amino)butanoic acid;
3-(1-(6-(N-(6-(o-tolyl)-5-(trifluoromethyl)pyridin-2-yl)sulfamoyl)pyridin-2-yl)piperidin-3-yl)propanoic acid;
3-(1-(6-(N-(5-(trifluoromethyl)-6-(2-vinylphenyl)pyridin-2-yl)sulfamoyl)pyridin-2-yl)piperidin-3-yl)propanoic acid;
4-(1-(6-(N-(6-(o-tolyl)-5-(trifluoromethyl)pyridin-2-yl)sulfamoyl)pyridin-2-yl)piperidin-3-yl)butanoic acid;
4-(1-(6-(N-(5-(trifluoromethyl)-6-(2-vinylphenyl)pyridin-2-yl)sulfamoyl)pyridin-2-yl)piperidin-3-yl)butanoic acid;
ethyl (R)-2-(1-(6-(N-(5-(trifluoromethyl)-6-(2-vinylphenyl)pyridin-2-yl)sulfamoyl)pyridin-2-yl)piperidin-3-yl)acetate;
methyl (S)-2-(1-(6-(N-(5-(trifluoromethyl)-6-(2-vinylphenyl)pyridin-2-yl)sulfamoyl)pyridin-2-yl)piperidin-3-yl)acetate;
2-(3-methyl-1-(6-(N-(6-(o-tolyl)-5-(trifluoromethyl)pyridin-2-yl)sulfamoyl)pyridin-2-yl)pyrrolidin-3-yl)acetic acid;
2-(1-(6-(N-(6-(3-fluoro-2-methylphenyl)-5-(trifluoromethyl)pyridin-2-yl)sulfamoyl)pyridin-2-yl)-3-methylpyrrolidin-3-yl)acetic acid;
2-(1-(6-(N-(6-(5-fluoro-2-methylphenyl)-5-(trifluoromethyl)pyridin-2-yl)sulfamoyl)pyridin-2-yl)-3-methylpyrrolidin-3-yl)acetic acid;
3-(ethyl(6-(N-(6-(3-fluoro-2-methylphenyl)-5-(trifluoromethyl)pyridin-2-yl)sulfamoyl)pyridin-2-yl)amino)propanoic acid;
3-(1-(6-(N-(6-(2-ethylphenyl)-5-(trifluoromethyl)pyridin-2-yl)sulfamoyl)pyridin-2-yl)piperidin-3-yl)propanoic acid;
4-(1-(6-(N-(6-(2-ethylphenyl)-5-(trifluoromethyl)pyridin-2-yl)sulfamoyl)pyridin-2-yl)piperidin-3-yl)butanoic acid;
(R)-2-(1-(6-(N-(5-(trifluoromethyl)-6-(2-vinylphenyl)pyridin-2-yl)sulfamoyl)pyridin-2-yl)piperidin-3-yl)acetic acid;
(S)-2-(1-(6-(N-(5-(trifluoromethyl)-6-(2-vinylphenyl)pyridin-2-yl)sulfamoyl)pyridin-2-yl)piperidin-3-yl)acetic acid;
(R)-2-(1-(6-(N-(6-(o-tolyl)-5-(trifluoromethyl)pyridin-2-yl)sulfamoyl)pyridin-2-yl)piperidin-3-yl)acetic acid;
(S)-2-(1-(6-(N-(6-(o-tolyl)-5-(trifluoromethyl)pyridin-2-yl)sulfamoyl)pyridin-2-yl)piperidin-3-yl)acetic acid;
3-(ethyl(6-(N-(6-(5-fluoro-2-methylphenyl)-5-(trifluoromethyl)pyridin-2-yl)sulfamoyl)pyridin-2-yl)amino)propanoic acid;
3-(1-(6-(N-(6-(o-tolyl)-5-(trifluoromethyl)pyridin-2-yl)sulfamoyl)pyridin-2-yl)piperidin-4-yl)propanoic acid;
(R)-3-(1-(6-(N-(6-(o-tolyl)-5-(trifluoromethyl)pyridin-2-yl)sulfamoyl)pyridin-2-yl)piperidin-3-yl)propanoic acid;
(S)-3-(1-(6-(N-(6-(o-tolyl)-5-(trifluoromethyl)pyridin-2-yl)sulfamoyl)pyridin-2-yl)piperidin-3-yl)propanoic acid;
3-(isopropyl(6-(N-(6-(o-tolyl)-5-(trifluoromethyl)pyridin-2-yl)sulfamoyl)pyridin-2-yl)amino)propanoic acid;
(R)-2-(1-(6-(N-(6-(2-ethylphenyl)-5-(trifluoromethyl)pyridin-2-yl)sulfamoyl)pyridin-2-yl)piperidin-3-yl)acetic acid;
(S)-2-(1-(6-(N-(6-(2-ethylphenyl)-5-(trifluoromethyl)pyridin-2-yl)sulfamoyl)pyridin-2-yl)piperidin-3-yl)acetic acid;
(S)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid;
(R)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid;
5-(methyl(6-(N-(6-(o-tolyl)-5-(trifluoromethyl)pyridin-2-yl)sulfamoyl)pyridin-2-yl)amino)pentanoic acid;
5-((6-(N-(6-(3-fluoro-2-methylphenyl)-5-(trifluoromethyl)pyridin-2-yl)sulfamoyl)pyridin-2-yl)(methyl)amino)pentanoic acid;
5-((6-(N-(6-(5-fluoro-2-methylphenyl)-5-(trifluoromethyl)pyridin-2-yl)sulfamoyl)pyridin-2-yl)(methyl)amino)pentanoic acid;
4-((6-(N-(6-(5-fluoro-2-methylphenyl)-5-(trifluoromethyl)pyridin-2-yl)sulfamoyl)pyridin-2-yl)(methyl)amino)butanoic acid;
4-((6-(N-(6-(2-ethyl-5-fluorophenyl)-5-(trifluoromethyl)pyridin-2-yl)sulfamoyl)pyridin-2-yl)(methyl)amino)butanoic acid;
4-(isopropyl(6-(N-(6-(o-tolyl)-5-(trifluoromethyl)pyridin-2-yl)sulfamoyl)pyridin-2-yl)amino)butanoic acid;
3-((cyclopropylmethyl)(6-(N-(6-(5-fluoro-2-methylphenyl)-5-(trifluoromethyl)pyridin-2-yl)sulfamoyl)pyridin-2-yl)amino)propanoic acid;
3-(ethyl(6-(N-(6-(2-isopropylphenyl)-5-(trifluoromethyl)pyridin-2-yl)sulfamoyl)pyridin-2-yl)amino)propanoic acid;
4-((6-(N-(6-(5-fluoro-2-methylphenyl)-5-(trifluoromethyl)pyridin-2-yl)sulfamoyl)pyridin-2-yl)(isopropyl)amino)butanoic acid;
4-((6-(N-(6-(2-ethylphenyl)-5-(trifluoromethyl)pyridin-2-yl)sulfamoyl)pyridin-2-yl)(isopropyl)amino)butanoic acid;
3-((6-(N-(6-(2-cyclopropylphenyl)-5-(trifluoromethyl)pyridin-2-yl)sulfamoyl)pyridin-2-yl)(ethyl)amino)propanoic acid;
4-(ethyl(6-(N-(6-(2-ethylphenyl)-5-(trifluoromethyl)pyridin-2-yl)sulfamoyl)pyridin-2-yl)amino)butanoic acid;
3-(1-(6-(N-(6-(o-tolyl)-5-(trifluoromethyl)pyridin-2-yl)sulfamoyl)pyridin-2-yl)piperidin-2-yl)propanoic acid;
3-(ethyl(6-(N-(6-(2-ethyl-5-fluorophenyl)-5-(trifluoromethyl)pyridin-2-yl)sulfamoyl)pyridin-2-yl)amino)propanoic acid;
5-((6-(N-(6-(2-ethyl-5-fluorophenyl)-5-(trifluoromethyl)pyridin-2-yl)sulfamoyl)pyridin-2-yl)(isopropyl)amino)pentanoic acid;
5-((6-(N-(6-(5-fluoro-2-methylphenyl)-5-(trifluoromethyl)pyridin-2-yl)sulfamoyl)pyridin-2-yl)(isopropyl)amino)pentanoic acid;
5-(isopropyl(6-(N-(6-(o-tolyl)-5-(trifluoromethyl)pyridin-2-yl)sulfamoyl)pyridin-2-yl)amino)pentanoic acid;
3-((cyclopropylmethyl)(6-(N-(6-(o-tolyl)-5-(trifluoromethyl)pyridin-2-yl)sulfamoyl)pyridin-2-yl)amino)propanoic acid;
4-(butyl(6-(N-(6-(o-tolyl)-5-(trifluoromethyl)pyridin-2-yl)sulfamoyl)pyridin-2-yl)amino)butanoic acid;
4-(butyl(6-(N-(6-(5-fluoro-2-methylphenyl)-5-(trifluoromethyl)pyridin-2-yl)sulfamoyl)pyridin-2-yl)amino)butanoic acid;
4-((cyclopropylmethyl)(6-(N-(6-(o-tolyl)-5-(trifluoromethyl)pyridin-2-yl)sulfamoyl)pyridin-2-yl)amino)butanoic acid;
4-((cyclopropylmethyl)(6-(N-(6-(5-fluoro-2-methylphenyl)-5-(trifluoromethyl)pyridin-2-yl)sulfamoyl)pyridin-2-yl)amino)butanoic acid;
3-((6-(N-(6-(o-tolyl)-5-(trifluoromethyl)pyridin-2-yl)sulfamoyl)pyridin-2-yl)amino)cyclohexane-1-carboxylic acid;
(1S,3R)-3-((6-(N-(6-(o-tolyl)-5-(trifluoromethyl)pyridin-2-yl)sulfamoyl)pyridin-2-yl)amino)cyclohexane-1-carboxylic acid;
(1R,3S)-3-((6-(N-(6-(o-tolyl)-5-(trifluoromethyl)pyridin-2-yl)sulfamoyl)pyridin-2-yl)amino)cyclohexane-1-carboxylic acid;
(1R,2S,3R,4S)-3-((6-(N-(6-(o-tolyl)-5-(trifluoromethyl)pyridin-2-yl)sulfamoyl)pyridin-2-yl)amino)bicyclo[2.2.1]hept-5-ene-2-carboxylic acid;
(1 S,2R,3S,4R)-3-((6-(N-(6-(o-tolyl)-5-(trifluoromethyl)pyridin-2-yl)sulfamoyl)pyridin-2-yl)amino)bicyclo[2.2.1]hept-5-ene-2-carboxylic acid;
(1R,2S,3R,4S)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
(1S,2S,3R,4R)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
(1R,2R,3S,4S)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
4-(ethyl(6-(N-(6-(2-ethyl-5-fluorophenyl)-5-(trifluoromethyl)pyridin-2-yl)sulfamoyl)pyridin-2-yl)amino)butanoic acid;
3-((6-(N-(6-(2-cyclopropyl-5-fluorophenyl)-5-(trifluoromethyl)pyridin-2-yl)sulfamoyl)pyridin-2-yl)(ethyl)amino)propanoic acid;
4-(butyl(6-(N-(3-chloro-2′-isopropyl-[2,3′-bipyridin]-6-yl)sulfamoyl)pyridin-2-yl)amino)butanoic acid;
3-(cyclopropyl(6-(N-(6-(2-ethyl-5-fluorophenyl)-5-(trifluoromethyl)pyridin-2-yl)sulfamoyl)pyridin-2-yl)amino)propanoic acid;
4-(ethyl(6-(N-(6-(o-tolyl)-5-(trifluoromethyl)pyridin-2-yl)sulfamoyl)pyridin-2-yl)amino)butanoic acid;
4-(ethyl(6-(N-(6-(5-fluoro-2-methylphenyl)-5-(trifluoromethyl)pyridin-2-yl)sulfamoyl)pyridin-2-yl)amino)butanoic acid;
3-((cyclopropylmethyl)(6-(N-(6-(2-ethyl-5-fluorophenyl)-5-(trifluoromethyl)pyridin-2-yl)sulfamoyl)pyridin-2-yl)amino)propanoic acid;
3-((6-(N-(6-(o-tolyl)-5-(trifluoromethyl)pyridin-2-yl)sulfamoyl)pyridin-2-yl)thio)propanoic acid;
3-(cyclopropyl(6-(N-(6-(o-tolyl)-5-(trifluoromethyl)pyridin-2-yl)sulfamoyl)pyridin-2-yl)amino)propanoic acid;
3-((6-(N-(6-(2-ethyl-5-fluorophenyl)-5-(trifluoromethyl)pyridin-2-yl)sulfamoyl)pyridin-2-yl)(isopropyl)amino)propanoic acid;
4-((6-(N-(6-(2-ethylphenyl)-5-(trifluoromethyl)pyridin-2-yl)sulfamoyl)pyridin-2-yl)ₙxy)cyclohexane-1-carboxylic acid;
3-((6-(N-(6-(o-tolyl)-5-(trifluoromethyl)pyridin-2-yl)sulfamoyl)pyridin-2-yl)ₙxy)propanoic acid;
(1 S,2R,3S,4R)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
3-(propyl(6-(N-(6-(o-tolyl)-5-(trifluoromethyl)pyridin-2-yl)sulfamoyl)pyridin-2-yl)amino)propanoic acid;
3-((6-(N-(6-(5-fluoro-2-methylphenyl)-5-(trifluoromethyl)pyridin-2-yl)sulfamoyl)pyridin-2-yl)(propyl)amino)propanoic acid;
(S)-2-(3-methyl-1-(6-(N-(6-(o-tolyl)-5-(trifluoromethyl)pyridin-2-yl)sulfamoyl)pyridin-2-yl)piperidin-3-yl)acetic acid;
(R)-2-(3-methyl-1-(6-(N-(6-(o-tolyl)-5-(trifluoromethyl)pyridin-2-yl)sulfamoyl)pyridin-2-yl)piperidin-3-yl)acetic acid;
(S)-6-(2-(1H-tetrazol-5-yl)pyrrolidin-1-yl)-N-(5-chloro-6-(2,6-dimethylphenyl)pyridin-2-yl)pyridine-2-sulfonamide;
4-(cyclopropyl(6-(N-(6-(o-tolyl)-5-(trifluoromethyl)pyridin-2-yl)sulfamoyl)pyridin-2-yl)amino)butanoic acid;
3-((6-(N-(6-(5-fluoro-2-methylphenyl)-5-(trifluoromethyl)pyridin-2-yl)sulfamoyl)pyridin-2-yl)(isobutyl)amino)propanoic acid;
4-((6-(N-(6-(5-fluoro-2-methylphenyl)-5-(trifluoromethyl)pyridin-2-yl)sulfamoyl)pyridin-2-yl)(isobutyl)amino)butanoic acid;
3-(isobutyl(6-(N-(6-(o-tolyl)-5-(trifluoromethyl)pyridin-2-yl)sulfamoyl)pyridin-2-yl)amino)propanoic acid;
4-(isobutyl(6-(N-(6-(o-tolyl)-5-(trifluoromethyl)pyridin-2-yl)sulfamoyl)pyridin-2-yl)amino)butanoic acid;
1-((methyl(6-(N-(6-(o-tolyl)-5-(trifluoromethyl)pyridin-2-yl)sulfamoyl)pyridin-2-yl)amino)methyl)cyclopentane-1-carboxylic acid;
4-((6-(N-(6-(5-fluoro-2-methylphenyl)-5-(trifluoromethyl)pyridin-2-yl)sulfamoyl)pyridin-2-yl)(propyl)amino)butanoic acid;
4-(propyl(6-(N-(6-(o-tolyl)-5-(trifluoromethyl)pyridin-2-yl)sulfamoyl)pyridin-2-yl)amino)butanoic acid;
4-((methyl(6-(N-(6-(o-tolyl)-5-(trifluoromethyl)pyridin-2-yl)sulfamoyl)pyridin-2-yl)amino)methyl)tetrahydro-2H-pyran-4-carboxylic acid;
4-((6-(N-(6-(o-tolyl)-5-(trifluoromethyl)pyridin-2-yl)sulfamoyl)pyridin-2-yl)(3,3,3-trifluoropropyl)amino)butanoic acid;
3-((6-(N-(6-(5-fluoro-2-methylphenyl)-5-(trifluoromethyl)pyridin-2-yl)sulfamoyl)pyridin-2-yl)(isopropyl)amino)propanoic acid;
2-(4-((6-(N-(5-chloro-6-(o-tolyl)pyridin-2-yl)sulfamoyl)pyridin-2-yl)amino)cyclohexane-1-carboxamido)butanoic acid;
3-((6-(N-(6-(o-tolyl)-5-(trifluoromethyl)pyridin-2-yl)sulfamoyl)pyridin-2-yl)(3,3,3-trifluoropropyl)amino)propanoic acid;
3-((6-(N-(6-(5-fluoro-2-methylphenyl)-5-(trifluoromethyl)pyridin-2-yl)sulfamoyl)pyridin-2-yl)(3,3,3-trifluoropropyl)amino)propanoic acid;
1-((methyl(6-(N-(6-(o-tolyl)-5-(trifluoromethyl)pyridin-2-yl)sulfamoyl)pyridin-2-yl)amino)methyl)cyclobutane-1-carboxylic acid;
4-(((6-(N-(6-(5-fluoro-2-methylphenyl)-5-(trifluoromethyl)pyridin-2-yl)sulfamoyl)pyridin-2-yl)(methyl)amino)methyl)tetrahydro-2H-pyran-4-carboxylic acid;
1-(((6-(N-(6-(5-fluoro-2-methylphenyl)-5-(trifluoromethyl)pyridin-2-yl)sulfamoyl)pyridin-2-yl)(methyl)amino)methyl)cyclobutane-1-carboxylic acid;
3-((4-(N-(5-chloro-6-(2,6-dimethylphenyl)pyridin-2-yl)sulfamoyl)pyridin-2-yl)(ethyl)amino)propanoic acid;
3-((4-(N-(5-chloro-6-(2,6-dimethylphenyl)pyridin-2-yl)sulfamoyl)pyridin-2-yl)amino)pentanoic acid;
3-((4-(N-(5-chloro-6-(2,6-dimethylphenyl)pyridin-2-yl)sulfamoyl)pyridin-2-yl)amino)butanoic acid;
2-(adamantan-1-yl)-2-((6-(N-(6-(o-tolyl)-5-(trifluoromethyl)pyridin-2-yl)sulfamoyl)pyridin-2-yl)amino)acetic acid;
3-((4-(N-(5-chloro-6-(5-fluoro-2-methylphenyl)pyridin-2-yl)sulfamoyl)pyridin-2-yl)(ethyl)amino)propanoic acid;
3-((6-(N-(6-(2,6-dimethylphenyl)-5-(trifluoromethyl)pyridin-2-yl)sulfamoyl)pyridin-2-yl)(ethyl)amino)propanoic acid;
(6-(N-(6-(o-tolyl)-5-(trifluoromethyl)pyridin-2-yl)sulfamoyl)pyridin-2-yl)glycine;
3-(butyl(6-(N-(6-(o-tolyl)-5-(trifluoromethyl)pyridin-2-yl)sulfamoyl)pyridin-2-yl)amino)propanoic acid;
3-(butyl(6-(N-(6-(5-fluoro-2-methylphenyl)-5-(trifluoromethyl)pyridin-2-yl)sulfamoyl)pyridin-2-yl)amino)propanoic acid;
3-(cyclobutyl(6-(N-(6-(o-tolyl)-5-(trifluoromethyl)pyridin-2-yl)sulfamoyl)pyridin-2-yl)amino)propanoic acid;
4-((6-(N-(5-chloro-6-(o-tolyl)pyridin-2-yl)sulfamoyl)pyridin-2-yl)(isobutyl)amino)butanoic acid;
4-((6-(N-(5-chloro-6-(2,6-dimethylphenyl)pyridin-2-yl)sulfamoyl)pyridin-2-yl)(isobutyl)amino)butanoic acid;
4-((6-(N-(5-chloro-6-(o-tolyl)pyridin-2-yl)sulfamoyl)pyridin-2-yl)(propyl)amino)butanoic acid;
4-((6-(N-(5-chloro-6-(2,6-dimethylphenyl)pyridin-2-yl)sulfamoyl)pyridin-2-yl)(propyl)amino)butanoic acid, and
4-((2-methoxyethyl)(6-(N-(6-(o-tolyl)-5-(trifluoromethyl)pyridin-2-yl)sulfamoyl)pyridin-2-yl)amino)butanoic acid

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The terms “pharmaceutically acceptable salt” or “pharmaceutically acceptable salts”, as used herein, refers to a salt or salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids. The organic acid or inorganic acids used to form pharmaceutically acceptable acid addition salts of compounds of the present invention include, but are not limited to, acetic acid, adipic acid, ascorbic acid, aspartic acid, benzoic acid, benzenesulfonic acid, carbonic acid, camphor sulfonic acid, capric acid, chlorotheophyllinate, citric acid, ethanedisulfonic acid, fumaric acid, D-glycero-D-gulo-Heptonicacid, galactaric aid, galactaric acid/mucic acid, gluceptic acid, glucoheptonoic acid, gluconic acid, glucuronic acid, glutamatic acid, glutaric acid, glycolic acid, hippuric acid, hydrobromic acid, hydrochloric acid, hydroiodic acid, isethionic acid, lactic acid, lactobionic acid, lauryl sulfuric acid, malic acid, maleic acid, malonic acid, mandelic acid, mesylic acid, methanesulfonic acid, mucic acid, naphthoic acid, 1-hydroxy-2-naphthoic acid, naphthalenesulfonic acid, 2-naphthalenesulfonic acid, nicotinic acid, nitric acid, octadecanoic acid, oleaic acid, oxalic acid, palmitic acid, pamoic acid, phosphoric acid, polygalacturonic acid, propionic acid, sebacic acid, stearic acid, succinic acid, sulfosalicylic acid, sulfuric acid, tartaric acid, p-toluenesulfonic acid, trifluoroacetic acid and triphenylacetic acid.

Salt forms of the compounds of the present invention can be converted into the free compounds by treatment with a suitable basic agent.

Pharmaceutically acceptable acid addition salts of compounds of the present invention include, but are not limited to, a acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlorotheophyllinate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete and xinafoate salt forms.

Pharmaceutically acceptable base addition salts can be formed with inorganic and organic bases. Organic bases used to form pharmaceutically acceptable base addition salts of compounds of the present invention include, but are not limited to, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine. Inorganic bases used to form pharmaceutically acceptable base addition salts of compounds of the present invention include, but are not limited to, sodium hydroxide, potassium hydroxide, ammonium hydroxide, ammonium salts and metals from columns I to XII of the periodic table. Pharmaceutically acceptable base addition salts of compounds of the present invention include, but are not limited to, sodium, potassium, ammonium, calcium, magnesium, iron, silver, zinc, and copper salts; particularly suitable salts include ammonium, potassium, sodium, calcium and magnesium salts.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the present invention include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the present invention include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

By way of example, compounds of the present invention can exist in a deuterated form as shown below:

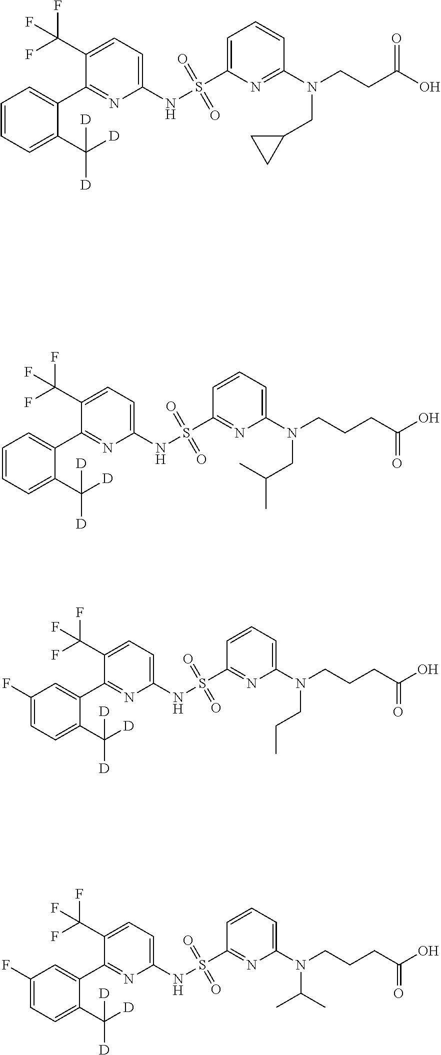 [SMILES: [2H]C([2H])([2H])c1ccccc1-c1nc(NS(=O)(=O)c2cccc(N(CCC(=O)O)CC3CC3)n2)ccc1C(F)(F)F ; [2H]C([2H])([2H])c1ccccc1-c1nc(NS(=O)(=O)c2cccc(N(CCCC(=O)O)CC(C)C)n2)ccc1C(F)(F)F ; [2H]C([2H])([2H])c1ccc(F)cc1-c1nc(NS(=O)(=O)c2cccc(N(CCC)CCCC(=O)O)n2)ccc1C(F)(F)F ; [2H]C([2H])([2H])c1ccc(F)cc1-c1nc(NS(=O)(=O)c2cccc(N(CCCC(=O)O)C(C)C)n2)ccc1C(F)(F)F]

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Processes for Making Compounds of Invention

General procedures for preparing compounds of the present invention are described herein. In the reactions described, reactive functional groups, for example hydroxy, amino, imino or carboxy groups, where these are desired in the final product, may be protected to avoid their unwanted participation in the reactions. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999.

Compounds of the present invention were made by processes described herein and as illustrated in the Examples. The combination of various building blocks and intermediates described herein can be applied to yield compounds of the invention. Non-limiting examples of synthetic schemes used to make compounds of the present invention are illustrated in Scheme 1.

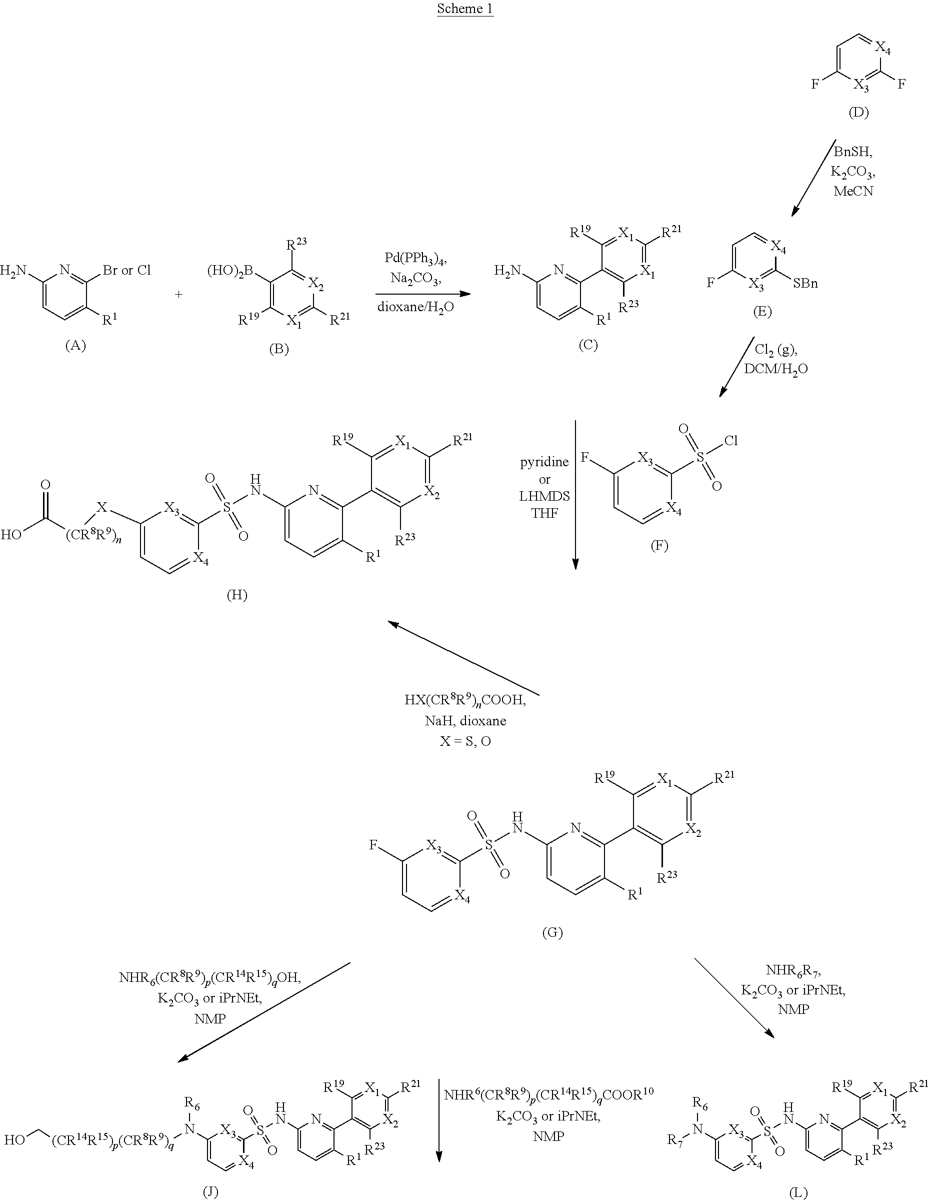

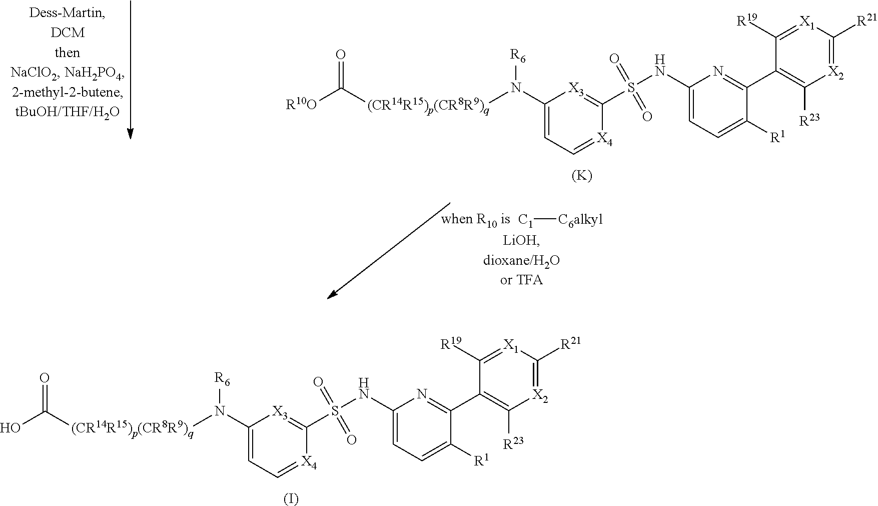

where: X₁, X₂, X₃, X₄, n, R¹, R⁶, R⁷, R⁸, R⁹, R¹⁰, R¹⁴, R¹⁵, R¹⁹, R²¹ and R²³ are as defined herein, p is 0 to n and q is 0 to m (where m, and n are as defined herein), however p and q can not both be 0.

The starting materials for the above reaction scheme are commercially available or can be prepared according to methods known to one skilled in the art or by methods disclosed herein. In general, the compounds of the invention are prepared in the above reaction Scheme I as follows: Suzuki cross-coupling of building block (A) with building block (B) provides intermediate (C). Separately, building block (D) is converted into intermediate (E) which is subsequently converted into building block (F). Intermediate (C) and building block (F) are combined to form intermediate (G). Intermediate (G) can then be converted into target compound (H) with an appropriate building block alcohol or thiol of the subtype HO(CR⁸R⁹)ₙCOOH or HS(CR⁸R⁹)ₙCOOH, respectively in the presence of a strong base. Alternatively, intermediate (G) can be combined with the appropriate building block amine NHR⁶R⁷ to yield target compound (L). Alternatively, intermediate (G) can be combined with a suitable aminoalcohol building block NHR⁶(CR⁸R⁹)ₚ(CR¹⁴R¹⁵)_{q}OH, where the resulting intermediate (J) can then be subsequently oxidized via a two-step procedure to obtain target compound (I). Similarly, intermediate (G) can be combined with an appropriate amino building block of the type NHR⁶(CR⁸R⁹)ₚ(CR¹⁴R¹⁵)_{q}COOR¹⁰ to achieve (K), which, when R¹⁰ is H, can then be subsequently hydrolyzed under either acidic or basic conditions to obtain target compound (I).

### Examples

The compounds of the present invention can be produced as shown in the following examples. The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art or can be produced by organic synthesis methods as described herein.

For illustrative purposes, the general reaction schemes depicted herein provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Abbreviations

Abbreviations used are those conventional in the art or the following:

| | |
|---|---|
| Ac: Acetyl | q: quartet |
| aq.: aqueous | s: singlet |
| br: broad | t: triplet |
| d: doublet; dd: doublet of doublets | THF: tetrahydrofuran |
| DCM: dichloromethane | TFA: trifluoroacetic acid |
| DMSO: dimethylsulfoxide | DMEM: Dulbecco's modified |
| ESI-MS: electrospray ionization mass | eagle medium |
| spectrometry | wt: weight |
| EtoAc: ethyl acetate | Isco, ISCO: Flash |
| HPLC: high pressure liquid | chromatography cartridge |
| chromatography | containing silica gel |
| h, hr: hour(s) | provided by Teledyne Isco |
| LC and LCMS: liquid chromatography | HMDS: hexamethyldisilazane |
| and liquid chromatography-mass | tBuOH: tert-butanol |
| spectrometry | Me: methyl |
| MeOH: methanol | m/z: mass to charge ratio |
| HEPES: 4-(2-hydroxyethyl)-1- | M and mM: molar and |
| piperazineethanesulfonic acid | millimolar |
| PBS: Phosphate Buffered Saline, | mg: milligram |
| pH 7.4 | μL, mL and L: microliter, |
| MS: mass | milliliter and liter |
| m: multiplet | N: equivalent per liter |
| NMP: N-methylpyrrolidinone | NMR: nuclear magnetic |
| LHMDS or LiHMDS: Lithium | resonance |
| hexamethyldilazane | Ph: phenyl |
| min(s): minute(s) | |

### Analytical Methods

ESI-MS data (also reported herein as simply MS) were recorded using Waters System (Acquity UPLC and a Micromass ZQ mass spectrometer); all masses reported are the m/z of the protonated parent ions unless recorded otherwise.

### LC/MS:

The sample is dissolved in suitable solvent such as MeCN, DMSO or MeOH and is injected directly into the column using an automated sample handler. The analysis is performed using one of the following methods:

### HPLC Conditions:

Condition 1: Agilent 1200 Series HPLC system:
Agilent Binary Gradient Manager with Degasser
Agilent Diode Array Detector
Agilent 6140 Quadrupole LC/MS
SoftA ELS Detector
   HPLC column: Waters Acquity HSS T3 C18 1.8 um, 2.1×50 mm
   Flow rate: 0.9 mL/min
   Temperature: 60° C. (column temp)
   Mobile phase compositions: A: 0.05% trifluoroacetic acid in water.
B: 0.035% trifluoroacetic acid in acetonitrile.

### Gradient:

| Time (min) | Flow (mL/min) | % A | % B |
|---|---|---|---|
| 0 | 0.9 | 90 | 10 |
| 0.15 | 0.9 | 90 | 10 |
| 1.50 | 0.9 | 0 | 100 |
| 1.95 | 0.9 | 0 | 100 |
| 2 | 0.9 | 90 | 10 |
| 2.25 | 0.9 | 90 | 10 |

### Synthesis of Intermediates

### Synthesis of Intermediate 6-fluoropyridine-2-sulfonyl Chloride (Int-a1)

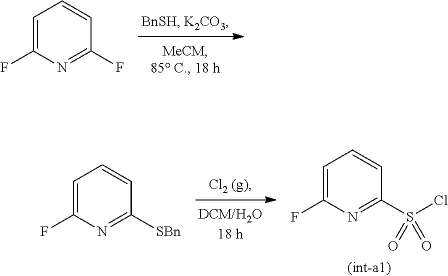

### Step 1. Synthesis of 2-(benzylthio)-6-fluoropyridine

In a flask, 2,6-difluoropyridine (12.7 g, 110 mmol), benzyl mercaptan (11.8 mL, 100 mmol), and potassium carbonate (20.8 g, 150 mmol) were suspended in MeCN (60 mL) and heated to 85° C. for 18 h. The mixture was cooled to room temperature, filtered, and concentrated. The crude product 2-(benzylthio)-6-fluoropyridine (20.3 g, 92.4 mmol, 92% yield) was used directly in the next reaction. ¹H NMR (400 MHz, Chloroform-d) δ 7.61-7.55 (m, 1H), 7.45-7.43 (m, 2H), 7.35-7.31 (m, 2H), 7.29-7.25 (m, 1H), 7.05 (dd, J=7.7, 2.2 Hz, 1H), 6.62 (dd, J=8.0, 2.7 Hz, 1H), 4.43 (s, 2H). ¹⁹F NMR (376 MHz, Chloroform-d) 6-67.44 (s, 1F).

### Step 2. Synthesis of 6-fluoropyridine-2-sulfonyl Chloride (Int-a1)

To a mixture of 2-(benzylthio)-6-fluoropyridine (20.3 g, 92.4 mmol) in DCM (250 mL) and water (80 mL) was added chlorine gas, which was bubbled through the mixture for 15 min until a light green color persisted. After stirring for 18 h, the reaction mixture was diluted with water and extracted with DCM (30 mL×2). The combined organics were washed with a dilute solution of NaHSO₃, dried over MgSO₄, and concentrated in vacuo to afford a crude oil. The crude material was purified by flash column chromatography (330 g silica gel column, 0-30% EtOAc/heptane) to afford 6-fluoropyridine-2-sulfonyl chloride (int-a1) as yellowish viscous oil: (11.8 g, 60.2 mmol, 65% yield). ¹H NMR (400 MHz, Chloroform-d) δ 8.23-8.13 (m, 1H), 8.02 (dd, J=7.4, 1.6 Hz, 1H), 7.37 (dd, J=8.2, 2.6 Hz, 1H). ¹⁹F NMR (376 MHz, Chloroform-d) 6-61.18 (s, 1F).

### Synthesis of Intermediate 1-((methylamino)methyl)cyclobutane-1-carboxylic Acid (Int-a2)

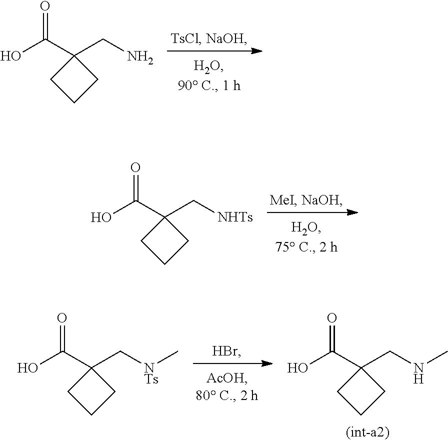

### Step 1. Synthesis of 1-((4-methylphenylsulfonamido)methyl)cyclobutanecarboxylic Acid

A slurry of 1-(aminomethyl)cyclobutanecarboxylic acid hydrochloride (1.01 g, 6.09 mmol), p-toluenesulfonyl chloride (1.51 g, 7.92 mmol) and 1 M aqueous NaOH (24.4 mL, 24.4 mmol) was heated in water at 90° C. for 1 h, after which the reaction mixture was cooled to 0° C. and acidified by the addition of 3 M HCl solution. The resulting white precipitate was filtered then washed successively with water and dried to afford the product 1-((4-methylphenylsulfonamido)methyl)cyclobutanecarboxylic acid (0.758 g, 2.68 mmol, 44% yield) as a white solid. LCMS (Condition 1): m/z 284.1 [M+H]⁺, 1.51 min. ¹H NMR (400 MHz, DMSO-d₆) δ 12.32 (s, 1H), 7.72-7.69 (m, 2H), 7.66 (t, J=6.7 Hz, 1H), 7.41-7.39 (m, 2H), 2.97 (d, J=6.5 Hz, 2H), 2.39 (s, 3H), 2.23-2.16 (m, 2H), 1.97-1.91 (m, 2H), 1.81-1.73 (m, 2H).

### Step 2. Synthesis of 1-((N,4-dimethylphenylsulfonamido)methyl)cyclobutanecarboxylic Acid

A solution of 1-((4-methylphenylsulfonamido)methyl)cyclobutanecarboxylic acid (756 mg, 2.67 mmol), iodomethane (0.526 mL, 8.40 mmol), and 1 M aqueous NaOH (10.7 mL, 10.7 mmol) in water (6.7 mL) was heated to 75° C. for 2 h. The reaction mixture was cooled to room temperature, washed with DCM (×3), acidified by the addition of 3 M aqueous HCl, extracted with diethyl ether (×3), dried (Na₂SO₄), filtered, and concentrated to afford the product 1-((N,4-dimethylphenylsulfonamido)methyl)cyclobutanecarboxylic acid (778 mg, 2.62 mmol, 98% yield) as a yellow-gold solid. LCMS (Condition 1): m/z 298.1 [M+H]⁺, 1.62 min. ¹H NMR (400 MHz, DMSO-d₆) δ 12.51 (s, 1H), 7.68-7.66 (m, 2H), 7.46-7.44 (m, 2H), 3.28 (s, 2H), 2.54 (s, 3H), 2.41 (s, 3H), 2.30-2.23 (m, 2H), 2.06-1.99 (m, 2H), 1.89-1.77 (m, 2H).

### Step 3. Synthesis of 1-((methylamino)methyl)cyclobutanecarboxylic Acid Hydrobromide

A solution of 1-((N,4-dimethylphenylsulfonamido)methyl)cyclobutanecarboxylic acid (775 mg, 2.61 mmol) was heated in HBr 33 wt. % in acetic acid (14.2 mL, 78.0 mmol) for 2 h at 80° C. The reaction was then cooled to room temperature, diluted with water (10 mL), and washed with diethyl ether (40 mL×3). The aqueous layer was concentrated to dryness and the resulting solid was recrystallized from acetone to afford 1-((methylamino)methyl)cyclobutane-1-carboxylic acid (int-a2) as a hydrobromide salt as a crystalline white solid: (334 mg, 1.49 mmol, 57% yield). ¹H NMR (400 MHz, DMSO-d₆) δ 8.27 (s, 2H), 3.28 (s, 2H), 2.59 (s, 3H), 2.38-2.31 (m, 2H), 2.08-1.88 (m, 4H).

### Synthesis of Intermediate tert-butyl 3-(propylamino)propanoate (Int-a3)

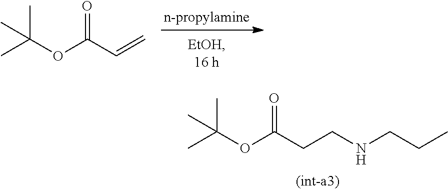

A mixture of tert-butyl acrylate (1.50 g, 11.7 mmol) and propan-1-amine (0.692 g, 11.7 mmol) in ethanol (10 mL) was stirred at room temperature overnight. The solvent was then removed and the residue was subjected to purification by flash column chromatography (0-50% EtOAc/heptane) to afford tert-butyl 3-(propylamino)propanoate (int-a3): (893 mg, 4.53 mmol, 39% yield). LCMS (Condition 1): m/z 188.1 [M+H]⁺, 0.59 min.

### Synthesis of Intermediate 4-((cyclopropylmethyl)amino)butan-1-ol (Int-a4)

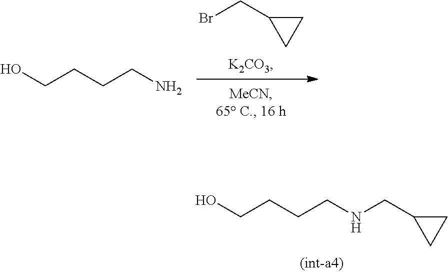 [SMILES: BrCC1CC1 ; NCCCCO ; OCCCCNCC1CC1]

A mixture of 4-aminobutan-1-ol (1.68 g, 18.9 mmol), (bromomethyl)cyclopropane (3.82 g, 28.3 mmol) and K₂CO₃ (5.21 g, 37.7 mmol) in 75 mL of acetonitrile (75 mL) was stirred at 65° C. overnight. The reaction was filtered through a Celite pad, the filtrate was concentrated, and the residue was subjected to purification by flash column chromatography (0-20% MeOH/DCM) to afford 4-((cyclopropylmethyl)amino)butan-1-ol (int-a4) as a clear oil: (1.20 g, 7.54 mmol, 40% yield). LCMS (Condition 1): m/z 144.2 [M+H]⁺, 0.32 min.

### Synthesis of Intermediate 2-(1-(tert-butoxycarbonyl)-3-methylpiperidin-3-yl)acetic Acid (Int-a5)

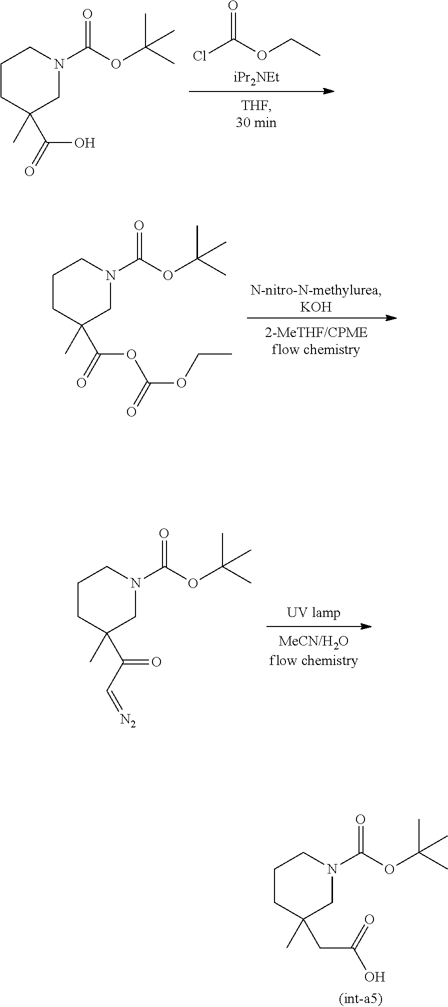

### Step 1. Synthesis of 1-(tert-butoxycarbonyl)-3-methylpiperidine-3-carboxylic (Ethyl Carbonic) Anhydride

In a flask, 1-(tert-butoxycarbonyl)-3-methylpiperidine-3-carboxylic acid (1.00 g, 4.11 mmol) and N,N-diisopropylethylamine (0.861 ml, 4.93 mmol) were dissolved in THE (8 mL) and cooled to 0° C. Then ethyl chloroformate (0.472 ml, 4.93 mmol) was added and reaction was allowed to stir for 30 min at room temperature. The crude reaction mixture was used directly in next reaction without further purification.

### Step 2. Synthesis of tert-butyl 3-(2-diazoacetyl)-3-methylpiperidine-1-carboxylate

A Vapourtec E-Series flow reactor system equipped with PFA (perfluoroalkoxy) tubing and Zaiput liquid-liquid separator was utilized. System parameters: System solvent pump A—2MeTHF, Reagent A: N-nitroso-N-methylurea (NMU) (0.39 g, 3.8 mmol, 0.4 M in 2MeTHF/CPME, 1:1), pump A flow rate 1.786 mL/min; system solvent pump B—H₂O, Reagent B: KOH (0.32 g, 5.7 mmol, 1.5 M in H₂O), pump B flow rate 0.714 mL/min; system solvent pump C—2MeTHF, crude 1-(tert-butoxycarbonyl)-3-methylpiperidine-3-carboxylic (ethyl carbonic) anhydride (1.2 g, 3.8 mmol, 0.25 M in THF), pump C flow rate 0.4 mL/min. The solutions of NMU and KOH were mixed (T-mixer) in a cooled reactor (10° C. 2 mL) with a residence time of 0.8 min. The aqueous phase was separated by a Zaiput liquid-liquid phase separator. The organic stream was mixed with the solution of crude 1-(tert-butoxycarbonyl)-3-methylpiperidine-3-carboxylic (ethyl carbonic) anhydride in a second reactor (10 mL) with a residence time of 3.4 min. The product stream was collected into a flask under magnetic stirring. The reaction mixture was stirred for 10 min. and subsequently quenched with AcOH. The crude mixture was concentrated to give the crude product tert-butyl 3-(2-diazoacetyl)-3-methylpiperidine-1-carboxylate that was used without further purification.

### Step 3. Synthesis of 2-(1-(tert-butoxycarbonyl)-3-methylpiperidin-3-yl)acetic Acid (Int-a5)

A Vapourtec E-Series flow reactor system equipped with PFA (perfluoroalkoxy) tubing a UV-150 photoreactor was utilized. System parameters: System solvent pump A—MeCN, Reagent A: crude tert-butyl 3-(2-diazoacetyl)-3-methylpiperidine-1-carboxylate (0.350 mg, 1.3 mmol, 0.05 M in MeCN/H₂O, 10:1), pump A flow rate 0.5 mL/min. The solution of crude tert-butyl 3-(2-diazoacetyl)-3-methylpiperidine-1-carboxylate was passed through a UV-150 photoreactor (32° C., 10 mL, lamp power 82 W, filter 300-2000 nm) with a residence time of 20 min. The crude product stream was concentrated to give the crude product 2-(1-(tert-butoxycarbonyl)-3-methylpiperidin-3-yl)acetic acid (int-a5) that was used without further purification. LCMS (Condition 1): m/z 202 [M−55]⁺, 1.28 min.

### Synthesis of Intermediate 4-(isobutylamino)butan-1-ol (Int-a6)

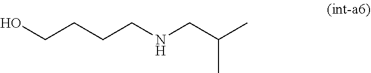

4-(isobutylamino)butan-1-ol (int-a6) was synthesized using a procedure adapted from the one described in (int-a4), except (bromomethyl)cyclopropane was replaced with 1-bromo-2-methylpropane. LCMS (Condition 1): m/z 146.2 [M+H]⁺, 0.32 min.

### Synthesis of Intermediate 1-((methylamino)methyl)cyclopentanecarboxylic Acid (Int-a7)

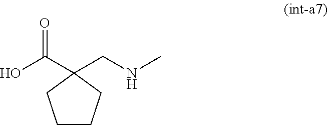

1-((methylamino)methyl)cyclopentanecarboxylic acid hydrobromide (int-a7) was synthesized using a procedure adapted from the one described in (int-a2), except in step 1, 1-(aminomethyl)cyclobutanecarboxylic acid hydrochloride was replaced with 1-(aminomethyl)cyclopentanecarboxylic acid. ¹H NMR (400 MHz, DMSO-d₆) δ 8.27 (s, 2H), 3.07 (s, 2H), 2.58 (s, 3H), 1.95 (m, 2H), 1.66 (m, 6H).

### Synthesis of Intermediate 4-(propylamino)butan-1-ol (Int-a8)

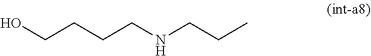

4-(propylamino)butan-1-ol (int-a8) was synthesized using a procedure adapted from the one described in (int-a4), except (bromomethyl)cyclopropane was replaced with 1-bromopropane. LCMS (Condition 1): m/z 132.2 [M+H]⁺, 0.31 min.

### Synthesis of Intermediate 4-((methylamino)methyl)tetrahydro-2H-pyran-4-carboxylic Acid (Int-a9)

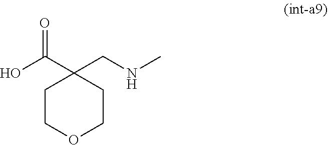

4-((methylamino)methyl)tetrahydro-2H-pyran-4-carboxylic acid hydrobromide (int-a9) was synthesized using a procedure adapted from the one described in (int-a2), except in step 1, 1-(aminomethyl)cyclobutanecarboxylic acid hydrochloride was replaced with 4-(aminomethyl)tetrahydro-2H-pyran-4-carboxylic acid. LCMS (Condition 1): m/z 174.2 [M+H]⁺, 0.21 min. ¹H NMR (400 MHz, DMSO-d₆) δ 8.31 (s, 2H), 3.72 (dt, J=11.7, 4.3 Hz, 2H), 3.45 (ddd, J=11.9, 9.2, 2.8 Hz, 2H), 3.17 (t, J=5.9 Hz, 2H), 2.59 (t, J=4.3 Hz, 3H), 1.93 (m, 2H), 1.54 (ddd, J=13.4, 9.1, 3.9 Hz, 2H).

### Synthesis of Intermediate 4-((3,3,3-trifluoropropyl)amino)butan-1-ol (Int-a10)

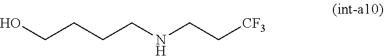 [SMILES: OCCCCNCCC(F)(F)F]

4-((3,3,3-trifluoropropyl)amino)butan-1-ol (int-a10) was synthesized using a procedure adapted from the one described in (int-a4), except (bromomethyl)cyclopropane was replaced with 3-bromo-1,1,1-trifluoropropane. The crude material was used directly without purification.

### Synthesis of Intermediate tert-butyl 3-(cyclobutylamino)propanoate (Int-a11)

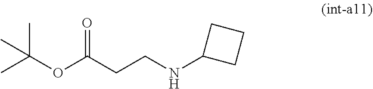

tert-butyl 3-(cyclobutylamino)propanoate (int-a11) was synthesized using a procedure adapted from the one described in (int-a3), except propan-1-amine was replaced with cyclobutanamine. The crude material was used directly without purification.

### Synthesis of Intermediate tert-butyl 3-((3,3,3-trifluoropropylamino)propanoate (Int-a12)

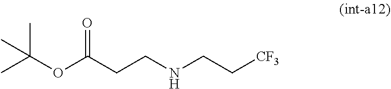 [SMILES: CC(C)(C)OC(=O)CCNCCC(F)(F)F]

tert-butyl 3-((3,3,3-trifluoropropylamino)propanoate (int-a12) was synthesized using a procedure adapted from the one described in (int-a3), except propan-1-amine was replaced with 3,3,3-trifluoropropan-1-amine. LCMS (Condition 1): m/z 242.2 [M+H]⁺, 1.17 min.

### Synthesis of Intermediate 2-fluoropyridine-4-sulfonyl Chloride (Int-a13)

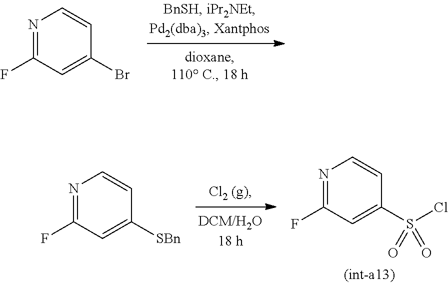

### Step 1. Synthesis of 4-(benzylthio)-2-fluoropyridine

In a flask, 4-bromo-2-fluoropyridine (1.0 g, 5.7 mmol) and benzyl mercaptan (0.67 mL, 5.7 mmol) were dissolved in dioxane (6 mL), and to the solution was added N,N-diisopropylethylamine (2.0 mL, 11 mmol) and (9,9-dimethyl-9H-xanthene-4,5-diyl)bis(diphenylphosphine) (Xantphos) (0.66 g, 1.1 mmol). The mixture was evacuated and backfilled with argon, Pd₂(dba)₃ (0.52 g, 0.57 mmol) was added, and the mixture was evacuated and backfilled with argon again. The reaction was stirred at 110° C. for 18 hours. The mixture was cooled and filtered, dried over Na₂SO₄, and concentrated in vacuo. The crude material was purified by flash column chromatography (40 g silica gel column, 0-30% EtOAc/heptane) to provide 4-(benzylthio)-2-fluoropyridine: (1.7 g, 5.7 mmol, 100% yield). LCMS (Condition 1): m/z 220.1 [M+H]⁺, 1.64 min. ¹⁹F NMR (376 MHz, Chloroform-d) δ −69.10 (s, 1F).

### Step 2. Synthesis of 2-fluoropyridine-4-sulfonyl Chloride (Int-a13)

To a mixture of 4-(benzylthio)-2-fluoropyridine (1.7 g, 5.7 mmol) in DCM (80 mL) and water (20 mL) was added chlorine gas, which was bubbled through the mixture for 15 min until a light green color persisted. After stirring for 36 h, the reaction mixture was diluted with water and extracted with DCM (30 mL×2). The combined organics were washed with a dilute solution of NaHSO₃, dried over MgSO₄, and concentrated in vacuo to afford a crude oil. The crude material was purified by flash column chromatography (120 g silica gel column, 0-30% EtOAc/heptane) to afford 2-fluoropyridine-4-sulfonyl chloride (int-a13): (0.55 g, 2.2 mmol, 30% yield). ¹⁹F NMR (376 MHz, Chloroform-d) 6-60.45 (s, 1F).

### Synthesis of Intermediate 6-fluoro-N-(6-(o-tolyl)-5-(trifluoromethyl)pyridin-2-yl)pyridine-2-sulfonamide (Int-b1)

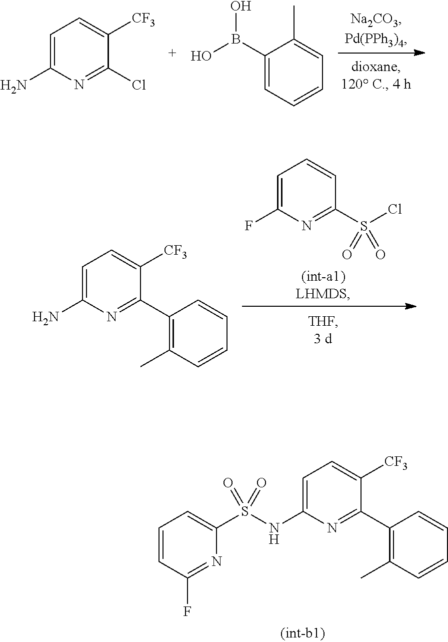

### Step 1. Synthesis of 6-(o-tolyl)-5-(trifluoromethyl)pyridin-2-amine

In a flask, 6-chloro-5-(trifluoromethyl)pyridin-2-amine (2.00 g, 10.2 mmol) and (2-methylphenyl)boronic acid (1.73 g, 12.7 mmol) were dissolved in dioxane (20 mL) and water (3 mL) and treated with sodium carbonate (4.31 g, 40.7 mmol). The mixture was subsequently evacuated and backfilled using argon, Pd(PPh₃)₄ (1.18 g, 1.02 mmol) was added, and the mixture was evacuated and backfilled with argon again. The reaction was stirred at 120° C. for 4 h. The reaction was repeated on the same scale to the same result and the batches were combined. The combined reactions were diluted with EtOAc, washed with water and brine, dried (Na₂SO₄), filtered, and concentrated. The resulting residue was purified by flash column chromatography to afford the desired product (5.02 g, 19.9 mmol, 98% yield). LCMS (Condition 1): m/z 253.2 [M+H]⁺, 1.34 min.

### Step 2. Synthesis of 6-fluoro-N-(6-(o-tolyl)-5-(trifluoromethyl)pyridin-2-yl)pyridine-2-sulfonamide (Int-b1)

A solution of 6-(o-tolyl)-5-(trifluoromethyl)pyridin-2-amine (5.02 g, 19.9 mmol) in THE (200 mL) was treated with 1.0 M LHMDS in THE (39.8 mL, 39.8 mmol) and was stirred for 10 minutes, then 6-fluoropyridine-2-sulfonyl chloride (int-a1) (5.06 g, 25.9 mmol) was added and the reaction was stirred for 3 days. The reaction mixture was diluted with EtOAc, washed with brine, dried (Na₂SO₄), filtered, and concentrated. The resulting residue was purified by flash column chromatography to afford the product which was contaminated with some starting material. The product containing fractions were combined, concentrated, and purified by reverse phase column chromatography to afford 6-fluoro-N-(6-(o-tolyl)-5-(trifluoromethyl)pyridin-2-yl)pyridine-2-sulfonamide (int-b1): (5.59 g, 13.6 mmol, 68% yield). LCMS (Condition 1): m/z 412.1 [M+H]⁺, 1.68 min.

### Synthesis of Intermediate 6-fluoro-N-(6-(2-isopropylphenyl)-5-(trifluoromethyl)pyridin-2-yl)pyridine-2-sulfonamide (Int-b2)

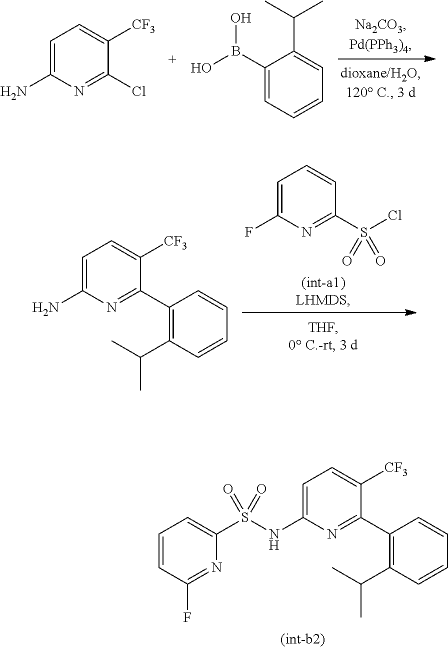 [SMILES: Nc1ccc(C(F)(F)F)c(Cl)n1 ; CC(C)c1ccccc1B(O)O ; CC(C)c1ccccc1-c1nc(N)ccc1C(F)(F)F ; O=S(=O)(Cl)c1cccc(F)n1 ; CC(C)c1ccccc1-c1nc(NS(=O)(=O)c2cccc(F)n2)ccc1C(F)(F)F]

### Step 1. Synthesis of 6-(2-isopropylphenyl)-5-(trifluoromethyl)pyridin-2-amine

In a vial, 6-chloro-5-(trifluoromethyl)pyridin-2-amine (2.00 g, 10.2 mmol), (2-isopropylphenyl)boronic acid (2.00 g, 12.2 mmol), sodium carbonate (3.24 g, 30.5 mmol), and Pd(Ph₃P)₄ (1.18 g, 1.02 mmol) were taken up in dioxane (20 mL) and water (3 mL), the mixture was sparged with argon, then the reaction was heated to 120° C. for 3 days. The crude reaction material was evaporated on silica gel and purified by flash column chromatography (80 g silica gel column, 0-6% MeOH/DCM, dry loading) to give the product 6-(2-isopropylphenyl)-5-(trifluoromethyl)pyridin-2-amine (2.61 g, 9.31 mmol, 92% yield) as a light yellow solid. LCMS (Condition 1): m/z 281.1 [M+H]⁺, 1.46 min.

### Step 2. Synthesis of 6-fluoro-N-(6-(2-isopropylphenyl)-5-(trifluoromethyl)pyridin-2-yl)pyridine-2-sulfonamide (Int-b2)

In a flask, 6-(2-isopropylphenyl)-5-(trifluoromethyl)pyridin-2-amine (2.61 g, 9.31 mmol) was taken up in THE (100 mL) and the solution was cooled to 0° C. To the solution was added 1.0 M LHMDS in THE (18.6 mL, 18.6 mmol), then a solution of 6-fluoropyridine-2-sulfonyl chloride (int-a1) (3.64 g, 18.6 mmol) that had been dissolved in THE (5 mL). After stirring overnight the reaction was quenched with 1 M HCl and extracted into EtOAc (25 mL×3). The organics were then washed with water and brine, dried over MgSO₄, and concentrated in vacuo. The crude material was purified by flash column chromatography (220 g silica gel column, 0-80% EtOAc/heptane) to give 6-fluoro-N-(6-(2-isopropylphenyl)-5-(trifluoromethyl)pyridin-2-yl)pyridine-2-sulfonamide (int-b2) as a light tan solid: (3.31 g, 7.53 mmol, 81% yield). LCMS (Condition 1): m/z 440.1 [M+H]⁺, 1.84 min. ¹H NMR (400 MHz, DMSO-d₆) δ 12.12 (s, 1H), 8.19 (d, J=8.9 Hz, 1H), 8.13-8.07 (m, 1H), 7.83 (dd, J=7.4, 2.1 Hz, 1H), 7.49-7.46 (m, 1H), 7.43-7.34 (m, 2H), 7.29 (s, 1H), 7.19-7.15 (m, 1H), 6.89 (d, J=7.6 Hz, 1H), 2.34-2.27 (m, 1H), 1.01 (d, J=6.8 Hz, 3H), 0.91 (d, J=6.8 Hz, 3H).

### Synthesis of Intermediate N-(6-(2-ethyl-5-fluorophenyl)-5-(trifluoromethyl)pyridin-2-yl)-6-fluoropyridine-2-sulfonamide (Int-b3)

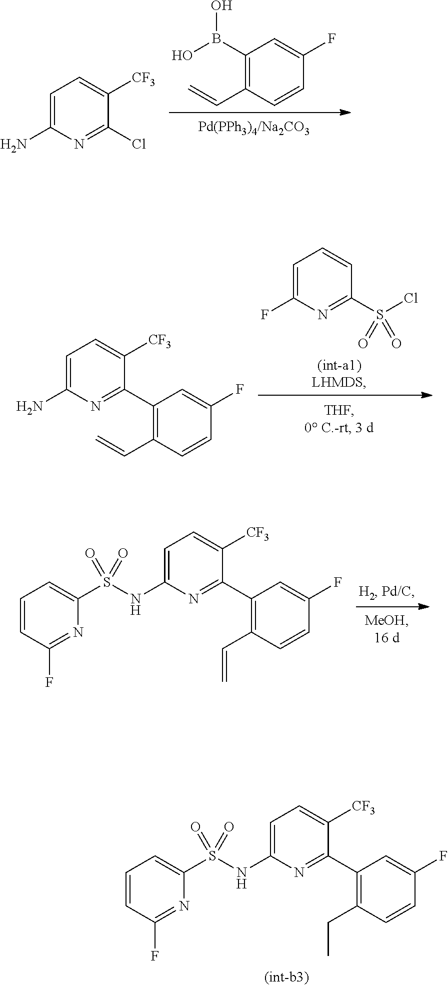 [SMILES: Nc1ccc(C(F)(F)F)c(Cl)n1 ; C=Cc1ccc(F)cc1B(O)O ; C=Cc1ccc(F)cc1-c1nc(N)ccc1C(F)(F)F ; O=S(=O)(Cl)c1cccc(F)n1 ; C=Cc1ccc(F)cc1-c1nc(NS(=O)(=O)c2cccc(F)n2)ccc1C(F)(F)F ; CCc1ccc(F)cc1-c1nc(NS(=O)(=O)c2cccc(F)n2)ccc1C(F)(F)F]

### Step 1. Synthesis of 6-(5-fluoro-2-vinylphenyl)-5-(trifluoromethyl)pyridin-2-amine

6-Chloro-5-(trifluoromethyl)pyridin-2-amine (5 g, 25.4 mmol) and (5-fluoro-2-vinylphenyl)boronic acid (5.28 g, 31.8 mmol) were dissolved in dioxane (60 mL) and water (9 mL) and treated with sodium carbonate (10.78 g, 102 mmol). The mixture was degassed using argon. Tetrakis(triphenylphosphino)palladium(0) (2.94 g, 2.54 mmol) was added and the mixture was degassed again. The mixture was stirred at 115° C. for 18 h. LCMS showed the reaction was complete. The mixture was cooled and filtered. The solids were washed with more dioxane (25 mL). The combined filtrate was dried over Na₂SO₄, filtered and concentrated to yield a reddish oil. The crude product was purified by silica gel chromatography (330 g ISCO column) (EtOAc/heptane 10-40%) to give the title compound as a yellow solid (6.10 g, 21.61 mmol, 85% yield). LCMS (Condition 1): m/z 283.2 [M+H]⁺, 1.28 min. ¹H NMR (400 MHz, Chloroform-d) δ 7.77 (d, J=8.7 Hz, 1H), 7.62 (dd, J=5.6, 8.7 Hz, 1H), 7.09 (td, J=2.7, 8.5 Hz, 1H), 6.94 (dd, J=2.7, 8.9 Hz, 1H), 6.54 (d, J=8.7 Hz, 1H), 6.32 (dd, J=11.0, 17.5 Hz, 1H), 5.59 (d, J=17.4 Hz, 1H), 5.12 (d, J=11.0 Hz, 1H), 4.88 (s, 2H). ¹⁹F NMR (376 MHz, Chloroform-d) 6-57.67 (s, 3F), −114.93 (s, 1F)

### Step 2. Synthesis of 6-fluoro-N-(6-(5-fluoro-2-vinylphenyl)-5-(trifluoromethyl)pyridin-2-yl)pyridine-2-sulfonamide

In a flask, 6-(5-fluoro-2-vinylphenyl)-5-(trifluoromethyl)pyridin-2-amine (5.74 g, 20.3 mmol) was taken up in THE (100 mL) and the solution was cooled to 0° C. To the solution was added 1 M LHMDS in THE (38.4 mL, 40.7 mmol), then a solution of 6-fluoropyridine-2-sulfonyl chloride (int-a1) (7.96 g, 40.7 mmol) that had been dissolved in THF. The reaction was then allowed to warm to room temperature and was stirred overnight. The reaction was quenched with 1 M HCl and extracted into EtOAc. The organics were then washed with water and brine, dried over MgSO₄, and concentrated in vacuo. The crude material was purified by flash column chromatography (220 g silica gel column, 0-60% EtOAc/heptane) to give the product 6-fluoro-N-(6-(5-fluoro-2-vinylphenyl)-5-(trifluoromethyl)pyridin-2-yl)pyridine-2-sulfonamide (5.07 g, 11.5 mmol, 57% yield) as a light tan/green solid. LCMS (Condition 1): m/z 441.9 [M+H]⁺, 1.69 min.

### Step 2. Synthesis of N-(6-(2-ethyl-5-fluorophenyl)-5-(trifluoromethyl)pyridin-2-yl)-6-fluoropyridine-2-sulfonamide (Int-b3)

In a vial, 6-fluoro-N-(6-(5-fluoro-2-vinylphenyl)-5-(trifluoromethyl)pyridin-2-yl)pyridine-2-sulfonamide (5.07 g, 11.5 mmol) was taken up in MeOH (100 mL) and Pd/C (0.611 g, 0.575 mmol) was added and the reaction was sparged with hydrogen. After 10 h, the reaction was filtered, another batch of Pd/C (0.611 g, 0.575 mmol) was added, and the reaction was again sparged with hydrogen. After 16 h, the reaction was filtered and the solvent removed in vacuo. The crude material was purified by flash column chromatography (330 g silica gel column, 0-80% EtOAc/heptane, dry loading) to give N-(6-(2-ethyl-5-fluorophenyl)-5-(trifluoromethyl)pyridin-2-yl)-6-fluoropyridine-2-sulfonamide (int-b3) as a white solid: (4.38 g, 9.88 mmol, 86% yield). LCMS (Condition 1): m/z 443.9 [M+H]⁺, 1.74 min. ¹H NMR (600 MHz, DMSO-d₆) δ 12.18 (s, 1H), 8.20 (d, J=8.8 Hz, 1H), 8.15-8.11 (m, 1H), 7.85 (dd, J=7.4, 1.9 Hz, 1H), 7.50 (dd, J=8.2, 2.1 Hz, 1H), 7.31-7.27 (m, 2H), 7.23-7.19 (m, 1H), 6.95-6.74 (m, 1H), 2.09-2.03 (m, 1H), 1.94-1.89 (m, 1H), 0.82 (t, J=7.6 Hz, 3H).

### Synthesis of Intermediate N-(3-chloro-2′-isopropyl-[2,3′-bipyridin]-6-yl)-6-fluoropyridine-2-sulfonamide (Int-b4)

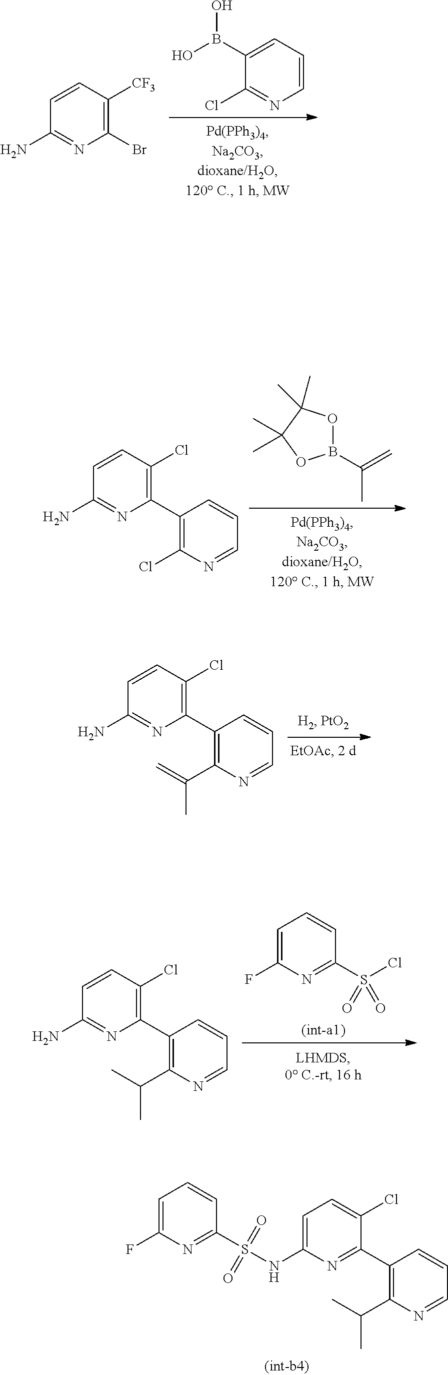

### Step 1. Synthesis of 2′,3-dichloro-[2,3′-bipyridin]-6-amine

In a microwave vial, 6-bromo-5-chloropyridin-2-ylamine (2.20 g, 10.6 mmol), 2-chloropyridine-3-boronic acid (2.00 g, 12.7 mmol), Na₂CO₃ (3.37 g, 31.8 mmol), and Pd(Ph₃P)₄ (1.23 g, 1.06 mmol) were suspended in dioxane (2 mL) and water (2 mL), the mixture was sparged with argon, then the reaction was heated in the microwave to 120° C. for 1 h. The crude reaction material was evaporated on silica gel and purified by flash column chromatography (80 g silica gel column, 0-6% MeOH/DCM, dry loading) to give the product 2′,3-dichloro-[2,3′-bipyridin]-6-amine (2.19 g, 9.14 mmol, 86% yield) as a light orange solid. LCMS (Condition 1): m/z 240.0 [M+H]⁺, 1.09 min. ¹H NMR (400 MHz, DMSO-d₆) δ 8.47 (dd, J=4.8, 1.9 Hz, 1H), 7.86 (dd, J=7.5, 2.0 Hz, 1H), 7.56 (d, J=8.8 Hz, 1H), 7.52 (dd, J=7.5, 4.8 Hz, 1H), 6.54 (d, J=8.8 Hz, 1H), 6.37 (s, 2H).

### Step 2. Synthesis of 3-chloro-2′-(prop-1-en-2-yl)-[2,3′-bipyridin]-6-amine

In a microwave vial, 2′,3-dichloro-[2,3′-bipyridin]-6-amine (1.19 g, 4.96 mmol), 2-isopropenylboronic acid, pinacol ester (1.12 mL, 5.95 mmol), Na₂CO₃ (1.58 g, 14.9 mmol), and Pd(Ph₃P)₄ (0.573 g, 0.496 mmol) were suspended in dioxane (12 mL) and water (2 mL), the solution was sparged with argon, then the reaction was heated in the microwave to 120° C. for 1 h. The crude reaction material was evaporated on silica gel and purified by flash column chromatography (80 g silica gel column, 0-8% MeOH/DCM, dry loading) to give the product 3-chloro-2′-(prop-1-en-2-yl)-[2,3′-bipyridin]-6-amine (489 mg, 1.99 mmol, 40% yield) as a yellow solid. LCMS (Condition 1): m/z 246.1 [M+H]⁺, 0.99 min.

### Step 3. Synthesis of 3-chloro-2′-isopropyl-[2,3′-bipyridin]-6-amine

In a vial, 3-chloro-2′-(prop-1-en-2-yl)-[2,3′-bipyridin]-6-amine (489 mg, 1.99 mmol) was taken up in EtOAc (5 mL) and platinum(IV) oxide (48.8 mg, 0.199 mmol) was added, then the suspension was sparged with hydrogen and allowed to stir for 2 days. The reaction was filtered via syringe and the resulting solution was concentrated in vacuo to give the product 3-chloro-2′-isopropyl-[2,3′-bipyridin]-6-amine (579.3 mg) that was taken on directly to the next reaction. LCMS (Condition 1): m/z 248.1 [M+H]⁺, 1.01 min. ¹H NMR (400 MHz, Methanol-d₄) δ 8.55 (dd, J=4.9, 1.8 Hz, 1H), 7.60 (dd, J=7.7, 1.8 Hz, 1H), 7.56 (d, J=8.9 Hz, 1H), 7.33 (dd, J=7.7, 4.9 Hz, 1H), 6.62 (d, J=8.9 Hz, 1H), 2.92 (hept, J=6.8 Hz, 1H), 1.24-1.11 (m, 6H).

### Step 4. Synthesis of N-(3-chloro-2′-isopropyl-[2,3′-bipyridin]-6-yl)-6-fluoropyridine-2-sulfonamide (Int-b4)

In a vial, 3-chloro-2′-isopropyl-[2,3′-bipyridin]-6-amine (579 mg, 2.34 mmol) was taken up in THE (20 mL) and the solution was cooled to 0° C. To the solution was added 1.0 M LHMDS in THE (4.68 mL, 4.68 mmol), then a solution of 6-fluoropyridine-2-sulfonyl chloride (int-a1) (915 mg, 4.68 mmol) that had been dissolved in THE (2 mL). After stirring overnight the reaction was quenched with sat. aq. NH₄Cl and extracted into EtOAc (10 mL×3). The organics were then washed with water and brine, dried over MgSO₄, and concentrated in vacuo. The crude material was purified by flash column chromatography (12 g silica gel column, 0-5% MeOH/DCM) to give N-(3-chloro-2′-isopropyl-[2,3′-bipyridin]-6-yl)-6-fluoropyridine-2-sulfonamide (int-b4) as a yellow solid: (716 mg, 1.76 mmol, 75% yield). LCMS (Condition 1): m/z 407.0 [M+H]⁺, 1.21 min. ¹H NMR (400 MHz, Methanol-d₄) δ 8.53 (dd, J=4.9, 1.8 Hz, 1H), 8.07-8.01 (m, 1H), 7.90-7.88 (m, 1H), 7.86 (d, J=8.8 Hz, 1H), 7.43 (dd, J=7.7, 1.8 Hz, 1H), 7.32-7.24 (m, 3H), 2.77-2.67 (m, 1H), 1.14 (br s, 3H), 1.03 (br s, 3H).

### Synthesis of Intermediate N-(6-([1,1′-biphenyl]-2-yl)-5-(trifluoromethyl)pyridin-2-yl)-6-fluoropyridine-2-sulfonamide (Int-b5)

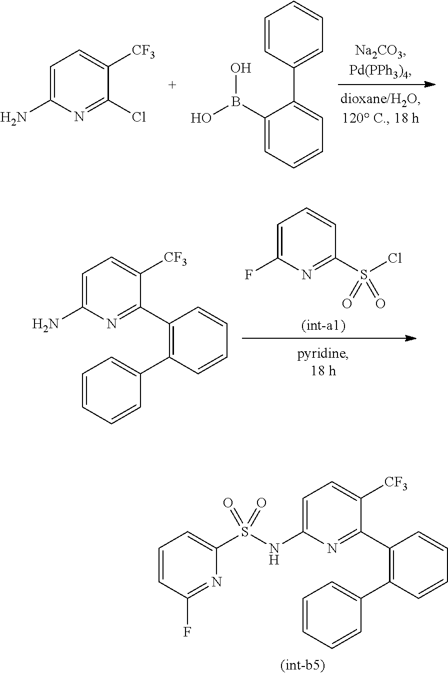

### Step 1. Synthesis of6-([1,1′-biphenyl]-2-yl)-5-(trifluoromethyl)pyridin-2-amine

In a vial, The 6-chloro-5-(trifluoromethyl)pyridin-2-amine (0.500 g, 2.54 mmol) and [1,1′-biphenyl]-2-ylboronic acid (0.756 g, 3.82 mmol) were dissolved in dioxane (20 mL) and water (3 mL) and treated with sodium carbonate (1.08 g, 10.2 mmol). The mixture was evacuated and backfilled using argon, Pd(PPh₃)₄ (0.294 g, 0.254 mmol) was added, and the mixture was evacuated and backfilled with argon again. After stirring at 120° C. for 18 h, the mixture was cooled, the aqueous layer was discarded, and enough ethyl acetate was added until all solids were dissolved. The resulting organics were dried over Na₂SO₄, filtered and concentrated to yield a reddish oil that was purified by silica gel chromatography (40 g silica gel column, 10-50% EtOAc/heptane) to yield the desired product 6-([1,1′-biphenyl]-2-yl)-5-(trifluoromethyl)pyridin-2-amine (0.660 g, 2.00 mmol, 78% yield). LCMS (Condition 1): m/z 315.1 [M+H]⁺, 1.40 min.

### Step 2. Synthesis of N-(6-([1,1′-biphenyl]-2-yl)-5-(trifluoromethyl)pyridin-2-yl)-6-fluoropyridine-2-sulfonamide (Int-b5)

In a vial, 6-([1,1′-biphenyl]-2-yl)-5-(trifluoromethyl)pyridin-2-amine (0.550 g, 1.75 mmol) and 6-fluoropyridine-2-sulfonyl chloride (int-a1) (0.377 g, 1.93 mmol) were dissolved in pyridine (5 mL). The resulting yellow solution was stirred for 18 hr. The reaction mixture was diluted with ethyl acetate, washed with water, sat. NH₄Cl, and brine, dried over Na₂SO₄ and concentrated. The oil obtained was treated with toluene (10 mL) and concentrated to yield a brown paste that was purified by silica gel chromatography (40 g silica gel column, 10-50% EtOAc/hexane) to afford N-(6-([1,1′-biphenyl]-2-yl)-5-(trifluoromethyl)pyridin-2-yl)-6-fluoropyridine-2-sulfonamide (int-b5): (0.330 g, 0.690 mmol, 39% yield). LCMS (Condition 1): m/z 474.1 [M+H]⁺, 1.75 min. ¹H NMR (400 MHz, Chloroform-d) δ 8.08-7.96 (m, 1H), 7.96-7.90 (m, 1H), 7.79 (d, J=9.1 Hz, 1H), 7.63-7.57 (m, 1H), 7.52-7.49 (m, 2H), 7.43 (d, J=7.6 Hz, 1H), 7.26-7.21 (m, 3H), 7.21-7.10 (m, 4H). ¹⁹F NMR (376 MHz, Chloroform-d) δ −58.09 (s, 3F), −63.94 (s, 1F).

### Synthesis of Intermediate N-(5-chloro-6-(2,6-dimethylphenyl)pyridin-2-yl)-6-fluoropyridine-2-sulfonamide (Int-b6)

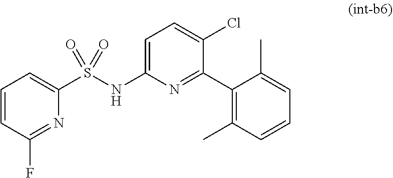 [SMILES: Cc1cccc(C)c1-c1nc(NS(=O)(=O)c2cccc(F)n2)ccc1Cl]

N-(5-chloro-6-(2,6-dimethylphenyl)pyridin-2-yl)-6-fluoropyridine-2-sulfonamide (int-b6) was synthesized using a procedure adapted from the one described in (int-b5), except in step 1, 6-chloro-5-(trifluoromethyl)pyridin-2-amine was replaced with 6-bromo-5-chloropyridin-2-amine and [1,1′-biphenyl]-2-ylboronic acid was replaced with (2,6-dimethylphenyl)boronic acid: LCMS (Condition 1): m/z 392.1 [M+H]⁺, 1.68 min.

### Synthesis of Intermediate N-(5-chloro-6-(2,6-dimethylphenyl)pyridin-2-yl)-2-fluoropyridine-4-sulfonamide (Int-b7)

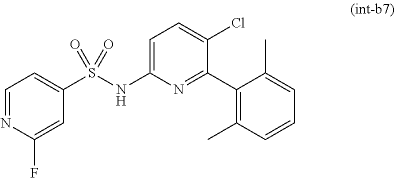 [SMILES: Cc1cccc(C)c1-c1nc(NS(=O)(=O)c2ccnc(F)c2)ccc1Cl]

N-(5-chloro-6-(2,6-dimethylphenyl)pyridin-2-yl)-2-fluoropyridine-4-sulfonamide (int-b7) was synthesized using a procedure adapted from the one described in (int-b2), except in step 1, 6-chloro-5-(trifluoromethyl)pyridin-2-amine was replaced with 6-bromo-5-chloropyridin-2-amine and (2-isopropylphenyl)boronic acid was replaced with (2,6-dimethylphenyl)boronic acid and in step 2, 6-fluoropyridine-2-sulfonyl chloride (int-a1) was replaced with 2-fluoropyridine-4-sulfonyl chloride (int-a13): LCMS (Condition 1): m/z 392.0 [M+H]⁺, 1.67 min.

### Synthesis of Intermediate N-(6-([1,1′-biphenyl]-2-yl)-5-chloropyridin-2-yl)-6-fluoropyridine-2-sulfonamide (Int-b8)

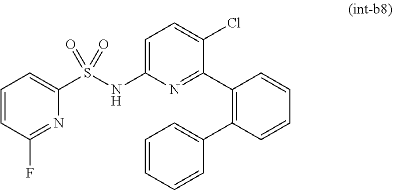 [SMILES: O=S(=O)(Nc1ccc(Cl)c(-c2ccccc2-c2ccccc2)n1)c1cccc(F)n1]

N-(6-([1,1′-biphenyl]-2-yl)-5-chloropyridin-2-yl)-6-fluoropyridine-2-sulfonamide (int-b8) was synthesized using a procedure adapted from the one described in (int-b5), except in step 1, 6-chloro-5-(trifluoromethyl)pyridin-2-amine was replaced with 6-bromo-5-chloropyridin-2-amine: LCMS (Condition 1): m/z 440.0 [M+H]⁺, 1.67 min.

### Synthesis of Intermediate N-(6-(2-chloro-5-methoxyphenyl)-5-(trifluoromethyl)pyridin-2-yl)-6-fluoropyridine-2-sulfonamide (Int-b9)

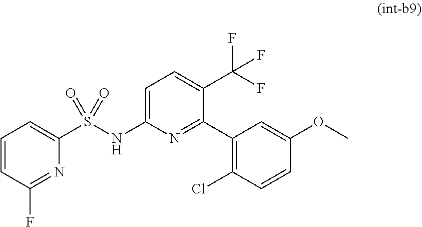 [SMILES: COc1ccc(Cl)c(-c2nc(NS(=O)(=O)c3cccc(F)n3)ccc2C(F)(F)F)c1]

N-(6-(2-chloro-5-methoxyphenyl)-5-(trifluoromethyl)pyridin-2-yl)-6-fluoropyridine-2-sulfonamide (int-b9) was synthesized using a procedure adapted from the one described in (int-b5), except in step 1, [1,1′-biphenyl]-2-ylboronic acid was replaced with (2-chloro-5-methoxyphenyl)boronic acid: LCMS (Condition 1): m/z 462.0 [M+H]⁺, 1.73 min.

### Synthesis of Intermediate 6-fluoro-N-(6-(5-fluoro-2-methylphenyl)-5-(trifluoromethyl)pyridin-2-yl)pyridine-2-sulfonamide (Int-b10)

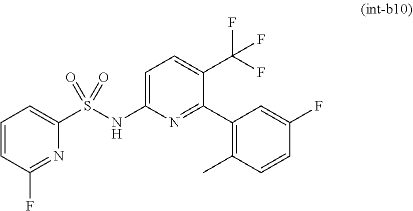 [SMILES: Cc1ccc(F)cc1-c1nc(NS(=O)(=O)c2cccc(F)n2)ccc1C(F)(F)F]

6-fluoro-N-(6-(5-fluoro-2-methylphenyl)-5-(trifluoromethyl)pyridin-2-yl)pyridine-2-sulfonamide (int-b10) was synthesized using a procedure adapted from the one described in (int-b5), except in step 1, [1,1′-biphenyl]-2-ylboronic acid was replaced with (5-fluoro-2-methylphenyl)boronic acid: LCMS (Condition 1): m/z 430.0 [M+H]⁺, 1.36 min.

### Synthesis of intermediate N-(6-(2-ethylphenyl)-5-(trifluoromethyl)pyridin-2-yl)-6-fluoropyridine-2-sulfonamide (Int-b11)

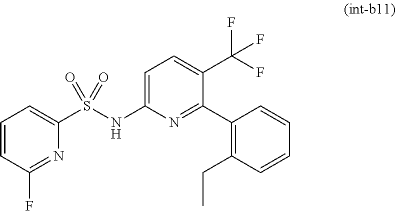 [SMILES: CCc1ccccc1-c1nc(NS(=O)(=O)c2cccc(F)n2)ccc1C(F)(F)F]

N-(6-(2-ethylphenyl)-5-(trifluoromethyl)pyridin-2-yl)-6-fluoropyridine-2-sulfonamide (int-b11) was synthesized using a procedure adapted from the one described in (int-b5), except in step 1, [1,1′-biphenyl]-2-ylboronic acid was replaced with (2-ethylphenyl)boronic acid: LCMS (Condition 1): m/z 426.0 [M+H]⁺, 1.64 min.

### Synthesis of Intermediate 6-fluoro-N-(6-(3-fluoro-2-methylphenyl)-5-(trifluoromethyl)pyridin-2-yl)pyridine-2-sulfonamide (Int-b12)

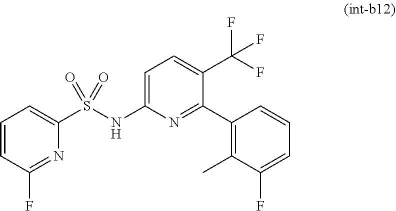 [SMILES: Cc1c(F)cccc1-c1nc(NS(=O)(=O)c2cccc(F)n2)ccc1C(F)(F)F]

6-fluoro-N-(6-(3-fluoro-2-methylphenyl)-5-(trifluoromethyl)pyridin-2-yl)pyridine-2-sulfonamide (int-b12) was synthesized using a procedure adapted from the one described in (int-b5), except in step 1, [1,1′-biphenyl]-2-ylboronic acid was replaced with (3-fluoro-2-methylphenyl)boronic acid, Pd(PPh₃)₄ was replaced with Pd(dba)2 and tri-tert-butylphosphine tetrafluoroborate, and sodium carbonate was replaced with potassium carbonate: LCMS (Condition 1): m/z 430.0 [M+H]⁺, 1.83 min.

### Synthesis of Intermediate N-(6-(2-cyclobutylphenyl)-5-(trifluoromethyl)pyridin-2-yl)-6-fluoropyridine-2-sulfonamide (Int-b13)

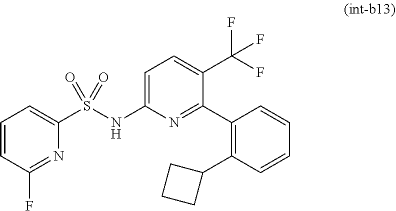 [SMILES: O=S(=O)(Nc1ccc(C(F)(F)F)c(-c2ccccc2C2CCC2)n1)c1cccc(F)n1]

N-(6-(2-cyclobutylphenyl)-5-(trifluoromethyl)pyridin-2-yl)-6-fluoropyridine-2-sulfonamide (int-b13) was synthesized using a procedure adapted from the one described in (int-b5), except in step 1, [1,1′-biphenyl]-2-ylboronic acid was replaced with (2-cyclobutylphenyl)boronic acid: LCMS (Condition 1): m/z 452.2 [M+H]⁺, 1.75 min.

### Synthesis of Intermediate N-(6-(2-(tert-butyl)phenyl)-5-(trifluoromethyl)pyridin-2-yl)-6-fluoropyridine-2-sulfonamide (Int-b14)

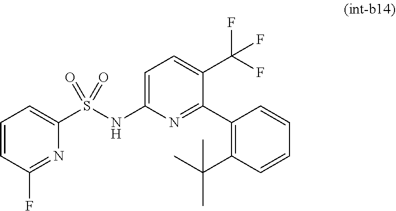 [SMILES: CC(C)(C)c1ccccc1-c1nc(NS(=O)(=O)c2cccc(F)n2)ccc1C(F)(F)F]

N-(6-(2-(tert-butyl)phenyl)-5-(trifluoromethyl)pyridin-2-yl)-6-fluoropyridine-2-sulfonamide (int-b14) was synthesized using a procedure adapted from the one described in (int-b5), except in step 1, [1,1′-biphenyl]-2-ylboronic acid was replaced with (2-(tert-butyl)phenyl)boronic acid: LCMS (Condition 1): m/z 454.2 [M+H]⁺, 1.75 min.

### Synthesis of Intermediate 6-fluoro-N-(6-(2-(pyrimidin-2-yl)phenyl)-5-(trifluoromethyl)pyridin-2-yl)pyridine-2-sulfonamide (Int-b15)

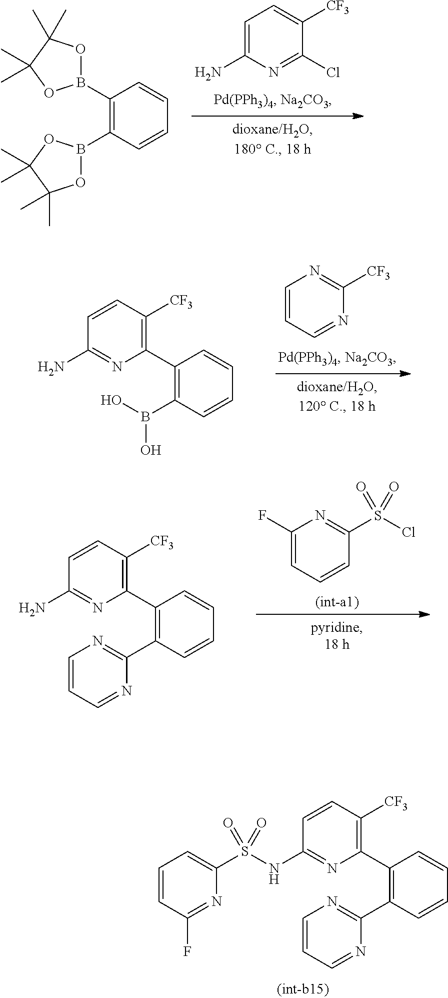

### Step 1. Synthesis of (2-(6-amino-3-(trifluoromethyl)pyridin-2-yl)phenyl)boronic Acid

In a flask, 6-chloro-5-(trifluoromethyl)pyridin-2-amine (1.00 g, 5.09 mmol) and 1,2-bis(4,4,5,5-tetramethyl-1,3,2-dioxaborolan-2-yl)benzene (3.02 g, 9.16 mmol) were dissolved in dioxane (20 mL) and water (3 mL) and treated with sodium carbonate (1.08 g, 10.2 mmol). The mixture was evacuated and backfilled with argon, Pd(PPh₃)₄ (0.588 g, 0.509 mmol) was added, and the mixture was evacuated and backfilled with argon again. The mixture was stirred at 120° C. for 18 h. The mixture was cooled, the aqueous layer was discarded and the dioxane mixture was filtered. The solids were washed with more dioxane, and the combined dioxane filtrates were washed with brine and dried over Na₂SO₄, filtered and concentrated to yield a reddish oil. The crude product was purified by silica gel chromatography (120 g silica gel column, 0-100% EtOAc/EtOH (3:1) mixture in heptane followed by a methanol wash) to provide the desired product (2-(6-amino-3-(trifluoromethyl)pyridin-2-yl)phenyl)boronic acid, as a mixture of boronic acid and anhydride(s) in ˜1:1 ratio. Condition 1, LCMS: m/z=283.2 [M+H]⁺, 1.12 min. ¹H NMR (400 MHz, DMSO-d6) δ 7.96 (d, J=9.1 Hz, 1H), 7.91-7.82 (m, 2H), 7.69 (d, J=7.7 Hz, 1H), 7.52 (d, J=7.0 Hz, 1H), 7.47 (d, J=6.9 Hz, 1H), 7.45-7.28 (m, 5H), 6.74 (d, J=9.0 Hz, 1H), 6.63 (d, J=8.9 Hz, 1H), 4.54 (d, J=5.9 Hz, 1H), 3.95 (s, 1H).

### Step 2. Synthesis of 6-(2-(pyrimidin-2-yl)phenyl)-5-(trifluoromethyl)pyridin-2-amine

In a vial, 2-bromopyrimidine (0.073 g, 0.46 mmol) and (2-(6-amino-3-(trifluoromethyl)pyridin-2-yl)phenyl)boronic acid (0.10 g, 0.36 mmol) were dissolved in dioxane (5 mL) and water (0.75 mL) and treated with sodium carbonate (0.15 g, 1.4 mmol). The mixture was evacuated and backfilled with argon, Pd(PPh₃)₄ (0.041 g, 0.035 mmol) was added, the mixture was evacuated and backfilled with argon again, and was subsequently stirred at 120° C. for 18 h. The mixture was cooled, the aqueous layer was discarded and the mixture was filtered. The solids were washed with more dioxane. The combined filtrate was dried over Na₂SO₄, filtered and concentrated to yield a reddish oil. The crude product was purified by silica gel chromatography (12 g silica gel column, 0-100% EtOAc/heptane) to give the product 6-(2-(pyrimidin-2-yl)phenyl)-5-(trifluoromethyl)pyridin-2-amine. Condition 1, LCMS: m/z=317.2 [M+H]⁺, 1.16 min. ¹H NMR (400 MHz, Chloroform-d) δ 8.57 (d, J=4.8 Hz, 2H), 8.35-8.21 (m, 1H), 7.99-7.85 (m, 1H), 7.72-7.66 (m, 4H), 7.62-7.53 (m, 3H), 7.51-7.46 (m, 3H), 7.43-7.30 (m, 2H), 7.04-7.02 (m, 1H), 6.47 (d, J=8.6 Hz, 1H), 4.74 (s, 2H). ¹⁹F NMR (376 MHz, Chloroform-d) δ −57.73 (s, 1F).

### Step 3. Synthesis of 6-fluoro-N-(6-(2-(pyrimidin-2-yl)phenyl)-5-(trifluoromethyl)pyridin-2-yl)pyridine-2-sulfonamide (Int-b15)

In a vial, 6-(2-(pyrimidin-2-yl)phenyl)-5-(trifluoromethyl)pyridin-2-amine (60.0 mg, 0.190 mmol) and 6-fluoropyridine-2-sulfonyl chloride (48.2 mg, 0.247 mmol) were dissolved in pyridine (3 mL). The resulting solution was stirred for 18 h. The reaction mixture was diluted with ethyl acetate, washed with water, sat. NH₄Cl, and brine, dried over Na₂SO₄ and concentrated. The oil obtained was treated with toluene (10 mL) and concentrated to yield a brown paste which was taken on directly to the next reaction. Condition 1, LCMS: m/z=476.0 [M+H]⁺, 1.58 min.

### Synthesis of Intermediate 6-fluoro-N-(6-(2-(pyridin-4-yl)phenyl)-5-(trifluoromethyl)pyridin-2-yl)pyridine-2-sulfonamide (Int-b16)

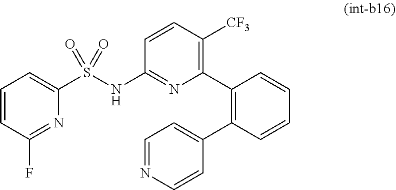 [SMILES: O=S(=O)(Nc1ccc(C(F)(F)F)c(-c2ccccc2-c2ccncc2)n1)c1cccc(F)n1]

6-fluoro-N-(6-(2-(pyridin-4-yl)phenyl)-5-(trifluoromethyl)pyridin-2-yl)pyridine-2-sulfonamide (int-b16) was synthesized using the procedure described for intermediate (int-b15) except in step 2, 2-bromopyrimidine was replaced with 4-bromopyridine: LCMS (Condition 1): m/z 475.1 [M+H]⁺, 1.31 min.

### Synthesis of Intermediate 6-fluoro-N-(6-(2-phenoxyphenyl)-5-(trifluoromethyl)pyridin-2-yl)pyridine-2-sulfonamide (Int-b17)

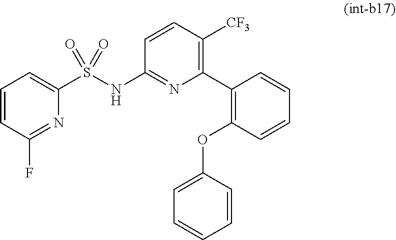 [SMILES: O=S(=O)(Nc1ccc(C(F)(F)F)c(-c2ccccc2Oc2ccccc2)n1)c1cccc(F)n1]

6-fluoro-N-(6-(2-phenoxyphenyl)-5-(trifluoromethyl)pyridin-2-yl)pyridine-2-sulfonamide (int-b17) was synthesized using a procedure adapted from the one described in (int-b5), except in step 1, [1,1′-biphenyl]-2-ylboronic acid was replaced with (2-phenoxyphenyl)boronic acid: LCMS (Condition 1): m/z 490.2 [M+H]⁺, 1.84 min.

### Synthesis of Intermediate 6-fluoro-N-(4′-isopropyl-3-(trifluoromethyl)-[2,3′-bipyridin]-6-yl)pyridine-2-sulfonamide (Int-b18)

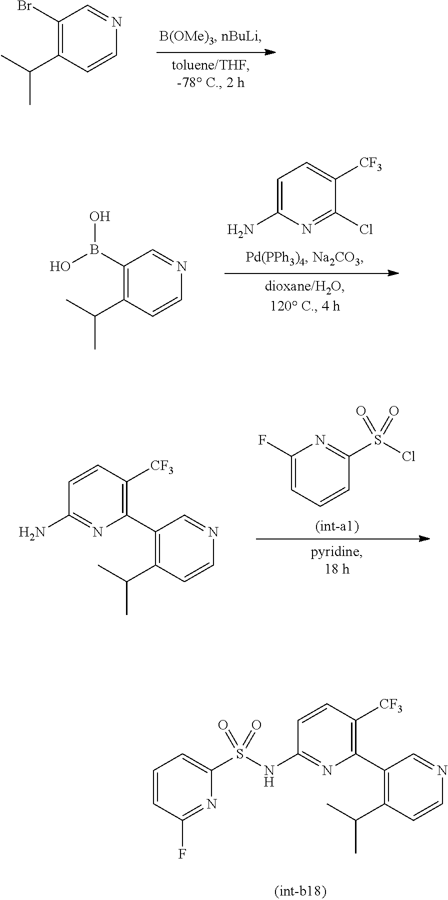

### Step 1. Synthesis of (4-isopropylpyridin-3-yl)boronic Acid

A solution of 3-bromo-4-isopropylpyridine (300 mg, 1.50 mmol) and B(OMe)₃ (203 mg, 1.95 mmol) in toluene (4 mL) and THE (1 mL) was cooled to −78° C. and nBuLi was added dropwise. After 2 hours, the reaction was allowed to warm to 0° C. and the mixture was then treated with 3 M HCl (5 mL) and stirred at room temperature for 10 minutes. The mixture was extracted with diethyl ether, and the ether layer was set aside. The aqueous layer was adjusted to ˜pH 7 with 1 M NaOH and extracted with EtOAc. The combined organics were dried with Na₂SO₄, filtered, and concentrated to afford the crude product (4-isopropylpyridin-3-yl)boronic acid (161 mg, 0.781 mmol, 52% yield) that was taken on directly to the next reaction. LCMS (Condition 1): m/z 166.2 [M+H]⁺, 0.57 min.

### Step 2. Synthesis of 4′-isopropyl-3-(trifluoromethyl)-[2,3′-bipyridin]-6-amine

In a vial, the 6-chloro-5-(trifluoromethyl)pyridin-2-amine (150 mg, 0.763 mmol) and (4-isopropylpyridin-3-yl)boronic acid (157 mg, 0.954 mmol) were dissolved in dioxane (6 mL) and water (1 mL) and treated with sodium carbonate (324 mg, 3.05 mmol). The mixture was evacuated and backfilled using argon, Pd(PPh₃)₄ (88 mg, 0.076 mmol) was added, and the mixture was evacuated and backfilled with argon again. After stirring at 120° C. for 4 h, the mixture was cooled, diluted with water, and extracted with EtOAc. The resulting organics were washed with brine, dried over Na₂SO₄, filtered, concentrated, and purified by silica gel chromatography to yield the desired product 4′-isopropyl-3-(trifluoromethyl)-[2,3′-bipyridin]-6-amine (160 mg, 0.54 mmol, 71% yield). LCMS (Condition 1): m/z 282.1 [M+H]⁺, 1.27 min.

### Step 3. Synthesis of 6-fluoro-N-(4′-isopropyl-3-(trifluoromethyl)-[2,3′-bipyridin]-6-yl)pyridine-2-sulfonamide (Int-b18)

In a vial, 4′-isopropyl-3-(trifluoromethyl)-[2,3′-bipyridin]-6-amine (160 mg, 0.57 mmol) and 6-fluoropyridine-2-sulfonyl chloride (int-a1) (145 mg, 0.74 mmol) were dissolved in pyridine (6 mL) and stirred for 5 days. The reaction mixture was concentrated and purified by silica gel chromatography to afford 6-fluoro-N-(4′-isopropyl-3-(trifluoromethyl)-[2,3′-bipyridin]-6-yl)pyridine-2-sulfonamide (int-b18): (70 mg, 0.16 mmol, 28% yield). LCMS (Condition 1): m/z 441.1 [M+H]⁺, 1.35 min.

### Synthesis of Intermediate N-(6-(2-cyclopropylphenyl)-5-(trifluoromethyl)pyridin-2-yl)-6-fluoropyridine-2-sulfonamide (Int-b19)

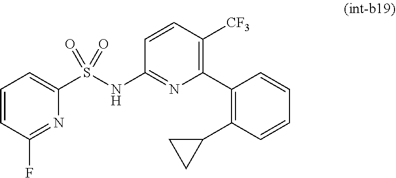 [SMILES: O=S(=O)(Nc1ccc(C(F)(F)F)c(-c2ccccc2C2CC2)n1)c1cccc(F)n1]

N-(6-(2-cyclopropylphenyl)-5-(trifluoromethyl)pyridin-2-yl)-6-fluoropyridine-2-sulfonamide (int-b19) was synthesized using a procedure adapted from the one described in (int-b2), except in step 1, (2-isopropylphenyl)boronic acid was replaced with (2-cyclopropylphenyl)boronic acid: LCMS (Condition 1): m/z 438.1 [M+H]⁺, 1.73 min.

### Synthesis of Intermediate 6-fluoro-N-(5-(trifluoromethyl)-6-(2-vinylphenyl)pyridin-2-yl)pyridine-2-sulfonamide (Int-b20)

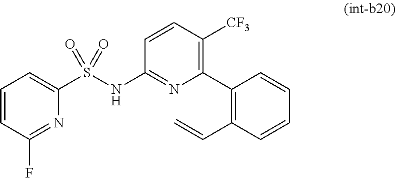 [SMILES: C=Cc1ccccc1-c1nc(NS(=O)(=O)c2cccc(F)n2)ccc1C(F)(F)F]

6-fluoro-N-(5-(trifluoromethyl)-6-(2-vinylphenyl)pyridin-2-yl)pyridine-2-sulfonamide (int-b20) was synthesized using a procedure adapted from the one described in (int-b5), except in step 1, [1,1′-biphenyl]-2-ylboronic acid was replaced with (2-vinylphenyl)boronic acid: LCMS (Condition 1): m/z 424.1 [M+H]⁺, 1.83 min.

### Synthesis of Intermediate N-(6-(2-cyclopropyl-5-fluorophenyl)-5-(trifluoromethyl)pyridin-2-yl)-6-fluoropyridine-2-sulfonamide (Int-b21)

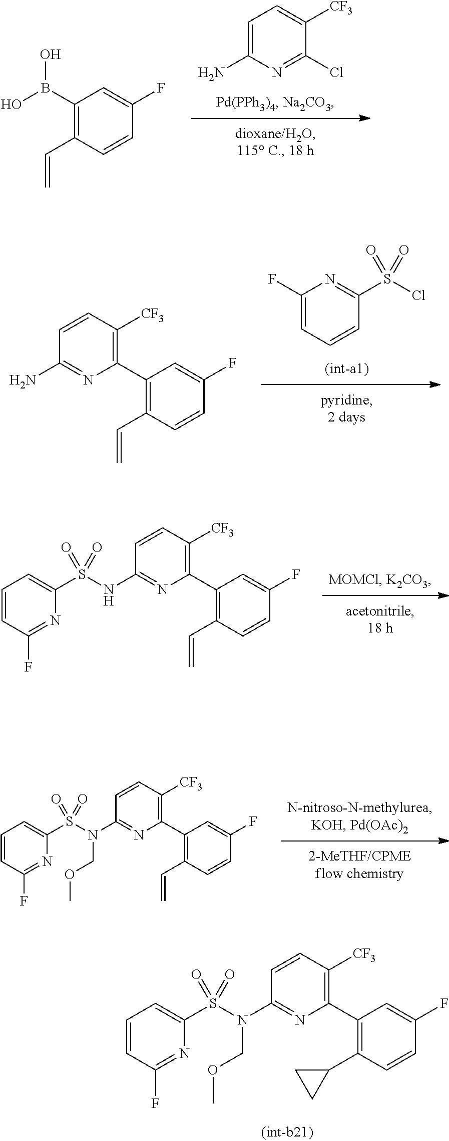

### Step 1. Synthesis of 6-(5-fluoro-2-vinylphenyl)-5-(trifluoromethyl)pyridin-2-amine

The 6-chloro-5-(trifluoromethyl)pyridin-2-amine (8.00 g, 40.7 mmol) and (5-fluoro-2-vinylphenyl)boronic acid (7.97 g, 48.0 mmol) were distributed evenly among 4 40 mL vials and to each vial was added dioxane (20 mL) and water (3 mL) and treated with one quarter of the total amount of sodium carbonate (15.1 g, 142 mmol). The mixtures were evacuated and backfilled using argon, one quarter of the total amount of Pd(PPh₃)₄ (3.76 g, 3.26 mmol) was added to each vial, and the mixtures were evacuated and backfilled with argon again. After stirring at 115° C. for 18 h, the mixtures were cooled, combined together, filtered, and the solids washed with dioxane. The resulting organics were concentrated and purified by silica gel chromatography (330 g silica gel column, 10-35% EtOAc/heptane) to yield the desired product 6-(5-fluoro-2-vinylphenyl)-5-(trifluoromethyl)pyridin-2-amine (10.1 g, 35.4 mmol, 87% yield). LCMS (Condition 1): m/z 283.0 [M+H]⁺, 1.46 min.

### Step 2. Synthesis of 6-fluoro-N-(6-(5-fluoro-2-vinylphenyl)-5-(trifluoromethyl)pyridin-2-yl)pyridine-2-sulfonamide

In a flask, 6-(5-fluoro-2-vinylphenyl)-5-(trifluoromethyl)pyridin-2-amine (10.1 g, 35.4 mmol) and 6-fluoropyridine-2-sulfonyl chloride (int-a1) (9.09 g, 46.5 mmol) were dissolved in pyridine (60 mL). The resulting solution was stirred for 2 days. The reaction mixture was quenched with 1 M HCl and extracted with EtOAc (×3). The organics were then dried over Na₂SO₄ and concentrated. The oil obtained was treated with toluene (70 mL) and concentrated to yield a brown paste which was then taken up in DCM (30 mL). The resulting precipitate was collected by filtration, washed with DCM, and air dried to give the desired product 6-fluoro-N-(6-(5-fluoro-2-vinylphenyl)-5-(trifluoromethyl)pyridin-2-yl)pyridine-2-sulfonamide (10.6 g, 23.8 mmol, 67% yield) as a gray powder. Condition 1, LCMS: m/z=441.9 [M+H]⁺, 1.73 min.

### Step 3. Synthesis of 6-fluoro-N-(6-(5-fluoro-2-vinylphenyl)-5-(trifluoromethyl)pyridin-2-yl)-N-(methoxymethyl)pyridine-2-sulfonamide

In a vial, 6-fluoro-N-(6-(5-fluoro-2-vinylphenyl)-5-(trifluoromethyl)pyridin-2-yl)pyridine-2-sulfonamide (430 mg, 0.97 mmol) was dissolved in acetonitrile (3 mL) and treated with K₂CO₃ (337 mg, 2.44 mmol), and after stirring for 15 min, MOMCI (0.096 mL, 1.27 mmol) was added. The resulting mixture was stirred for 18 hours, then filtered and concentrated. The resulting residue was then purified by flash column chromatography (12 g silica gel column, 0-30% EtOAc/heptane) to give the desired product 6-fluoro-N-(6-(5-fluoro-2-vinylphenyl)-5-(trifluoromethyl)pyridin-2-yl)-N-(methoxymethyl)pyridine-2-sulfonamide. Condition 1, LCMS: m/z=486.0 [M+H]⁺, 1.36 min.

### Step 4. Synthesis of N-(6-(2-cyclopropyl-5-fluorophenyl)-5-(trifluoromethyl)pyridin-2-yl)-6-fluoro-N-(methoxymethyl)pyridine-2-sulfonamide (Int-b21)

A Vapourtec R-Series flow reactor system equipped with PFA (perfluoroalkoxy) tubing and Zaiput liquid-liquid separator was utilized. System parameters: System solvent pump A—2MeTHF, Reagent A: N-nitroso-N-methylurea (NMU) (2.58 mL, 1.03 mmol, 0.4 M in 2MeTHF/CPME, 3:7), pump A flow rate 1.786 mL/min; system solvent pump B—H₂O, Reagent B: KOH (0.82 mL, 1.24 mmol, 1.5 M in H₂O), pump B flow rate 0.714 mL/min. The solution of NMU and KOH were mixed in a cooled reactor (10° C., 2 mL) for 0.8 min. The aqueous phase was separated by a Zaiput liquid-liquid phase separator. Both the aqueous and organic lines were held under pressure with 4 and 4.5 bar back pressure regulators, respectively. The organic stream was collected into a stirring solution of Pd(OAc)₂ (4.6 mg, 0.021 mmol) and 6-fluoro-N-(6-(5-fluoro-2-vinylphenyl)-5-(trifluoromethyl)pyridin-2-yl)-N-(methoxymethyl)pyridine-2-sulfonamide (200 mg, 0.412 mmol) in THE (0.5 M). The reaction mixture was stirred for 10 min and subsequently quenched with AcOH. The mixture was then filtered and concentrated to give the crude product N-(6-(2-cyclopropyl-5-fluorophenyl)-5-(trifluoromethyl)pyridin-2-yl)-6-fluoro-N-(methoxymethyl)pyridine-2-sulfonamide (int-b21) that was used without further purification. Condition 1, LCMS: m/z=500.0 [M+H]⁺, 1.70 min.

### Synthesis of Intermediate N-(5-chloro-6-(o-tolyl)pyridin-2-yl)-6-fluoropyridine-2-sulfonamide (Int-b22)

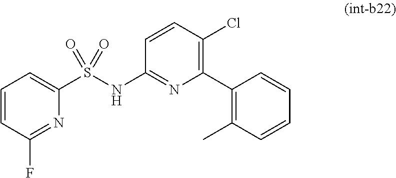 [SMILES: Cc1ccccc1-c1nc(NS(=O)(=O)c2cccc(F)n2)ccc1Cl]

N-(5-chloro-6-(o-tolyl)pyridin-2-yl)-6-fluoropyridine-2-sulfonamide (int-b22) was synthesized using a procedure adapted from the one described in (int-b2), except in step 1, 6-chloro-5-(trifluoromethyl)pyridin-2-amine was replaced with 6-bromo-5-chloropyridin-2-amine, (2-isopropylphenyl)boronic acid was replaced with o-tolylboronic acid, Pd(PPh₃)₄ was replaced with Pd(dba)2 and tri-tert-butylphosphine tetrafluoroborate, and sodium carbonate was replaced with potassium carbonate: LCMS (Condition 1): m/z 378.1 [M+H]⁺, 1.55 min.

### Synthesis of Intermediate 2-bromo-N-(5-chloro-6-(2,6-dimethylphenyl)pyridin-2-yl)pyridine-4-sulfonamide (Int-b23)

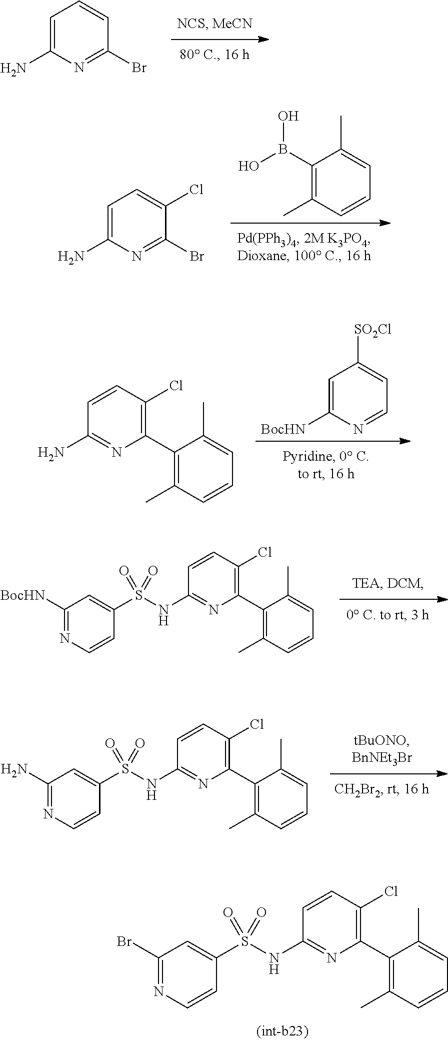

### Step 1. Synthesis of 6-bromo-5-chloropyridin-2-amine

A solution of 6-bromopyridin-2-amine (20.0 g, 115.60 mmol) and N-chlorosuccinimide (15.43 g, 115.60 mmol) in acetonitrile (250 mL) was heated at 80° C. for 16 h. Reaction mixture was cooled, concentrated under reduced pressure. The residue was diluted with sat. NaHCO₃ solution (100 mL) and extracted with EtOAc thrice. The combined organic portion was washed with brine, dried over anhydrous Na₂SO₄, and concentrated in vacuo. The crude product mass was stirred with hexane, the solid separated was collected by filtration and dried in vacuo to afford 6-bromo-5-chloropyridin-2-amine as an off white solid (16.0 g, 61%). LCMS: m/z 210.9 [M+H]⁺, 1.48 min.

### Step 2: Synthesis of 5-chloro-6-(2,6-dimethylphenyl)pyridin-2-amine

To a stirred solution of 6-bromo-5-chloropyridin-2-amine (5.0 g, 24.10 mmol) in dioxane (60 mL) were added (2,6-dimethylphenyl)boronic acid (5.42 g, 36.15 mmol) and 2 M K₃PO₄ solution (36.3 mL) under N₂. Then Pd(PPh₃)₄ (2.8 g, 2.41 mmol) was added, degassed and the reaction mixture was stirred at 100° C. for 16 h. Then the reaction mixture was cooled to rt, diluted with water and extracted with EtOAc twice. Combined organic portion was washed with brine solution, dried over anhydrous Na₂SO₄ and concentrated. The residue was purified by flash column chromatography (12 g SiliCycle column, 14% EtOAc in hexane elution) to afford 5-chloro-6-(2,6-dimethylphenyl)pyridin-2-amine as an off white solid (2.7 g, 48%). LCMS: m/z 233.2 [M+H]⁺, 1.18 min. ¹H NMR (300 MHz, CDCl₃) δ 7.52 (d, J=8.4 Hz, 1H), 7.21-7.17 (m, 1H), 7.10-7.08 (m, 2H), 6.47 (d, J=8.7 Hz, 1H), 4.54 (brs, 2H), 2.06 (s, 3H).

### Step 3: Synthesis of tert-butyl (4-(N-(5-chloro-6-(2,6-dimethylphenyl)pyridin-2-yl)sulfamoyl)pyridin-2-yl)carbamate

The solution of 5-chloro-6-(2,6-dimethylphenyl)pyridin-2-amine (2.7 g, 11.60 mmol) in pyridine (45 mL) was cooled to −10° C. and tert-butyl (4-(chlorosulfonyl)pyridin-2-yl)carbamate (6.79 g, 23.20 mmol) was added portion wise. Reaction mixture was allowed to stir at rt for 16 h. The reaction mixture was concentrated under reduced pressure and was partioned between DCM and water. The organic portion was separated, over anhydrous Na₂SO₄ and concentrated in vacuo. The crude product was purified by flash chromatography (12 g SiliCycle column, 3-5% MeOH in CHCl₃ elution) to provide by tert-butyl (4-(N-(5-chloro-6-(2,6-dimethylphenyl)pyridin-2-yl)sulfamoyl)pyridin-2-yl)carbamate as a pale yellow solid (4.0 g, 70.5%). LCMS: m/z 489.0 [M+H]⁺, 1.66 min.

### Step 4: Synthesis of 2-amino-N-(5-chloro-6-(2,6-dimethylphenyl)pyridin-2-yl)pyridine-4-sulfonamide

TFA (20 mL) was added to the stirred solution of tert-butyl (4-(N-(5-chloro-6-(2,6-dimethylphenyl)pyridin-2-yl)sulfamoyl)pyridin-2-yl)carbamate (4.0 g, 8.19 mmol) in DCM (100 mL) at 0° C. and stirred at rt for 3 h. The reaction mixture was concentrated and the residue was partitioned between DCM and saturated NaHCO₃ solution. The combined organic portion was washed with brine solution, dried over anhydrous Na₂SO₄ and concentrated in vacuo to provide 2-amino-N-(5-chloro-6-(2,6-dimethylphenyl)pyridin-2-yl)pyridine-4-sulfonamide as a pale yellow solid (3.2 g crude). LCMS: m/z 387.4 [M−H]*, 1.44 min.

### Step 5: Synthesis of 2-bromo-N-(5-chloro-6-(2,6-dimethylphenyl)pyridin-2-yl)pyridine-4-sulfonamide (Int-b23)

To the solution of 2-amino-N-(5-chloro-6-(2,6-dimethylphenyl)pyridin-2-yl)pyridine-4-sulfonamide (3.2 g, 8.23 mmol) and benzyltriethylammoniumbromide (3.36 g, 12.34 mmol) in CH₂Br₂ (50 mL), t-butyl nitrite (1.27 g, 12.34 mmol) was added dropwise and stirred at rt for 16 h. Reaction mixture diluted with DCM, washed with water followed by brine. The combined organic portion was washed with brine solution, dried over anhydrous Na₂SO₄ and concentrated in vacuo. The crude product was purified by flash chromatography (12 g SiliCycle column, 25-30% EtOAc in hexane elution) to provide by 2-bromo-N-(5-chloro-6-(2,6-dimethylphenyl)pyridin-2-yl)pyridine-4-sulfonamide (int-b23) as a pale yellow solid (1.15 g, 31% over two steps). LCMS: m/z 453.9 [M+H]⁺, 1.64 min. ¹H NMR (400 MHz, CDCl₃) δ 8.49 (d, J=5.6 Hz, 1H), 7.86 (d, J=1.2 Hz, 1H), 7.81 (d, J=8.8 Hz, 1H), 7.62 (dd, J=4.8, 1.2 Hz, 1H), 7.28-7.23 (m, 2H), 7.12 (d, J=7.6 Hz, 2H), 1.93 (s, 6H).

### Synthesis of Intermediate 2-bromo-N-(5-chloro-6-(5-fluoro-2-methylphenyl)pyridin-2-yl)pyridine-4-sulfonamide (Int-b24)

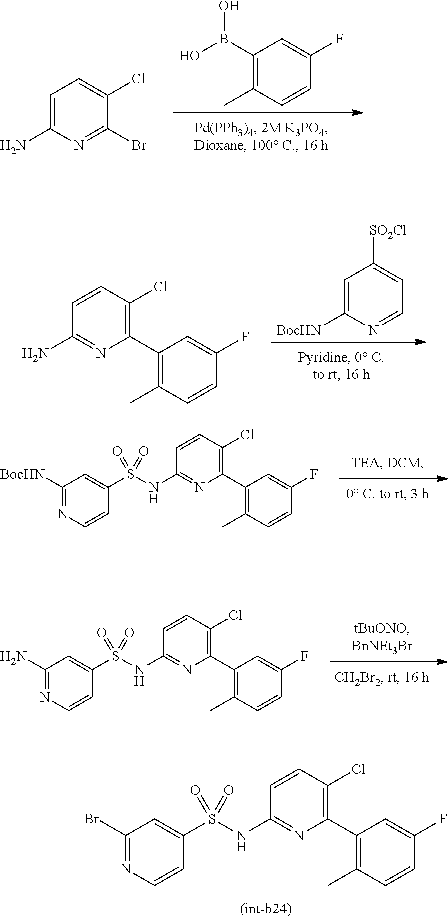

### Step 1. 5-chloro-6-(5-fluoro-2-methylphenyl)pyridin-2-amine

To a stirred solution of 6-bromo-5-chloropyridin-2-amine (7.5 g, 36.15 mmol) in dioxane (120 mL) were added (5-fluoro-2-methylphenyl)boronic acid (6.7 g, 43.38 mmol) and 2 M K₃PO₄ solution (55 mL) under N₂. Then Pd(PPh₃)₄ (4.2 g, 3.62 mmol) was added, degassed and the reaction mixture was stirred at 100° C. for 16 h. The reaction mixture was cooled to rt, filtered through a celite bed, which was thoroughly washed with EtOAc. The organic portion was separated and washed with 10% HCl solution thrice. The combined (acidic) aqueous portion was basified with saturated NaHCO₃ solution and extracted with EtOAc thrice. The combined EtOAc extract was washed with brine solution, dried over anhydrous Na₂SO₄ and concentrated. The residue was stirred with hexane, filtered and solid collected was dried in vacuo to yield 5-chloro-6-(5-fluoro-2-methylphenyl)pyridin-2-amine as an off white solid (7.2 g, 84%). LCMS: m/z 237.0 [M+H]⁺, 1.47 min. ¹H NMR (300 MHz, CDCl₃) δ 7.50 (d, J=8.7 Hz, 1H), 7.24-7.19 (m, 1H), 7.03-6.94 (m, 2H), 6.49 (d, J=8.7 Hz, 1H), 4.55 (brs, 2H), 2.15 (s, 3H).

### Step 2: Synthesis of tert-butyl (4-(N-(5-chloro-6-(5-fluoro-2-methylphenyl)pyridin-2-yl)sulfamoyl)pyridin-2-yl)carbamate

The solution of 5-chloro-6-(5-fluoro-2-methylphenyl)pyridin-2-amine (3.3 g, 13.94 mmol) in pyridine (50 mL) was cooled to −10° C. and tert-butyl (4-(chlorosulfonyl)pyridin-2-yl)carbamate (8.16 g, 27.88 mmol) was added portion wise. Reaction mixture was allowed to stir at rt for 16 h. The reaction mixture was concentrated under reduced pressure and was partioned between CH₂Cl₂ and water. The organic portion was separated, over anhydrous Na₂SO₄ and concentrated in vacuo. The crude product was purified by flash chromatography (12 g SiliCycle column, 3-5% MeOH in CHCl₃ elution) to provide by tert-butyl (4-(N-(5-chloro-6-(5-fluoro-2-methylphenyl)pyridin-2-yl)sulfamoyl)pyridin-2-yl)carbamate as a pale yellow solid (5.0 g, 72.7%). LCMS: m/z 493.0 [M+H]⁺, 1.65 min.

### Step 3: Synthesis of 2-amino-N-(5-chloro-6-(5-fluoro-2-methylphenyl)pyridin-2-yl)pyridine-4-sulfonamide (Int-b24)

TFA (25 mL) was added to the stirred solution of tert-butyl (4-(N-(5-chloro-6-(5-fluoro-2-methylphenyl)pyridin-2-yl)sulfamoyl)pyridin-2-yl)carbamate (5.0 g, 10.14 mmol) in DCM (100 mL) at 0° C. and stirred at rt for 3 h. On completion the reaction mixture was concentrated and the residue was partitioned between DCM and saturated NaHCO₃ solution. The combined organic portion was washed with brine solution, dried over anhydrous Na₂SO₄ and concentrated in vacuo to provide 2-amino-N-(5-chloro-6-(5-fluoro-2-methylphenyl)pyridin-2-yl)pyridine-4-sulfonamide (int-b24) as a pale yellow solid (4.0 g crude). LCMS: m/z 393.0 [M+H]⁺, 1.44 min.

### Synthesis of Intermediate N-(6-(2,6-dimethylphenyl)-5-(trifluoromethyl)pyridin-2-yl)-6-fluoropyridine-2-sulfonamide (Int-b25)

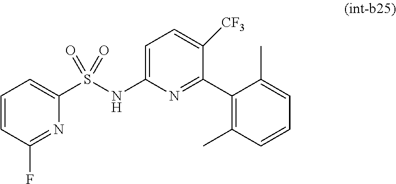 [SMILES: Cc1cccc(C)c1-c1nc(NS(=O)(=O)c2cccc(F)n2)ccc1C(F)(F)F]

N-(6-(2,6-dimethylphenyl)-5-(trifluoromethyl)pyridin-2-yl)-6-fluoropyridine-2-sulfonamide (int-b25) was synthesized using a procedure adapted from the one described in (int-b5), except in step 1, [1,1′-biphenyl]-2-ylboronic acid was replaced with (2,6-dimethylphenyl)boronic acid: LCMS (Condition 1): m/z 425.9 [M+H]⁺, 1.69 min.

### Synthesis of Examplary Compounds

### Example 1: 3-((6-(N-(5-chloro-6-(2,6-dimethylphenyl)pyridin-2-yl)sulfamoyl)pyridin-2-yl)amino)propanoic Acid (1)

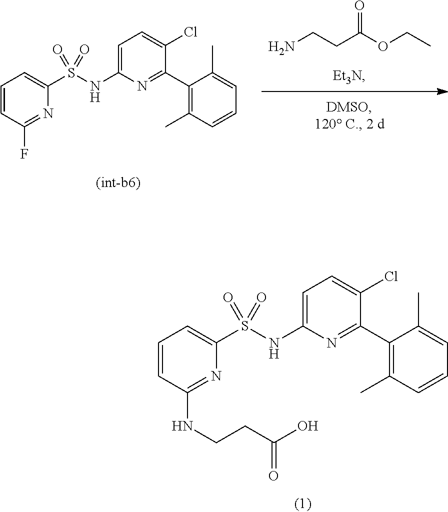

In a sealed reaction vessel, N-(5-chloro-6-(2,6-dimethylphenyl)pyridin-2-yl)-6-fluoropyridine-2-sulfonamide (int-b6) (60 mg, 0.15 mmol), ethyl 3-aminopropanoate hydrogen chloride (70 mg, 0.46 mmol), and triethylamine (120 mg, 1.2 mmol) were taken up in DMSO and heated to 120° C. with stirring for 48 hours. The reaction was cooled to room temperature and 1 M HCl was added to make the mixture pH ˜3 and the mixture was then purified by preparatory HPLC. The product was collected and solvent was removed under reduced pressure, and the resulting solid was dissolved in DCM. The organics were then washed with NaHCO₃ solution, 1 M HCl solution, dried over sodium sulfate, filtered, and solvent was removed under reduced pressure providing 3-((6-(N-(5-chloro-6-(2,6-dimethylphenyl)pyridin-2-yl)sulfamoyl)pyridin-2-yl)amino)propanoic acid (1). LCMS (Condition 1): m/z 461.2 [M+H]⁺, 1.59 min. ¹H NMR (400 MHz, DMSO-d₆) δ 12.14 (s, 1H), 11.16 (s, 1H), 7.92 (d, J=8.5 Hz, 1H), 7.53 (dd, J=7.2, 8.5 Hz, 1H), 7.41 (dd, J=6.8, 8.0 Hz, 2H), 7.21 (d, J=7.6 Hz, 2H), 7.08 (dd, J=0.8, 7.6 Hz, 1H), 7.02 (d, J=8 Hz, 1H), 6.67 (dd, J=0.8, 8.5 Hz, 1H), 3.31 (q, J=6.5 Hz, 2H), 2.35 (t, J=6.8 Hz, 2H), 1.79 (s, 6H).

### Example 2: 3-((4-(N-(5-chloro-6-(2,6-dimethylphenyl)pyridin-2-yl)sulfamoyl)pyridin-2-yl)amino)propanoic Acid (2)

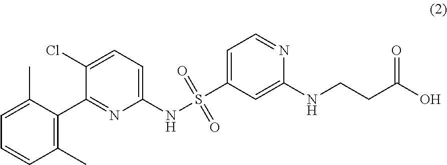 [SMILES: Cc1cccc(C)c1-c1nc(NS(=O)(=O)c2ccnc(NCCC(=O)O)c2)ccc1Cl]

3-((4-(N-(5-chloro-6-(2,6-dimethylphenyl)pyridin-2-yl)sulfamoyl)pyridin-2-yl)amino)propanoic acid (2) was synthesized using the procedure described in Example 1, except N-(5-chloro-6-(2,6-dimethylphenyl)pyridin-2-yl)-6-fluoropyridine-2-sulfonamide (int-b6) was replaced with N-(5-chloro-6-(2,6-dimethylphenyl)pyridin-2-yl)-2-fluoropyridine-4-sulfonamide (int-b7) and trimethylamine was replaced with N,N-diisopropylethylamine. LCMS (Condition 1): m/z 461.0 [M+H]⁺, 1.37 min. ¹H NMR (400 MHz, Methanol-d₄) δ 8.03 (d, J=5.2 Hz, 1H), 7.84 (d, J=8.7 Hz, 1H), 7.18 (dd, J=8.0, 3.2 Hz, 2H), 7.07 (d, J=7.6 Hz, 2H), 6.88 (s, 1H), 6.84 (dd, J=5.4, 1.4 Hz, 1H), 3.48 (s, 2H), 2.55 (d, J=6.6 Hz, 2H), 1.83 (s, 6H).

### Example 3: 4-((6-(N-(5-chloro-6-(2,6-dimethylphenyl)pyridin-2-yl)sulfamoyl)pyridin-2-yl)amino)butanoic Acid (3)

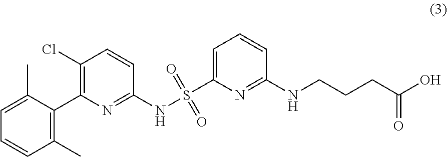 [SMILES: Cc1cccc(C)c1-c1nc(NS(=O)(=O)c2cccc(NCCCC(=O)O)n2)ccc1Cl]

4-((6-(N-(5-chloro-6-(2,6-dimethylphenyl)pyridin-2-yl)sulfamoyl)pyridin-2-yl)amino)butanoic acid (3) was synthesized using the procedure described in Example 1, except ethyl 3-aminopropanoate was replaced with ethyl 4-aminobutanoic acid. LCMS (Condition 1): m/z 475.1 [M+H]⁺, 1.57 min. ¹H NMR (400 MHz, DMSO-d₆) δ 11.10 (s, 1H), 7.94 (d, J=8.0 Hz, 1H), 7.51 (dd, J=8.0, 8.8 Hz, 1H), 7.40 (d, J=8.0 Hz, 1H), 7.19 (m, 2H), 7.07 (d, J=8.0 Hz, 2H), 6.99 (d, J=8.0 Hz, 1H), 6.64 (d, J=8.0 Hz, 1H), 2.21 (t, J=8.0 Hz, 2H), 1.78 (s, 6H), 1.61 (t, J=8.0 Hz, 2H), 1.18-1.32 (m, 3H).

### Example 4: 5-((6-(N-(5-chloro-6-(2,6-dimethylphenyl)pyridin-2-yl)sulfamoyl)pyridin-2-yl)amino)pentanoic Acid (4)

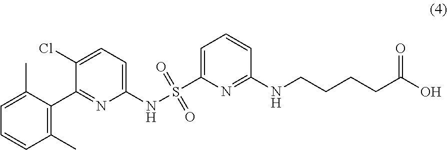 [SMILES: Cc1cccc(C)c1-c1nc(NS(=O)(=O)c2cccc(NCCCCC(=O)O)n2)ccc1Cl]

5-((6-(N-(5-chloro-6-(2,6-dimethylphenyl)pyridin-2-yl)sulfamoyl)pyridin-2-yl)amino)pentanoic acid (4) was synthesized using the procedure described in Example 1, except ethyl 3-aminopropanoate was replaced with 5-aminopentanoic acid and trimethylamine was replaced with N,N-diisopropylethylamine. LCMS (Condition 1): m/z 489.2 [M+H]⁺, 1.54 min. ¹H NMR (400 MHz, DMSO-d₆) δ 12.07 (s, 1H), 11.16 (s, 1H), 7.98 (d, J=8.9 Hz, 1H), 7.54 (dd, J=7.2, 8.5 Hz, 1H), 7.44 (d, J=8.9 Hz, 1H), 7.24 (d, J=6.9, 8.2 Hz, 1H), 7.13 (d, J=7.6 Hz, 3H), 7.03 (d, J=7.1 Hz, 1H), 6.68 (d, J=8.5 Hz, 1H), 3.10 (q, J=6.5 Hz, 2H), 2.22 (t, J=7.3 Hz, 2H), 1.84 (s, 6H), 1.59-1.48 (m, 2H), 1.36-1.46 (m, 2H).

### Example 5: 2-(1-(6-(N-(5-chloro-6-(2,6-dimethylphenyl)pyridin-2-yl)sulfamoyl)pyridin-2-yl)piperidin-4-yl)acetic Acid (5)

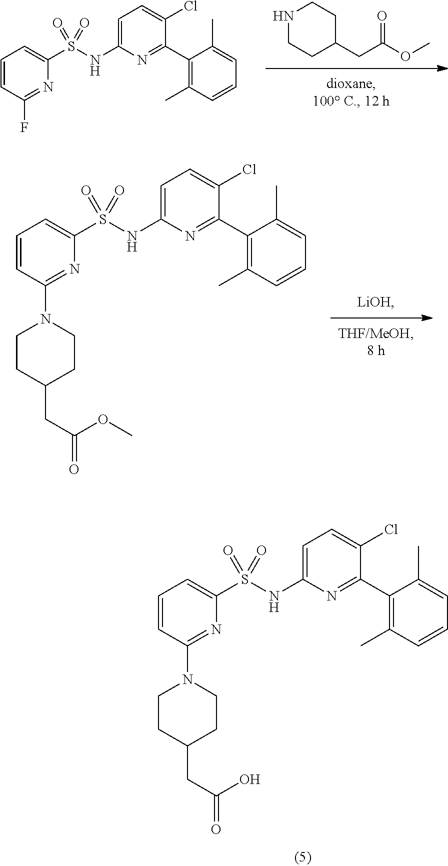 [SMILES: Cc1cccc(C)c1-c1nc(NS(=O)(=O)c2cccc(F)n2)ccc1Cl ; COC(=O)CC1CCNCC1 ; COC(=O)CC1CCN(c2cccc(S(=O)(=O)Nc3ccc(Cl)c(-c4c(C)cccc4C)n3)n2)CC1 ; Cc1cccc(C)c1-c1nc(NS(=O)(=O)c2cccc(N3CCC(CC(=O)O)CC3)n2)ccc1Cl]

### Step 1. Synthesis of methyl 2-(1-(6-(N-(5-chloro-6-(2,6-dimethylphenyl)pyridin-2-yl)sulfamoyl)pyridin-2-yl)piperidin-4-yl)acetate

In a vial, N-(5-chloro-6-(2,6-dimethylphenyl)pyridin-2-yl)-6-fluoropyridine-2-sulfonamide (int-b6) (50 mg, 0.13 mmol), methyl 2-(piperidin-4-yl)acetate (20 mg, 0.13 mmol) were dissolved in dioxane and the mixture was heated to 100° C. for 12 hours. The reaction was cooled to room temperature and DCM (20 mL) was added followed by 3 mL of water. The mixture was treated with 1 M HCl to pH ˜4, then the organics were separated, dried over Na₂SO₄, filtered, and concentrated under reduced pressure. The crude material was purified by flash column chromatography (4 g silica gel column, 0-30% EtOAc/hexanes) to provide the desired product methyl 2-(1-(6-(N-(5-chloro-6-(2,6-dimethylphenyl)pyridin-2-yl)sulfamoyl)pyridin-2-yl)piperidin-4-yl)acetate. LCMS (Condition 1): m/z 529.3 [M+H]⁺, 1.61 min.

### Step 2. Synthesis of 2-(1-(6-(N-(5-chloro-6-(2,6-dimethylphenyl)pyridin-2-yl)sulfamoyl)pyridin-2-yl)piperidin-4-yl)acetic Acid

In a vial, methyl 2-(1-(6-(N-(5-chloro-6-(2,6-dimethylphenyl)pyridin-2-yl)sulfamoyl)pyridin-2-yl)piperidin-4-yl)acetate (48 mg, 0.091 mmol) was dissolved in THE (2 mL) and MeOH (2 mL). Water (1 mL) was added followed by solid LiOH (2.2 mg, 0.091 mmol) and the mixture was stirred for 8 h. The solvent was removed under reduced pressure and then water (3 mL) was added and 1 M HCl to make solution pH ˜4. The material was then extracted with DCM (5 mL×3), the organics were combined, washed with brine, dried over sodium sulfate, filtered, and solvent was removed under reduced pressure to give 2-(1-(6-(N-(5-chloro-6-(2,6-dimethylphenyl)pyridin-2-yl)sulfamoyl)pyridin-2-yl)piperidin-4-yl)acetic acid (5). LCMS (Condition 1): m/z 515.2 [M+H]⁺, 1.58 min. ¹H NMR (400 MHz, DMSO-d₆), δ 12.47 (s, 1H), 11.57 (s, 1H), 8.37 (d, J=8.8 Hz, 1H), 8.08 (dd, J=7.3, 8.7 Hz, 1H), 7.77 (d, J=8.7 Hz, 1H), 7.61 (dd, J=7.0, 8.1 Hz, 1H), 7.53-7.35 (m, 4H), 4.57 (d, J=13.3 Hz, 2H), 3.17 (t, J=12.6 Hz, 2H), 2.51 (d, J=7.1 Hz, 2H), 2.29 (m, 1H), 2.18 (s, 6H), 2.04 (d, J=12.9 Hz, 2H), 1.44-1.29 (m, 2H).

### Example 6: 2-(1-(6-(N-(6-(5-fluoro-2-methylphenyl)-5-(trifluoromethyl)pyridin-2-yl)sulfamoyl)pyridin-2-yl)-4-methylpiperidin-4-yl)acetic Acid (6)

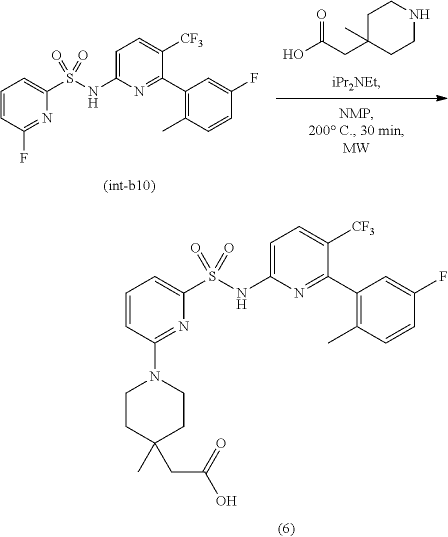 [SMILES: Cc1ccc(F)cc1-c1nc(NS(=O)(=O)c2cccc(F)n2)ccc1C(F)(F)F ; CC1(CC(=O)O)CCNCC1 ; Cc1ccc(F)cc1-c1nc(NS(=O)(=O)c2cccc(N3CCC(C)(CC(=O)O)CC3)n2)ccc1C(F)(F)F]

To a microwave vial was added 6-fluoro-N-(6-(5-fluoro-2-methylphenyl)-5-(trifluoromethyl)pyridin-2-yl)pyridine-2-sulfonamide (int-b10) (50 mg, 0.12 mmol), 2-(4-methylpiperidin-4-yl)acetic acid hydrochloride (68 mg, 0.35 mmol), N,N-diisopropylethylamine (150 mg, 1.2 mmol), and NMP (1 mL). The mixture was heated to 200° C. for 30 minutes in the microwave. Following aqueous workup, the resulting residue was purified by flash column chromatography (0-10% MeOH/DCM to provide the anticipated product 2-(1-(6-(N-(6-(5-fluoro-2-methylphenyl)-5-(trifluoromethyl)pyridin-2-yl)sulfamoyl)pyridin-2-yl)-4-methylpiperidin-4-yl)acetic acid (6) as a white solid. LCMS (Condition 1): m/z 567.2 [M+H]⁺, 1.80 min. ¹H NMR (400 MHz, DMSO-d₆) δ 12.01 (s, 1H), 11.57 (s, 1H), 8.19 (d, J=8.9 Hz, 1H), 7.67 (dd, J=8.7, 7.3 Hz, 1H), 7.49 (d, J=8.8 Hz, 1H), 7.27 (dd, J=8.6, 5.7 Hz, 1H), 7.19-7.14 (m, 1H), 7.09 (d, J=7.3 Hz, 1H), 7.04 (d, J=8.8 Hz, 1H), 6.92 (d, J=9.2 Hz, 1H), 3.51-3.40 (m, 2H), 3.40-3.34 (m, 2H), 2.17 (s, 2H), 1.72 (s, 3H), 1.43-1.31 (m, 2H), 1.29-1.13 (m, 2H), 0.98 (s, 3H).

### Example 7: 2-(4-methyl-1-(6-(N-(6-(o-tolyl)-5-(trifluoromethyl)pyridin-2-yl)sulfamoyl)pyridin-2-yl)piperidin-4-yl)acetic Acid (7)

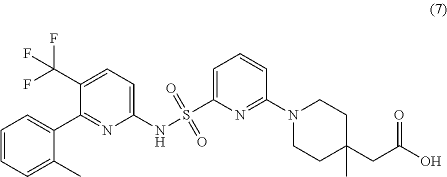 [SMILES: Cc1ccccc1-c1nc(NS(=O)(=O)c2cccc(N3CCC(C)(CC(=O)O)CC3)n2)ccc1C(F)(F)F]

2-(4-methyl-1-(6-(N-(6-(o-tolyl)-5-(trifluoromethyl)pyridin-2-yl)sulfamoyl)pyridin-2-yl)piperidin-4-yl)acetic acid (7) was synthesized using the procedure described in Example 6, except 6-fluoro-N-(6-(5-fluoro-2-methylphenyl)-5-(trifluoromethyl)pyridin-2-yl)pyridine-2-sulfonamide (int-b10) was replaced with 6-fluoro-N-(6-(o-tolyl)-5-(trifluoromethyl)pyridin-2-yl)pyridine-2-sulfonamide (int-b1). LCMS (Condition 1): m/z 549.2 [M+H]⁺, 1.83 min. ¹H NMR (400 MHz, DMSO-d₆) δ 12.03 (s, 1H), 11.52 (s, 1H), 8.18 (d, J=8.9 Hz, 1H), 7.67 (dd, J=8.7, 7.3 Hz, 1H), 7.49 (d, J=8.9 Hz, 1H), 7.33-7.29 (m, 1H), 7.25-7.15 (m, 2H), 7.08 (d, J=7.2 Hz, 1H), 7.07-7.01 (m, 2H), 3.53-3.40 (m, 2H), 3.40-3.34 (m, 2H), 2.17 (s, 2H), 1.77 (s, 3H), 1.43-1.31 (m, 2H), 1.29-1.15 (m, 2H), 0.98 (s, 3H).

### Example 8: 2,2-dimethyl-3-((6-(N-(6-(o-tolyl)-5-(trifluoromethyl)pyridin-2-yl)sulfamoyl)pyridin-2-yl)amino)propanoic Acid (8)

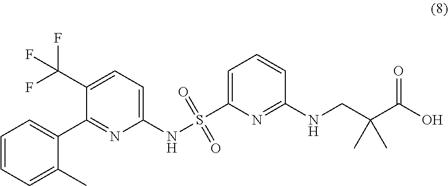 [SMILES: Cc1ccccc1-c1nc(NS(=O)(=O)c2cccc(NCC(C)(C)C(=O)O)n2)ccc1C(F)(F)F]

2,2-dimethyl-3-((6-(N-(6-(o-tolyl)-5-(trifluoromethyl)pyridin-2-yl)sulfamoyl)pyridin-2-yl)amino)propanoic acid (8) was synthesized using the procedure described in Example 6, except 6-fluoro-N-(6-(5-fluoro-2-methylphenyl)-5-(trifluoromethyl)pyridin-2-yl)pyridine-2-sulfonamide (int-b10) was replaced with 6-fluoro-N-(6-(o-tolyl)-5-(trifluoromethyl)pyridin-2-yl)pyridine-2-sulfonamide (int-b1) and 2-(4-methylpiperidin-4-yl)acetic acid hydrochloride was replaced with ethyl 3-amino-2,2-dimethylpropanoate hydrochloride. LCMS (Condition 1): m/z 509.1 [M+H]⁺, 1.79 min. ¹H NMR (400 MHz, DMSO-d₆) δ 12.27 (s, 1H), 11.52 (s, 1H), 8.16 (d, J=8.9 Hz, 1H), 7.54 (d, J=8.9 Hz, 1H), 7.49 (dd, J=8.5, 7.2 Hz, 1H), 7.33-7.29 (m, 1H), 7.25-7.16 (m, 2H), 7.04 (d, J=7.4 Hz, 1H), 7.00 (d, J=7.2 Hz, 1H), 6.99-6.90 (m, 1H), 6.80 (d, J=8.5 Hz, 1H), 3.31-3.19 (m, 2H), 1.81 (s, 3H), 0.96-0.88 (m, 6H).

### Example 9: 2-(4-hydroxy-1-(6-(N-(6-(o-tolyl)-5-(trifluoromethyl)pyridin-2-yl)sulfamoyl)pyridin-2-yl)piperidin-4-yl)-2-methylpropanoic Acid (9)

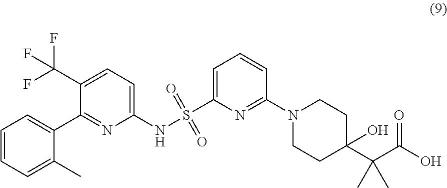 [SMILES: Cc1ccccc1-c1nc(NS(=O)(=O)c2cccc(N3CCC(O)(C(C)(C)C(=O)O)CC3)n2)ccc1C(F)(F)F]

2-(4-hydroxy-1-(6-(N-(6-(o-tolyl)-5-(trifluoromethyl)pyridin-2-yl)sulfamoyl)pyridin-2-yl)piperidin-4-yl)-2-methylpropanoic acid (9) was synthesized using the procedure described in Example 6, except 6-fluoro-N-(6-(5-fluoro-2-methylphenyl)-5-(trifluoromethyl)pyridin-2-yl)pyridine-2-sulfonamide (int-b10) was replaced with 6-fluoro-N-(6-(o-tolyl)-5-(trifluoromethyl)pyridin-2-yl)pyridine-2-sulfonamide (int-b1) and 2-(4-methylpiperidin-4-yl)acetic acid hydrochloride was replaced with 2-(4-hydroxypiperidin-4-yl)-2-methylpropanoic acid hydrochloride. LCMS (Condition 1): m/z 579.2 [M+H]⁺, 1.79 min. ¹H NMR (400 MHz, DMSO-d₆) δ 12.19 (s, 1H), 11.51 (s, 1H), 8.18 (d, J=8.9 Hz, 1H), 7.66 (dd, J=8.7, 7.3 Hz, 1H), 7.53 (d, J=8.8 Hz, 1H), 7.34-7.30 (m, 1H), 7.26-7.16 (m, 2H), 7.13-6.97 (m, 3H), 4.54 (s, 1H), 4.04-3.83 (m, 2H), 3.06-2.87 (m, 2H), 1.81 (s, 3H), 1.57-1.47 (m, 2H), 1.47-1.33 (m, 2H), 1.03 (s, 6H).

### Example 10: 2-methyl-2-(1-(6-(N-(6-(o-tolyl)-5-(trifluoromethyl)pyridin-2-yl)sulfamoyl)pyridin-2-yl)piperidin-4-yl)propanoic Acid (10)

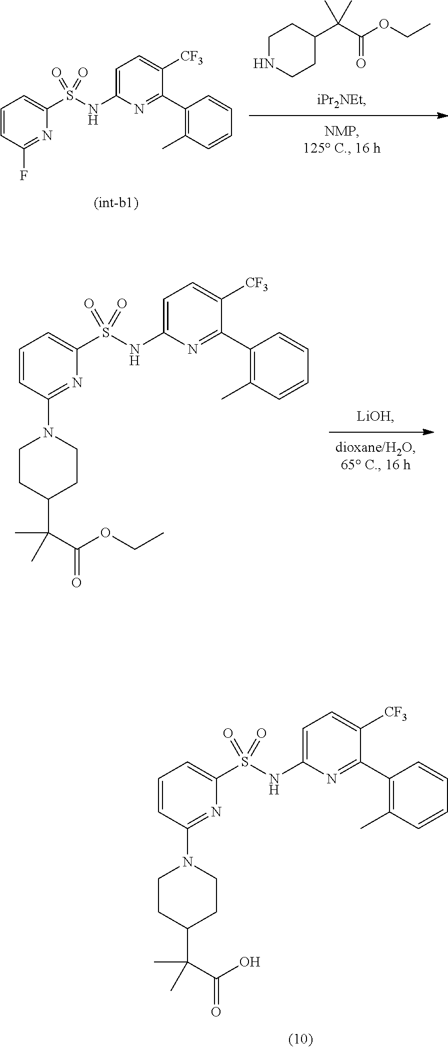 [SMILES: Cc1ccccc1-c1nc(NS(=O)(=O)c2cccc(F)n2)ccc1C(F)(F)F ; CCOC(=O)C(C)(C)C1CCNCC1 ; CCOC(=O)C(C)(C)C1CCN(c2cccc(S(=O)(=O)Nc3ccc(C(F)(F)F)c(-c4ccccc4C)n3)n2)CC1 ; Cc1ccccc1-c1nc(NS(=O)(=O)c2cccc(N3CCC(C(C)(C)C(=O)O)CC3)n2)ccc1C(F)(F)F]

### Step 1. Synthesis of ethyl 2-methyl-2-(1-(6-(N-(6-(o-tolyl)-5-(trifluoromethyl)pyridin-2-yl)sulfamoyl)pyridin-2-yl)piperidin-4-yl)propanoate

A mixture of ethyl 2-methyl-2-(piperidin-4-yl)propanoate (86 mg, 0.37 mmol) and 6-fluoro-N-(6-(o-tolyl)-5-(trifluoromethyl)pyridin-2-yl)pyridine-2-sulfonamide (int-b1) (50 mg, 0.12 mmol) in NMP (1.5 mL) was stirred at 125° C. overnight. The reaction was filtered and the filtrate was subjected to HPLC purification to provide the product ethyl 2-methyl-2-(1-(6-(N-(6-(o-tolyl)-5-(trifluoromethyl)pyridin-2-yl)sulfamoyl)pyridin-2-yl)piperidin-4-yl)propanoate as a white solid. ¹H NMR (400 MHz, DMSO-d₆) δ 11.51 (s, 1H), 8.20 (d, J=8.8 Hz, 1H), 7.67 (dd, J=8.7, 7.3 Hz, 1H), 7.53 (d, J=8.8 Hz, 1H), 7.34-7.30 (m, 1H), 7.26-7.16 (m, 2H), 7.11-6.95 (m, 3H), 4.30-4.07 (m, 2H), 4.03 (q, J=7.1 Hz, 2H), 2.73-2.58 (m, 2H), 1.87-1.65 (m, 4H), 1.47-1.33 (m, 2H), 1.13 (t, J=7.1 Hz, 3H), 1.04-0.84 (m, 8H).

### Step 2. Synthesis of 2-methyl-2-(1-(6-(N-(6-(o-tolyl)-5-(trifluoromethyl)pyridin-2-yl)sulfamoyl)pyridin-2-yl)piperidin-4-yl)propanoic Acid (10)

A mixture of ethyl 2-methyl-2-(1-(6-(N-(6-(o-tolyl)-5-(trifluoromethyl)pyridin-2-yl)sulfamoyl)pyridin-2-yl)piperidin-4-yl)propanoate (65 mg, 0.11 mmol) and LiOH (120 mg, 5.0 mmol) in dioxane (3 mL) and water (3 mL) was stirred at 65° C. overnight. The reaction mixture was acidified with 10% citric acid. Aqueous work-up followed by flash column chromatography (0-100% EtOAc/hexanes) provided 2-methyl-2-(1-(6-(N-(6-(o-tolyl)-5-(trifluoromethyl)pyridin-2-yl)sulfamoyl)pyridin-2-yl)piperidin-4-yl)propanoic acid (10) as a white solid. LCMS (Condition 1): m/z 563.20 [M+H]⁺, 1.87 min. ¹H NMR (400 MHz, DMSO-d₆) δ 12.18 (s, 1H), 11.51 (s, 1H), 8.20 (d, J=8.9 Hz, 1H), 7.67 (dd, J=8.7, 7.3 Hz, 1H), 7.52 (d, J=8.7 Hz, 1H), 7.34-7.29 (m, 1H), 7.26-7.16 (m, 2H), 7.10-7.00 (m, 3H), 4.30-4.06 (m, 2H), 2.75-2.59 (m, 2H), 1.83-1.63 (m, 5H), 1.50-1.38 (m, 2H), 1.05-0.87 (m, 7H).

### Example 11: 1-(((6-(N-(6-(o-tolyl)-5-(trifluoromethyl)pyridin-2-yl)sulfamoyl)pyridin-2-yl)amino)methyl)cyclopropane-1-carboxylic Acid (11)

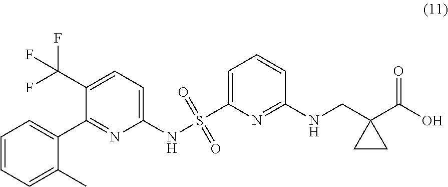 [SMILES: Cc1ccccc1-c1nc(NS(=O)(=O)c2cccc(NCC3(C(=O)O)CC3)n2)ccc1C(F)(F)F]

1-(((6-(N-(6-(o-tolyl)-5-(trifluoromethyl)pyridin-2-yl)sulfamoyl)pyridin-2-yl)amino)methyl)cyclopropane-1-carboxylic acid (11) was synthesized using the procedure described in Example 10, except in step 1, ethyl 2-methyl-2-(piperidin-4-yl)propanoate hydrochloride was replaced with methyl 1-(aminomethyl)cyclopropane-1-carboxylate hydrochloride. LCMS (Condition 1): m/z 507.2 [M+H]⁺, 1.75 min. ¹H NMR (400 MHz, DMSO-d₆) δ 12.28 (s, 1H), 11.49 (s, 1H), 8.15 (d, J=8.8 Hz, 1H), 7.58-7.41 (m, 2H), 7.33-7.29 (m, 1H), 7.26-7.17 (m, 2H), 7.17-7.03 (m, 2H), 6.99 (d, J=7.2 Hz, 1H), 6.76 (d, J=8.4 Hz, 1H), 3.30-3.20 (m, 2H), 1.82 (s, 3H), 0.93-0.88 (m, 2H), 0.77-0.59 (m, 2H).

### Example 12: 1-(((6-(N-(6-(o-tolyl)-5-(trifluoromethyl)pyridin-2-yl)sulfamoyl)pyridin-2-yl)amino)methyl)cyclobutane-1-carboxylic Acid (12)

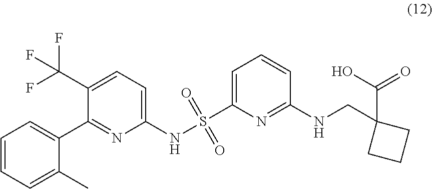 [SMILES: Cc1ccccc1-c1nc(NS(=O)(=O)c2cccc(NCC3(C(=O)O)CCC3)n2)ccc1C(F)(F)F]

1-(((6-(N-(6-(o-tolyl)-5-(trifluoromethyl)pyridin-2-yl)sulfamoyl)pyridin-2-yl)amino)methyl)cyclobutane-1-carboxylic acid (12) was synthesized using the procedure described in Example 10, except in step 1, ethyl 2-methyl-2-(piperidin-4-yl)propanoate hydrochloride was replaced with methyl 1-(aminomethyl)cyclobutane-1-carboxylate hydrochloride. LCMS (Condition 1): m/z 521.2 [M+H]⁺, 1.80 min. ¹H NMR (400 MHz, DMSO-d₆) δ 12.32 (s, 1H), 11.54 (s, 1H), 8.16 (d, J=8.9 Hz, 1H), 7.60-7.43 (m, 2H), 7.33-7.28 (m, 1H), 7.25-7.14 (m, 2H), 7.06-6.96 (m, 3H), 6.78 (d, J=8.4 Hz, 1H), 3.54-3.36 (m, 2H), 2.23-2.06 (m, 2H), 1.86-1.65 (m, 7H).

### Example 13: 2-(1-(6-(N-(6-(3-fluoro-2-methylphenyl)-5-(trifluoromethyl)pyridin-2-yl)sulfamoyl)pyridin-2-yl)-4-methylpiperidin-4-yl)acetic Acid (13)

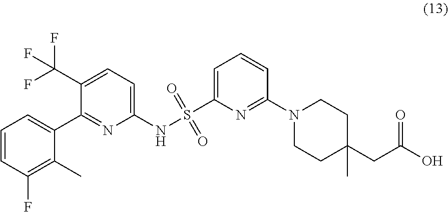 [SMILES: Cc1c(F)cccc1-c1nc(NS(=O)(=O)c2cccc(N3CCC(C)(CC(=O)O)CC3)n2)ccc1C(F)(F)F]

2-(1-(6-(N-(6-(3-fluoro-2-methylphenyl)-5-(trifluoromethyl)pyridin-2-yl)sulfamoyl)pyridin-2-yl)-4-methylpiperidin-4-yl)acetic acid (13) was synthesized using the procedure described in step 1 of Example 10 except 6-fluoro-N-(6-(o-tolyl)-5-(trifluoromethyl)pyridin-2-yl)pyridine-2-sulfonamide (int-b1) was replaced with 6-fluoro-N-(6-(3-fluoro-2-methylphenyl)-5-(trifluoromethyl)pyridin-2-yl)pyridine-2-sulfonamide (int-b12) and ethyl 2-methyl-2-(piperidin-4-yl)propanoate hydrochloride was replaced with 2-(4-methylpiperidin-4-yl)acetic acid hydrochloride. LCMS (Condition 1): m/z 567.2 [M+H]⁺, 1.84 min. ¹H NMR (400 MHz, DMSO-d₆) δ 12.03 (s, 1H), 11.58 (s, 1H), 8.20 (d, J=8.8 Hz, 1H), 7.68 (dd, J=8.7, 7.3 Hz, 1H), 7.50 (d, J=8.9 Hz, 1H), 7.30-7.17 (m, 2H), 7.09 (d, J=7.2 Hz, 1H), 7.06 (s, 1H), 6.92 (d, J=7.1 Hz, 1H), 3.52-3.40 (m, 2H), 3.40-3.34 (m, 2H), 2.17 (s, 2H), 1.65 (d, J=2.1 Hz, 3H), 1.44-1.29 (m, 2H), 1.29-1.15 (m, 2H), 0.98 (s, 3H).

### Example 14: 2-(1-(6-(N-(6-(o-tolyl)-5-(trifluoromethyl)pyridin-2-yl)sulfamoyl)pyridin-2-yl)piperidin-4-yl)acetic Acid (14)

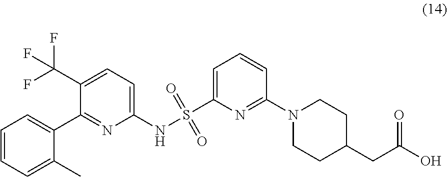 [SMILES: Cc1ccccc1-c1nc(NS(=O)(=O)c2cccc(N3CCC(CC(=O)O)CC3)n2)ccc1C(F)(F)F]

2-(1-(6-(N-(6-(o-tolyl)-5-(trifluoromethyl)pyridin-2-yl)sulfamoyl)pyridin-2-yl)piperidin-4-yl)acetic acid (14) was synthesized using the procedure described in Example 10, except in step 1, ethyl 2-methyl-2-(piperidin-4-yl)propanoate hydrochloride was replaced with ethyl 2-(piperidin-4-yl)acetate, N,N-diisopropylethylamine was replaced with K₂CO₃, and NMP was replaced with dioxane, and in step 2, dioxane was replaced with DME. LCMS (Condition 1): m/z=535.2 [M+H]⁺, 1.54 min. ¹H NMR (400 MHz, Chloroform-d) δ 7.98 (d, J=8.9 Hz, 1H), 7.66 (d, J=8.9 Hz, 1H), 7.60 (dd, J=7.3, 8.7 Hz, 1H), 7.39-7.33 (m, 1H), 7.32 (d, J=7.2 Hz, 1H), 7.24 (dd, J=7.8, 15.9 Hz, 2H), 7.11 (d, J=7.5 Hz, 1H), 6.79 (d, J=8.7 Hz, 1H), 4.20 (d, J=13.2 Hz, 2H), 2.91-2.72 (m, 2H), 2.23 (d, J=7.0 Hz, 2H), 2.01 (s, 4H), 1.71 (d, J=11.2 Hz, 2H), 1.13 (qd, J=4.0, 12.7 Hz, 2H). ¹⁹F NMR (376 MHz, Chloroform-d) δ −58.73 (s, 3F).

### Example 15: 2-(1-(6-(N-(6-(2-ethylphenyl)-5-(trifluoromethyl)pyridin-2-yl)sulfamoyl)pyridin-2-yl)piperidin-4-yl)acetic Acid (15)

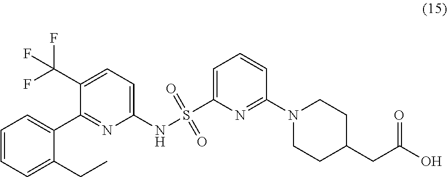 [SMILES: CCc1ccccc1-c1nc(NS(=O)(=O)c2cccc(N3CCC(CC(=O)O)CC3)n2)ccc1C(F)(F)F]

2-(1-(6-(N-(6-(2-ethylphenyl)-5-(trifluoromethyl)pyridin-2-yl)sulfamoyl)pyridin-2-yl)piperidin-4-yl)acetic acid (15) was synthesized using the procedure described in Example 10, except in step 1, 6-fluoro-N-(6-(o-tolyl)-5-(trifluoromethyl)pyridin-2-yl)pyridine-2-sulfonamide (int-b1) was replaced with N-(6-(2-ethylphenyl)-5-(trifluoromethyl)pyridin-2-yl)-6-fluoropyridine-2-sulfonamide (int-b11), ethyl 2-methyl-2-(piperidin-4-yl)propanoate hydrochloride was replaced with ethyl 2-(piperidin-4-yl)acetate, N,N-diisopropylethylamine was replaced with K₂CO₃, and NMP was replaced with dioxane, and in step 2, dioxane was replaced with DME. LCMS (Condition 1): m/z=549.2 [M+H]⁺, 1.57 min. ¹H NMR (400 MHz, Chloroform-d) δ 7.75 (d, J=8.9 Hz, 1H), 7.44 (d, J=8.8 Hz, 1H), 7.36 (dd, J=7.3, 8.7 Hz, 1H), 7.17 (td, J=1.3, 7.6 Hz, 1H), 7.11-7.06 (m, 2H), 7.00 (td, J=1.0, 7.5 Hz, 1H), 6.87 (d, J=7.5 Hz, 1H), 6.56 (d, J=8.7 Hz, 1H), 3.95 (d, J=13.7 Hz, 2H), 2.64-2.50 (m, 2H), 2.10 (m, 2H), 1.87-1.69 (m, 2H), 1.50 (d, J=12.2 Hz, 3H), 0.99-0.84 (m, 3H), 0.81 (t, J=7.6 Hz, 3H). ¹⁹F NMR (376 MHz, Chloroform-d) δ −58.32 (s, 3F).

### Example 16: 2-(1-(6-(N-(6-([1,1′-biphenyl]-2-yl)-5-(trifluoromethyl)pyridin-2-yl)sulfamoyl)pyridin-2-yl)piperidin-4-yl)acetic Acid (16)

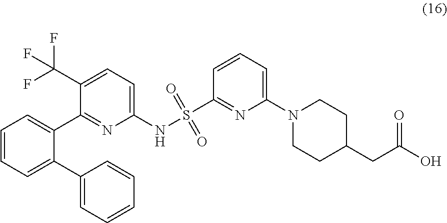 [SMILES: O=C(O)CC1CCN(c2cccc(S(=O)(=O)Nc3ccc(C(F)(F)F)c(-c4ccccc4-c4ccccc4)n3)n2)CC1]

2-(1-(6-(N-(6-([1,1′-biphenyl]-2-yl)-5-(trifluoromethyl)pyridin-2-yl)sulfamoyl)pyridin-2-yl)piperidin-4-yl)acetic acid (16) was synthesized using the procedure described in Example 10, except in step 1, 6-fluoro-N-(6-(o-tolyl)-5-(trifluoromethyl)pyridin-2-yl)pyridine-2-sulfonamide (int-b1) was replaced with N-(6-([1,1′-biphenyl]-2-yl)-5-(trifluoromethyl)pyridin-2-yl)-6-fluoropyridine-2-sulfonamide (int-b5), ethyl 2-methyl-2-(piperidin-4-yl)propanoate hydrochloride was replaced with ethyl 2-(piperidin-4-yl)acetate, N,N-diisopropylethylamine was replaced with K₂CO₃, and NMP was replaced with dioxane, and in step 2, dioxane was replaced with DME. LCMS (Condition 1): m/z=597.2 [M+H]⁺, 1.61 min. ¹H NMR (400 MHz, Chloroform-d) δ 7.81 (d, J=8.9 Hz, 1H), 7.58 (m, 2H), 7.52-7.46 (m, 1H), 7.46-7.43 (m, 1H), 7.40 (td, J=1.3, 7.5 Hz, 1H), 7.28-7.23 (m, 2H), 7.17-7.08 (m, 3H), 7.05 (dd, J=1.7, 7.7 Hz, 2H), 6.80 (d, J=8.7 Hz, 1H), 4.17 (d, J=12.5 Hz, 2H), 2.85-2.73 (m, 2H), 2.24 (d, J=7.0 Hz, 2H), 2.02 (m, 1H), 1.72 (d, J=11.5 Hz, 3H), 1.12 (qd, J=4.0, 12.5 Hz, 2H). ¹⁹F NMR (376 MHz, Chloroform-d) δ −57.99 (s, 3F).

### Example 17: 2-(1-(6-(N-(6-(2-cyclobutylphenyl)-5-(trifluoromethyl)pyridin-2-yl)sulfamoyl)pyridin-2-yl)piperidin-4-yl)acetic Acid (17)

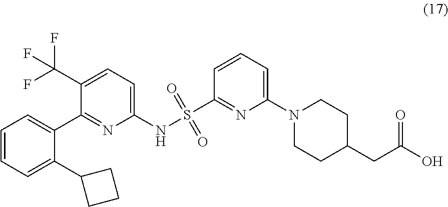 [SMILES: O=C(O)CC1CCN(c2cccc(S(=O)(=O)Nc3ccc(C(F)(F)F)c(-c4ccccc4C4CCC4)n3)n2)CC1]

2-(1-(6-(N-(6-(2-cyclobutylphenyl)-5-(trifluoromethyl)pyridin-2-yl)sulfamoyl)pyridin-2-yl)piperidin-4-yl)acetic acid (17) was synthesized using the procedure described in Example 10, except in step 1, 6-fluoro-N-(6-(o-tolyl)-5-(trifluoromethyl)pyridin-2-yl)pyridine-2-sulfonamide (int-b1) was replaced N-(6-(2-cyclobutylphenyl)-5-(trifluoromethyl)pyridin-2-yl)-6-fluoropyridine-2-sulfonamide (int-b13), ethyl 2-methyl-2-(piperidin-4-yl)propanoate hydrochloride was replaced with ethyl 2-(piperidin-4-yl)acetate, N,N-diisopropylethylamine was replaced with K₂CO₃, and NMP was replaced with dioxane, and in step 2, dioxane was replaced with DME. LCMS (Condition 1): m/z=575.2 [M+H]⁺, 1.77 min. ¹H NMR (400 MHz, Chloroform-d) δ 9.52 (s, 2H), 7.97 (d, J=8.9 Hz, 1H), 7.67 (d, J=8.9 Hz, 1H), 7.60 (dd, J=7.3, 8.7 Hz, 1H), 7.44 (dd, J=1.5, 6.7 Hz, 2H), 7.31 (d, J=7.2 Hz, 1H), 7.23 (ddd, J=2.2, 6.4, 8.5 Hz, 1H), 7.09 (d, J=7.6 Hz, 1H), 6.79 (d, J=8.7 Hz, 1H), 4.21 (t, J=12.8 Hz, 2H), 3.23 (p, J=9.0 Hz, 1H), 2.88-2.73 (m, 2H), 2.24 (d, J=7.0 Hz, 2H), 2.09-1.95 (m, 3H), 1.90 (d, J=3.1 Hz, 1H), 1.80-1.63 (m, 4H, obscured by water peak), 1.14 (q, J=12.3 Hz, 2H). ¹⁹F NMR (376 MHz, Chloroform-d) δ −58.02 (s, 3F).

### Example 18: 4-methyl-1-(6-(N-(6-(2-(pyrimidin-2-yl)phenyl)-5-(trifluoromethyl)pyridin-2-yl)sulfamoyl)pyridin-2-yl)piperidine-4-carboxylic Acid (18)

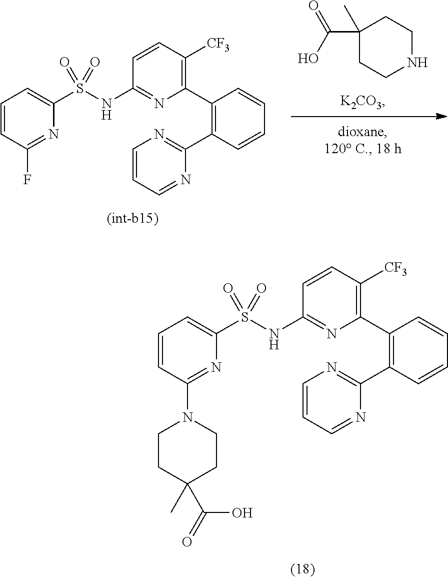

In a vial, 6-fluoro-N-(6-(2-(pyrimidin-2-yl)phenyl)-5-(trifluoromethyl)pyridin-2-yl)pyridine-2-sulfonamide (int-b15) (100 mg, 0.210 mmol), 4-methylpiperidine-4-carboxylic acid hydrochloride (56.7 mg, 0.316 mmol) and K₂CO₃ (87 mg, 0.63 mmol) were suspended in dioxane (8 mL) and the resulting mixture was stirred at 120° C. for 18 h. The mixture was diluted with water and pH adjusted to 1 with 1 M aq. HCl and then extracted with EtOAc (30 mL×2). The combined extracts were washed with brine, dried over Na₂SO₄, filtered and concentrated. The crude product was purified by silica gel chromatography (12 g silica gel column, 0-30% EtOAc/EtOH (3:1) mixture in heptane) to yield 4-methyl-1-(6-(N-(6-(2-(pyrimidin-2-yl)phenyl)-5-(trifluoromethyl)pyridin-2-yl)sulfamoyl)pyridin-2-yl)piperidine-4-carboxylic acid (18) as a white solid. LCMS (Condition 1): m/z=599.1 [M+H]⁺, 1.59 min. ¹H NMR (400 MHz, Chloroform-d) δ 8.37 (dd, J=1.3, 7.9 Hz, 1H), 8.35 (d, J=4.8 Hz, 2H), 7.89 (d, J=9.0 Hz, 1H), 7.65-7.57 (m, 3H), 7.51 (td, J=1.4, 7.5 Hz, 1H), 7.34-7.30 (m, 2H), 6.95 (t, J=4.8 Hz, 1H), 6.77 (d, J=8.6 Hz, 1H), 3.87 (d, J=13.9 Hz, 3H), 2.81 (d, J=12.8 Hz, 2H), 1.97 (d, J=13.5 Hz, 2H), 1.32-1.22 (m, 3H), 1.17 (s, 3H). ¹⁹F NMR (376 MHz, Chloroform-d) δ −58.60 (s, 3F).

### Example 19: 1-(6-(N-(6-([1,1′-biphenyl]-2-yl)-5-chloropyridin-2-yl)sulfamoyl)pyridin-2-yl)-4-methylpiperidine-4-carboxylic Acid (19)

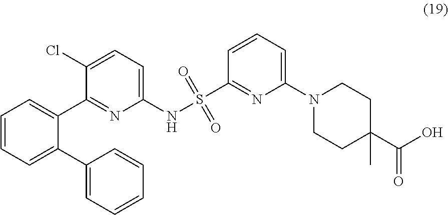 [SMILES: CC1(C(=O)O)CCN(c2cccc(S(=O)(=O)Nc3ccc(Cl)c(-c4ccccc4-c4ccccc4)n3)n2)CC1]

1-(6-(N-(6-([1,1′-biphenyl]-2-yl)-5-chloropyridin-2-yl)sulfamoyl)pyridin-2-yl)-4-methylpiperidine-4-carboxylic acid (19) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-([1,1′-biphenyl]-2-yl)-5-chloropyridin-2-yl)-6-fluoropyridine-2-sulfonamide (int-b8). LCMS (Condition 1): m/z=563.1 [M+H]⁺, 1.75 min. ¹H NMR (400 MHz, Chloroform-d) δ 7.58 (dd, J=7.3, 8.7 Hz, 1H), 7.55-7.48 (m, 1H), 7.47-7.43 (m, 3H), 7.40 (td, J=1.5, 7.4 Hz, 1H), 7.32-7.29 (m, 1H), 7.26 (d, J=7.2 Hz, 1H), 7.20-7.08 (m, 3H), 7.05-6.97 (m, 2H), 6.77 (d, J=8.7 Hz, 1H), 3.96 (d, J=13.6 Hz, 2H), 3.05-2.92 (m, 2H), 2.05 (d, J=13.6 Hz, 3H), 1.38-1.30 (m, 2H), 1.21 (s, 3H).

### Example 20: 1-(6-(N-(6-([1,1′-biphenyl]-2-yl)-5-(trifluoromethyl)pyridin-2-yl)sulfamoyl)pyridin-2-yl)-4-methylpiperidine-4-carboxylic Acid (20)

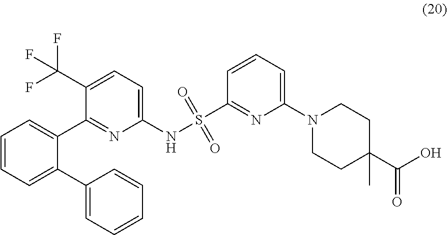 [SMILES: CC1(C(=O)O)CCN(c2cccc(S(=O)(=O)Nc3ccc(C(F)(F)F)c(-c4ccccc4-c4ccccc4)n3)n2)CC1]

1-(6-(N-(6-([1,1′-biphenyl]-2-yl)-5-(trifluoromethyl)pyridin-2-yl)sulfamoyl)pyridin-2-yl)-4-methylpiperidine-4-carboxylic acid (20) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-([1,1′-biphenyl]-2-yl)-5-(trifluoromethyl)pyridin-2-yl)-6-fluoropyridine-2-sulfonamide (int-b5). LCMS (Condition 1): m/z=597.2 [M+H]⁺, 1.77 min. ¹H NMR (400 MHz, Chloroform-d) δ 7.82 (d, J=8.9 Hz, 1H), 7.63-7.48 (m, 3H), 7.47-7.42 (m, 1H), 7.39 (td, J=1.3, 7.5 Hz, 1H), 7.30 (s, 1H), 7.26 (d, J=7.2 Hz, 1H), 7.15-7.08 (m, 3H), 7.04 (dd, J=2.0, 7.5 Hz, 2H), 6.77 (d, J=8.7 Hz, 1H), 3.92 (d, J=11.2 Hz, 2H), 3.00-2.83 (m, 2H), 2.03 (d, J=13.5 Hz, 2H), 1.38-1.25 (m, 2H), 1.22 (s, 3H). ¹⁹F NMR (376 MHz, Chloroform-d) δ −57.98 (s, 3F).

### Example 21: 4-(4-(6-(N-(6-(2-chloro-5-methoxyphenyl)-5-(trifluoromethyl)pyridin-2-yl)sulfamoyl)pyridin-2-yl)piperazin-1-yl)benzoic Acid (21)

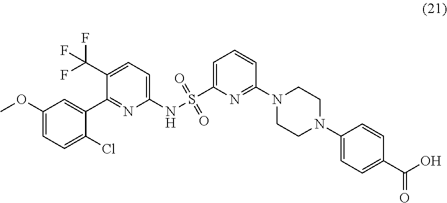 [SMILES: COc1ccc(Cl)c(-c2nc(NS(=O)(=O)c3cccc(N4CCN(c5ccc(C(=O)O)cc5)CC4)n3)ccc2C(F)(F)F)c1]

4-(4-(6-(N-(6-(2-chloro-5-methoxyphenyl)-5-(trifluoromethyl)pyridin-2-yl)sulfamoyl)pyridin-2-yl)piperazin-1-yl)benzoic acid (21) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-chloro-5-methoxyphenyl)-5-(trifluoromethyl)pyridin-2-yl)-6-fluoropyridine-2-sulfonamide (int-b9) and 4-methylpiperidine-4-carboxylic acid hydrochloride was replaced with 4-(piperazin-1-yl)benzoic acid. LCMS (Condition 1): m/z=648.1 [M+H]⁺, 1.62 min. ¹H NMR (400 MHz, Chloroform-d) δ 8.02 (d, J=8.9 Hz, 1H), 7.90 (d, J=8.8 Hz, 2H), 7.79 (d, J=8.8 Hz, 1H), 7.60 (t, J=7.9 Hz, 1H), 7.33 (dd, J=6.1, 8.0 Hz, 2H), 6.96 (dd, J=3.0, 8.9 Hz, 1H), 6.84-6.75 (m, 2H), 6.70 (d, J=8.5 Hz, 2H), 3.79 (s, 3H), 3.69-3.54 (m, 4H), 3.28 (s, 4H). ¹⁹F NMR (376 MHz, Chloroform-d) δ −58.89 (s, 3F).

### Example 22: 2-(1-(6-(N-(6-(2-ethylphenyl)-5-(trifluoromethyl)pyridin-2-yl)sulfamoyl)pyridin-2-yl)-4-methylpiperidin-4-yl)acetic Acid (22)

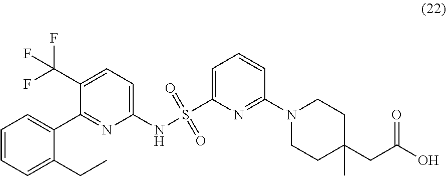 [SMILES: CCc1ccccc1-c1nc(NS(=O)(=O)c2cccc(N3CCC(C)(CC(=O)O)CC3)n2)ccc1C(F)(F)F]

2-(1-(6-(N-(6-(2-ethylphenyl)-5-(trifluoromethyl)pyridin-2-yl)sulfamoyl)pyridin-2-yl)-4-methylpiperidin-4-yl)acetic acid (22) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-ethylphenyl)-5-(trifluoromethyl)pyridin-2-yl)-6-fluoropyridine-2-sulfonamide (int-b11) and 4-methylpiperidine-4-carboxylic acid hydrochloride was replaced with 2-(4-methylpiperidin-4-yl)acetic acid. LCMS (Condition 1): m/z=563.2 [M+H]⁺, 1.75 min. ¹H NMR (400 MHz, DMSO-d₆) δ 12.10 (s, 1H), 11.46 (s, 1H), 8.20 (d, J=8.7 Hz, 1H), 7.70-7.56 (m, 2H), 7.36 (t, J=7.4 Hz, 1H), 7.28 (d, J=7.6 Hz, 1H), 7.21 (t, J=7.5 Hz, 1H), 7.07 (d, J=7.2 Hz, 2H), 6.66 (d, J=8.5 Hz, 1H), 3.28 (s, 2H), 2.99 (s, 2H), 2.29-2.14 (m, 3H), 2.12-2.00 (m, 1H), 1.79-1.65 (m, 2H), 1.62 (t, J=8.1 Hz, 2H), 0.96-0.92 (m, 3H), 0.92-0.81 (m, 3H). ¹⁹F NMR (376 MHz, DMSO-d₆) δ −56.58 (d, J=7.2 Hz, 3F).

### Example 23: 2-(1-(6-(N-(6-([1,1′-biphenyl]-2-yl)-5-(trifluoromethyl)pyridin-2-yl)sulfamoyl)pyridin-2-yl)-4-methylpiperidin-4-yl)acetic acid (23)

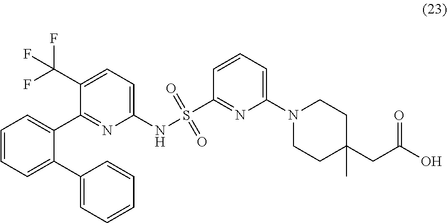 [SMILES: CC1(CC(=O)O)CCN(c2cccc(S(=O)(=O)Nc3ccc(C(F)(F)F)c(-c4ccccc4-c4ccccc4)n3)n2)CC1]

2-(1-(6-(N-(6-([1,1′-biphenyl]-2-yl)-5-(trifluoromethyl)pyridin-2-yl)sulfamoyl)pyridin-2-yl)-4-methylpiperidin-4-yl)acetic acid (23) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-([1,1′-biphenyl]-2-yl)-5-(trifluoromethyl)pyridin-2-yl)-6-fluoropyridine-2-sulfonamide (int-b5) and 4-methylpiperidine-4-carboxylic acid hydrochloride was replaced with 2-(4-methylpiperidin-4-yl)acetic acid. LCMS (Condition 1): m/z=611.2 [M+H]⁺, 1.76 min. ¹H NMR (400 MHz, DMSO-d₆) δ 12.09 (s, 1H), 11.47 (s, 1H), 7.98 (d, J=8.7 Hz, 1H), 7.65 (t, J=7.9 Hz, 1H), 7.53 (t, J=7.3 Hz, 2H), 7.42 (q, J=7.6 Hz, 2H), 7.17 (d, J=11.7 Hz, 1H), 7.13 (d, J=7.2 Hz, 1H), 7.06 (t, J=8.0 Hz, 3H), 7.01 (s, 2H), 6.67 (d, J=8.6 Hz, 1H), 3.25 (s, 2H), 3.01 (d, J=9.8 Hz, 1H), 2.93 (s, 1H), 2.22 (d, J=5.6 Hz, 2H), 1.82-1.52 (m, 4H), 0.93 (d, J=14.9 Hz, 3H). ¹⁹F NMR (376 MHz, DMSO-d₆) δ −56.98 (s, 3F).

### Example 24: 2-(1-(6-(N-(6-(2-cyclobutylphenyl)-5-(trifluoromethyl)pyridin-2-yl)sulfamoyl)pyridin-2-yl)-4-methylpiperidin-4-yl)acetic Acid (24)

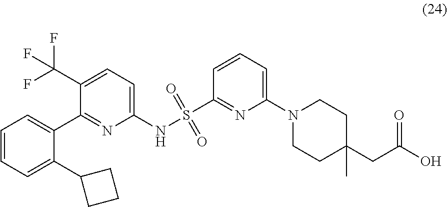 [SMILES: CC1(CC(=O)O)CCN(c2cccc(S(=O)(=O)Nc3ccc(C(F)(F)F)c(-c4ccccc4C4CCC4)n3)n2)CC1]

2-(1-(6-(N-(6-(2-cyclobutylphenyl)-5-(trifluoromethyl)pyridin-2-yl)sulfamoyl)pyridin-2-yl)-4-methylpiperidin-4-yl)acetic acid (24) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclobutylphenyl)-5-(trifluoromethyl)pyridin-2-yl)-6-fluoropyridine-2-sulfonamide (int-b13) and 4-methylpiperidine-4-carboxylic acid hydrochloride was replaced with 2-(4-methylpiperidin-4-yl)acetic acid. LCMS (Condition 1): m/z=589.2 [M+H]⁺, 1.81 min. ¹H NMR (400 MHz, DMSO-d₆) δ 12.10 (s, 1H), 11.47 (s, 1H), 8.18 (dd, J=2.3, 8.9 Hz, 1H), 7.66 (t, J=7.9 Hz, 1H), 7.61 (d, J=5.4 Hz, 1H), 7.40 (t, J=7.5 Hz, 1H), 7.33 (d, J=7.6 Hz, 1H), 7.22 (t, J=7.4 Hz, 1H), 7.08 (d, J=7.3 Hz, 1H), 7.04 (t, J=6.9 Hz, 1H), 6.68 (d, J=8.6 Hz, 1H), 3.30 (d, J=8.3 Hz, 2H), 3.04 (d, J=10.1 Hz, 3H), 2.30-2.14 (m, J=7.9 Hz, 2H), 1.92 (q, J=9.8 Hz, 1H), 1.84-1.67 (m, 4H), 1.62 (dd, J=4.7, 8.3 Hz, 2H), 1.56 (dd, J=4.6, 8.6 Hz, 2H), 1.43 (s, 1H), 0.96 (d, J=6.9 Hz, 3H). ¹⁹F NMR (376 MHz, DMSO-d₆) δ −56.17 (d, J=6.0 Hz, 3F).

### Example 25: 2-(1-(6-(N-(6-(2-isopropylphenyl)-5-(trifluoromethyl)pyridin-2-yl)sulfamoyl)pyridin-2-yl)-4-methylpiperidin-4-yl)acetic Acid (25)

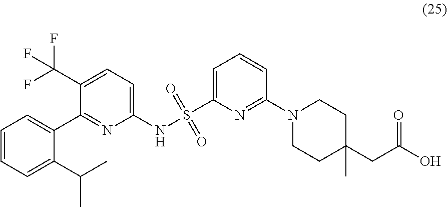 [SMILES: CC(C)c1ccccc1-c1nc(NS(=O)(=O)c2cccc(N3CCC(C)(CC(=O)O)CC3)n2)ccc1C(F)(F)F]

2-(1-(6-(N-(6-(2-isopropylphenyl)-5-(trifluoromethyl)pyridin-2-yl)sulfamoyl)pyridin-2-yl)-4-methylpiperidin-4-yl)acetic acid (25) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(2-isopropylphenyl)-5-(trifluoromethyl)pyridin-2-yl)pyridine-2-sulfonamide (int-b2) and 4-methylpiperidine-4-carboxylic acid hydrochloride was replaced with 2-(4-methylpiperidin-4-yl)acetic acid. LCMS (Condition 1): m/z=577.2 [M+H]⁺, 1.79 min. ¹H NMR (400 MHz, DMSO-d₆) δ 12.11 (s, 1H), 11.42 (s, 1H), 8.20 (dd, J=1.4, 8.9 Hz, 1H), 7.76-7.59 (m, 2H), 7.39 (d, J=5.9 Hz, 2H), 7.26-7.13 (m, 1H), 7.07 (d, J=7.2 Hz, 1H), 7.01 (t, J=7.5 Hz, 1H), 6.66 (d, J=8.6 Hz, 1H), 3.26 (s, 2H), 2.98 (t, J=10.8 Hz, 2H), 2.34 (p, J=6.8 Hz, 1H), 2.22 (m, 2H), 1.83-1.66 (m, 2H), 1.62 (t, J=7.9 Hz, 2H), 1.08-1.00 (m, 3H), 0.98-0.90 (m, 4H). ¹⁹F NMR (376 MHz, DMSO-d₆) δ −56.68 (d, J=7.1 Hz, 3F).

### Example 26: 2-(1-(6-(N-(6-(2-(tert-butyl)phenyl)-5-(trifluoromethyl)pyridin-2-yl)sulfamoyl)pyridin-2-yl)-4-methylpiperidin-4-yl)acetic Acid (26)

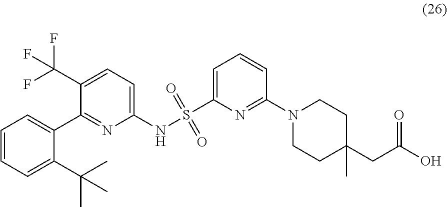 [SMILES: CC1(CC(=O)O)CCN(c2cccc(S(=O)(=O)Nc3ccc(C(F)(F)F)c(-c4ccccc4C(C)(C)C)n3)n2)CC1]

2-(1-(6-(N-(6-(2-(tert-butyl)phenyl)-5-(trifluoromethyl)pyridin-2-yl)sulfamoyl)pyridin-2-yl)-4-methylpiperidin-4-yl)acetic acid (26) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-(tert-butyl)phenyl)-5-(trifluoromethyl)pyridin-2-yl)-6-fluoropyridine-2-sulfonamide (int-b14) and 4-methylpiperidine-4-carboxylic acid hydrochloride was replaced with 2-(4-methylpiperidin-4-yl)acetic acid. LCMS (Condition 1): m/z=577.3 [M+H]⁺, 1.79 min. ¹H NMR (400 MHz, DMSO-d₆) δ 12.10 (s, 1H), 11.42 (s, 1H), 8.16 (d, J=8.8 Hz, 1H), 7.64 (t, J=7.8 Hz, 2H), 7.55 (d, J=8.1 Hz, 1H), 7.36 (t, J=7.7 Hz, 1H), 7.17 (t, J=7.4 Hz, 1H), 7.07 (d, J=7.2 Hz, 1H), 6.86 (t, J=7.2 Hz, 1H), 6.65 (d, J=8.6 Hz, 1H), 3.29 (s, 2H), 3.01 (s, 2H), 2.31-2.11 (m, J=7.9 Hz, 2H), 1.70 (dd, J=7.7, 14.3 Hz, 2H), 1.63 (td, J=3.2, 8.3 Hz, 2H), 0.99 (s, 9H), 0.96 (d, J=6.2 Hz, 3H). ¹⁹F NMR (376 MHz, DMSO-d₆) δ −55.88 (d, J=3.5 Hz, 3F).

### Example 27: 4-methyl-1-(6-(N-(6-(2-(pyridin-4-yl)phenyl)-5-(trifluoromethyl)pyridin-2-yl)sulfamoyl)pyridin-2-yl)piperidine-4-carboxylic Acid (27)

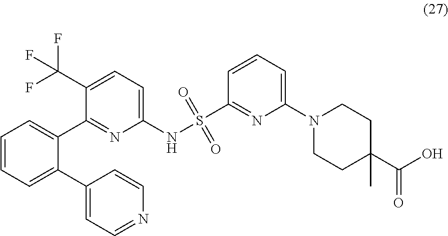 [SMILES: CC1(C(=O)O)CCN(c2cccc(S(=O)(=O)Nc3ccc(C(F)(F)F)c(-c4ccccc4-c4ccncc4)n3)n2)CC1]

4-methyl-1-(6-(N-(6-(2-(pyridin-4-yl)phenyl)-5-(trifluoromethyl)pyridin-2-yl)sulfamoyl)pyridin-2-yl)piperidine-4-carboxylic acid (27) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced 6-fluoro-N-(6-(2-(pyridin-4-yl)phenyl)-5-(trifluoromethyl)pyridin-2-yl)pyridine-2-sulfonamide (int-b16). LCMS (Condition 1): m/z=598.1 [M+H]⁺, 1.36 min. ¹H NMR (400 MHz, Chloroform-d) δ 8.35-8.23 (m, 2H), 7.84 (d, J=8.9 Hz, 1H), 7.65-7.58 (m, 2H), 7.56 (dd, J=1.4, 7.6 Hz, 1H), 7.51 (td, J=1.4, 7.6 Hz, 1H), 7.41 (dd, J=1.4, 7.6 Hz, 1H), 7.36 (d, J=7.6 Hz, 1H), 7.31 (d, J=7.2 Hz, 2H), 7.04-6.95 (m, 2H), 6.88 (d, J=8.7 Hz, 1H), 3.93 (d, J=13.5 Hz, 2H), 2.99 (ddd, J=3.0, 11.3, 13.8 Hz, 2H), 2.11 (d, J=13.6 Hz, 2H), 1.48-1.31 (m, 2H), 1.26 (s, 3H). ¹⁹F NMR (376 MHz, Chloroform-d) δ −58.08 (s, 3F).

### Example 28: 4-methyl-1-(6-(N-(6-(2-phenoxyphenyl)-5-(trifluoromethyl)pyridin-2-yl)sulfamoyl)pyridin-2-yl)piperidine-4-carboxylic Acid (28)

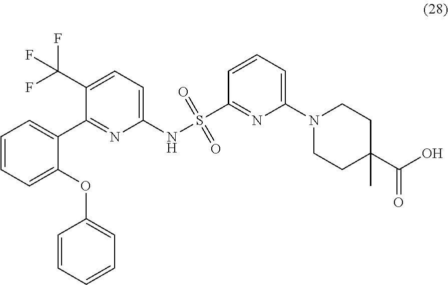 [SMILES: CC1(C(=O)O)CCN(c2cccc(S(=O)(=O)Nc3ccc(C(F)(F)F)c(-c4ccccc4Oc4ccccc4)n3)n2)CC1]

4-methyl-1-(6-(N-(6-(2-phenoxyphenyl)-5-(trifluoromethyl)pyridin-2-yl)sulfamoyl)pyridin-2-yl)piperidine-4-carboxylic acid (28) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(2-phenoxyphenyl)-5-(trifluoromethyl)pyridin-2-yl)pyridine-2-sulfonamide (int-b17). LCMS (Condition 1): m/z=613.2 [M+H]⁺, 1.91 min. Rotamers are present in both NMR spectra. ¹H NMR (400 MHz, Chloroform-d) δ 7.95 (d, J=9.0 Hz, 1H), 7.67 (d, J=8.9 Hz, 1H), 7.61-7.50 (m, 1H), 7.37-7.32 (m, 1H), 7.26-7.17 (m, 3H), 7.17-7.07 (m, 1H), 7.05-6.98 (m, 1H), 6.95-6.82 (m, 3H), 6.74-6.66 (m, 1H), 3.97-3.83 (m, 1.5H), 3.70-3.60 (m, 0.5H), 2.98-2.85 (m, 2H), 2.05-1.96 (m, 2H), 1.35-1.29 (m, 3H), 1.20 (s, 2H), 1.13 (s, 1H). ¹⁹F NMR (376 MHz, Chloroform-d) δ −58.37 (s, 1F), −58.51 (s, 4F).

### Example 29: 1-(6-(N-(6-([1,1′-biphenyl]-2-yl)-5-chloropyridin-2-yl)sulfamoyl)pyridin-2-yl)-3-methylpiperidine-3-carboxylic Acid (29)

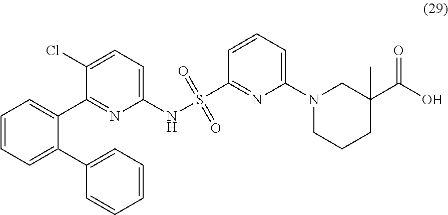 [SMILES: CC1(C(=O)O)CCCN(c2cccc(S(=O)(=O)Nc3ccc(Cl)c(-c4ccccc4-c4ccccc4)n3)n2)C1]

1-(6-(N-(6-([1,1′-biphenyl]-2-yl)-5-chloropyridin-2-yl)sulfamoyl)pyridin-2-yl)-3-methylpiperidine-3-carboxylic acid (29) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-([1,1′-biphenyl]-2-yl)-5-chloropyridin-2-yl)-6-fluoropyridine-2-sulfonamide (int-b8) and 4-methylpiperidine-4-carboxylic acid hydrochloride was replaced with 3-methylpiperidine-3-carboxylic acid hydrochloride. LCMS (Condition 1): m/z 563.2 [M+H]⁺, 1.60 min. ¹H NMR (400 MHz, DMSO-d₆) δ 12.34 (s, 1H), 11.17 (s, 1H), 7.67 (dd, J=8.7, 7.3 Hz, 1H), 7.62 (d, J=8.8 Hz, 1H), 7.53 (td, J=7.5, 1.4 Hz, 1H), 7.47-7.40 (m, 2H), 7.27 (d, J=8.7 Hz, 1H), 7.22-7.08 (m, 6H), 7.00-6.95 (m, 2H), 3.91 (d, J=13.1 Hz, 1H), 3.76-3.67 (m, 1H), 3.19-3.13 (m, 1H), 3.08 (s, 1H), 2.04-1.96 (m, 1H), 1.56-1.36 (m, 3H), 1.03 (s, 3H).

### Example 30: 1-(6-(N-(4′-isopropyl-3-(trifluoromethyl)-[2,3′-bipyridin]-6-yl)sulfamoyl)pyridin-2-yl)-3-methylpiperidine-3-carboxylic Acid (30)

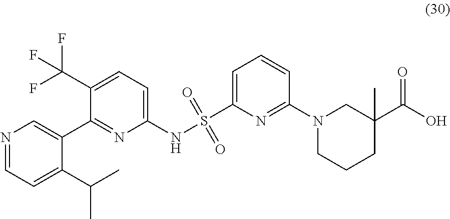 [SMILES: CC(C)c1ccncc1-c1nc(NS(=O)(=O)c2cccc(N3CCCC(C)(C(=O)O)C3)n2)ccc1C(F)(F)F]

1-(6-(N-(4′-isopropyl-3-(trifluoromethyl)-[2,3′-bipyridin]-6-yl)sulfamoyl)pyridin-2-yl)-3-methylpiperidine-3-carboxylic acid (30) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(4′-isopropyl-3-(trifluoromethyl)-[2,3′-bipyridin]-6-yl)pyridine-2-sulfonamide (int-b18) and 4-methylpiperidine-4-carboxylic acid hydrochloride was replaced with 3-methylpiperidine-3-carboxylic acid hydrochloride. LCMS (Condition 1): m/z 564.2 [M+H]⁺, 1.49 min. ¹H NMR (400 MHz, DMSO-d₆) δ 12.33 (s, 1H), 11.60 (s, 1H), 8.58 (s, 1H), 8.22 (d, J=8.8 Hz, 2H), 7.66-7.57 (m, 2H), 7.46 (s, 1H), 7.12-7.01 (m, 2H), 3.86 (dd, J=12.9, 8.9 Hz, 1H), 3.66-3.56 (m, 1H), 3.11 (dd, J=13.0, 9.1 Hz, 1H), 3.07-2.97 (m, 1H), 2.41-2.33 (m, 1H), 2.01-1.95 (m, 1H), 1.49-1.36 (m, 3H), 1.05-0.93 (m, 9H).

### Example 31: 2-((1-(6-(N-(6-(2-isopropylphenyl)-5-(trifluoromethyl)pyridin-2-yl)sulfamoyl)pyridin-2-yl)piperidin-4-yl)ₙxy)acetic Acid (31)

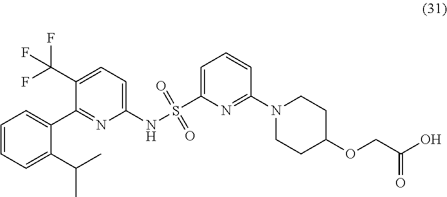 [SMILES: CC(C)c1ccccc1-c1nc(NS(=O)(=O)c2cccc(N3CCC(OCC(=O)O)CC3)n2)ccc1C(F)(F)F]

2-((1-(6-(N-(6-(2-isopropylphenyl)-5-(trifluoromethyl)pyridin-2-yl)sulfamoyl)pyridin-2-yl)piperidin-4-yl)ₙxy)acetic acid (31) was synthesized using the procedure described in Example 6, except 6-fluoro-N-(6-(5-fluoro-2-methylphenyl)-5-(trifluoromethyl)pyridin-2-yl)pyridine-2-sulfonamide (int-b10) was replaced with 6-fluoro-N-(6-(2-isopropylphenyl)-5-(trifluoromethyl)pyridin-2-yl)pyridine-2-sulfonamide (int-b2) and 2-(4-methylpiperidin-4-yl)acetic acid hydrochloride was replaced with 2-(piperidin-4-yloxy)acetic acid hydrochloride. LCMS (Condition 1): m/z 579.1 [M+H]⁺, 1.77 min. ¹H NMR (400 MHz, Methanol-d₄) δ 8.07 (d, J=8.9 Hz, 1H), 7.72-7.65 (m, 1H), 7.62 (dd, J=8.7, 7.3 Hz, 1H), 7.42-7.34 (m, 2H), 7.23-7.13 (m, 2H), 7.01 (d, J=7.3 Hz, 1H), 6.97 (d, J=8.7 Hz, 1H), 4.12 (s, 2H), 3.91-3.79 (m, 2H), 3.65-3.62 (m, 1H), 3.23-3.10 (m, 2H), 2.45-2.42 (m, 1H), 1.84-1.81 (m, 2H), 1.49-1.44 (m, 2H), 1.10 (d, J=6.9 Hz, 3H), 1.04 (d, J=6.8 Hz, 3H).

### Example 32: 2-(1-(6-(N-(6-(o-tolyl)-5-(trifluoromethyl)pyridin-2-yl)sulfamoyl)pyridin-2-yl)pyrrolidin-3-yl)acetic Acid (32)

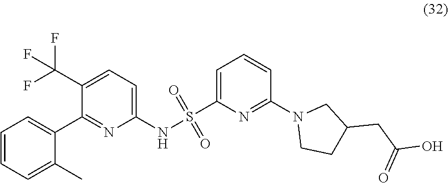 [SMILES: Cc1ccccc1-c1nc(NS(=O)(=O)c2cccc(N3CCC(CC(=O)O)C3)n2)ccc1C(F)(F)F]

2-(1-(6-(N-(6-(o-tolyl)-5-(trifluoromethyl)pyridin-2-yl)sulfamoyl)pyridin-2-yl)pyrrolidin-3-yl)acetic acid (32) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 2-(pyrrolidin-3-yl)acetic acid. LCMS (Condition 1): m/z 521.0 [M+H]⁺, 1.56 min. ¹H NMR (400 MHz, DMSO-d₆) δ 12.22 (s, 1H), 11.52 (s, 1H), 8.18 (d, J=8.9 Hz, 1H), 7.67-7.60 (m, 1H), 7.58 (d, J=8.1 Hz, 1H), 7.32 (t, J=7.5 Hz, 1H), 7.27-7.16 (m, 2H), 7.07 (d, J=7.2 Hz, 2H), 6.65 (d, J=8.6 Hz, 1H), 3.55-3.46 (m, 1H), 3.38 (q, J=7.0 Hz, 1H), 3.22-3.10 (m, 1H), 2.85 (t, J=8.9 Hz, 1H), 2.38-2.32 (m, 2H), 2.13-2.03 (m, 1H), 1.82 (s, 3H), 1.66-1.52 (m, 1H), 1.09 (t, J=7.0 Hz, 1H).

### Example 33: 2-(1-(6-(N-(6-(2-cyclopropylphenyl)-5-(trifluoromethyl)pyridin-2-yl)sulfamoyl)pyridin-2-yl)pyrrolidin-3-yl)acetic Acid (33)

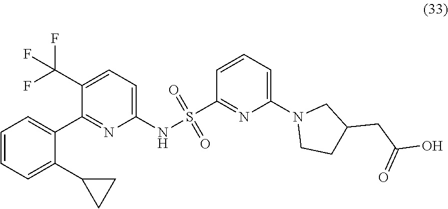 [SMILES: O=C(O)CC1CCN(c2cccc(S(=O)(=O)Nc3ccc(C(F)(F)F)c(-c4ccccc4C4CC4)n3)n2)C1]

2-(1-(6-(N-(6-(2-cyclopropylphenyl)-5-(trifluoromethyl)pyridin-2-yl)sulfamoyl)pyridin-2-yl)pyrrolidin-3-yl)acetic acid (33) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropylphenyl)-5-(trifluoromethyl)pyridin-2-yl)-6-fluoropyridine-2-sulfonamide (int-b19) and 4-methylpiperidine-4-carboxylic acid hydrochloride was replaced with 2-(pyrrolidin-3-yl)acetic acid. LCMS (Condition 1): m/z 547.2 [M+H]⁺, 1.60 min. ¹H NMR (400 MHz, DMSO-d₆) δ 12.23 (s, 1H), 11.52 (s, 1H), 8.19 (d, J=8.9 Hz, 1H), 7.68-7.58 (m, 2H), 7.32 (t, J=7.6 Hz, 1H), 7.18 (t, J=7.5 Hz, 1H), 7.09 (d, J=7.2 Hz, 1H), 7.07-7.01 (m, 1H), 6.93 (d, J=7.9 Hz, 1H), 6.65 (d, J=8.6 Hz, 1H), 3.55-3.45 (m, 1H), 3.33-3.26 (m, 2H), 3.22-3.08 (m, 1H), 2.90-2.78 (m, 1H), 2.40-2.32 (m, 2H), 2.14-2.03 (m, 1H), 1.65-1.52 (m, 1H), 1.22 (d, J=16.8 Hz, 1H), 0.69-0.48 (m, 3H), 0.40 (s, 1H).

### Example 34: 3-(methyl(6-(N-(6-(o-tolyl)-5-(trifluoromethyl)pyridin-2-yl)sulfamoyl)pyridin-2-yl)amino)propanoic Acid (34)

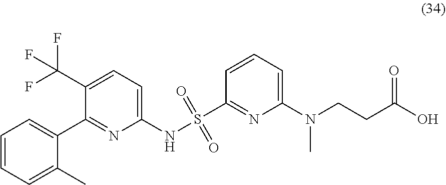 [SMILES: Cc1ccccc1-c1nc(NS(=O)(=O)c2cccc(N(C)CCC(=O)O)n2)ccc1C(F)(F)F]

3-(methyl(6-(N-(6-(o-tolyl)-5-(trifluoromethyl)pyridin-2-yl)sulfamoyl)pyridin-2-yl)amino)propanoic acid (34) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1), 4-methylpiperidine-4-carboxylic acid hydrochloride was replaced with 3-(methylamino)propanoic acid, and dioxane was replaced with DMA. LCMS (Condition 1): m/z 495.2 [M+H]⁺, 1.55 min. ¹H NMR (500 MHz, DMSO-d₆) δ 12.22 (s, 1H), 11.59 (s, 1H), 8.15 (d, J=8.9 Hz, 1H), 7.71-7.64 (m, 1H), 7.52 (d, J=8.7 Hz, 1H), 7.32 (t, J=7.5 Hz, 1H), 7.25-7.18 (m, 2H), 7.11 (d, J=7.3 Hz, 1H), 7.07 (d, J=7.5 Hz, 1H), 6.86 (d, J=8.7 Hz, 1H), 3.69-3.49 (m, 2H), 2.91 (s, 3H), 2.32 (t, J=7.0 Hz, 2H), 1.78 (s, 3H).

### Example 35: 3-((6-(N-(6-(o-tolyl)-5-(trifluoromethyl)pyridin-2-yl)sulfamoyl)pyridin-2-yl)amino)propanoic acid (35)

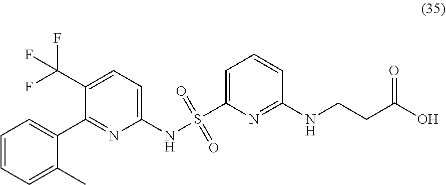 [SMILES: Cc1ccccc1-c1nc(NS(=O)(=O)c2cccc(NCCC(=O)O)n2)ccc1C(F)(F)F]

3-((6-(N-(6-(o-tolyl)-5-(trifluoromethyl)pyridin-2-yl)sulfamoyl)pyridin-2-yl)amino)propanoic acid (35) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1), 4-methylpiperidine-4-carboxylic acid hydrochloride was replaced with 3-aminopropanoic acid, and dioxane was replaced with DMA. LCMS (Condition 1): m/z 481.0 [M+H]⁺, 1.47 min. ¹H NMR (500 MHz, DMSO-d₆) δ 12.16 (s, 1H), 11.52 (s, 1H), 8.15 (d, J=8.9 Hz, 1H), 7.58-7.46 (m, 2H), 7.34-7.28 (m, 1H), 7.26-7.17 (m, 3H), 7.07 (d, J=7.6 Hz, 1H), 7.05-6.99 (m, 1H), 6.71-6.66 (m, 1H), 3.29-3.21 (m, 2H), 2.28 (t, J=6.7 Hz, 2H), 1.81 (s, 3H).

### Example 36: 4-((6-(N-(6-(o-tolyl)-5-(trifluoromethyl)pyridin-2-yl)sulfamoyl)pyridin-2-yl)amino)butanoic Acid (36)

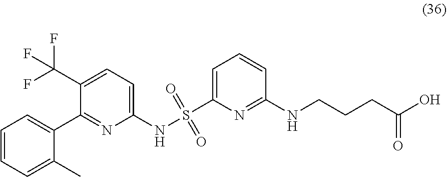 [SMILES: Cc1ccccc1-c1nc(NS(=O)(=O)c2cccc(NCCCC(=O)O)n2)ccc1C(F)(F)F]

4-((6-(N-(6-(o-tolyl)-5-(trifluoromethyl)pyridin-2-yl)sulfamoyl)pyridin-2-yl)amino)butanoic acid (36) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1), and 4-methylpiperidine-4-carboxylic acid hydrochloride was replaced with 4-aminobutanoic acid, and dioxane was replaced with DMA. LCMS (Condition 1): m/z 495.0 [M+H]⁺, 1.49 min. ¹H NMR (400 MHz, Chloroform-d) δ 10.05 (s, 1H), 8.01 (d, J=8.9 Hz, 1H), 7.65 (d, J=8.8 Hz, 1H), 7.54-7.48 (m, 1H), 7.41-7.35 (m, 1H), 7.32-7.22 (m, 3H), 7.16 (d, J=7.5 Hz, 1H), 6.53 (d, J=8.4 Hz, 1H), 4.71 (s, 1H), 3.45-3.23 (m, 2H), 2.33-2.25 (m, 2H), 2.08-2.03 (m, 3H), 1.91-1.81 (m, 2H).

### Example 37: 4-(methyl(6-(N-(6-(o-tolyl)-5-(trifluoromethyl)pyridin-2-yl)sulfamoyl)pyridin-2-yl)amino)butanoic Acid (37)

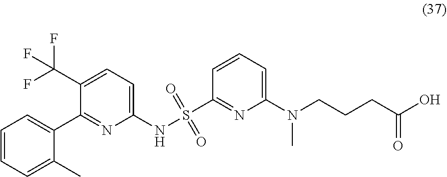 [SMILES: Cc1ccccc1-c1nc(NS(=O)(=O)c2cccc(N(C)CCCC(=O)O)n2)ccc1C(F)(F)F]

4-(methyl(6-(N-(6-(o-tolyl)-5-(trifluoromethyl)pyridin-2-yl)sulfamoyl)pyridin-2-yl)amino)butanoic acid (37) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1), 4-methylpiperidine-4-carboxylic acid hydrochloride was replaced with 4-(methylamino)butanoic acid hydrochloride, and dioxane was replaced with DMA. LCMS (Condition 1): m/z 509.0 [M+H]⁺, 1.56 min. ¹H NMR (400 MHz, Chloroform-d) δ 10.12 (s, 1H), 8.05 (d, J=8.9 Hz, 1H), 7.76 (d, J=8.8 Hz, 1H), 7.61-7.53 (m, 1H), 7.38-7.33 (m, 1H), 7.27-7.17 (m, 3H), 7.11 (d, J=7.5 Hz, 1H), 6.63 (d, J=8.6 Hz, 1H), 3.99-3.85 (m, 1H), 3.72-3.56 (m, 1H), 2.97 (s, 3H), 2.21-2.14 (m, 2H), 2.00 (s, 3H), 1.97-1.83 (m, 2H).

### Example 38: 3-((6-(N-(6-(2-cyclopropylphenyl)-5-(trifluoromethyl)pyridin-2-yl)sulfamoyl)pyridin-2-yl)(methyl)amino)propanoic Acid (38)

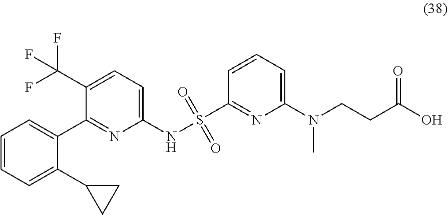 [SMILES: CN(CCC(=O)O)c1cccc(S(=O)(=O)Nc2ccc(C(F)(F)F)c(-c3ccccc3C3CC3)n2)n1]

3-((6-(N-(6-(2-cyclopropylphenyl)-5-(trifluoromethyl)pyridin-2-yl)sulfamoyl)pyridin-2-yl)(methyl)amino)propanoic acid (38) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropylphenyl)-5-(trifluoromethyl)pyridin-2-yl)-6-fluoropyridine-2-sulfonamide (int-b19), 4-methylpiperidine-4-carboxylic acid hydrochloride was replaced with 3-(methylamino)propanoic acid, and dioxane was replaced with DMA. LCMS (Condition 1): m/z 521.1 [M+H]⁺, 1.58 min. ¹H NMR (400 MHz, DMSO-d₆) δ 12.28 (s, 1H), 11.58 (s, 1H), 8.16 (d, J=8.9 Hz, 1H), 7.72-7.65 (m, 1H), 7.57 (d, J=8.6 Hz, 1H), 7.34-7.28 (m, 1H), 7.20-7.14 (m, 1H), 7.12 (d, J=7.2 Hz, 1H), 7.04 (d, J=7.5 Hz, 1H), 6.92 (d, J=7.8 Hz, 1H), 6.86 (d, J=8.7 Hz, 1H), 3.67-3.47 (m, 2H), 2.90 (s, 3H), 2.32 (t, J=7.1 Hz, 2H), 1.27-1.15 (m, 1H), 0.68-0.48 (m, 3H), 0.43-0.31 (m, 1H).

### Example 39: 3-((6-(N-(6-(2-cyclopropylphenyl)-5-(trifluoromethyl)pyridin-2-yl)sulfamoyl)pyridin-2-yl)amino)propanoic Acid (39)

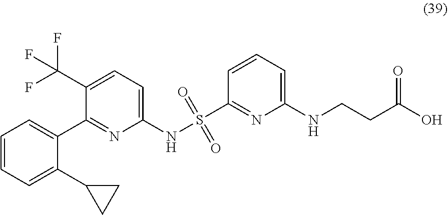 [SMILES: O=C(O)CCNc1cccc(S(=O)(=O)Nc2ccc(C(F)(F)F)c(-c3ccccc3C3CC3)n2)n1]

3-((6-(N-(6-(2-cyclopropylphenyl)-5-(trifluoromethyl)pyridin-2-yl)sulfamoyl)pyridin-2-yl)amino)propanoic acid (39) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropylphenyl)-5-(trifluoromethyl)pyridin-2-yl)-6-fluoropyridine-2-sulfonamide (int-b19), 4-methylpiperidine-4-carboxylic acid hydrochloride was replaced with 3-aminopropanoic acid, and dioxane was replaced with DMA. LCMS (Condition 1): m/z 507.0 [M+H]⁺, 1.52 min. ¹H NMR (400 MHz, Chloroform-d) δ 10.72 (s, 1H), 8.09 (d, J=8.8 Hz, 1H), 7.77 (d, J=8.7 Hz, 1H), 7.42-7.36 (m, 1H), 7.36-7.30 (m, 1H), 7.21-7.14 (m, 1H), 7.10-7.03 (m, 2H), 6.98 (d, J=7.8 Hz, 1H), 6.48 (d, J=8.4 Hz, 1H), 4.98 (t, J=5.9 Hz, 1H), 3.87-3.68 (m, 2H), 2.38-2.25 (m, 2H), 1.31-1.18 (m, 1H), 0.56-0.44 (m, 4H).

### Example 40: 4-((6-(N-(6-(2-cyclopropylphenyl)-5-(trifluoromethyl)pyridin-2-yl)sulfamoyl)pyridin-2-yl)amino)butanoic Acid (40)

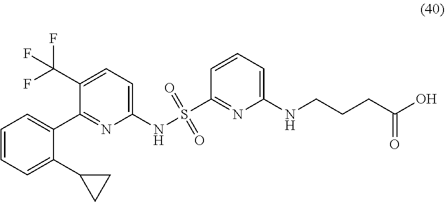 [SMILES: O=C(O)CCCNc1cccc(S(=O)(=O)Nc2ccc(C(F)(F)F)c(-c3ccccc3C3CC3)n2)n1]

4-((6-(N-(6-(2-cyclopropylphenyl)-5-(trifluoromethyl)pyridin-2-yl)sulfamoyl)pyridin-2-yl)amino)butanoic acid (40) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropylphenyl)-5-(trifluoromethyl)pyridin-2-yl)-6-fluoropyridine-2-sulfonamide (int-b19), 4-methylpiperidine-4-carboxylic acid hydrochloride was replaced with 4-aminobutanoic acid, and dioxane was replaced with DMA. LCMS (Condition 1): m/z 521.1 [M+H]⁺, 1.54 min. ¹H NMR (400 MHz, Chloroform-d) δ 9.68 (s, 1H), 8.00 (d, J=9.0 Hz, 1H), 7.64 (d, J=8.9 Hz, 1H), 7.53-7.45 (m, 1H), 7.40-7.33 (m, 1H), 7.30-7.26 (m, 1H), 7.24-7.18 (m, 1H), 7.14 (d, J=7.5 Hz, 1H), 7.00 (d, J=7.8 Hz, 1H), 6.57-6.52 (m, 1H), 4.98 (br s, 1H), 3.26-3.09 (m, 2H), 2.28 (t, J=6.9 Hz, 2H), 1.76 (p, J=6.9 Hz, 2H), 1.56-1.42 (m, 1H), 0.79-0.60 (m, 3H), 0.60-0.49 (m, 1H).

### Example 41: 4-((6-(N-(6-(2-cyclopropylphenyl)-5-(trifluoromethyl)pyridin-2-yl)sulfamoyl)pyridin-2-yl)(methyl)amino)butanoic Acid (41)

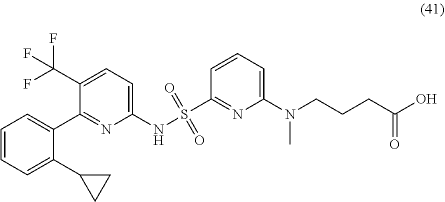 [SMILES: CN(CCCC(=O)O)c1cccc(S(=O)(=O)Nc2ccc(C(F)(F)F)c(-c3ccccc3C3CC3)n2)n1]

4-((6-(N-(6-(2-cyclopropylphenyl)-5-(trifluoromethyl)pyridin-2-yl)sulfamoyl)pyridin-2-yl)(methyl)amino)butanoic acid (41) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropylphenyl)-5-(trifluoromethyl)pyridin-2-yl)-6-fluoropyridine-2-sulfonamide (int-b19), 4-methylpiperidine-4-carboxylic acid hydrochloride was replaced with 4-(methylamino)butanoic acid hydrochloride, and dioxane was replaced with DMA. LCMS (Condition 1): m/z 535.2 [M+H]⁺, 1.54 min. ¹H NMR (400 MHz, Chloroform-d) δ 10.28 (s, 1H), 8.06 (d, J=8.9 Hz, 1H), 7.77 (d, J=8.8 Hz, 1H), 7.59-7.53 (m, 1H), 7.39-7.33 (m, 1H), 7.24-7.16 (m, 2H), 7.10 (d, J=7.6 Hz, 1H), 6.98 (d, J=7.8 Hz, 1H), 6.63 (d, J=8.6 Hz, 1H), 4.00-3.87 (m, 1H), 3.66-3.55 (m, 1H), 2.96 (s, 3H), 2.25-2.10 (m, 2H), 2.00-1.80 (m, 2H), 1.45-1.35 (m, 1H), 0.73-0.51 (m, 4H).

### Example 42: 5-((6-(N-(6-(2-cyclopropylphenyl)-5-(trifluoromethyl)pyridin-2-yl)sulfamoyl)pyridin-2-yl)amino)pentanoic Acid (42)

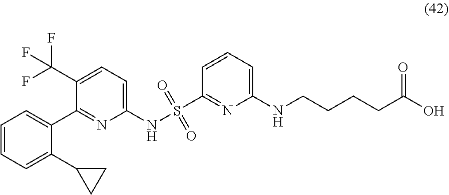 [SMILES: O=C(O)CCCCNc1cccc(S(=O)(=O)Nc2ccc(C(F)(F)F)c(-c3ccccc3C3CC3)n2)n1]

5-((6-(N-(6-(2-cyclopropylphenyl)-5-(trifluoromethyl)pyridin-2-yl)sulfamoyl)pyridin-2-yl)amino)pentanoic acid (42) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropylphenyl)-5-(trifluoromethyl)pyridin-2-yl)-6-fluoropyridine-2-sulfonamide (int-b19), 4-methylpiperidine-4-carboxylic acid hydrochloride was replaced with 5-aminopentanoic acid, and dioxane was replaced with DMA. LCMS (Condition 1): m/z 535.1 [M+H]⁺, 1.57 min. ¹H NMR (400 MHz, Chloroform-d) δ 10.74 (s, 1H), 7.99 (d, J=9.0 Hz, 1H), 7.71 (d, J=8.9 Hz, 1H), 7.49-7.42 (m, 1H), 7.39-7.33 (m, 1H), 7.30 (d, J=7.3 Hz, 1H), 7.19 (t, J=7.5 Hz, 1H), 7.10 (d, J=7.5 Hz, 1H), 6.96 (d, J=7.8 Hz, 1H), 6.46 (d, J=8.4 Hz, 1H), 4.78-4.63 (m, 1H), 3.47-3.24 (m, 2H), 2.38-2.25 (m, 2H), 1.73-1.58 (m, 4H), 1.45-1.35 (m, 1H), 0.74-0.51 (m, 4H).

### Example 43: 5-((6-(N-(6-(o-tolyl)-5-(trifluoromethyl)pyridin-2-yl)sulfamoyl)pyridin-2-yl)amino)pentanoic Acid (43)

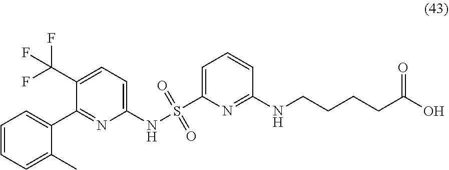 [SMILES: Cc1ccccc1-c1nc(NS(=O)(=O)c2cccc(NCCCCC(=O)O)n2)ccc1C(F)(F)F]

5-((6-(N-(6-(o-tolyl)-5-(trifluoromethyl)pyridin-2-yl)sulfamoyl)pyridin-2-yl)amino)pentanoic acid (43) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1), 4-methylpiperidine-4-carboxylic acid hydrochloride was replaced with 5-aminopentanoic acid, and dioxane was replaced with DMA. LCMS (Condition 1): m/z 509.0 [M+H]⁺, 1.52 min. ¹H NMR (400 MHz, Chloroform-d) δ 10.70 (s, 1H), 8.00 (d, J=9.0 Hz, 1H), 7.71 (d, J=8.9 Hz, 1H), 7.51-7.46 (m, 1H), 7.41-7.35 (m, 1H), 7.35-7.31 (m, 1H), 7.27-7.21 (m, 2H), 7.13 (d, J=7.6 Hz, 1H), 6.50 (d, J=8.4 Hz, 1H), 4.74 (s, 1H), 3.37 (s, 2H), 2.40-2.26 (m, 2H), 2.03 (s, 3H), 1.75-1.43 (m, 4H).

### Example 44: 3-((6-(N-(6-(2-isopropylphenyl)-5-(trifluoromethyl)pyridin-2-yl)sulfamoyl)pyridin-2-yl)(methyl)amino)propanoic Acid (44)

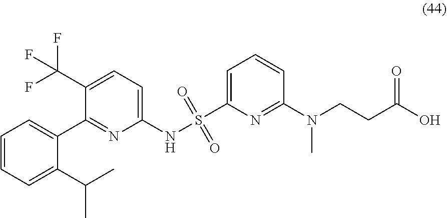 [SMILES: CC(C)c1ccccc1-c1nc(NS(=O)(=O)c2cccc(N(C)CCC(=O)O)n2)ccc1C(F)(F)F]

3-((6-(N-(6-(2-isopropylphenyl)-5-(trifluoromethyl)pyridin-2-yl)sulfamoyl)pyridin-2-yl)(methyl)amino)propanoic acid (44) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(2-isopropylphenyl)-5-(trifluoromethyl)pyridin-2-yl)pyridine-2-sulfonamide (int-b2), 4-methylpiperidine-4-carboxylic acid hydrochloride was replaced with 3-(methylamino)propanoic acid, and dioxane was replaced with DMA. LCMS (Condition 1): m/z 523.1 [M+H]⁺, 1.71 min. ¹H NMR (400 MHz, Methanol-d₄) δ 8.05 (d, J=8.9 Hz, 1H), 7.69-7.57 (m, 2H), 7.41-7.31 (m, 2H), 7.23-7.11 (m, 2H), 7.04-6.96 (m, 1H), 6.79 (d, J=8.6 Hz, 1H), 3.78-3.62 (m, 2H), 3.00 (s, 3H), 2.55-2.35 (m, 3H), 1.09 (d, J=6.9 Hz, 3H), 1.02 (d, J=6.8 Hz, 3H).

### Example 45: 3-((6-(N-(6-(5-fluoro-2-methylphenyl)-5-(trifluoromethyl)pyridin-2-yl)sulfamoyl)pyridin-2-yl)(methyl)amino)-2,2-dimethylpropanoic Acid (45)

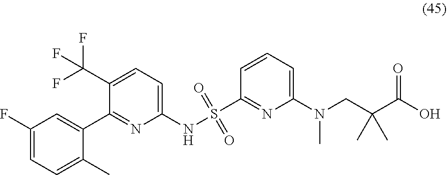 [SMILES: Cc1ccc(F)cc1-c1nc(NS(=O)(=O)c2cccc(N(C)CC(C)(C)C(=O)O)n2)ccc1C(F)(F)F]

3-((6-(N-(6-(5-fluoro-2-methylphenyl)-5-(trifluoromethyl)pyridin-2-yl)sulfamoyl)pyridin-2-yl)(methyl)amino)-2,2-dimethylpropanoic acid (45) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with 2,2-dimethyl-3-(methylamino)propanoic acid hydrochloride. LCMS (Condition 1): m/z 541.1 [M+H]⁺, 1.66 min. ¹H NMR (400 MHz, Methanol-d₄) δ 8.07 (d, J=8.8 Hz, 1H), 7.64-7.55 (m, 1H), 7.49 (d, J=8.8 Hz, 1H), 7.25-7.14 (m, 2H), 7.04 (td, J=8.6, 2.7 Hz, 1H), 6.81 (d, J=8.7 Hz, 1H), 6.77 (dd, J=9.0, 2.7 Hz, 1H), 3.80 (d, J=2.0 Hz, 2H), 2.97 (d, J=2.0 Hz, 3H), 1.83 (s, 3H), 1.04 (s, 6H).

### Example 46: 2,2-dimethyl-3-(methyl(6-(N-(6-(o-tolyl)-5-(trifluoromethyl)pyridin-2-yl)sulfamoyl)pyridin-2-yl)amino)propanoic Acid (46)

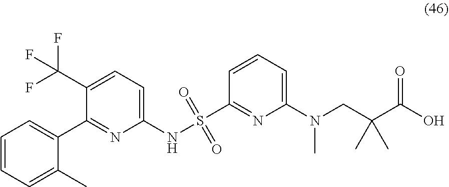 [SMILES: Cc1ccccc1-c1nc(NS(=O)(=O)c2cccc(N(C)CC(C)(C)C(=O)O)n2)ccc1C(F)(F)F]

2,2-dimethyl-3-(methyl(6-(N-(6-(o-tolyl)-5-(trifluoromethyl)pyridin-2-yl)sulfamoyl)pyridin-2-yl)amino)propanoic acid (46) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 2,2-dimethyl-3-(methylamino)propanoic acid hydrochloride. LCMS (Condition 1): m/z 523.2 [M+H]⁺, 1.64 min. ¹H NMR (400 MHz, Methanol-d₄) δ 7.96 (d, J=8.9 Hz, 1H), 7.54-7.47 (m, 1H), 7.42 (dt, J=8.9, 0.9 Hz, 1H), 7.20 (td, J=7.5, 1.4 Hz, 1H), 7.14-7.05 (m, 3H), 6.93 (d, J=7.6 Hz, 1H), 6.71 (dd, J=8.7, 0.6 Hz, 1H), 3.71-3.67 (m, 2H), 2.88 (s, 3H), 1.79 (s, 3H), 0.98-0.90 (m, 6H).

### Example 47: 3-((6-(N-(6-(3-fluoro-2-methylphenyl)-5-(trifluoromethyl)pyridin-2-yl)sulfamoyl)pyridin-2-yl)(methyl)amino)-2,2-dimethylpropanoic Acid (47)

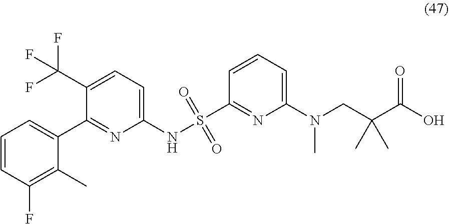 [SMILES: Cc1c(F)cccc1-c1nc(NS(=O)(=O)c2cccc(N(C)CC(C)(C)C(=O)O)n2)ccc1C(F)(F)F]

3-((6-(N-(6-(3-fluoro-2-methylphenyl)-5-(trifluoromethyl)pyridin-2-yl)sulfamoyl)pyridin-2-yl)(methyl)amino)-2,2-dimethylpropanoic acid (47) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(3-fluoro-2-methylphenyl)-5-(trifluoromethyl)pyridin-2-yl)pyridine-2-sulfonamide (int-b12) and 4-methylpiperidine-4-carboxylic acid hydrochloride was replaced with 2,2-dimethyl-3-(methylamino)propanoic acid hydrochloride. LCMS (Condition 1): m/z 541.2 [M+H]⁺, 1.65 min. ¹H NMR (400 MHz, Chloroform-d) δ 8.33-7.89 (m, 2H), 7.84 (d, J=8.7 Hz, 1H), 7.52-7.46 (m, 1H), 7.21-7.14 (m, 1H), 7.09 (t, J=8.9 Hz, 1H), 7.01 (d, J=7.2 Hz, 1H), 6.86 (d, J=7.5 Hz, 1H), 6.62 (d, J=8.7 Hz, 1H), 4.03 (d, J=14.2 Hz, 1H), 3.45 (d, J=14.2 Hz, 1H), 3.14 (s, 3H), 1.59 (d, J=2.0 Hz, 3H), 1.18 (d, J=3.3 Hz, 6H).

### Example 48: 3-((6-(N-(6-(5-fluoro-2-methylphenyl)-5-(trifluoromethyl)pyridin-2-yl)sulfamoyl)pyridin-2-yl)(methyl)amino)propanoic Acid (48)

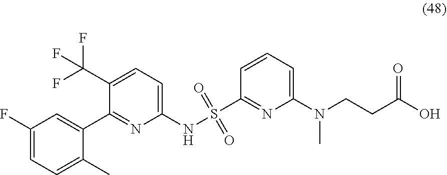 [SMILES: Cc1ccc(F)cc1-c1nc(NS(=O)(=O)c2cccc(N(C)CCC(=O)O)n2)ccc1C(F)(F)F]

3-((6-(N-(6-(5-fluoro-2-methylphenyl)-5-(trifluoromethyl)pyridin-2-yl)sulfamoyl)pyridin-2-yl)(methyl)amino)propanoic acid (48) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with 3-(methylamino)propanoic acid hydrochloride. LCMS (Condition 1): m/z 513.0 [M+H]⁺, 1.54 min. ¹H NMR (400 MHz, Chloroform-d) δ 10.48 (s, 1H), 8.04 (d, J=8.8 Hz, 1H), 7.76 (d, J=8.7 Hz, 1H), 7.44-7.37 (m, 1H), 7.11-7.05 (m, 1H), 6.99-6.91 (m, 2H), 6.70 (dd, J=8.8, 2.5 Hz, 1H), 6.53 (d, J=8.7 Hz, 1H), 4.04-3.86 (m, 2H), 2.98 (s, 3H), 2.33-2.21 (m, 2H), 1.68 (s, 3H).

### Example 49: 3-((6-(N-(6-(3-fluoro-2-methylphenyl)-5-(trifluoromethyl)pyridin-2-yl)sulfamoyl)pyridin-2-yl)(methyl)amino)propanoic Acid (49)

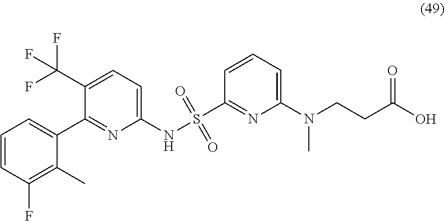 [SMILES: Cc1c(F)cccc1-c1nc(NS(=O)(=O)c2cccc(N(C)CCC(=O)O)n2)ccc1C(F)(F)F]

3-((6-(N-(6-(3-fluoro-2-methylphenyl)-5-(trifluoromethyl)pyridin-2-yl)sulfamoyl)pyridin-2-yl)(methyl)amino)propanoic acid (49) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(3-fluoro-2-methylphenyl)-5-(trifluoromethyl)pyridin-2-yl)pyridine-2-sulfonamide (int-b12) and 4-methylpiperidine-4-carboxylic acid hydrochloride was replaced with 3-(methylamino)propanoic acid hydrochloride. LCMS (Condition 1): m/z 513.0 [M+H]⁺, 1.55 min. ¹H NMR (400 MHz, Chloroform-d) δ 10.67 (s, 1H), 8.13 (d, J=8.8 Hz, 1H), 7.84 (d, J=8.7 Hz, 1H), 7.52-7.47 (m, 1H), 7.22-7.15 (m, 1H), 7.10 (t, J=8.4 Hz, 1H), 7.02 (d, J=7.2 Hz, 1H), 6.88 (d, J=7.5 Hz, 1H), 6.63 (d, J=8.7 Hz, 1H), 4.23-4.14 (m, 1H), 3.94-3.84 (m, 1H), 3.07 (s, 3H), 2.45-2.29 (m, 2H), 1.66 (d, J=2.1 Hz, 3H).

### Example 50: 2-(1-(6-(N-(6-(3-fluoro-2-methylphenyl)-5-(trifluoromethyl)pyridin-2-yl)sulfamoyl)pyridin-2-yl)-3-methylpiperidin-3-yl)acetic Acid (50)

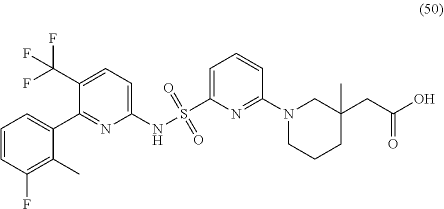 [SMILES: Cc1c(F)cccc1-c1nc(NS(=O)(=O)c2cccc(N3CCCC(C)(CC(=O)O)C3)n2)ccc1C(F)(F)F]

2-(1-(6-(N-(6-(3-fluoro-2-methylphenyl)-5-(trifluoromethyl)pyridin-2-yl)sulfamoyl)pyridin-2-yl)-3-methylpiperidin-3-yl)acetic acid (50) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(3-fluoro-2-methylphenyl)-5-(trifluoromethyl)pyridin-2-yl)pyridine-2-sulfonamide (int-b12) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). LCMS (Condition 1): m/z 567.2 [M+H]⁺, 1.65 min. ¹H NMR (400 MHz, Chloroform-d) δ 10.04 (s, 1H), 7.97 (d, J=8.8 Hz, 1H), 7.74 (t, J=9.8 Hz, 1H), 7.44 (t, J=7.9 Hz, 1H), 7.14-7.04 (m, 2H), 7.01 (t, J=8.8 Hz, 1H), 6.82 (d, J=6.3 Hz, 1H), 6.71 (t, J=7.8 Hz, 1H), 4.35-4.09 (m, 1H), 3.62 (d, J=14.0 Hz, 1H), 3.05-2.91 (m, 1H), 2.79 (d, J=13.3 Hz, 1H), 2.71 (d, J=13.3 Hz, 1H), 2.26 (t, J=15.4 Hz, 1H), 2.00-1.90 (m, 1H), 1.88-1.74 (m, 3H), 1.48 (s, 2H), 1.38-1.27 (m, 1H), 1.26-1.13 (m, 2H), 0.99-0.87 (m, 3H).

### Example 51: 2-(3-methyl-1-(6-(N-(6-(o-tolyl)-5-(trifluoromethyl)pyridin-2-yl)sulfamoyl)pyridin-2-yl)piperidin-3-yl)acetic Acid (51)

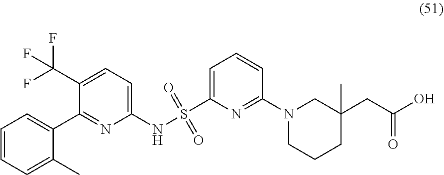 [SMILES: Cc1ccccc1-c1nc(NS(=O)(=O)c2cccc(N3CCCC(C)(CC(=O)O)C3)n2)ccc1C(F)(F)F]

2-(3-methyl-1-(6-(N-(6-(o-tolyl)-5-(trifluoromethyl)pyridin-2-yl)sulfamoyl)pyridin-2-yl)piperidin-3-yl)acetic acid (51) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). LCMS (Condition 1): m/z 549.2 [M+H]⁺, 1.58 min. ¹H NMR (400 MHz, Chloroform-d) δ 10.19 (s, 1H), 8.05 (d, J=8.8 Hz, 1H), 7.82 (d, J=7.8 Hz, 1H), 7.53 (t, J=8.0 Hz, 1H), 7.37-7.31 (m, 1H), 7.27-7.08 (m, 4H), 6.78 (d, J=7.8 Hz, 1H), 4.55-4.09 (m, 1H), 3.77-3.64 (m, 1H), 3.15-2.96 (m, 1H), 2.93-2.72 (m, 1H), 2.41-2.28 (m, 1H), 2.06-1.93 (m, 3H), 1.61-1.49 (m, 2H), 1.46-1.37 (m, 1H), 1.35-1.24 (m, 2H), 1.08-0.96 (m, 3H).

### Example 52: 2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic Acid (52)

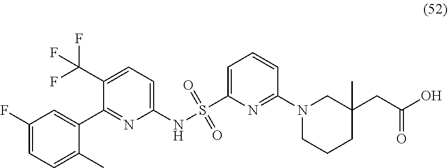 [SMILES: Cc1ccc(F)cc1-c1nc(NS(=O)(=O)c2cccc(N3CCCC(C)(CC(=O)O)C3)n2)ccc1C(F)(F)F]

2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid (52) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). LCMS (Condition 1): m/z 567.2 [M+H]⁺, 1.67 min. ¹H NMR (400 MHz, Chloroform-d) δ 9.58 (s, 1H), 8.03 (d, J=8.9 Hz, 1H), 7.87-7.73 (m, 1H), 7.55-7.47 (m, 1H), 7.22-7.10 (m, 2H), 7.02 (td, J=8.4, 2.7 Hz, 1H), 6.86-6.74 (m, 2H), 4.56-4.40 (m, 1H), 4.24-4.10 (m, 1H), 3.76-3.62 (m, 1H), 3.14-2.96 (m, 1H), 2.91-2.69 (m, 1H), 2.36 (d, J=15.2 Hz, 1H), 2.09-1.85 (m, 4H), 1.46-1.34 (m, 1H), 1.34-1.19 (m, 3H), 1.07-0.95 (m, 3H).

### Example 53: 2-(3-methyl-1-(6-(N-(6-(o-tolyl)-5-(trifluoromethyl)pyridin-2-yl)sulfamoyl)pyridin-2-yl)pyrrolidin-3-yl)acetic Acid (53)

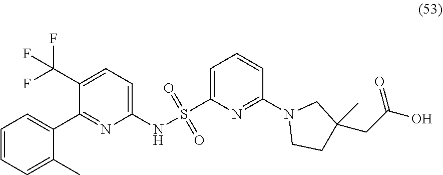 [SMILES: Cc1ccccc1-c1nc(NS(=O)(=O)c2cccc(N3CCC(C)(CC(=O)O)C3)n2)ccc1C(F)(F)F]

2-(3-methyl-1-(6-(N-(6-(o-tolyl)-5-(trifluoromethyl)pyridin-2-yl)sulfamoyl)pyridin-2-yl)pyrrolidin-3-yl)acetic acid (53) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with Boc-deprotected (using TFA) 2-(1-(tert-butoxycarbonyl)-3-methylpyrrolidin-3-yl)acetic acid. LCMS (Condition 1): m/z 535.0 [M+H]⁺, 1.61 min. ¹H NMR (400 MHz, DMSO-d₆) δ 12.20 (s, 1H), 11.51 (s, 1H), 8.19 (d, J=8.9 Hz, 1H), 7.67-7.56 (m, 2H), 7.31 (t, J=7.4 Hz, 1H), 7.26-7.17 (m, 2H), 7.07 (d, J=7.1 Hz, 2H), 6.64 (d, J=8.6 Hz, 1H), 3.31-3.05 (m, 4H), 2.33 (s, 2H), 1.95-1.68 (m, 5H), 1.05 (d, J=7.4 Hz, 3H).

### Example 54: 2-(1-(6-(N-(6-(3-fluoro-2-methylphenyl)-5-(trifluoromethyl)pyridin-2-yl)sulfamoyl)pyridin-2-yl)-3-methylpyrrolidin-3-yl)acetic Acid (54)

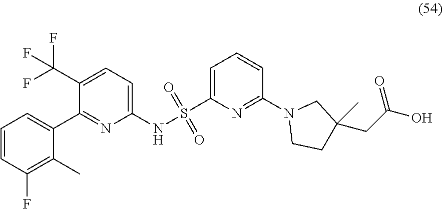 [SMILES: Cc1c(F)cccc1-c1nc(NS(=O)(=O)c2cccc(N3CCC(C)(CC(=O)O)C3)n2)ccc1C(F)(F)F]

2-(1-(6-(N-(6-(3-fluoro-2-methylphenyl)-5-(trifluoromethyl)pyridin-2-yl)sulfamoyl)pyridin-2-yl)-3-methylpyrrolidin-3-yl)acetic acid (554) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(3-fluoro-2-methylphenyl)-5-(trifluoromethyl)pyridin-2-yl)pyridine-2-sulfonamide (int-b12) and 4-methylpiperidine-4-carboxylic acid hydrochloride was replaced with Boc-deprotected (using TFA) 2-(1-(tert-butoxycarbonyl)-3-methylpyrrolidin-3-yl)acetic acid. LCMS (Condition 1): m/z 553.1 [M+H]⁺, 1.62 min. ¹H NMR (400 MHz, DMSO-d₆) δ 12.20 (s, 1H), 11.58 (s, 1H), 8.21 (d, J=9.0 Hz, 1H), 7.68-7.56 (m, 2H), 7.31-7.23 (m, 1H), 7.17 (td, J=8.6, 2.6 Hz, 1H), 7.07 (d, J=7.2 Hz, 1H), 6.98-6.91 (m, 1H), 6.64 (d, J=8.6 Hz, 1H), 3.34-3.32 (m, 3H), 3.28-3.07 (m, 3H), 2.36-2.31 (m, 2H), 1.94-1.84 (m, 1H), 1.81-1.68 (m, 3H), 1.09-0.99 (m, 2H).

### Example 55: 2-(1-(6-(N-(6-(5-fluoro-2-methylphenyl)-5-(trifluoromethyl)pyridin-2-yl)sulfamoyl)pyridin-2-yl)-3-methylpyrrolidin-3-yl)acetic Acid (55)

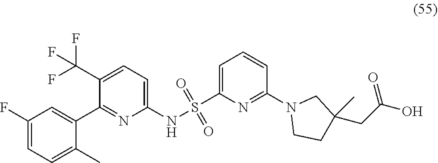 [SMILES: Cc1ccc(F)cc1-c1nc(NS(=O)(=O)c2cccc(N3CCC(C)(CC(=O)O)C3)n2)ccc1C(F)(F)F]

2-(1-(6-(N-(6-(5-fluoro-2-methylphenyl)-5-(trifluoromethyl)pyridin-2-yl)sulfamoyl)pyridin-2-yl)-3-methylpyrrolidin-3-yl)acetic acid (55) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with Boc-deprotected (using TFA) 2-(1-(tert-butoxycarbonyl)-3-methylpyrrolidin-3-yl)acetic acid. LCMS (Condition 1): m/z 553.2 [M+H]⁺, 1.61 min. ¹H NMR (400 MHz, DMSO-d₆) δ 12.20 (s, 1H), 11.58 (s, 1H), 8.21 (d, J=9.0 Hz, 1H), 7.68-7.56 (m, 2H), 7.31-7.23 (m, 1H), 7.17 (td, J=8.6, 2.6 Hz, 1H), 7.07 (d, J=7.2 Hz, 1H), 6.98-6.91 (m, 1H), 6.64 (d, J=8.6 Hz, 1H), 3.34-3.32 (m, 3H), 3.28-3.07 (m, 3H), 2.36-2.31 (m, 2H), 1.94-1.84 (m, 1H), 1.81-1.68 (m, 3H), 1.09-0.99 (m, 2H).

### Example 56: 3-(ethyl(6-(N-(6-(3-fluoro-2-methylphenyl)-5-(trifluoromethyl)pyridin-2-yl)sulfamoyl)pyridin-2-yl)amino)propanoic Acid (56)

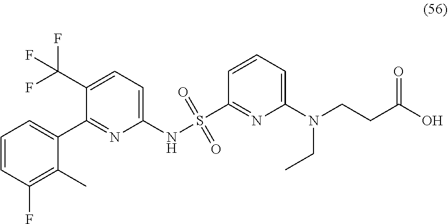 [SMILES: CCN(CCC(=O)O)c1cccc(S(=O)(=O)Nc2ccc(C(F)(F)F)c(-c3cccc(F)c3C)n2)n1]

3-(ethyl(6-(N-(6-(3-fluoro-2-methylphenyl)-5-(trifluoromethyl)pyridin-2-yl)sulfamoyl)pyridin-2-yl)amino)propanoic acid (56) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(3-fluoro-2-methylphenyl)-5-(trifluoromethyl)pyridin-2-yl)pyridine-2-sulfonamide (int-b12) and 4-methylpiperidine-4-carboxylic acid hydrochloride was replaced with 3-(ethylamino)propanoic acid hydrochloride. LCMS (Condition 1): m/z 527.1 [M+H]⁺, 1.60 min. ¹H NMR (400 MHz, DMSO-d₆) δ 12.25 (s, 1H), 11.63 (s, 1H), 8.17 (d, J=8.9 Hz, 1H), 7.68-7.62 (m, 1H), 7.50 (d, J=8.7 Hz, 1H), 7.29-7.18 (m, 2H), 7.07 (d, J=7.2 Hz, 1H), 6.92 (d, J=7.1 Hz, 1H), 6.86 (d, J=8.7 Hz, 1H), 3.61-3.42 (m, 2H), 3.42-3.34 (m, 2H), 2.35 (t, J=7.3 Hz, 2H), 1.62 (s, 3H), 0.91 (t, J=6.9 Hz, 3H).

### Example 57: (R)-2-(1-(6-(N-(5-(trifluoromethyl)-6-(2-vinylphenyl)pyridin-2-yl)sulfamoyl)pyridin-2-yl)piperidin-3-yl)acetic Acid (57)

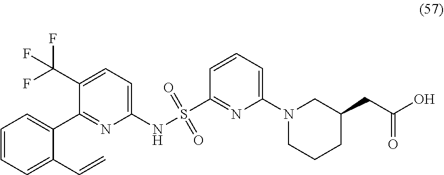 [SMILES: C=Cc1ccccc1-c1nc(NS(=O)(=O)c2cccc(N3CCC[C@H](CC(=O)O)C3)n2)ccc1C(F)(F)F]

(R)-2-(1-(6-(N-(5-(trifluoromethyl)-6-(2-vinylphenyl)pyridin-2-yl)sulfamoyl)pyridin-2-yl)piperidin-3-yl)acetic acid (57) was synthesized using the procedure described in Example 10, except in step 1, 6-fluoro-N-(6-(o-tolyl)-5-(trifluoromethyl)pyridin-2-yl)pyridine-2-sulfonamide (int-b1) was replaced with 6-fluoro-N-(5-(trifluoromethyl)-6-(2-vinylphenyl)pyridin-2-yl)pyridine-2-sulfonamide (int-b20) and ethyl 2-methyl-2-(piperidin-4-yl)propanoate hydrochloride was replaced with ethyl (R)-2-(piperidin-3-yl)acetate hydrochloride. LCMS (Condition 1): m/z 547.2 [M+H]⁺, 1.78 min. ¹H NMR (400 MHz, DMSO-d₆) δ 12.17 (s, 1H), 11.52 (s, 1H), 8.20 (d, J=8.9 Hz, 1H), 7.72-7.65 (m, 2H), 7.65-7.52 (m, 1H), 7.43 (td, J=7.7, 1.4 Hz, 1H), 7.31 (td, J=7.5, 1.2 Hz, 1H), 7.12-6.97 (m, 3H), 6.00 (q, J=13.5, 12.5 Hz, 1H), 5.65 (dd, J=17.5, 1.2 Hz, 1H), 5.11-4.97 (m, 1H), 4.08-3.91 (m, 2H), 2.83-2.69 (m, 1H), 2.69-2.56 (m, 1H), 2.23-1.99 (m, 1H), 1.82-1.66 (m, 2H), 1.57-1.43 (m, 1H), 1.34-1.12 (m, 3H).

### Example 58: (S)-2-(1-(6-(N-(5-(trifluoromethyl)-6-(2-vinylphenyl)pyridin-2-yl)sulfamoyl)pyridin-2-yl)piperidin-3-yl)acetic Acid (58)

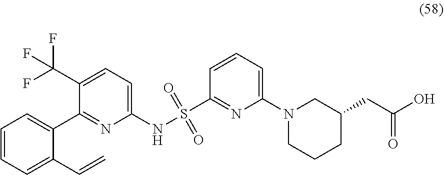 [SMILES: C=Cc1ccccc1-c1nc(NS(=O)(=O)c2cccc(N3CCC[C@@H](CC(=O)O)C3)n2)ccc1C(F)(F)F]

(S)-2-(1-(6-(N-(5-(trifluoromethyl)-6-(2-vinylphenyl)pyridin-2-yl)sulfamoyl)pyridin-2-yl)piperidin-3-yl)acetic acid (58) was synthesized using the procedure described in Example 10, except in step 1, 6-fluoro-N-(6-(o-tolyl)-5-(trifluoromethyl)pyridin-2-yl)pyridine-2-sulfonamide (int-b1) was replaced with 6-fluoro-N-(5-(trifluoromethyl)-6-(2-vinylphenyl)pyridin-2-yl)pyridine-2-sulfonamide (int-b20) and ethyl 2-methyl-2-(piperidin-4-yl)propanoate hydrochloride was replaced with methyl (S)-2-(piperidin-3-yl)acetate hydrochloride. LCMS (Condition 1): m/z 547.2 [M+H]⁺, 1.79 min. ¹H NMR (400 MHz, DMSO-d₆) δ 12.18 (s, 1H), 11.52 (s, 1H), 8.20 (d, J=8.8 Hz, 1H), 7.73-7.64 (m, 2H), 7.64-7.52 (m, 1H), 7.43 (td, J=7.6, 1.4 Hz, 1H), 7.31 (td, J=7.5, 1.2 Hz, 1H), 7.16-6.96 (m, 3H), 6.01 (q, J=13.3, 12.5 Hz, 1H), 5.65 (dd, J=17.5, 1.2 Hz, 1H), 5.11-4.95 (m, 1H), 3.99 (d, J=13.0 Hz, 2H), 2.85-2.69 (m, 1H), 2.68-2.57 (m, 1H), 2.24-2.13 (m, 1H), 2.12-2.00 (m, 1H), 1.82-1.66 (m, 2H), 1.56-1.42 (m, 1H), 1.33-1.13 (m, 2H).

### Example 59: (R)-2-(1-(6-(N-(6-(o-tolyl)-5-(trifluoromethyl)pyridin-2-yl)sulfamoyl)pyridin-2-yl)piperidin-3-yl)acetic Acid (59)

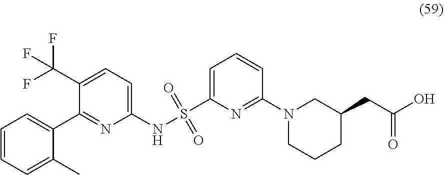 [SMILES: Cc1ccccc1-c1nc(NS(=O)(=O)c2cccc(N3CCC[C@H](CC(=O)O)C3)n2)ccc1C(F)(F)F]

(R)-2-(1-(6-(N-(6-(o-tolyl)-5-(trifluoromethyl)pyridin-2-yl)sulfamoyl)pyridin-2-yl)piperidin-3-yl)acetic acid (59) was synthesized using the procedure described in Example 10, except in step 1, ethyl 2-methyl-2-(piperidin-4-yl)propanoate hydrochloride was replaced with ethyl (R)-2-(piperidin-3-yl)acetate hydrochloride. LCMS (Condition 1): m/z 535.2 [M+H]⁺, 1.78 min. ¹H NMR (400 MHz, DMSO-d₆) δ 12.17 (s, 1H), 11.52 (s, 1H), 8.18 (d, J=8.9 Hz, 1H), 7.68 (t, J=8.1 Hz, 1H), 7.52 (d, J=8.9 Hz, 1H), 7.31 (td, J=7.5, 1.4 Hz, 1H), 7.25-7.16 (m, 2H), 7.11-6.96 (m, 3H), 3.99 (t, J=12.7 Hz, 2H), 2.82-2.70 (m, 1H), 2.62 (dd, J=13.1, 10.2 Hz, 1H), 2.17 (dd, J=15.9, 6.0 Hz, 1H), 2.11-1.99 (m, 1H), 1.86-1.62 (m, 4H), 1.49 (s, 1H), 1.33-1.12 (m, 3H).

### Example 60: (S)-2-(1-(6-(N-(6-(o-tolyl)-5-(trifluoromethyl)pyridin-2-yl)sulfamoyl)pyridin-2-yl)piperidin-3-yl)acetic Acid (60)

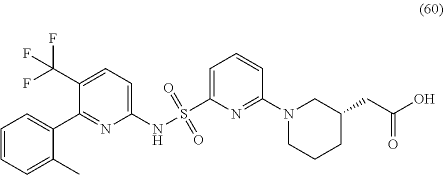 [SMILES: Cc1ccccc1-c1nc(NS(=O)(=O)c2cccc(N3CCC[C@@H](CC(=O)O)C3)n2)ccc1C(F)(F)F]

(S)-2-(1-(6-(N-(6-(o-tolyl)-5-(trifluoromethyl)pyridin-2-yl)sulfamoyl)pyridin-2-yl)piperidin-3-yl)acetic acid (60) was synthesized using the procedure described in Example 10, except in step 1, ethyl 2-methyl-2-(piperidin-4-yl)propanoate hydrochloride was replaced with methyl (S)-2-(piperidin-3-yl)acetate hydrochloride. LCMS (Condition 1): m/z 535.2 [M+H]⁺, 1.78 min. ¹H NMR (400 MHz, DMSO-d₆) δ 12.16 (s, 1H), 11.52 (s, 1H), 8.18 (d, J=8.9 Hz, 1H), 7.68 (t, J=7.9 Hz, 1H), 7.52 (d, J=8.9 Hz, 1H), 7.31 (td, J=7.5, 1.4 Hz, 1H), 7.25-7.15 (m, 2H), 7.10-6.96 (m, 3H), 4.07-3.87 (m, 2H), 2.81-2.68 (m, 1H), 2.62 (dd, J=13.1, 10.3 Hz, 1H), 2.17 (dd, J=15.8, 6.0 Hz, 1H), 2.11-1.99 (m, 1H), 1.77 (d, J=12.5 Hz, 4H), 1.55-1.41 (m, 1H), 1.35-1.11 (m, 3H).

### Example 61: 3-(ethyl(6-(N-(6-(5-fluoro-2-methylphenyl)-5-(trifluoromethyl)pyridin-2-yl)sulfamoyl)pyridin-2-yl)amino)propanoic Acid (61)

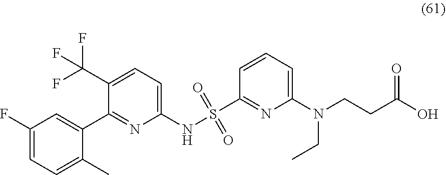 [SMILES: CCN(CCC(=O)O)c1cccc(S(=O)(=O)Nc2ccc(C(F)(F)F)c(-c3cc(F)ccc3C)n2)n1]

3-(ethyl(6-(N-(6-(5-fluoro-2-methylphenyl)-5-(trifluoromethyl)pyridin-2-yl)sulfamoyl)pyridin-2-yl)amino)propanoic acid (61) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with 3-(ethylamino)propanoic acid hydrochloride. LCMS (Condition 1): m/z 527.0 [M+H]⁺, 1.59 min. ¹H NMR (400 MHz, DMSO-d₆) δ 12.26 (s, 1H), 11.63 (s, 1H), 8.16 (d, J=8.9 Hz, 1H), 7.65 (dd, J=8.7, 7.3 Hz, 1H), 7.50 (d, J=8.7 Hz, 1H), 7.25 (dd, J=8.5, 5.8 Hz, 1H), 7.16 (td, J=8.6, 2.8 Hz, 1H), 7.07 (d, J=7.2 Hz, 1H), 6.95-6.90 (m, 1H), 6.85 (d, J=8.7 Hz, 1H), 3.51 (dt, J=14.7, 6.9 Hz, 2H), 3.43-3.35 (m, 2H), 2.34 (t, J=7.0 Hz, 2H), 1.70 (s, 3H), 0.91 (t, J=6.9 Hz, 3H).

### Example 62: (R)-3-(1-(6-(N-(6-(o-tolyl)-5-(trifluoromethyl)pyridin-2-yl)sulfamoyl)pyridin-2-yl)piperidin-3-yl)propanoic Acid (62)

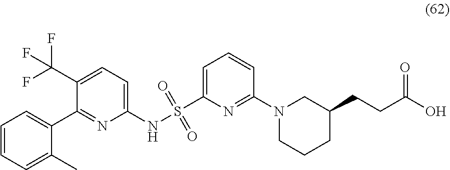 [SMILES: Cc1ccccc1-c1nc(NS(=O)(=O)c2cccc(N3CCC[C@H](CCC(=O)O)C3)n2)ccc1C(F)(F)F]

(R)-3-(1-(6-(N-(6-(o-tolyl)-5-(trifluoromethyl)pyridin-2-yl)sulfamoyl)pyridin-2-yl)piperidin-3-yl)propanoic acid (62) was synthesized from 3-[1-(6-{[6-(2-methylphenyl)-5-(trifluoromethyl)pyridin-2-yl]sulfamoyl}pyridin-2-yl)piperidin-3-yl]propanoic acid (58), where a single enantiomer of unknown absolute stereochemistry was obtained from separation by chiral supercritical fluid chromatography. LCMS (Condition 1): m/z 549.2 [M+H]⁺, 1.82 min. ¹H NMR (600 MHz, DMSO-d₆) δ 12.09 (s, 1H), 11.54 (s, 1H), 8.17 (d, J=8.9 Hz, 1H), 7.65 (dd, J=8.7, 7.3 Hz, 1H), 7.49 (d, J=8.9 Hz, 1H), 7.31 (td, J=7.5, 1.4 Hz, 1H), 7.24-7.18 (m, 2H), 7.09-7.00 (m, 3H), 4.03-3.97 (m, 2H), 2.75-2.66 (m, 1H), 2.57-2.51 (m, 1H), 2.27-2.17 (m, 2H), 1.79-1.70 (m, 4H), 1.56-1.40 (m, 2H), 1.39-1.22 (m, 2H), 1.22-1.01 (m, 2H).

### Example 63: (S)-3-(1-(6-(N-(6-(o-tolyl)-5-(trifluoromethyl)pyridin-2-yl)sulfamoyl)pyridin-2-yl)piperidin-3-yl)propanoic Acid (63)

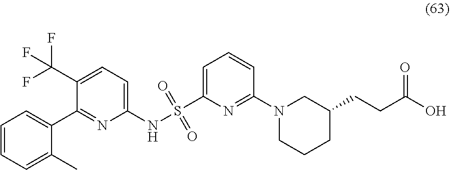 [SMILES: Cc1ccccc1-c1nc(NS(=O)(=O)c2cccc(N3CCC[C@@H](CCC(=O)O)C3)n2)ccc1C(F)(F)F]

(S)-3-(1-(6-(N-(6-(o-tolyl)-5-(trifluoromethyl)pyridin-2-yl)sulfamoyl)pyridin-2-yl)piperidin-3-yl)propanoic acid (63) was synthesized from 3-[1-(6-{[6-(2-methylphenyl)-5-(trifluoromethyl)pyridin-2-yl]sulfamoyl}pyridin-2-yl)piperidin-3-yl]propanoic acid (58), where a single enantiomer of unknown absolute stereochemistry was obtained from separation by chiral supercritical fluid chromatography. LCMS (Condition 1): m/z 549.2 [M+H]⁺, 1.87 min. ¹H NMR (600 MHz, DMSO-d₆) δ 12.10 (s, 1H), 11.54 (s, 1H), 8.17 (d, J=8.8 Hz, 1H), 7.65 (dd, J=8.7, 7.3 Hz, 1H), 7.49 (d, J=8.9 Hz, 1H), 7.31 (td, J=7.5, 1.4 Hz, 1H), 7.24-7.17 (m, 2H), 7.09-7.00 (m, 3H), 4.03-3.97 (m, 2H), 2.75-2.66 (m, 1H), 2.55-2.51 (m, 1H), 2.27-2.17 (m, 2H), 1.81-1.68 (m, 4H), 1.56-1.41 (m, 2H), 1.40-1.21 (m, 2H), 1.21-1.04 (m, 2H).

### Example 64: (S)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid (64)

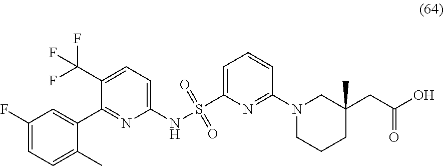 [SMILES: Cc1ccc(F)cc1-c1nc(NS(=O)(=O)c2cccc(N3CCC[C@@](C)(CC(=O)O)C3)n2)ccc1C(F)(F)F]

(S)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid (64) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). Single enantiomer of unknown absolute stereochemistry obtained from chiral supercritical fluid chromatography. LCMS (Condition 1): m/z 567.0 [M+H]⁺, 1.57 min. ¹H NMR (400 MHz, DMSO-d₆) δ 12.05 (s, 1H), 11.59 (s, 1H), 8.21 (d, J=8.6 Hz, 1H), 7.67-7.60 (m, 1H), 7.58-7.49 (m, 1H), 7.29-7.23 (m, 1H), 7.16 (td, J=8.6, 2.8 Hz, 1H), 7.07-6.99 (m, 2H), 6.95-6.89 (m, 1H), 3.52-3.37 (m, 2H), 3.32-3.26 (m, 1H), 3.24-3.08 (m, 1H), 2.11-2.03 (m, 1H), 1.95 (s, 1H), 1.73 (s, 3H), 1.65-1.57 (m, 1H), 1.38 (d, J=9.6 Hz, 3H), 0.84 (d, J=8.6 Hz, 3H).

### Example 65: (R)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic Acid (65)

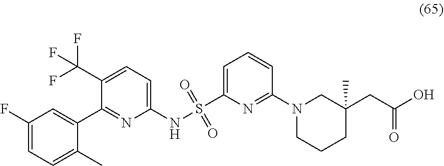 [SMILES: Cc1ccc(F)cc1-c1nc(NS(=O)(=O)c2cccc(N3CCC[C@](C)(CC(=O)O)C3)n2)ccc1C(F)(F)F]

(R)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid (65) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). Single enantiomer of unknown absolute stereochemistry obtained from chiral supercritical fluid chromatography. LCMS (Condition 1): m/z 567.2 [M+H]⁺, 1.57 min. ¹H NMR (400 MHz, DMSO-d₆) δ 12.04 (s, 1H), 11.59 (s, 1H), 8.21 (d, J=8.6 Hz, 1H), 7.64 (t, J=8.0 Hz, 1H), 7.58-7.47 (m, 1H), 7.29-7.23 (m, 1H), 7.16 (td, J=8.6, 2.6 Hz, 1H), 7.07-6.99 (m, 2H), 6.92 (d, J=8.0 Hz, 1H), 3.52-3.38 (m, 3H), 3.19 (t, J=12.4 Hz, 1H), 2.13-2.01 (m, 1H), 2.01-1.89 (m, 1H), 1.73 (s, 3H), 1.65-1.55 (m, 1H), 1.47-1.33 (m, 3H), 0.84 (d, J=7.6 Hz, 3H).

### Example 66: 5-(methyl(6-(N-(6-(o-tolyl)-5-(trifluoromethyl)pyridin-2-yl)sulfamoyl)pyridin-2-yl)amino)pentanoic Acid (66)

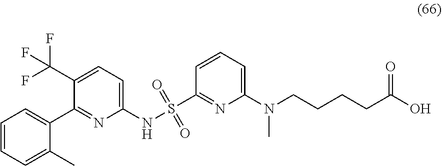 [SMILES: Cc1ccccc1-c1nc(NS(=O)(=O)c2cccc(N(C)CCCCC(=O)O)n2)ccc1C(F)(F)F]

5-(methyl(6-(N-(6-(o-tolyl)-5-(trifluoromethyl)pyridin-2-yl)sulfamoyl)pyridin-2-yl)amino)pentanoic acid (66) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 5-(methylamino)pentanoic acid. LCMS (Condition 1): m/z 523.2 [M+H]+, 1.59 min. 1H NMR (500 MHz, Chloroform-d) δ 10.58 (s, 1H), 7.99 (d, J=9.0 Hz, 1H), 7.71 (d, J=8.8 Hz, 1H), 7.59-7.54 (m, 1H), 7.36 (td, J=7.5, 1.3 Hz, 1H), 7.30 (d, J=7.0 Hz, 1H), 7.28-7.21 (m, 2H), 7.10 (d, J=7.5 Hz, 1H), 6.61 (d, J=8.5 Hz, 1H), 3.60 (s, 1H), 3.50 (s, 1H), 2.98 (s, 3H), 2.32 (t, J=6.6 Hz, 2H), 2.01 (s, 3H), 1.68-1.52 (m, 4H), 1.36-1.25 (m, 1H).

### Example 67: 5-((6-(N-(6-(3-fluoro-2-methylphenyl)-5-(trifluoromethyl)pyridin-2-yl)sulfamoyl)pyridin-2-yl)(methyl)amino)pentanoic Acid (67)

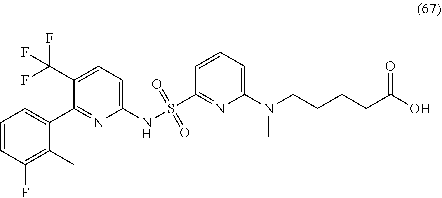 [SMILES: Cc1c(F)cccc1-c1nc(NS(=O)(=O)c2cccc(N(C)CCCCC(=O)O)n2)ccc1C(F)(F)F]

5-((6-(N-(6-(3-fluoro-2-methylphenyl)-5-(trifluoromethyl)pyridin-2-yl)sulfamoyl)pyridin-2-yl)(methyl)amino)pentanoic acid (67) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(3-fluoro-2-methylphenyl)-5-(trifluoromethyl)pyridin-2-yl)pyridine-2-sulfonamide (int-b12) and 4-methylpiperidine-4-carboxylic acid hydrochloride was replaced with 5-(methylamino)pentanoic acid. LCMS (Condition 1): m/z 541.2 [M+H]⁺, 1.63 min. ¹H NMR (400 MHz, DMSO-d₆) δ 12.03 (s, 1H), 11.61 (s, 1H), 8.20 (d, J=8.8 Hz, 1H), 7.69-7.61 (m, 1H), 7.50 (d, J=8.7 Hz, 1H), 7.31-7.18 (m, 2H), 7.05 (d, J=7.2 Hz, 1H), 6.92 (d, J=7.1 Hz, 1H), 6.84 (d, J=8.6 Hz, 1H), 3.49-3.37 (m, 1H), 3.34 (s, 3H), 2.86 (s, 2H), 2.51 (s, 3H), 2.14 (s, 1H), 1.65-1.60 (m, 2H), 1.41-1.32 (m, 2H).

### Example 68: 5-((6-(N-(6-(5-fluoro-2-methylphenyl)-5-(trifluoromethyl)pyridin-2-yl)sulfamoyl)pyridin-2-yl)(methyl)amino)pentanoic Acid (68)

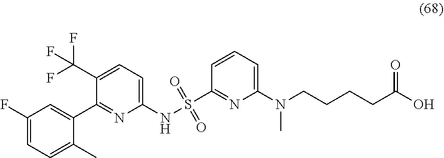 [SMILES: Cc1ccc(F)cc1-c1nc(NS(=O)(=O)c2cccc(N(C)CCCCC(=O)O)n2)ccc1C(F)(F)F]

5-((6-(N-(6-(5-fluoro-2-methylphenyl)-5-(trifluoromethyl)pyridin-2-yl)sulfamoyl)pyridin-2-yl)(methyl)amino)pentanoic acid (68) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(5-fluoro-2-methylphenyl)-5-(trifluoromethyl)pyridin-2-yl)pyridine-2-sulfonamide (int-b10) and 4-methylpiperidine-4-carboxylic acid hydrochloride was replaced with 5-(methylamino)pentanoic acid. LCMS (Condition 1): m/z 541.2 [M+H]⁺, 1.60 min. ¹H NMR (500 MHz, Chloroform-d) δ 10.53 (s, 1H), 8.00 (d, J=9.0 Hz, 1H), 7.73 (d, J=8.9 Hz, 1H), 7.57 (dd, J=8.7, 7.3 Hz, 1H), 7.31-7.27 (m, 1H), 7.21 (dd, 1H), 7.06 (td, J=8.5, 2.7 Hz, 1H), 6.83 (dd, J=8.9, 2.6 Hz, 1H), 6.61 (d, J=8.6 Hz, 1H), 3.69-3.39 (m, 2H), 2.98 (s, 3H), 2.34 (t, J=6.7 Hz, 2H), 1.97 (s, 3H), 1.68-1.53 (m, 4H).

### Example 69: 3-((cyclopropylmethyl)(6-(N-(6-(5-fluoro-2-methylphenyl)-5-(trifluoromethyl)pyridin-2-yl)sulfamoyl)pyridin-2-yl)amino)propanoic Acid (69)

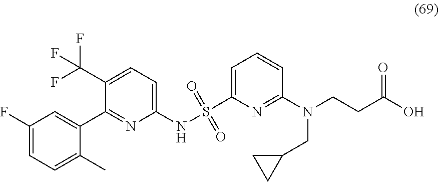 [SMILES: Cc1ccc(F)cc1-c1nc(NS(=O)(=O)c2cccc(N(CCC(=O)O)CC3CC3)n2)ccc1C(F)(F)F]

3-((cyclopropylmethyl)(6-(N-(6-(5-fluoro-2-methylphenyl)-5-(trifluoromethyl)pyridin-2-yl)sulfamoyl)pyridin-2-yl)amino)propanoic acid (69) was synthesized using the procedure described in Example 5, except in step 1, N-(5-chloro-6-(2,6-dimethylphenyl)ₙpyridin-2-yl)-6-fluoropyridine-2-sulfonamide (int-b6) was replaced with 6-fluoro-N-(6-(5-fluoro-2-methylphenyl)-5-(trifluoromethyl)pyridin-2-yl)pyridine-2-sulfonamide (int-b10) and methyl 2-(piperidin-4-yl)acetate was replaced with ethyl 3-((cyclopropylmethyl)amino)propanoate. LCMS (Condition 1): m/z 553.1 [M+H]⁺, 1.41 min. ¹H NMR (400 MHz, Methanol-d₄) δ 8.07 (d, J=8.9 Hz, 1H), 7.64-7.52 (m, 2H), 7.24-7.13 (m, 2H), 7.06-6.99 (m, 1H), 6.87 (d, J=8.7 Hz, 1H), 6.79 (dd, J=9.2, 2.8 Hz, 1H), 3.78-3.70 (m, 2H), 3.35 (d, J=6.5 Hz, 2H), 2.50 (t, J=7.3 Hz, 2H), 1.83 (s, 3H), 1.01-0.91 (m, 1H), 0.45 (dd, J=8.2, 1.6 Hz, 2H), 0.30-0.17 (m, 2H).

### Example 70: 3-(ethyl(6-(N-(6-(2-isopropylphenyl)-5-(trifluoromethyl)pyridin-2-yl)sulfamoyl)pyridin-2-yl)amino)propanoic Acid (70)

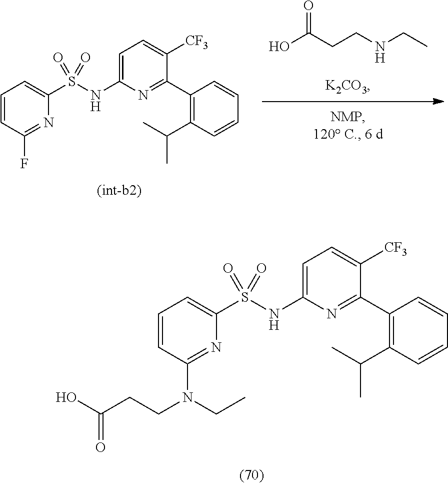

In a vial, 6-fluoro-N-(6-(2-isopropylphenyl)-5-(trifluoromethyl)pyridin-2-yl)pyridine-2-sulfonamide (int-b2) (750 mg, 1.71 mmol) was taken up in NMP (5 mL), 3-(ethylamino)propanoic acid hydrochloride (393 mg, 2.56 mmol) and K₂CO₃ (708 mg, 5.12 mmol) were added, and the reaction was heated to 120° C. After 6 days, the reaction was poured into 1 M HCl and extracted with EtOAc (×3). The organics were then washed with water and brine, dried over MgSO₄, and concentrated in vacuo. The crude material was purified by flash column chromatography (120 g silica gel column, 0-60% EtOAc/heptane) to give 3-(ethyl(6-(N-(6-(2-isopropylphenyl)-5-(trifluoromethyl)pyridin-2-yl)sulfamoyl)pyridin-2-yl)amino)propanoic acid (70) as an off-white solid. LCMS (Condition 1): m/z 536.9 [M+H]⁺, 1.77 min. ¹H NMR (400 MHz, Methanol-d₄) δ 8.05 (d, J=8.9 Hz, 1H), 7.66 (dd, J=9.0, 1.0 Hz, 1H), 7.60 (dd, J=8.7, 7.3 Hz, 1H), 7.41-7.33 (m, 2H), 7.20-7.11 (m, 2H), 7.00 (dd, J=7.4, 1.1 Hz, 1H), 6.84-6.77 (m, 1H), 3.68-3.61 (m, 2H), 3.50-3.44 (m, 2H), 2.53-2.46 (m, 2H), 2.45-2.37 (m, 1H), 1.14-0.98 (m, 9H).

### Example 71: 3-(ethyl(6-(N-(6-(o-tolyl)-5-(trifluoromethyl)pyridin-2-yl)sulfamoyl)pyridin-2-yl)amino)propanoic Acid (71)

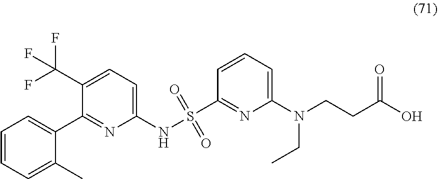 [SMILES: CCN(CCC(=O)O)c1cccc(S(=O)(=O)Nc2ccc(C(F)(F)F)c(-c3ccccc3C)n2)n1]

3-(ethyl(6-(N-(6-(o-tolyl)-5-(trifluoromethyl)pyridin-2-yl)sulfamoyl)pyridin-2-yl)amino)propanoic acid (71) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(6-(o-tolyl)-5-(trifluoromethyl)pyridin-2-yl)pyridine-2-sulfonamide (int-b1). LCMS (Condition 1): m/z 509.1 [M+H]⁺, 1.66 min. ¹H NMR (400 MHz, DMSO-d₆) δ 12.28 (br s, 1H), 11.58 (br s, 1H), 8.15 (d, J=8.9 Hz, 1H), 7.65 (dd, J=8.7, 7.3 Hz, 1H), 7.51 (d, J=8.8 Hz, 1H), 7.33-7.28 (m, 1H), 7.23-7.18 (m, 2H), 7.06 (dd, J=10.3, 7.4 Hz, 2H), 6.85 (d, J=8.7 Hz, 1H), 3.55-3.47 (m, 2H), 3.40-3.36 (m, 2H), 2.35 (t, J=7.0 Hz, 2H), 1.75 (s, 3H), 0.91 (t, J=6.9 Hz, 3H).

### Example 72: 3-(ethyl(6-(N-(6-(2-ethylphenyl)-5-(trifluoromethyl)pyridin-2-yl)sulfamoyl)pyridin-2-yl)amino)propanoic Acid (72)

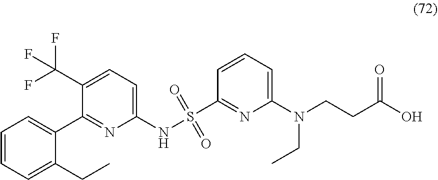 [SMILES: CCc1ccccc1-c1nc(NS(=O)(=O)c2cccc(N(CC)CCC(=O)O)n2)ccc1C(F)(F)F]

3-(ethyl(6-(N-(6-(2-ethylphenyl)-5-(trifluoromethyl)pyridin-2-yl)sulfamoyl)pyridin-2-yl)amino)propanoic acid (72) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with N-(6-(2-ethylphenyl)-5-(trifluoromethyl)pyridin-2-yl)-6-fluoropyridine-2-sulfonamide (int-b11). LCMS (Condition 1): m/z 522.9 [M+H]⁺, 1.71 min. ¹H NMR (400 MHz, DMSO-d₆) δ 12.28 (br s, 1H), 11.56 (br s, 1H), 8.15 (d, J=8.9 Hz, 1H), 7.65 (dd, J=8.7, 7.3 Hz, 1H), 7.51 (d, J=8.8 Hz, 1H), 7.37-7.33 (m, 1H), 7.27-7.23 (m, 1H), 7.19 (dd, J=7.4, 1.3 Hz, 1H), 7.07 (d, J=7.2 Hz, 1H), 7.03 (d, J=7.6 Hz, 1H), 6.86 (d, J=8.7 Hz, 1H), 3.59-3.53 (m, 1H), 3.51-3.45 (m, 1H), 3.42-3.33 (m, 2H), 2.35 (t, J=7.2 Hz, 2H), 2.15-2.08 (m, 1H), 2.02-1.97 (m, 1H), 0.91 (t, J=6.9 Hz, 3H), 0.85 (t, J=7.6 Hz, 3H).

### Example 73: 3-(1-(6-(N-(6-(o-tolyl)-5-(trifluoromethyl)pyridin-2-yl)sulfamoyl)pyridin-2-yl)piperidin-3-yl)propanoic acid (73)

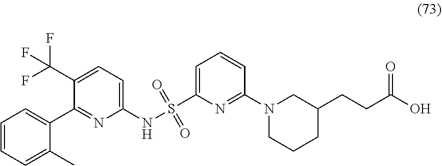 [SMILES: Cc1ccccc1-c1nc(NS(=O)(=O)c2cccc(N3CCCC(CCC(=O)O)C3)n2)ccc1C(F)(F)F]

3-(1-(6-(N-(6-(o-tolyl)-5-(trifluoromethyl)pyridin-2-yl)sulfamoyl)pyridin-2-yl)piperidin-3-yl)propanoic acid (73) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(6-(o-tolyl)-5-(trifluoromethyl)pyridin-2-yl)pyridine-2-sulfonamide (int-b1), 3-(ethylamino)propanoic acid was replaced with Boc-deprotected (using HCl) 3-(1-(tert-butoxycarbonyl)piperidin-3-yl)propanoic acid, and K₂CO₃ was replaced with N,N-diisopropylethylamine. LCMS (Condition 1): m/z 549.2 [M+H]⁺, 1.82 min. ¹H NMR (400 MHz, DMSO-d₆) δ 12.06 (s, 1H), 11.58 (s, 1H), 8.17 (d, J=8.8 Hz, 1H), 7.65 (dd, J=8.7, 7.3 Hz, 1H), 7.49 (d, J=8.8 Hz, 1H), 7.31 (td, J=7.5, 1.4 Hz, 1H), 7.25-7.15 (m, 2H), 7.09-6.98 (m, 3H), 4.10-3.89 (m, 2H), 2.77-2.63 (m, 1H), 2.56-2.51 (m, 1H), 2.29-2.14 (m, 2H), 1.80-1.63 (m, 4H), 1.56-1.03 (m, 6H).

### Example 74: 3-(1-(6-(N-(5-(trifluoromethyl)-6-(2-vinylphenyl)pyridin-2-yl)sulfamoyl)pyridin-2-yl)piperidin-3-yl)propanoic Acid (74)

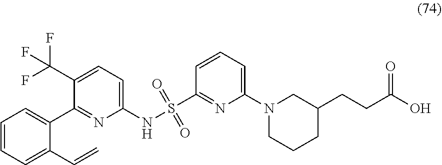 [SMILES: C=Cc1ccccc1-c1nc(NS(=O)(=O)c2cccc(N3CCCC(CCC(=O)O)C3)n2)ccc1C(F)(F)F]

3-(1-(6-(N-(5-(trifluoromethyl)-6-(2-vinylphenyl)pyridin-2-yl)sulfamoyl)pyridin-2-yl)piperidin-3-yl)propanoic acid (74) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(5-(trifluoromethyl)-6-(2-vinylphenyl)pyridin-2-yl)pyridine-2-sulfonamide (int-b20), 3-(ethylamino)propanoic acid was replaced with Boc-deprotected (using HCl) 3-(1-(tert-butoxycarbonyl)piperidin-3-yl)propanoic acid, and K₂CO₃ was replaced with N,N-diisopropylethylamine. LCMS (Condition 1): m/z 561.2 [M+H]⁺, 1.82 min. ¹H NMR (400 MHz, DMSO-d₆) δ 12.08 (s, 1H), 11.56 (s, 1H), 8.19 (d, J=8.9 Hz, 1H), 7.71-7.61 (m, 2H), 7.61-7.53 (m, 1H), 7.43 (td, J=7.7, 1.4 Hz, 1H), 7.31 (td, J=7.5, 1.2 Hz, 1H), 7.11-7.01 (m, 3H), 6.07-5.91 (m, 1H), 5.64 (dd, J=17.5, 1.2 Hz, 1H), 5.03 (dd, J=11.1, 6.5 Hz, 1H), 3.99 (d, J=13.1 Hz, 2H), 2.76-2.64 (m, 1H), 2.58-2.51 (m, 1H), 2.24 (t, J=7.5 Hz, 2H), 1.80-1.68 (m, 1H), 1.57-1.01 (m, 6H).

### Example 75: 4-(1-(6-(N-(6-(o-tolyl)-5-(trifluoromethyl)pyridin-2-yl)sulfamoyl)pyridin-2-yl)piperidin-3-yl)butanoic Acid (75)

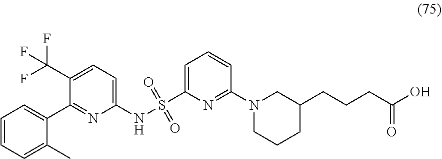 [SMILES: Cc1ccccc1-c1nc(NS(=O)(=O)c2cccc(N3CCCC(CCCC(=O)O)C3)n2)ccc1C(F)(F)F]

4-(1-(6-(N-(6-(o-tolyl)-5-(trifluoromethyl)pyridin-2-yl)sulfamoyl)pyridin-2-yl)piperidin-3-yl)butanoic acid (75) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(6-(o-tolyl)-5-(trifluoromethyl)pyridin-2-yl)pyridine-2-sulfonamide (int-b1), 3-(ethylamino)propanoic acid was replaced with Boc-deprotected (using HCl) 4-(1-(tert-butoxycarbonyl)piperidin-3-yl)butanoic acid, and K₂CO₃ was replaced with N,N-diisopropylethylamine. LCMS (Condition 1): m/z 563.2 [M+H]⁺, 1.81 min. ¹H NMR (400 MHz, DMSO-d₆) δ 12.02 (s, 1H), 11.54 (s, 1H), 8.19 (d, J=8.9 Hz, 1H), 7.65 (dd, J=8.7, 7.3 Hz, 1H), 7.55-7.43 (m, 1H), 7.31 (td, J=7.5, 1.4 Hz, 1H), 7.25-7.16 (m, 2H), 7.09-6.98 (m, 3H), 4.10-3.89 (m, 2H), 2.76-2.64 (m, 1H), 2.48-2.39 (m, 1H), 2.23-2.13 (m, 2H), 1.80-1.69 (m, 4H), 1.57-1.39 (m, 3H), 1.32-0.97 (m, 5H).

### Example 76: 4-(1-(6-(N-(5-(trifluoromethyl)-6-(2-vinylphenyl)pyridin-2-yl)sulfamoyl)pyridin-2-yl)piperidin-3-yl)butanoic Acid (76)

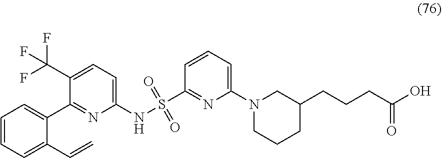 [SMILES: C=Cc1ccccc1-c1nc(NS(=O)(=O)c2cccc(N3CCCC(CCCC(=O)O)C3)n2)ccc1C(F)(F)F]

4-(1-(6-(N-(5-(trifluoromethyl)-6-(2-vinylphenyl)pyridin-2-yl)sulfamoyl)pyridin-2-yl)piperidin-3-yl)butanoic acid (76) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(5-(trifluoromethyl)-6-(2-vinylphenyl)pyridin-2-yl)pyridine-2-sulfonamide (int-b20), 3-(ethylamino)propanoic acid was replaced with Boc-deprotected (using HCl) 4-(1-(tert-butoxycarbonyl)piperidin-3-yl)butanoic acid, and K₂CO₃ was replaced with N,N-diisopropylethylamine. LCMS (Condition 1): m/z 575.2 [M+H]⁺, 1.82 min. ¹H NMR (400 MHz, DMSO-d₆) δ 12.01 (s, 1H), 11.52 (s, 1H), 8.20 (d, J=8.9 Hz, 1H), 7.71-7.62 (m, 2H), 7.62-7.53 (m, 1H), 7.42 (td, J=7.7, 1.4 Hz, 1H), 7.31 (td, J=7.5, 1.2 Hz, 1H), 7.11-7.01 (m, 3H), 5.99 (ddd, J=17.4, 10.4, 2.4 Hz, 1H), 5.64 (dd, J=17.6, 3.1 Hz, 1H), 5.02 (dd, J=11.1, 3.4 Hz, 1H), 4.08-3.89 (m, 2H), 2.76-2.64 (m, 1H), 2.45-2.38 (m, 1H), 2.21-2.14 (m, 2H), 1.81-1.71 (m, 1H), 1.59-1.41 (m, 3H), 1.38-0.97 (m, 5H).

### Example 77: ethyl (R)-2-(1-(6-(N-(5-(trifluoromethyl)-6-(2-vinylphenyl)pyridin-2-yl)sulfamoyl)pyridin-2-yl)piperidin-3-yl)acetate (77)

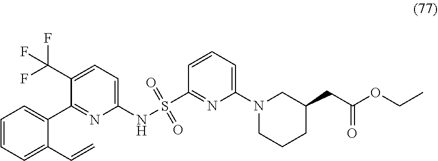 [SMILES: C=Cc1ccccc1-c1nc(NS(=O)(=O)c2cccc(N3CCC[C@H](CC(=O)OCC)C3)n2)ccc1C(F)(F)F]

ethyl (R)-2-(1-(6-(N-(5-(trifluoromethyl)-6-(2-vinylphenyl)pyridin-2-yl)sulfamoyl)pyridin-2-yl)piperidin-3-yl)acetate (77) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(5-(trifluoromethyl)-6-(2-vinylphenyl)pyridin-2-yl)pyridine-2-sulfonamide (int-b20), 3-(ethylamino)propanoic acid was replaced ethyl (R)-2-(piperidin-3-yl)acetate, and K₂CO₃ was replaced with N,N-diisopropylethylamine. LCMS (Condition 1): m/z 575.2 [M+H]⁺, 1.95 min. ¹H NMR (400 MHz, DMSO-d₆) δ 11.52 (s, 1H), 8.20 (d, J=8.9 Hz, 1H), 7.72-7.64 (m, 2H), 7.64-7.52 (m, 1H), 7.43 (td, J=7.7, 1.4 Hz, 1H), 7.31 (t, J=7.5 Hz, 1H), 7.13-6.98 (m, 3H), 6.10-5.89 (m, 1H), 5.65 (d, J=17.5 Hz, 1H), 5.10-4.97 (m, 1H), 4.07 (q, J=7.1 Hz, 2H), 4.02-3.90 (m, 2H), 2.84-2.71 (m, 1H), 2.71-2.61 (m, 1H), 2.31-2.20 (m, 1H), 2.20-2.09 (m, 1H), 1.83-1.64 (m, 2H), 1.56-1.42 (m, 1H), 1.28-1.10 (m, 5H).

### Example 78: methyl (S)-2-(1-(6-(N-(5-(trifluoromethyl)-6-(2-vinylphenyl)pyridin-2-yl)sulfamoyl)pyridin-2-yl)piperidin-3-yl)acetate (78)

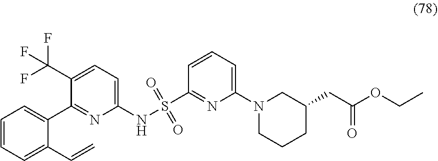 [SMILES: C=Cc1ccccc1-c1nc(NS(=O)(=O)c2cccc(N3CCC[C@@H](CC(=O)OCC)C3)n2)ccc1C(F)(F)F]

methyl (S)-2-(1-(6-(N-(5-(trifluoromethyl)-6-(2-vinylphenyl)pyridin-2-yl)sulfamoyl)pyridin-2-yl)piperidin-3-yl)acetate (78) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(5-(trifluoromethyl)-6-(2-vinylphenyl)pyridin-2-yl)pyridine-2-sulfonamide (int-b20), 3-(ethylamino)propanoic acid was replaced methyl (S)-2-(piperidin-3-yl)acetate hydrochloride, and K₂CO₃ was replaced with N,N-diisopropylethylamine. LCMS (Condition 1): m/z 561.2 [M+H]⁺, 1.91 min. ¹H NMR (400 MHz, DMSO-d₆) δ 11.52 (s, 1H), 8.20 (d, J=8.9 Hz, 1H), 7.74-7.64 (m, 2H), 7.64-7.51 (m, 1H), 7.43 (td, J=7.7, 1.4 Hz, 1H), 7.31 (td, J=7.5, 1.2 Hz, 1H), 7.11-7.00 (m, 3H), 6.11-5.89 (m, 1H), 5.71-5.56 (m, 1H), 5.11-4.95 (m, 1H), 4.06-3.88 (m, 2H), 3.60 (s, 3H), 2.84-2.72 (m, 1H), 2.72-2.60 (m, 1H), 2.31-2.22 (m, 1H), 2.22-2.10 (m, 1H), 1.83-1.65 (m, 2H), 1.58-1.41 (m, 1H), 1.32-1.09 (m, 2H).

### Example 79: 3-(1-(6-(N-(6-(o-tolyl)-5-(trifluoromethyl)pyridin-2-yl)sulfamoyl)pyridin-2-yl)piperidin-4-yl)propanoic Acid (79)

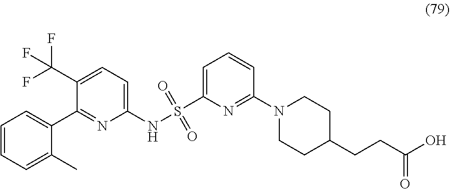 [SMILES: Cc1ccccc1-c1nc(NS(=O)(=O)c2cccc(N3CCC(CCC(=O)O)CC3)n2)ccc1C(F)(F)F]

3-(1-(6-(N-(6-(o-tolyl)-5-(trifluoromethyl)pyridin-2-yl)sulfamoyl)pyridin-2-yl)piperidin-4-yl)propanoic acid (79) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(6-(o-tolyl)-5-(trifluoromethyl)pyridin-2-yl)pyridine-2-sulfonamide (int-b1), 3-(ethylamino)propanoic acid was replaced with 3-(piperidin-4-yl)propanoic acid, and K₂CO₃ was replaced with N,N-diisopropylethylamine. LCMS (Condition 1): m/z 549.2 [M+H]⁺, 1.75 min. ¹H NMR (500 MHz, DMSO-d₆) δ 11.99 (s, 1H), 11.50 (s, 1H), 8.19 (d, J=8.8 Hz, 1H), 7.66 (dd, J=8.6, 7.1 Hz, 1H), 7.51 (d, J=8.9 Hz, 1H), 7.37-7.28 (m, 1H), 7.26-7.18 (m, 2H), 7.07 (d, J=7.2 Hz, 1H), 7.06-7.01 (m, 2H), 4.19-4.05 (m, 2H), 2.71 (t, J=12.8 Hz, 2H), 2.19 (t, J=7.6 Hz, 2H), 1.77 (s, 3H), 1.61-1.50 (m, 2H), 1.50-1.40 (m, 1H), 1.35 (q, J=7.6 Hz, 2H), 0.91-0.69 (m, 2H). ¹⁹F NMR (471 MHz, DMSO-d₆) δ −57.06 (s).

### Example 80: 3-((6-(N-(6-(2-cyclopropylphenyl)-5-(trifluoromethyl)pyridin-2-yl)sulfamoyl)pyridin-2-yl)(ethyl)amino)propanoic Acid (80)

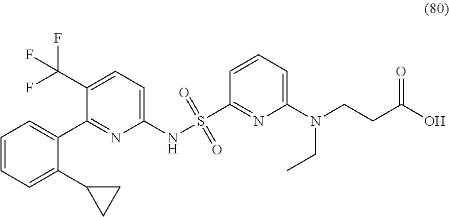 [SMILES: CCN(CCC(=O)O)c1cccc(S(=O)(=O)Nc2ccc(C(F)(F)F)c(-c3ccccc3C3CC3)n2)n1]

3-((6-(N-(6-(2-cyclopropylphenyl)-5-(trifluoromethyl)pyridin-2-yl)sulfamoyl)pyridin-2-yl)(ethyl)amino)propanoic acid (80) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with N-(6-(2-cyclopropylphenyl)-5-(trifluoromethyl)pyridin-2-yl)-6-fluoropyridine-2-sulfonamide (int-b19). LCMS (Condition 1): m/z 534.9 [M+H]⁺, 1.72 min. ¹H NMR (400 MHz, Methanol-d₄) δ 8.06 (d, J=8.9 Hz, 1H), 7.64 (dd, J=8.9, 0.9 Hz, 1H), 7.61 (dd, J=8.7, 7.3 Hz, 1H), 7.33-7.29 (m, 1H), 7.20-7.13 (m, 2H), 7.05 (dd, J=7.5, 1.5 Hz, 1H), 7.00-6.95 (m, 1H), 6.84-6.77 (m, 1H), 3.75-3.56 (m, 2H), 3.50-3.44 (m, 2H), 2.49 (t, J=7.1 Hz, 2H), 1.44-1.37 (m, 1H), 1.06 (t, J=7.0 Hz, 3H), 0.66-0.50 (m, 3H), 0.48-0.38 (m, 1H).

### Example 81: 3-(ethyl(6-(N-(6-(2-ethyl-5-fluorophenyl)-5-(trifluoromethyl)pyridin-2-yl)sulfamoyl)pyridin-2-yl)amino)propanoic Acid (81)

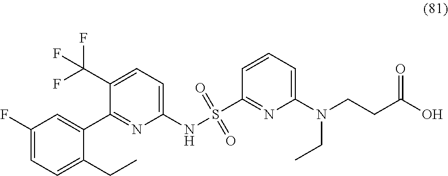 [SMILES: CCc1ccc(F)cc1-c1nc(NS(=O)(=O)c2cccc(N(CC)CCC(=O)O)n2)ccc1C(F)(F)F]

3-(ethyl(6-(N-(6-(2-ethyl-5-fluorophenyl)-5-(trifluoromethyl)pyridin-2-yl)sulfamoyl)pyridin-2-yl)amino)propanoic acid (81) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with N-(6-(2-ethyl-5-fluorophenyl)-5-(trifluoromethyl)pyridin-2-yl)-6-fluoropyridine-2-sulfonamide (int-b3). LCMS (Condition 1): m/z 540.9 [M+H]⁺, 1.72 min. ¹H NMR (500 MHz, Methanol-d₄) δ 8.06 (d, J=8.9 Hz, 1H), 7.60 (dd, J=8.7, 7.3 Hz, 1H), 7.57 (dd, J=8.8, 0.9 Hz, 1H), 7.25 (dd, J=8.6, 5.6 Hz, 1H), 7.17 (d, J=7.2 Hz, 1H), 7.09-7.05 (m, 1H), 6.81 (d, J=8.7 Hz, 1H), 6.77 (dd, J=9.2, 2.8 Hz, 1H), 3.72-3.60 (m, 2H), 3.53-3.40 (m, 2H), 2.47 (t, J=7.1 Hz, 2H), 2.28-2.21 (m, 1H), 2.15-2.07 (m, 1H), 1.07 (t, J=7.0 Hz, 3H), 0.93 (t, J=7.6 Hz, 3H).

### Example 82: 3-(1-(6-(N-(6-(2-ethylphenyl)-5-(trifluoromethyl)pyridin-2-yl)sulfamoyl)pyridin-2-yl)piperidin-3-yl)propanoic Acid (82)

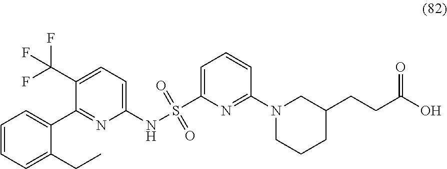 [SMILES: CCc1ccccc1-c1nc(NS(=O)(=O)c2cccc(N3CCCC(CCC(=O)O)C3)n2)ccc1C(F)(F)F]

A mixture of 3-(1-(6-(N-(5-(trifluoromethyl)-6-(2-vinylphenyl)pyridin-2-yl)sulfamoyl)pyridin-2-yl)piperidin-3-yl)propanoic acid (74) (165 mg, 0.294 mmol) and platinum(IV) oxide (33.4 mg, 0.147 mmol) in EtOAc (24 mL) was stirred under hydrogen at room temperature overnight. The reaction was then filtered through a pad of Celite and the filtrate was concentrated. The residue was subjected to purification by flash column chromatography (0-10% MeOH/DCM) to provide 3-(1-(6-(N-(6-(2-ethylphenyl)-5-(trifluoromethyl)pyridin-2-yl)sulfamoyl)pyridin-2-yl)piperidin-3-yl)propanoic acid (82) as a white solid. LCMS (Condition 1): m/z 563.2 [M+H]⁺, 1.84 min. ¹H NMR (400 MHz, DMSO-d₆) δ 12.09 (s, 1H), 11.53 (s, 1H), 8.17 (dd, J=8.8, 2.5 Hz, 1H), 7.65 (dd, J=8.7, 7.3 Hz, 1H), 7.51 (dd, J=8.9, 4.4 Hz, 1H), 7.35 (dd, J=7.5, 1.4 Hz, 1H), 7.27 (dd, J=7.8, 1.3 Hz, 1H), 7.20 (td, J=7.4, 1.3 Hz, 1H), 7.11-7.04 (m, 2H), 7.02 (t, J=6.5 Hz, 1H), 4.08-3.91 (m, 2H), 2.76-2.64 (m, 1H), 2.23 (q, J=7.2 Hz, 2H), 2.17-2.06 (m, 1H), 2.06-1.92 (m, 1H), 1.82-1.69 (m, 1H), 1.56-1.41 (m, 2H), 1.39-1.00 (m, 5H), 0.90-0.82 (m, 3H).

### Example 83: 4-(1-(6-(N-(6-(2-ethylphenyl)-5-(trifluoromethyl)pyridin-2-yl)sulfamoyl)pyridin-2-yl)piperidin-3-yl)butanoic Acid (83)

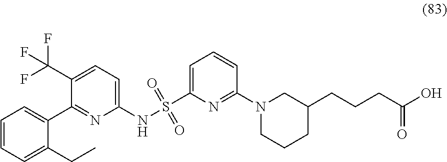 [SMILES: CCc1ccccc1-c1nc(NS(=O)(=O)c2cccc(N3CCCC(CCCC(=O)O)C3)n2)ccc1C(F)(F)F]

4-(1-(6-(N-(6-(2-ethylphenyl)-5-(trifluoromethyl)pyridin-2-yl)sulfamoyl)pyridin-2-yl)piperidin-3-yl)butanoic acid (83) was synthesized using the procedure described in Example 82, except 3-(1-(6-(N-(5-(trifluoromethyl)-6-(2-vinylphenyl)pyridin-2-yl)sulfamoyl)pyridin-2-yl)piperidin-3-yl)propanoic acid (74) was replaced with 4-(1-(6-(N-(5-(trifluoromethyl)-6-(2-vinylphenyl)pyridin-2-yl)sulfamoyl)pyridin-2-yl)piperidin-3-yl)butanoic acid (76). LCMS (Condition 1): m/z 577.2 [M+H]⁺, 1.86 min. ¹H NMR (400 MHz, DMSO-d₆) δ 12.05 (s, 1H), 11.51 (s, 1H), 8.18 (d, J=8.9 Hz, 1H), 7.65 (ddd, J=8.4, 7.3, 0.8 Hz, 1H), 7.52 (t, J=9.1 Hz, 1H), 7.39-7.33 (m, 1H), 7.26 (d, J=7.7 Hz, 1H), 7.20 (td, J=7.5, 1.3 Hz, 1H), 7.08-6.97 (m, 3H), 4.12-3.89 (m, 2H), 2.77-2.64 (m, 1H), 2.46-2.37 (m, 1H), 2.18 (t, J=7.3 Hz, 2H), 2.15-2.04 (m, 1H), 2.04-1.93 (m, 1H), 1.76 (d, J=12.0 Hz, 1H), 1.59-1.39 (m, 3H), 1.34-0.96 (m, 5H), 0.86 (td, J=7.6, 1.7 Hz, 3H).

### Example 84: (R)-2-(1-(6-(N-(6-(2-ethylphenyl)-5-(trifluoromethyl)pyridin-2-yl)sulfamoyl)pyridin-2-yl)piperidin-3-yl)acetic Acid (84)

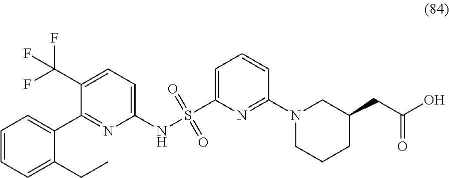 [SMILES: CCc1ccccc1-c1nc(NS(=O)(=O)c2cccc(N3CCC[C@H](CC(=O)O)C3)n2)ccc1C(F)(F)F]

(R)-2-(1-(6-(N-(6-(2-ethylphenyl)-5-(trifluoromethyl)pyridin-2-yl)sulfamoyl)pyridin-2-yl)piperidin-3-yl)acetic acid (84) was synthesized using the procedure described in Example 82, except 3-(1-(6-(N-(5-(trifluoromethyl)-6-(2-vinylphenyl)pyridin-2-yl)sulfamoyl)pyridin-2-yl)piperidin-3-yl)propanoic acid (74) was replaced with (R)-2-(1-(6-(N-(5-(trifluoromethyl)-6-(2-vinylphenyl)pyridin-2-yl)sulfamoyl)pyridin-2-yl)piperidin-3-yl)acetic acid (57). LCMS (Condition 1): m/z 549.2 [M+H]⁺, 1.86 min. ¹H NMR (600 MHz, DMSO-d₆) δ 12.16 (s, 1H), 11.48 (s, 1H), 8.25-8.10 (m, 1H), 7.68 (ddd, J=8.7, 7.2, 5.2 Hz, 1H), 7.59-7.45 (m, 1H), 7.35 (td, J=7.5, 1.4 Hz, 1H), 7.29-7.25 (m, 1H), 7.20 (td, J=7.5, 1.3 Hz, 1H), 7.08 (dd, J=7.3, 1.3 Hz, 1H), 7.07-6.95 (m, 2H), 4.05-3.92 (m, 2H), 2.80-2.69 (m, 1H), 2.65-2.57 (m, 1H), 2.22-1.95 (m, 2H), 1.81-1.67 (m, 2H), 1.55-1.43 (m, 1H), 1.32-1.12 (m, 3H), 0.92-0.83 (m, 4H).

### Example 85: (S)-2-(1-(6-(N-(6-(2-ethylphenyl)-5-(trifluoromethyl)pyridin-2-yl)sulfamoyl)pyridin-2-yl)piperidin-3-yl)acetic Acid (85)

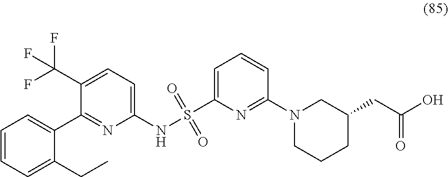 [SMILES: CCc1ccccc1-c1nc(NS(=O)(=O)c2cccc(N3CCC[C@@H](CC(=O)O)C3)n2)ccc1C(F)(F)F]

(S)-2-(1-(6-(N-(6-(2-ethylphenyl)-5-(trifluoromethyl)pyridin-2-yl)sulfamoyl)pyridin-2-yl)piperidin-3-yl)acetic acid (85) was synthesized using the procedure described in Example 82, except 3-(1-(6-(N-(5-(trifluoromethyl)-6-(2-vinylphenyl)pyridin-2-yl)sulfamoyl)pyridin-2-yl)piperidin-3-yl)propanoic acid (74) was replaced with (S)-2-(1-(6-(N-(5-(trifluoromethyl)-6-(2-vinylphenyl)pyridin-2-yl)sulfamoyl)pyridin-2-yl)piperidin-3-yl)acetic acid (58). LCMS (Condition 1): m/z 549.20 [M+H]⁺, 1.84 min. ¹H NMR (600 MHz, DMSO-d₆) δ 12.16 (s, 1H), 11.49 (s, 1H), 8.18 (dd, J=9.4, 3.1 Hz, 1H), 7.68 (ddd, J=8.7, 7.2, 5.2 Hz, 1H), 7.59-7.43 (m, 1H), 7.35 (td, J=7.5, 1.4 Hz, 1H), 7.30-7.24 (m, 1H), 7.20 (td, J=7.4, 1.3 Hz, 1H), 7.08 (dd, J=7.2, 1.3 Hz, 1H), 7.04-6.97 (m, 2H), 4.06-3.93 (m, 2H), 2.84-2.68 (m, 1H), 2.66-2.57 (m, 1H), 2.22-1.95 (m, 2H), 1.80-1.67 (m, 2H), 1.55-1.43 (m, 1H), 1.31-1.13 (m, 3H), 0.92-0.82 (m, 4H).

### Example 86: 3-((cyclopropylmethyl)(6-(N-(6-(o-tolyl)-5-(trifluoromethyl)pyridin-2-yl)sulfamoyl)pyridin-2-yl)amino)propanoic Acid (86)

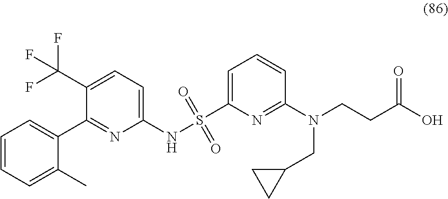 [SMILES: Cc1ccccc1-c1nc(NS(=O)(=O)c2cccc(N(CCC(=O)O)CC3CC3)n2)ccc1C(F)(F)F]

3-((cyclopropylmethyl)(6-(N-(6-(o-tolyl)-5-(trifluoromethyl)pyridin-2-yl)sulfamoyl)pyridin-2-yl)amino)propanoic acid (86) was synthesized using the procedure described in Example 5, except in step 1, N-(5-chloro-6-(2,6-dimethylphenyl)pyridin-2-yl)-6-fluoropyridine-2-sulfonamide (int-b6) was replaced with 6-fluoro-N-(6-(o-tolyl)-5-(trifluoromethyl)pyridin-2-yl)pyridine-2-sulfonamide (int-b1) and methyl 2-(piperidin-4-yl)acetate was replaced with ethyl 3-((cyclopropylmethyl)amino)propanoate. LCMS (Condition 1): m/z 535.1 [M+H]⁺, 1.39 min. ¹H NMR (400 MHz, Methanol-d₄) δ 8.06 (d, J=8.9 Hz, 1H), 7.64-7.53 (m, 2H), 7.32-7.25 (td, J=7.5, 1.5 Hz, 1H), 7.25-7.12 (m, 3H), 7.05 (d, J=7.5 Hz, 1H), 6.86 (d, J=8.7 Hz, 1H), 3.80-3.66 (m, 2H), 3.40-3.32 (m, 2H), 2.51 (t, J=7.3 Hz, 2H), 1.89 (s, 3H), 1.03-0.92 (m, 1H), 0.52-0.37 (m, 2H), 0.32-0.16 (m, 2H).

### Example 87: 4-(butyl(6-(N-(6-(o-tolyl)-5-(trifluoromethyl)pyridin-2-yl)sulfamoyl)pyridin-2-yl)amino)butanoic Acid (87)

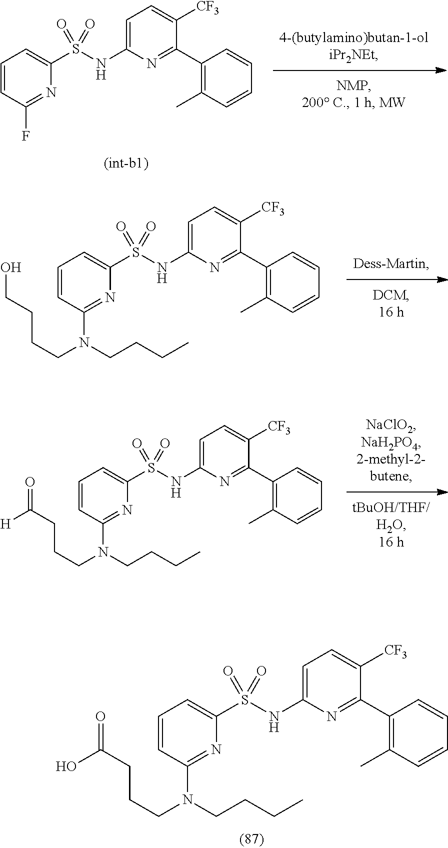 [SMILES: Cc1ccccc1-c1nc(NS(=O)(=O)c2cccc(F)n2)ccc1C(F)(F)F ; CCCCN(CCCCO)c1cccc(S(=O)(=O)Nc2ccc(C(F)(F)F)c(-c3ccccc3C)n2)n1 ; CCCCN(CCCC=O)c1cccc(S(=O)(=O)Nc2ccc(C(F)(F)F)c(-c3ccccc3C)n2)n1 ; CCCCN(CCCC(=O)O)c1cccc(S(=O)(=O)Nc2ccc(C(F)(F)F)c(-c3ccccc3C)n2)n1]

### Step 1. Synthesis of 6-(butyl(4-hydroxybutyl)amino)-N-(6-(o-tolyl)-5-(trifluoromethyl)pyridin-2-yl)pyridine-2-sulfonamide

In a microwave vial, 6-fluoro-N-(6-(o-tolyl)-5-(trifluoromethyl)pyridin-2-yl)pyridine-2-sulfonamide (int-b1) (100 mg, 0.243 mmol) was taken up in NMP (3 mL), 4-(butylamino)butan-1-ol (70.6 mg, 0.486 mmol) and N,N-diisopropylethylamine (0.170 mL, 0.972 mmol) were added and the reaction was heated to 150° C. in the microwave for 3 h. Additional 4-(butylamino)butan-1-ol (70.6 mg, 0.486 mmol) was added and the reaction was heated to 200° C. for 1 h in microwave. The reaction was poured into 1 M HCl and extracted with EtOAc (×3). The organics were then washed with water and brine, dried over MgSO₄, and concentrated in vacuo. The crude material was purified by flash column chromatography (4 g RediSep Rf Gold silica gel column, 0-60% EtOAc/heptane) to give 6-(butyl(4-hydroxybutyl)amino)-N-(6-(o-tolyl)-5-(trifluoromethyl)pyridin-2-yl)pyridine-2-sulfonamide as an off-white solid. Condition 1, LCMS: m/z 537.0 [M+H]⁺, 1.84 min.

### Step 2. Synthesis of 6-(butyl(4-oxobutyl)amino)-N-(6-(o-tolyl)-5-(trifluoromethyl)pyridin-2-yl)pyridine-2-sulfonamide

In a vial, 6-(butyl(4-hydroxybutyl)amino)-N-(6-(o-tolyl)-5-(trifluoromethyl)pyridin-2-yl)pyridine-2-sulfonamide (75.7 mg, 0.141 mmol) was taken up in DCM (4 mL) and Dess-Martin periodinane (71.8 mg, 0.169 mmol) was added. After 16 h, the reaction was diluted with EtOAc and quenched with sat. aq. Na₂S₂O₃ and sat. aq. NaHCO₃. The organics were then separated and washed with water and brine. The crude product was taken on directly to the next reaction. Condition 1, LCMS: m/z 535.0 [M+H]⁺, 1.87 min.

### Step 3. Synthesis of 4-(butyl(6-(N-(6-(o-tolyl)-5-(trifluoromethyl)pyridin-2-yl)sulfamoyl)pyridin-2-yl)amino)butanoic Acid

In a vial, crude 6-(butyl(4-oxobutyl)amino)-N-(6-(o-tolyl)-5-(trifluoromethyl)pyridin-2-yl)pyridine-2-sulfonamide (71.6 mg, 0.134 mmol) was taken up in tBuOH (1 mL) and THE (1 mL), then 2-methyl-2-butene (0.851 mL, 8.04 mmol) was added, followed by sodium dihydrogen phosphate (96 mg, 0.80 mmol) in water (0.25 mL), and sodium chlorite (73 mg, 0.80 mmol) in water (0.25 mL). After 16 h, the reaction was diluted with EtOAc and quenched with sat. aq. Na₂S₂O₃. The organics were then separated and washed with water and brine. The crude material was purified by flash column chromatography (4 g RediSep Rf Gold silica gel column, 0-40% EtOAc/heptane) to give 4-(butyl(6-(N-(6-(o-tolyl)-5-(trifluoromethyl)pyridin-2-yl)sulfamoyl)pyridin-2-yl)amino)butanoic acid (87) as a white solid. Condition 1, LCMS: m/z 550.9 [M+H]⁺, 1.77 min. ¹H NMR (400 MHz, Methanol-d₄) δ 8.04 (d, J=8.9 Hz, 1H), 7.56 (dd, J=8.7, 7.2 Hz, 1H), 7.48 (dd, J=8.9, 0.9 Hz, 1H), 7.31-7.27 (m, 1H), 7.23-7.14 (m, 2H), 7.12 (d, J=7.2 Hz, 1H), 7.03 (d, J=7.6 Hz, 1H), 6.79 (d, J=8.7 Hz, 1H), 3.51-3.33 (m, 4H), 2.24 (t, J=7.1 Hz, 2H), 1.86 (s, 3H), 1.80-1.72 (m, 2H), 1.54-1.40 (m, 2H), 1.35-1.19 (m, 2H), 0.90 (t, J=7.3 Hz, 3H).

### Example 88: 2-(1-(6-(N-(6-(o-tolyl)-5-(trifluoromethyl)pyridin-2-yl)sulfamoyl)pyridin-2-yl)piperidin-3-yl)acetic Acid (88)

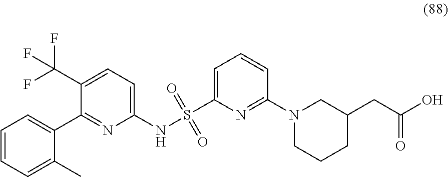 [SMILES: Cc1ccccc1-c1nc(NS(=O)(=O)c2cccc(N3CCCC(CC(=O)O)C3)n2)ccc1C(F)(F)F]

2-(1-(6-(N-(6-(o-tolyl)-5-(trifluoromethyl)pyridin-2-yl)sulfamoyl)pyridin-2-yl)piperidin-3-yl)acetic acid (88) was synthesized using the procedure described in Example 87, except in step 1, 4-(butylamino)butan-1-ol was replaced with 2-(piperidin-3-yl)ethanol, N,N-diisopropylethylamine was replaced with potassium carbonate, NMP was replaced with DMA, and microwave heating at 200° C. was replaced by oil bath heating at 120° C. LCMS (Condition 1): m/z 535.2 [M+H]⁺, 1.51 min. ¹H NMR (400 MHz, Chloroform-d) δ 7.99 (d, J=8.9 Hz, 1H), 7.71 (d, J=8.8 Hz, 1H), 7.57 (dd, J=8.7, 7.3 Hz, 1H), 7.32 (td, J=7.6, 1.2 Hz, 1H), 7.26-7.17 (m, 3H), 7.09 (d, J=7.5 Hz, 1H), 6.81 (d, J=8.7 Hz, 1H), 4.21-3.98 (m, 1H), 3.87 (d, J=10.7 Hz, 1H), 3.04-2.67 (m, 2H), 2.16 (d, J=6.4 Hz, 2H), 2.00 (s, 4H), 1.85-1.72 (m, 1H), 1.60 (dd, J=8.7, 4.2 Hz, 1H), 1.49-1.17 (m, 3H).

### Example 89: 4-(ethyl(6-(N-(6-(2-isopropylphenyl)-5-(trifluoromethyl)pyridin-2-yl)sulfamoyl)pyridin-2-yl)amino)butanoic Acid (89)

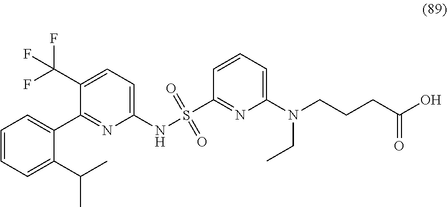 [SMILES: CCN(CCCC(=O)O)c1cccc(S(=O)(=O)Nc2ccc(C(F)(F)F)c(-c3ccccc3C(C)C)n2)n1]

4-(ethyl(6-(N-(6-(2-isopropylphenyl)-5-(trifluoromethyl)pyridin-2-yl)sulfamoyl)pyridin-2-yl)amino)butanoic acid (89) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(2-isopropylphenyl)-5-(trifluoromethyl)pyridin-2-yl)pyridine-2-sulfonamide (int-b2) and 4-(butylamino)butan-1-ol was replaced with 4-(ethylamino)butan-1-ol. LCMS (Condition 1): m/z 551.1 [M+H]⁺, 1.77 min. ¹H NMR (400 MHz, DMSO-d₆) δ 12.11 (br s, 1H), 11.47 (br s, 1H), 8.18 (d, J=8.9 Hz, 1H), 7.63 (dd, J=8.7, 7.2 Hz, 1H), 7.57 (d, J=8.9 Hz, 1H), 7.43-7.30 (m, 2H), 7.20-7.16 (m, 1H), 7.04 (d, J=7.2 Hz, 1H), 6.97 (d, J=7.6 Hz, 1H), 6.85 (d, J=8.7 Hz, 1H), 3.87-3.70 (m, 1H), 3.32-3.27 (m, 3H), 2.37-2.27 (m, 1H), 2.17 (t, J=7.3 Hz, 2H), 1.66-1.59 (m, 2H), 1.01 (d, J=6.9 Hz, 3H), 0.91 (d, J=6.7 Hz, 3H), 0.88 (t, J=7.0 Hz, 3H).

### Example 90: 4-((6-(N-(6-(2-isopropylphenyl)-5-(trifluoromethyl)pyridin-2-yl)sulfamoyl)pyridin-2-yl)(methyl)amino)butanoic Acid (90)

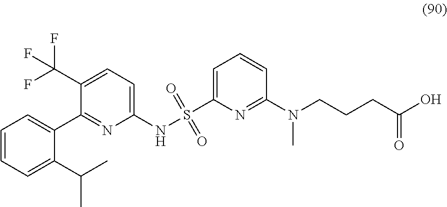 [SMILES: CC(C)c1ccccc1-c1nc(NS(=O)(=O)c2cccc(N(C)CCCC(=O)O)n2)ccc1C(F)(F)F]

4-((6-(N-(6-(2-isopropylphenyl)-5-(trifluoromethyl)pyridin-2-yl)sulfamoyl)pyridin-2-yl)(methyl)amino)butanoic acid (90) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(2-isopropylphenyl)-5-(trifluoromethyl)pyridin-2-yl)pyridine-2-sulfonamide (int-b2) and 4-(butylamino)butan-1-ol was replaced with 4-(methylamino)butan-1-ol. LCMS (Condition 1): m/z 536.9 [M+H]⁺, 1.74 min. ¹H NMR (400 MHz, DMSO-d₆) δ 12.11 (br s, 1H), 11.47 (br s, 1H), 8.17 (d, J=8.9 Hz, 1H), 7.65 (dd, J=8.7, 7.3 Hz, 1H), 7.56 (d, J=8.8 Hz, 1H), 7.42-7.31 (m, 2H), 7.20-7.16 (m, 1H), 7.06 (d, J=7.2 Hz, 1H), 6.99 (d, J=7.6 Hz, 1H), 6.85 (d, J=8.7 Hz, 1H), 3.38-3.33 (m, 2H), 2.85 (s, 3H), 2.37-2.26 (m, 1H), 2.11 (t, J=7.4 Hz, 2H), 1.61-1.58 (m, 2H), 1.01 (d, J=6.9 Hz, 3H), 0.92 (d, J=6.8 Hz, 3H).

### Example 91: 3-(isopropyl(6-(N-(6-(o-tolyl)-5-(trifluoromethyl)pyridin-2-yl)sulfamoyl)pyridin-2-yl)amino)propanoic Acid (91)

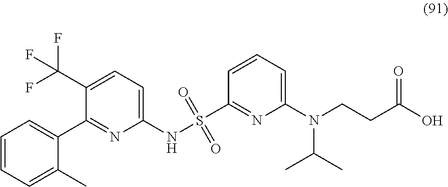 [SMILES: Cc1ccccc1-c1nc(NS(=O)(=O)c2cccc(N(CCC(=O)O)C(C)C)n2)ccc1C(F)(F)F]

3-(isopropyl(6-(N-(6-(o-tolyl)-5-(trifluoromethyl)pyridin-2-yl)sulfamoyl)pyridin-2-yl)amino)propanoic acid (91) was synthesized using the procedure described in Example 87, except in step 1, 4-(butylamino)butan-1-ol was replaced with 3-(isopropylamino)propan-1-ol and heating in microwave at 200° C. for 1 hour was replaced with heating in oil bath at 120° C. for 5 days. LCMS (Condition 1): m/z 522.9 [M+H]⁺, 1.68 min. ¹H NMR (400 MHz, DMSO-d₆) δ 12.33 (br s, 1H), 11.58 (br s, 1H), 8.15 (d, J=8.9 Hz, 1H), 7.67 (dd, J=8.7, 7.3 Hz, 1H), 7.50 (d, J=8.9 Hz, 1H), 7.32-7.28 (m, 1H), 7.25-7.15 (m, 2H), 7.08 (d, J=7.2 Hz, 1H), 7.03 (d, J=7.5 Hz, 1H), 6.86 (d, J=8.8 Hz, 1H), 4.41-4.36 (m, 1H), 3.46-3.37 (m, 2H), 2.37 (t, J=7.6 Hz, 2H), 1.73 (s, 3H), 1.18-0.80 (m, 6H).

### Example 92: 4-((6-(N-(6-(5-fluoro-2-methylphenyl)-5-(trifluoromethyl)pyridin-2-yl)sulfamoyl)pyridin-2-yl)(methyl)amino)butanoic Acid (92)

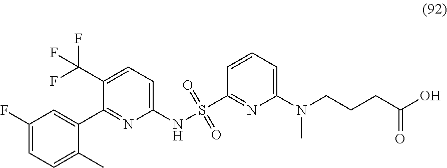 [SMILES: Cc1ccc(F)cc1-c1nc(NS(=O)(=O)c2cccc(N(C)CCCC(=O)O)n2)ccc1C(F)(F)F]

4-((6-(N-(6-(5-fluoro-2-methylphenyl)-5-(trifluoromethyl)pyridin-2-yl)sulfamoyl)pyridin-2-yl)(methyl)amino)butanoic acid (92) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 4-(methylamino)butan-1-ol, and heating in microwave at 200° C. for 1 hour was replaced with heating in oil bath at 60° C. for 18 hours. LCMS (Condition 1): m/z 527.0 [M+H]⁺, 1.58 min. ¹H NMR (500 MHz, Chloroform-d) δ 8.05 (d, J=9.0 Hz, 1H), 7.79-7.75 (m, 1H), 7.58 (dd, J=8.6, 7.3 Hz, 1H), 7.23-7.19 (m, 2H), 7.05 (td, J=8.5, 2.7 Hz, 1H), 6.84 (dd, J=8.8, 2.6 Hz, 1H), 6.65 (d, J=8.5 Hz, 1H), 3.91-3.82 (m, 1H), 3.67-3.58 (m, 1H), 2.98 (s, 3H), 2.22 (t, J=6.2 Hz, 2H), 1.96-1.87 (m, 5H).

### Example 93: 4-((6-(N-(6-(2-ethyl-5-fluorophenyl)-5-(trifluoromethyl)pyridin-2-yl)sulfamoyl)pyridin-2-yl)(methyl)amino)butanoic Acid (93)

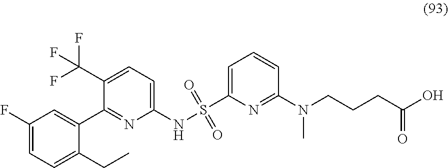 [SMILES: CCc1ccc(F)cc1-c1nc(NS(=O)(=O)c2cccc(N(C)CCCC(=O)O)n2)ccc1C(F)(F)F]

4-((6-(N-(6-(2-ethyl-5-fluorophenyl)-5-(trifluoromethyl)pyridin-2-yl)sulfamoyl)pyridin-2-yl)(methyl)amino)butanoic acid (93) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(6-(2-ethyl-5-fluorophenyl)-5-(trifluoromethyl)pyridin-2-yl)-6-fluoropyridine-2-sulfonamide (int-b3), and 4-(butylamino)butan-1-ol was replaced with 4-(methylamino)butan-1-ol, and heating in microwave at 200° C. for 1 hour was replaced with heating in oil bath at 60° C. for 18 hours. LCMS (Condition 1): m/z 541.0 [M+H]⁺, 1.62 min. ¹H NMR (400 MHz, Chloroform-d) δ 7.95 (d, J=8.9 Hz, 1H), 7.68 (d, 1H), 7.52-7.46 (m, 1H), 7.19-7.15 (m, 1H), 7.11 (d, 1H), 7.01 (td, J=8.5, 2.7 Hz, 1H), 6.73 (dd, J=8.9, 2.7 Hz, 1H), 6.55 (d, J=8.6 Hz, 1H), 3.86-3.74 (m, 1H), 3.55-3.44 (m, 1H), 2.88 (s, 3H), 2.27-2.05 (m, 4H), 1.88-1.73 (m, 2H), 0.93 (t, J=7.6 Hz, 3H).

### Example 94: 4-(isopropyl(6-(N-(6-(o-tolyl)-5-(trifluoromethyl)pyridin-2-yl)sulfamoyl)pyridin-2-yl)amino)butanoic acid (94)

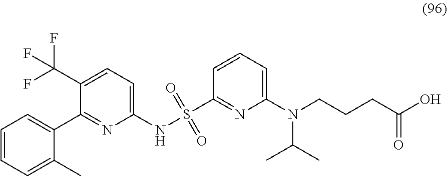 [SMILES: Cc1ccccc1-c1nc(NS(=O)(=O)c2cccc(N(CCCC(=O)O)C(C)C)n2)ccc1C(F)(F)F]

4-(isopropyl(6-(N-(6-(o-tolyl)-5-(trifluoromethyl)pyridin-2-yl)sulfamoyl)pyridin-2-yl)amino)butanoic acid (94) was synthesized using the procedure described in Example 87, except in step 1, 4-(butylamino)butan-1-ol was replaced with 4-(isopropylamino)butan-1-ol and heating in microwave at 200° C. for 1 hour was replaced with heating in oil bath at 120° C. for 6 days. LCMS (Condition 1): m/z 536.9 [M+H]⁺, 1.74 min. ¹H NMR (400 MHz, DMSO-d₆) δ 12.16 (br s, 1H), 11.53 (br s, 1H), 8.18 (d, J=8.9 Hz, 1H), 7.65 (dd, J=8.7, 7.2 Hz, 1H), 7.48 (d, J=8.8 Hz, 1H), 7.32-7.28 (m, 1H), 7.24-7.14 (m, 2H), 7.03 (dd, J=11.4, 7.4 Hz, 2H), 6.89 (d, J=8.8 Hz, 1H), 4.49-4.43 (m, 1H), 3.19-3.14 (m, 2H), 2.26 (t, J=7.0 Hz, 2H), 1.72 (s, 3H), 1.60-1.57 (m, 2H), 0.99 (d, J=6.7 Hz, 3H), 0.94 (d, J=6.7 Hz, 3H).

### Example 95: 4-((6-(N-(6-(5-fluoro-2-methylphenyl)-5-(trifluoromethyl)pyridin-2-yl)sulfamoyl)pyridin-2-yl)(isopropyl)amino)butanoic Acid (95)

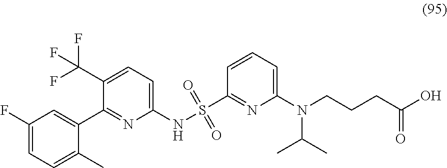 [SMILES: Cc1ccc(F)cc1-c1nc(NS(=O)(=O)c2cccc(N(CCCC(=O)O)C(C)C)n2)ccc1C(F)(F)F]

4-((6-(N-(6-(5-fluoro-2-methylphenyl)-5-(trifluoromethyl)pyridin-2-yl)sulfamoyl)pyridin-2-yl)(isopropyl)amino)butanoic acid (95) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 4-(isopropylamino)butan-1-ol, and heating in microwave at 200° C. for 1 hour was replaced with heating in oil bath at 120° C. for 6 days. LCMS (Condition 1): m/z 554.9 [M+H]⁺, 1.75 min. ¹H NMR (400 MHz, Methanol-d₄) δ 8.07 (d, J=8.8 Hz, 1H), 7.59 (dd, J=8.8, 7.3 Hz, 1H), 7.55-7.48 (m, 1H), 7.20 (dd, J=8.5, 5.6 Hz, 1H), 7.13 (d, J=7.2 Hz, 1H), 7.05-7.00 (m, 1H), 6.88 (d, J=8.7 Hz, 1H), 6.77 (dd, J=9.2, 2.7 Hz, 1H), 4.58-4.51 (m, 1H), 3.30-3.21 (m, 2H), 2.32 (t, J=6.9 Hz, 2H), 1.82 (s, 3H), 1.79-1.72 (m, 2H), 1.09 (s, 6H).

### Example 96: 4-((6-(N-(6-(2-ethylphenyl)-5-(trifluoromethyl)pyridin-2-yl)sulfamoyl)pyridin-2-yl)(isopropyl)amino)butanoic Acid (96)

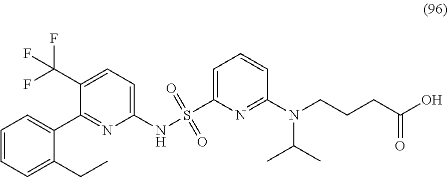 [SMILES: CCc1ccccc1-c1nc(NS(=O)(=O)c2cccc(N(CCCC(=O)O)C(C)C)n2)ccc1C(F)(F)F]

4-((6-(N-(6-(2-ethylphenyl)-5-(trifluoromethyl)pyridin-2-yl)sulfamoyl)pyridin-2-yl)(isopropyl)amino)butanoic acid (96) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(6-(2-ethylphenyl)-5-(trifluoromethyl)pyridin-2-yl)-6-fluoropyridine-2-sulfonamide (int-b11), 4-(butylamino)butan-1-ol was replaced with 4-(isopropylamino)butan-1-ol, and heating in microwave at 200° C. for 1 hour was replaced with heating in oil bath at 120° C. for 5 days. LCMS (Condition 1): m/z 550.9 [M+H]⁺, 1.78 min. ¹H NMR (400 MHz, Methanol-d₄) δ 8.05 (d, J=8.9 Hz, 1H), 7.62-7.53 (m, 2H), 7.36-7.32 (m, 1H), 7.26 (dd, J=7.8, 1.3 Hz, 1H), 7.21-7.11 (m, 2H), 7.02 (d, J=7.6 Hz, 1H), 6.88 (d, J=8.7 Hz, 1H), 4.56-4.53 (m, 1H), 3.30-3.25 (m, 2H), 2.32 (t, J=6.9 Hz, 2H), 2.29-2.23 (m, 1H), 2.18-2.10 (m, 1H), 1.81-1.73 (m, 2H), 1.11-1.07 (m, 6H), 0.95 (t, J=7.6 Hz, 3H).

### Example 97: 4-(ethyl(6-(N-(6-(2-ethylphenyl)-5-(trifluoromethyl)pyridin-2-yl)sulfamoyl)pyridin-2-yl)amino)butanoic Acid (97)

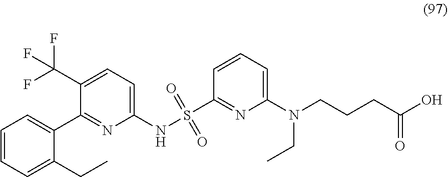 [SMILES: CCc1ccccc1-c1nc(NS(=O)(=O)c2cccc(N(CC)CCCC(=O)O)n2)ccc1C(F)(F)F]

4-(ethyl(6-(N-(6-(2-ethylphenyl)-5-(trifluoromethyl)pyridin-2-yl)sulfamoyl)pyridin-2-yl)amino)butanoic acid (97) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(6-(2-ethylphenyl)-5-(trifluoromethyl)pyridin-2-yl)-6-fluoropyridine-2-sulfonamide (int-b11), 4-(butylamino)butan-1-ol was replaced with 4-(ethylamino)butan-1-ol, and microwave heating at 200° C. for 1 hour was replaced with microwave heating at 150° C. for 3 hours. LCMS (Condition 1): m/z 536.9 [M+H]⁺, 1.74 min. ¹H NMR (400 MHz, DMSO-d₆) δ 12.10 (br s, 1H), 11.54 (br s, 1H), 8.17 (d, J=8.9 Hz, 1H), 7.64 (dd, J=8.7, 7.3 Hz, 1H), 7.50 (d, J=8.8 Hz, 1H), 7.37-7.33 (m, 1H), 7.25 (dd, J=7.9, 1.3 Hz, 1H), 7.22-7.18 (m, 1H), 7.03 (dd, J=9.3, 7.3 Hz, 2H), 6.86 (d, J=8.7 Hz, 1H), 3.42-3.35 (m, 2H), 3.33-3.20 (m, 2H), 2.17 (t, J=7.3 Hz, 2H), 2.14-2.06 (m, 1H), 2.04-1.94 (m, 1H), 1.65-1.57 (m, 2H), 0.90-0.82 (m, 6H).

### Example 98: 3-(1-(6-(N-(6-(o-tolyl)-5-(trifluoromethyl)pyridin-2-yl)sulfamoyl)pyridin-2-yl)piperidin-2-yl)propanoic Acid (98)

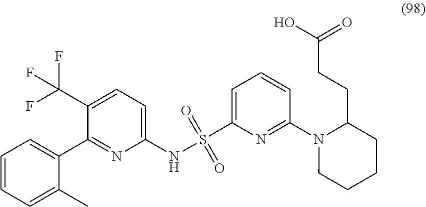 [SMILES: Cc1ccccc1-c1nc(NS(=O)(=O)c2cccc(N3CCCCC3CCC(=O)O)n2)ccc1C(F)(F)F]

3-(1-(6-(N-(6-(o-tolyl)-5-(trifluoromethyl)pyridin-2-yl)sulfamoyl)pyridin-2-yl)piperidin-2-yl)propanoic acid (98) was synthesized using the procedure described in Example 87, except in step 1, 4-(butylamino)butan-1-ol was replaced with 3-(piperidin-2-yl)propan-1-ol hydrochloride and heating in microwave at 200° C. for 1 hour was replaced with heating in an oil bath at 160° C. for 18 hours. LCMS (Condition 1): m/z 549.2 [M+H]⁺, 1.86 min. ¹H NMR (400 MHz, DMSO-d₆) δ 12.05 (s, 1H), 11.55 (s, 1H), 8.23-8.09 (m, 1H), 7.64 (dd, J=8.8, 7.2 Hz, 1H), 7.53-7.45 (m, 1H), 7.31 (td, J=7.5, 1.5 Hz, 1H), 7.24-7.14 (m, 2H), 7.08-7.01 (m, 2H), 6.99 (d, J=8.8 Hz, 1H), 4.45-4.28 (m, 1H), 4.05-3.92 (m, 1H), 2.82-2.69 (m, 1H), 2.16-1.92 (m, 2H), 1.91-1.03 (m, 11H).

### Example 99: 5-((6-(N-(6-(2-ethyl-5-fluorophenyl)-5-(trifluoromethyl)pyridin-2-yl)sulfamoyl)pyridin-2-yl)(isopropyl)amino)pentanoic Acid (99)

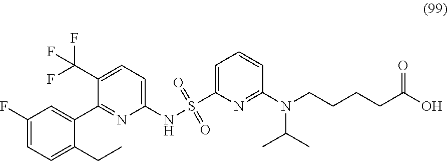 [SMILES: CCc1ccc(F)cc1-c1nc(NS(=O)(=O)c2cccc(N(CCCCC(=O)O)C(C)C)n2)ccc1C(F)(F)F]

5-((6-(N-(6-(2-ethyl-5-fluorophenyl)-5-(trifluoromethyl)pyridin-2-yl)sulfamoyl)pyridin-2-yl)(isopropyl)amino)pentanoic acid (99) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(6-(2-ethyl-5-fluorophenyl)-5-(trifluoromethyl)pyridin-2-yl)-6-fluoropyridine-2-sulfonamide (int-b3), 4-(butylamino)butan-1-ol was replaced with 5-(isopropylamino)pentan-1-ol, NMP was replaced with a 6:1 mixture of dioxane:DMA, and heating in microwave at 200° C. for 1 hour was replaced with heating in an oil bath at 120° C. for 5 days. LCMS (Condition 1): m/z 583.2 [M+H]⁺, 1.60 min. ¹H NMR (500 MHz, Methanol-d₄) δ 7.98-7.95 (m, 1H), 7.50-7.42 (m, 2H), 7.15 (dd, J=8.5, 5.6 Hz, 1H), 7.02 (d, J=7.0 Hz, 1H), 6.97 (td, J=8.6, 2.8 Hz, 1H), 6.66-6.61 (m, 2H), 4.46-4.37 (m, 1H), 3.17-3.11 (m, 2H), 2.23-2.18 (m, 2H), 2.18-1.94 (m, 3H), 1.56-1.39 (m, 4H), 1.03-0.93 (m, 6H), 0.84-0.80 (m, 2H).

### Example 100: 5-((6-(N-(6-(5-fluoro-2-methylphenyl)-5-(trifluoromethyl)pyridin-2-yl)sulfamoyl)pyridin-2-yl)(isopropyl)amino)pentanoic Acid (100)

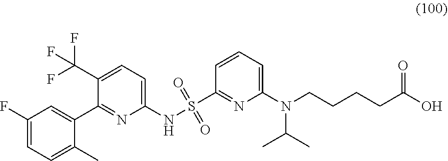 [SMILES: Cc1ccc(F)cc1-c1nc(NS(=O)(=O)c2cccc(N(CCCCC(=O)O)C(C)C)n2)ccc1C(F)(F)F]

5-((6-(N-(6-(5-fluoro-2-methylphenyl)-5-(trifluoromethyl)pyridin-2-yl)sulfamoyl)pyridin-2-yl)(isopropyl)amino)pentanoic acid (100) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 5-(isopropylamino)pentan-1-ol, NMP was replaced with a 6:1 mixture of dioxane:DMA, and heating in microwave at 200° C. for 1 hour was replaced with heating in an oil bath at 120° C. for 5 days. LCMS (Condition 1): m/z 569.2 [M+H]⁺, 1.57 min. Rotamers are present in ¹H NMR spectra. ¹H NMR (500 MHz, Acetonitrile-d₃) δ 8.09-8.03 (m, 1H), 7.59-7.50 (m, 1H), 7.29-7.26 (m, 1H), 7.25-7.20 (m, 1H), 7.11-7.04 (m, 1H), 6.87-6.81 (m, 1H), 6.75-6.66 (m, 1H), 4.46-4.16 (m, 1H), 3.22-3.04 (m, 2H), 2.31-2.23 (m, 2H), 1.97-1.96 (m, 3H), 1.77-1.74 (m, 3H), 1.59-1.44 (m, 4H), 1.09-0.93 (m, 6H).

### Example 101: 5-(isopropyl(6-(N-(6-(o-tolyl)-5-(trifluoromethyl)pyridin-2-yl)sulfamoyl)pyridin-2-yl)amino)pentanoic Acid (101)

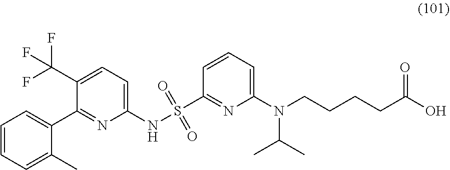 [SMILES: Cc1ccccc1-c1nc(NS(=O)(=O)c2cccc(N(CCCCC(=O)O)C(C)C)n2)ccc1C(F)(F)F]

5-(isopropyl(6-(N-(6-(o-tolyl)-5-(trifluoromethyl)pyridin-2-yl)sulfamoyl)pyridin-2-yl)amino)pentanoic acid (101) was synthesized using the procedure described in Example 87, except in step 1, 4-(butylamino)butan-1-ol was replaced with 5-(isopropylamino)pentan-1-ol, NMP was replaced with a 6:1 mixture of dioxane:DMA, and heating in microwave at 200° C. for 1 hour was replaced with heating in an oil bath at 120° C. for 4 days. LCMS (Condition 1): m/z 551.2 [M+H]⁺, 1.56 min. ¹H NMR (500 MHz, Acetonitrile-d₃) δ 8.06 (d, J=8.9 Hz, 1H), 7.58 (dd, J=8.8, 7.2 Hz, 1H), 7.51 (d, J=8.8 Hz, 1H), 7.32 (td, J=7.5, 1.3 Hz, 1H), 7.25-7.18 (m, 2H), 7.10 (d, J=7.0 Hz, 1H), 7.06 (d, J=7.5 Hz, 1H), 6.73 (d, J=8.7 Hz, 1H), 4.47-4.34 (m, 1H), 3.19 (t, J=7.6 Hz, 2H), 2.27 (t, J=7.0 Hz, 2H), 1.80 (s, 3H), 1.60-1.44 (m, 4H), 1.05 (s, 6H).

### Example 102: 4-(butyl(6-(N-(6-(5-fluoro-2-methylphenyl)-5-(trifluoromethyl)pyridin-2-yl)sulfamoyl)pyridin-2-yl)amino)butanoic Acid (102)

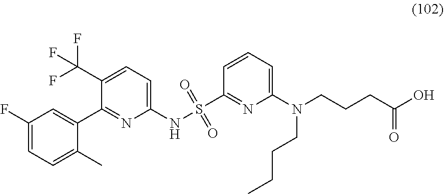 [SMILES: CCCCN(CCCC(=O)O)c1cccc(S(=O)(=O)Nc2ccc(C(F)(F)F)c(-c3cc(F)ccc3C)n2)n1]

4-(butyl(6-(N-(6-(5-fluoro-2-methylphenyl)-5-(trifluoromethyl)pyridin-2-yl)sulfamoyl)pyridin-2-yl)amino)butanoic acid (102) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10). LCMS (Condition 1): m/z 568.9 [M+H]⁺, 1.78 min. ¹H NMR (400 MHz, Methanol-d₄) δ 8.06 (d, J=8.8 Hz, 1H), 7.56 (dd, J=8.7, 7.3 Hz, 1H), 7.45 (dd, J=8.8, 0.9 Hz, 1H), 7.19 (dd, J=8.5, 5.6 Hz, 1H), 7.11 (d, J=7.2 Hz, 1H), 7.05-7.00 (m, 1H), 6.80-6.75 (m, 2H), 3.52-3.33 (m, 4H), 2.24 (t, J=7.1 Hz, 2H), 1.80 (s, 3H), 1.77-1.72 (m, 2H), 1.50-1.43 (m, 2H), 1.32-1.23 (m, 2H), 0.90 (t, J=7.3 Hz, 3H).

### Example 103: 4-((cyclopropylmethyl)(6-(N-(6-(o-tolyl)-5-(trifluoromethyl)pyridin-2-yl)sulfamoyl)pyridin-2-yl)amino)butanoic Acid (103)

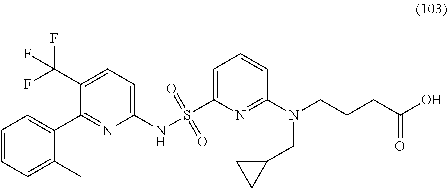 [SMILES: Cc1ccccc1-c1nc(NS(=O)(=O)c2cccc(N(CCCC(=O)O)CC3CC3)n2)ccc1C(F)(F)F]

4-((cyclopropylmethyl)(6-(N-(6-(o-tolyl)-5-(trifluoromethyl)pyridin-2-yl)sulfamoyl)pyridin-2-yl)amino)butanoic acid (103) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(o-tolyl)-5-(trifluoromethyl)pyridin-2-yl)pyridine-2-sulfonamide (int-b1), 4-(butylamino)butan-1-ol was replaced with 4-((cyclopropylmethyl)amino)butan-1-ol (int-a4), NMP was replaced with dioxane, N,N-diisopropylethylamine was replaced with K₂CO₃, and heating in microwave at 200° C. for 1 hour was replaced with heating in an oil bath at 120° C. for 18 hours. LCMS (Condition 1): m/z 549.1 [M+H]⁺, 1.44 min. ¹H NMR (400 MHz, Methanol-d₄) δ 8.04 (dd, J=8.9, 7.3 Hz, 1H), 7.61-7.55 (m, 1H), 7.51 (dd, J=8.9, 0.9 Hz, 1H), 7.32-7.25 (m, 1H), 7.23-7.11 (m, 3H), 7.03 (d, J=7.6 Hz, 1H), 6.85 (dd, J=18.7, 8.7 Hz, 1H), 5.06-4.92 (m, 2H), 3.54-3.37 (m, 3H), 2.32-2.16 (m, 3H), 1.88 (s, 3H), 1.82-1.72 (m, 2H), 1.35-1.29 (m, 3H).

### Example 104: 4-((cyclopropylmethyl)(6-(N-(6-(5-fluoro-2-methylphenyl)-5-(trifluoromethyl)pyridin-2-yl)sulfamoyl)pyridin-2-yl)amino)butanoic Acid (104)

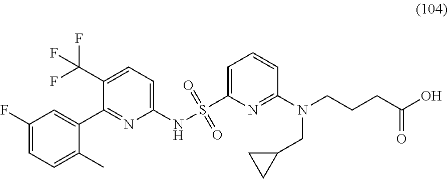 [SMILES: Cc1ccc(F)cc1-c1nc(NS(=O)(=O)c2cccc(N(CCCC(=O)O)CC3CC3)n2)ccc1C(F)(F)F]

4-((cyclopropylmethyl)(6-(N-(6-(5-fluoro-2-methylphenyl)-5-(trifluoromethyl)pyridin-2-yl)sulfamoyl)pyridin-2-yl)amino)butanoic acid (104) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 4-((cyclopropylmethyl)amino)butan-1-ol (int-a4), NMP was replaced with dioxane, N,N-diisopropylethylamine was replaced with K₂CO₃, and heating in microwave at 200° C. for 1 hour was replaced with heating in an oil bath at 120° C. for 18 hours. LCMS (Condition 1): m/z 567.1 [M+H]⁺, 1.45 min. ¹H NMR (400 MHz, Methanol-d₄) δ 8.05 (dd, J=8.9, 7.0 Hz, 1H), 7.57 (dd, J=8.7, 7.3 Hz, 1H), 7.50-7.45 (m, 1H), 7.20 (dd, J=8.6, 5.6 Hz, 1H), 7.13 (dd, J=7.2, 4.2 Hz, 1H), 7.06-6.98 (m, 1H), 6.92-6.71 (m, 2H), 5.06-4.91 (m, 2H), 3.56-3.36 (m, 4H), 2.22 (d, J=7.1 Hz, 2H), 1.81 (s, 3H), 1.80-1.73 (m, 1H), 1.38-1.22 (m, 2H), 1.05-0.80 (m, 2H).

### Example 105: 3-((6-(N-(6-(o-tolyl)-5-(trifluoromethyl)pyridin-2-yl)sulfamoyl)pyridin-2-yl)amino)cyclohexane-1-carboxylic Acid (105)

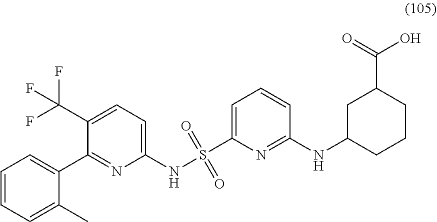 [SMILES: Cc1ccccc1-c1nc(NS(=O)(=O)c2cccc(NC3CCCC(C(=O)O)C3)n2)ccc1C(F)(F)F]

3-((6-(N-(6-(o-tolyl)-5-(trifluoromethyl)pyridin-2-yl)sulfamoyl)pyridin-2-yl)amino)cyclohexane-1-carboxylic acid (105) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 3-aminocyclohexane-1-carboxylic acid. LCMS (Condition 1): m/z 535.1 [M+H]⁺, 1.72 min. Mixture of geometric isomers. ¹H NMR (400 MHz, Chloroform-d) δ 7.97 (d, J=8.9 Hz, 1H), 7.59 (d, J=8.6 Hz, 1H), 7.52 (dd, J=7.3, 8.4 Hz, 1H), 7.39-7.32 (m, 1H), 7.31-7.27 (m, 1H, obscured by solvent peak), 7.23 (d, J=7.6 Hz, 1H), 7.13 (d, J=7.5 Hz, 1H), 6.49 (d, J=8.4 Hz, 1H), 4.52 (s, 1H), 3.53 (d, J=6.9 Hz, 1H), 2.38 (td, J=3.6, 11.7 Hz, 1H), 2.24 (d, J=12.6 Hz, 1H), 2.05 (s, 3H), 1.96 (d, J=12.5 Hz, 2H), 1.83 (s, 1H), 1.41-1.28 (m, 3H), 1.15-0.99 (m, 1H). ¹⁹F NMR (376 MHz, Chloroform-d) δ −58.63 (s, 3F).

### Example 106: Racemic Mixture of (1 S,3R)-3-((6-(N-(6-(o-tolyl)-5-(trifluoromethyl)pyridin-2-yl)sulfamoyl)pyridin-2-yl)amino)cyclohexane-1-carboxylic acid (106a) and (1R,3S)-3-((6-(N-(6-(o-tolyl)-5-(trifluoromethyl)pyridin-2-yl)sulfamoyl)pyridin-2-yl)amino)cyclohexane-1-carboxylic Acid (106b)

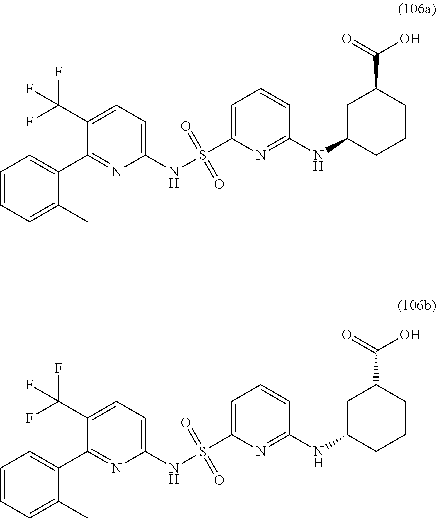

A mixture of compounds (106a) and (106b) was obtained using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with cis-3-aminocyclohexane-1-carboxylic acid. LCMS (Condition 1): m/z 535.2 [M+H]⁺, 1.72 min. ¹H NMR (400 MHz, Chloroform-d) δ 7.97 (d, J=9.0 Hz, 1H), 7.59 (d, J=8.8 Hz, 1H), 7.52 (dd, J=7.3, 8.4 Hz, 1H), 7.35 (td, J=1.2, 7.5 Hz, 1H), 7.29 (d, J=7.1 Hz, 1H), 7.26-7.24 (m, 1H), 7.22 (d, J=7.0 Hz, 1H), 7.13 (d, J=7.5 Hz, 1H), 6.49 (d, J=8.4 Hz, 1H), 4.57 (s, 1H), 3.51 (s, 1H), 2.37 (dt, J=5.8, 11.5 Hz, 1H), 2.22 (d, J=11.8 Hz, 1H), 2.04 (d, J=2.5 Hz, 3H), 1.96 (s, 2H), 1.85 (d, J=19.3 Hz, 1H), 1.42-1.28 (m, 3H), 1.05 (q, J=9.9, 10.4 Hz, 1H). ¹⁹F NMR (376 MHz, Chloroform-d) δ −58.64 (s, 3F).

### Example 107: Racemic Mixture of (1R,2S,3R,4S)-3-((6-(N-(6-(o-tolyl)-5-(trifluoromethyl)pyridin-2-yl)sulfamoyl)pyridin-2-yl)amino)bicyclo[2.2.1]hept-5-ene-2-carboxylic Acid (107a) and (1 S,2R,3S,4R)-3-((6-(N-(6-(o-tolyl)-5-(trifluoromethyl)pyridin-2-yl)sulfamoyl)pyridin-2-yl)amino)bicyclo[2.2.1]hept-5-ene-2-carboxylic Acid (107b)

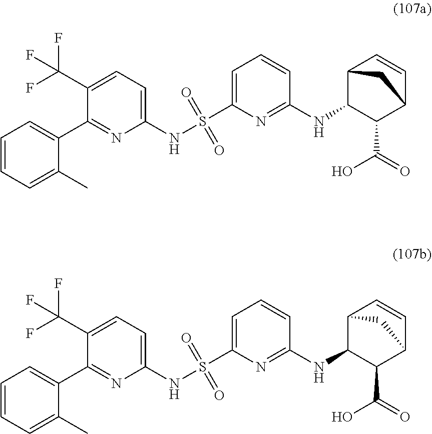 [SMILES: Cc1ccccc1-c1nc(NS(=O)(=O)c2cccc(N[C@H]3[C@@H](C(=O)O)[C@H]4C=C[C@@H]3C4)n2)ccc1C(F)(F)F ; Cc1ccccc1-c1nc(NS(=O)(=O)c2cccc(N[C@@H]3[C@H](C(=O)O)[C@@H]4C=C[C@H]3C4)n2)ccc1C(F)(F)F]

A mixture of compounds (109a) and (107b), was obtained using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 3-endo-aminobicyclo[2.2.1]hept-5-ene-2-endo-carboxylic acid. LCMS (Condition 1): m/z 545.1 [M+H]⁺, 1.77 min. ¹H NMR (400 MHz, Chloroform-d) δ 8.00 (d, J=8.9 Hz, 1H), 7.86-7.74 (m, 1H), 7.46-7.39 (m, 1H), 7.36 (td, J=1.2, 7.5 Hz, 1H), 7.24 (dd, J=4.7, 10.3 Hz, 3H), 7.12 (d, J=7.5 Hz, 1H), 6.49 (d, J=8.4 Hz, 1H), 6.33-6.21 (m, 1H), 6.10 (s, 1H), 5.47 (d, J=6.8 Hz, 1H), 4.56 (s, 1H), 3.17-2.94 (m, 3H), 2.06-1.91 (m, 4H), 1.45 (s, 1H). ¹⁹F NMR (376 MHz, Chloroform-d) δ −58.77 (d, J=5.4 Hz, 3F).

### Example 108: Racemic Mixture of (1R,2S,3R,4S)-3-((6-(N-(6-(o-tolyl)-5-(trifluoromethyl)pyridin-2-yl)sulfamoyl)pyridin-2-yl)amino)bicyclo[2.2.1]heptane-2-carboxylic Acid (108a) and (1 S,2R,3S,4R)-3-((6-(N-(6-(o-tolyl)-5-(trifluoromethyl)pyridin-2-yl)sulfamoyl)pyridin-2-yl)amino)bicyclo[2.2.1]heptane-2-carboxylic Acid (108b)

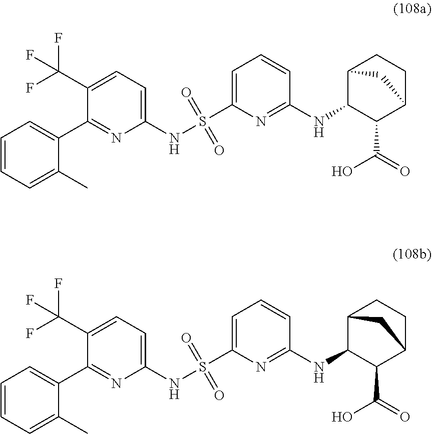 [SMILES: Cc1ccccc1-c1nc(NS(=O)(=O)c2cccc(N[C@@H]3[C@H]4CC[C@H](C4)[C@@H]3C(=O)O)n2)ccc1C(F)(F)F ; Cc1ccccc1-c1nc(NS(=O)(=O)c2cccc(N[C@H]3[C@@H]4CC[C@@H](C4)[C@H]3C(=O)O)n2)ccc1C(F)(F)F]

A mixture of compounds (108a) and (108b), was obtained using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 3-exo-aminobicyclo[2.2.1]heptane-2-exo-carboxylic acid. LCMS (Condition 1): m/z 547.1 [M+H]⁺, 1.79 min. ¹H NMR (400 MHz, Chloroform-d) δ 7.97 (d, J=8.9 Hz, 1H), 7.65 (d, J=7.8 Hz, 1H), 7.45-7.39 (m, 1H), 7.33 (td, J=1.3, 7.6 Hz, 1H), 7.25-7.16 (m, 3H), 7.09 (d, J=7.5 Hz, 1H), 6.51 (d, J=8.5 Hz, 1H), 5.38 (s, 1H), 3.96 (d, J=6.9 Hz, 1H), 2.56 (d, J=7.9 Hz, 1H), 2.40 (s, 1H), 2.10 (s, 1H), 1.98 (m, 3H), 1.85 (d, J=10.4 Hz, 1H), 1.50-1.41 (m, 1H), 1.21 (d, J=10.8 Hz, 3H). ¹⁹F NMR (376 MHz, Chloroform-d) δ −58.80 (s, 3F).

### Example 109: Racemic Mixture of (1 S,2S,3R,4R)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid (109a) and (1R,2R,3S,4S)-3-((6-(N-(6-(o-tolyl)-5-(trifluoromethyl)pyridin-2-yl)sulfamoyl)pyridin-2-yl)amino)bicyclo[2.2.1]heptane-2-carboxylic Acid (109b)

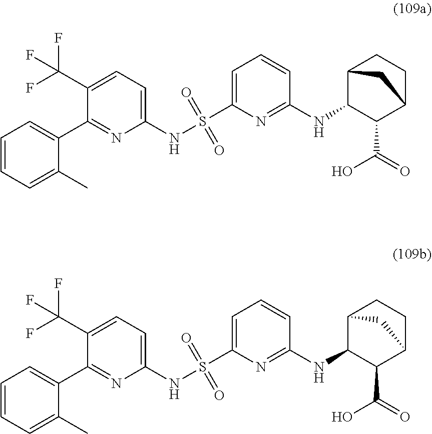 [SMILES: Cc1ccccc1-c1nc(NS(=O)(=O)c2cccc(N[C@@H]3[C@@H]4CC[C@@H](C4)[C@@H]3C(=O)O)n2)ccc1C(F)(F)F ; Cc1ccccc1-c1nc(NS(=O)(=O)c2cccc(N[C@H]3[C@H]4CC[C@H](C4)[C@H]3C(=O)O)n2)ccc1C(F)(F)F]

A mixture of compounds (109a) and (109b), was obtained using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with 3-endo-aminobicyclo[2.2.1]heptane-2-endo-carboxylic acid. LCMS (Condition 1): m/z 546.9 [M+H]⁺, 1.73 min. ¹H NMR (400 MHz, Chloroform-d) δ 8.00 (d, J=8.9 Hz, 1H), 7.68 (d, J=8.7 Hz, 1H), 7.49-7.41 (m, 1H), 7.35 (td, J=1.3, 7.6 Hz, 1H), 7.27-7.18 (m, 3H), 7.12 (d, J=7.5 Hz, 1H), 6.54 (d, J=8.5 Hz, 1H), 5.36 (s, 1H), 3.98 (t, J=7.3 Hz, 1H), 2.61 (d, J=7.9 Hz, 1H), 2.44 (s, 1H), 2.13 (s, 1H), 2.05-1.92 (m, 3H), 1.89 (d, J=10.5 Hz, 1H), 1.50 (m, 1H), 1.36-1.30 (m, 1H), 1.26-1.09 (m, 3H). ¹⁹F NMR (376 MHz, Chloroform-d) δ −58.81 (s, 3F).

### Example 110: 4-(ethyl(6-(N-(6-(2-ethyl-5-fluorophenyl)-5-(trifluoromethyl)pyridin-2-yl)sulfamoyl)pyridin-2-yl)amino)butanoic Acid (110)

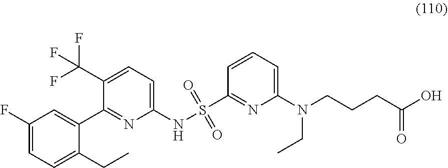 [SMILES: CCc1ccc(F)cc1-c1nc(NS(=O)(=O)c2cccc(N(CC)CCCC(=O)O)n2)ccc1C(F)(F)F]

4-(ethyl(6-(N-(6-(2-ethyl-5-fluorophenyl)-5-(trifluoromethyl)pyridin-2-yl)sulfamoyl)pyridin-2-yl)amino)butanoic acid (110) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(6-(2-ethyl-5-fluorophenyl)-5-(trifluoromethyl)pyridin-2-yl)-6-fluoropyridine-2-sulfonamide (int-b3) and 4-(butylamino)butan-1-ol was replaced with 4-(ethylamino)butan-1-ol. LCMS (Condition 1): m/z 554.9 [M+H]⁺, 1.73 min. ¹H NMR (400 MHz, Methanol-d₄) δ 8.06 (d, J=8.8 Hz, 1H), 7.62-7.50 (m, 2H), 7.26 (dd, J=8.6, 5.6 Hz, 1H), 7.13 (d, J=7.2 Hz, 1H), 7.10-7.05 (m, 1H), 6.82 (d, J=8.7 Hz, 1H), 6.76 (dd, J=9.2, 2.8 Hz, 1H), 3.52-3.36 (m, 4H), 2.32-2.17 (m, 3H), 2.15-2.06 (m, 1H), 1.81-1.74 (m, 2H), 1.03 (t, J=7.0 Hz, 3H), 0.93 (t, J=7.6 Hz, 3H).

### Example 111: 3-((6-(N-(6-(2-cyclopropyl-5-fluorophenyl)-5-(trifluoromethyl)pyridin-2-yl)sulfamoyl)pyridin-2-yl)(ethyl)amino)propanoic Acid (111)

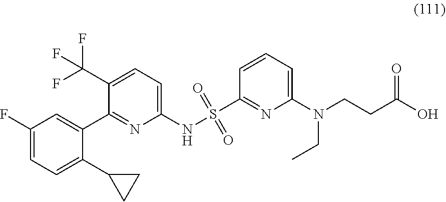 [SMILES: CCN(CCC(=O)O)c1cccc(S(=O)(=O)Nc2ccc(C(F)(F)F)c(-c3cc(F)ccc3C3CC3)n2)n1]

3-((6-(N-(6-(2-cyclopropyl-5-fluorophenyl)-5-(trifluoromethyl)pyridin-2-yl)sulfamoyl)pyridin-2-yl)(ethyl)amino)propanoic acid (111) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-cyclopropyl-5-fluorophenyl)-5-(trifluoromethyl)pyridin-2-yl)-6-fluoro-N-(methoxymethyl)pyridine-2-sulfonamide (int-b21) and 4-methylpiperidine-4-carboxylic acid hydrochloride was replaced with 3-(ethylamino)propanoic acid hydrochloride. LCMS (Condition 1): m/z 553.1 [M+H]⁺, 1.43 min. ¹H NMR (400 MHz, Methanol-d₄) δ 8.07 (d, J=8.9 Hz, 1H), 7.60 (dd, J=8.7, 7.3 Hz, 2H), 7.18 (d, J=7.1 Hz, 1H), 7.07-6.93 (m, 2H), 6.87-6.68 (m, 2H), 3.76-3.56 (m, 2H), 3.53-3.37 (m, 2H), 2.47 (t, J=7.1 Hz, 2H), 1.41-1.23 (m, 1H), 1.06 (t, J=7.0 Hz, 3H), 0.61-0.47 (m, 3H), 0.40-0.28 (m, 1H).

### Example 112: 4-(butyl(6-(N-(3-chloro-2′-isopropyl-[2,3′-bipyridin]-6-yl)sulfamoyl)pyridin-2-yl)amino)butanoic Acid (112)

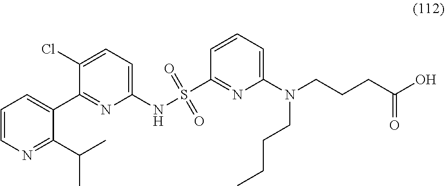 [SMILES: CCCCN(CCCC(=O)O)c1cccc(S(=O)(=O)Nc2ccc(Cl)c(-c3cccnc3C(C)C)n2)n1]

4-(butyl(6-(N-(3-chloro-2′-isopropyl-[2,3′-bipyridin]-6-yl)sulfamoyl)pyridin-2-yl)amino)butanoic acid (112) was synthesized using the procedure described in Example 87, except 6-fluoro-N-(6-(o-tolyl)-5-(trifluoromethyl)pyridin-2-yl)pyridine-2-sulfonamide (int-b1) was replaced with N-(3-chloro-2′-isopropyl-[2,3′-bipyridin]-6-yl)-6-fluoropyridine-2-sulfonamide (int-b4). LCMS (Condition 1): m/z 545.9 [M+H]⁺, 1.40 min. ¹H NMR (400 MHz, Methanol-d₄) δ 8.52 (dd, J=4.9, 1.7 Hz, 1H), 7.84-7.82 (m, 1H), 7.58-7.54 (m, 1H), 7.48-7.45 (m, 1H), 7.42-7.39 (m, 1H), 7.30-7.26 (m, 1H), 7.12-7.10 (m, 1H), 6.78 (d, J=8.6 Hz, 1H), 3.50-3.36 (m, 4H), 2.79-2.65 (m, 1H), 2.25 (t, J=7.1 Hz, 2H), 1.80-1.73 (m, 2H), 1.52-1.44 (m, 2H), 1.37-1.23 (m, 2H), 1.08 (br s, 6H), 0.91 (t, J=7.3 Hz, 3H).

### Example 113: 3-(cyclopropyl(6-(N-(6-(2-ethyl-5-fluorophenyl)-5-(trifluoromethyl)pyridin-2-yl)sulfamoyl)pyridin-2-yl)amino)propanoic Acid (113)

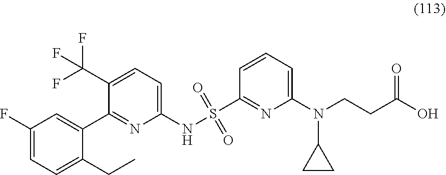 [SMILES: CCc1ccc(F)cc1-c1nc(NS(=O)(=O)c2cccc(N(CCC(=O)O)C3CC3)n2)ccc1C(F)(F)F]

3-(cyclopropyl(6-(N-(6-(2-ethyl-5-fluorophenyl)-5-(trifluoromethyl)pyridin-2-yl)sulfamoyl)pyridin-2-yl)amino)propanoic acid (113) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with N-(6-(2-ethyl-5-fluorophenyl)-5-(trifluoromethyl)pyridin-2-yl)-6-fluoropyridine-2-sulfonamide (int-b3), 3-(ethylamino)propanoic acid hydrochloride was replaced with tert-butyl 3-(cyclopropylamino)propanoate, and D-α-tocopherol polyethylene glycol 1000 succinate (TPGS) and water (0.5 mL) were added to the reaction mixture after 18 hours. LCMS (Condition 1): m/z 552.9 [M+H]⁺, 1.75 min. ¹H NMR (400 MHz, Methanol-d₄) δ 8.05 (d, J=8.8 Hz, 1H), 7.66 (dd, J=8.7, 7.3 Hz, 1H), 7.54-7.52 (m, 1H), 7.31-7.19 (m, 3H), 7.10-7.05 (m, 1H), 6.76 (dd, J=9.3, 2.8 Hz, 1H), 3.86-3.65 (m, 2H), 2.61-2.55 (m, 1H), 2.38 (t, J=7.3 Hz, 2H), 2.28-2.18 (m, 1H), 2.14-2.04 (m, 1H), 1.01-0.86 (m, 5H), 0.71-0.60 (m, 2H).

### Example 114: 4-(ethyl(6-(N-(6-(o-tolyl)-5-(trifluoromethyl)pyridin-2-yl)sulfamoyl)pyridin-2-yl)amino)butanoic Acid (114)

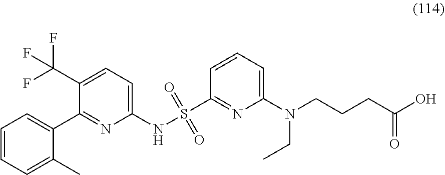 [SMILES: CCN(CCCC(=O)O)c1cccc(S(=O)(=O)Nc2ccc(C(F)(F)F)c(-c3ccccc3C)n2)n1]

4-(ethyl(6-(N-(6-(o-tolyl)-5-(trifluoromethyl)pyridin-2-yl)sulfamoyl)pyridin-2-yl)amino)butanoic acid (114) was synthesized using the procedure described in Example 87, except in step 1, 4-(butylamino)butan-1-ol was replaced with 4-(ethylamino)butan-1-ol. LCMS (Condition 1): m/z 522.9 [M+H]⁺, 1.66 min. ¹H NMR (500 MHz, Methanol-d₄) δ 8.05 (d, J=8.9 Hz, 1H), 7.61-7.51 (m, 2H), 7.31-7.27 (m, 1H), 7.24-7.15 (m, 2H), 7.13 (d, J=7.1 Hz, 1H), 7.04 (d, J=7.6 Hz, 1H), 6.81 (d, J=8.6 Hz, 1H), 3.50-3.37 (m, 4H), 2.25 (t, J=7.2 Hz, 2H), 1.89 (s, 3H), 1.81-1.75 (m, 2H), 1.03 (t, J=7.0 Hz, 3H).

### Example 115: 4-(ethyl(6-(N-(6-(5-fluoro-2-methylphenyl)-5-(trifluoromethyl)pyridin-2-yl)sulfamoyl)pyridin-2-yl)amino)butanoic Acid (115)

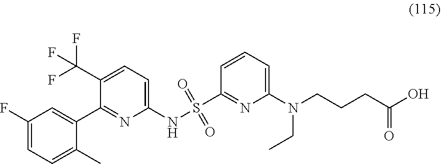 [SMILES: CCN(CCCC(=O)O)c1cccc(S(=O)(=O)Nc2ccc(C(F)(F)F)c(-c3cc(F)ccc3C)n2)n1]

4-(ethyl(6-(N-(6-(5-fluoro-2-methylphenyl)-5-(trifluoromethyl)pyridin-2-yl)sulfamoyl)pyridin-2-yl)amino)butanoic acid (115) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10) and 4-(butylamino)butan-1-ol was replaced with 4-(ethylamino)butan-1-ol. LCMS (Condition 1): m/z 540.9 [M+H]⁺, 1.68 min. ¹H NMR (500 MHz, Methanol-d₄) δ 8.06 (d, J=8.8 Hz, 1H), 7.57 (dd, J=8.7, 7.2 Hz, 1H), 7.52 (dd, J=8.8, 0.9 Hz, 1H), 7.20 (dd, J=8.5, 5.6 Hz, 1H), 7.12 (d, J=7.1 Hz, 1H), 7.05-7.01 (m, 1H), 6.81 (d, J=8.6 Hz, 1H), 6.78 (dd, J=9.2, 2.8 Hz, 1H), 3.50-3.37 (m, 4H), 2.24 (t, J=7.2 Hz, 2H), 1.83 (s, 3H), 1.80-1.74 (m, 2H), 1.03 (t, J=7.0 Hz, 3H).

### Example 116: 3-((cyclopropylmethyl)(6-(N-(6-(2-ethyl-5-fluorophenyl)-5-(trifluoromethyl)pyridin-2-yl)sulfamoyl)pyridin-2-yl)amino)propanoic Acid (116)

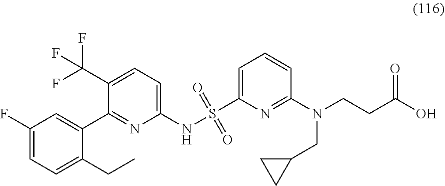 [SMILES: CCc1ccc(F)cc1-c1nc(NS(=O)(=O)c2cccc(N(CCC(=O)O)CC3CC3)n2)ccc1C(F)(F)F]

3-((cyclopropylmethyl)(6-(N-(6-(2-ethyl-5-fluorophenyl)-5-(trifluoromethyl)pyridin-2-yl)sulfamoyl)pyridin-2-yl)amino)propanoic acid (116) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with N-(6-(2-ethyl-5-fluorophenyl)-5-(trifluoromethyl)pyridin-2-yl)-6-fluoropyridine-2-sulfonamide (int-b3) and 4-methylpiperidine-4-carboxylic acid hydrochloride was replaced with ethyl 3-((cyclopropylmethyl)amino)propanoate. LCMS (Condition 1): m/z 566.9 [M+H]⁺, 1.77 min. ¹H NMR (400 MHz, Methanol-d₄) δ 8.06 (d, J=8.9 Hz, 1H), 7.64-7.53 (m, 2H), 7.25 (dd, J=8.6, 5.6 Hz, 1H), 7.17 (d, J=7.1 Hz, 1H), 7.11-7.02 (m, 1H), 6.87 (d, J=8.6 Hz, 1H), 6.77 (dd, J=9.2, 2.8 Hz, 1H), 3.83-3.64 (m, 2H), 3.42-3.33 (m, 1H), 2.51 (t, J=7.3 Hz, 2H), 2.30-2.17 (m, 1H), 2.16-2.03 (m, 1H), 1.34-1.28 (m, 3H), 1.02-0.94 (m, 2H), 0.50-0.41 (m, 2H), 0.29-0.21 (m, 2H).

### Example 117: 3-((6-(N-(6-(o-tolyl)-5-(trifluoromethyl)pyridin-2-yl)sulfamoyl)pyridin-2-yl)thio)propanoic Acid (117)

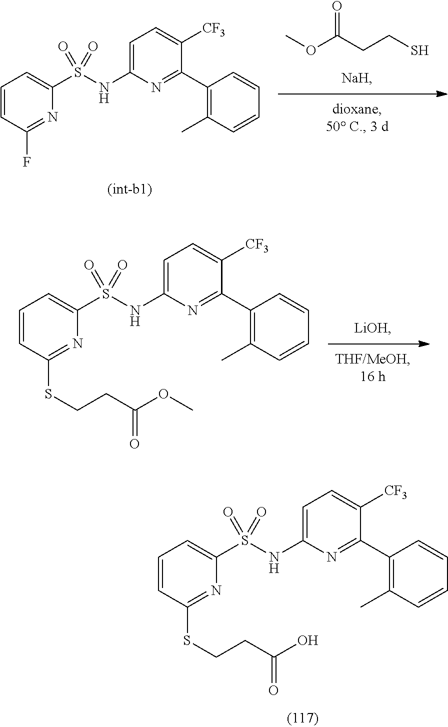

### Step 1. Synthesis of methyl 3-((6-(N-(6-(o-tolyl)-5-(trifluoromethyl)pyridin-2-yl)sulfamoyl)pyridin-2-yl)thio)propanoate

A mixture of 6-fluoro-N-(6-(o-tolyl)-5-(trifluoromethyl)pyridin-2-yl)pyridine-2-sulfonamide (int-b1) (15 mg, 0.036 mmol), sodium hydride (4.38 mg, 0.109 mmol) and methyl 3-mercaptopropanoate (5.9 μl, 0.055 mmol) was suspended in dioxane (1 mL), and subsequently heated to 50° C. for 3 days. The reaction was quenched with 1.0 M HCl and then extracted with DCM (×3). The organics were combined, evaporated to dryness, and purified by flash column chromatography (0-100% EtOAc/heptane) to give methyl 3-((6-(N-(6-(o-tolyl)-5-(trifluoromethyl)pyridin-2-yl)sulfamoyl)pyridin-2-yl)thio)propanoate. LCMS (Condition 1): m/z 512.0 [M+H]⁺, 1.57 min.

### Step 2. 3-((6-(N-(6-(o-tolyl)-5-(trifluoromethyl)pyridin-2-yl)sulfamoyl)pyridin-2-yl)thio)propanoic Acid

To a solution of methyl 3-((6-(N-(6-(o-tolyl)-5-(trifluoromethyl)pyridin-2-yl)sulfamoyl)pyridin-2-yl)thio)propanoate (15 mg, 0.029 mmol) in THE (29 μL), was added 2 M aqueous lithium hydroxide (147 μL, 0.293 mmol), and methanol until the solution became homogeneous. After stirring overnight, the reaction was quenched with conc. HCl dropwise, diluted with water, and extracted into EtOAc. The resulting organics were dried, filtered, concentrated, and purified by flash column chromatography (0-100% EtOAc/heptane) to give 3-((6-(N-(6-(o-tolyl)-5-(trifluoromethyl)pyridin-2-yl)sulfamoyl)pyridin-2-yl)thio)propanoic acid (117). LCMS (Condition 1): m/z 498.0 [M+H]⁺, 1.45 min. ¹H NMR (400 MHz, Chloroform-d) δ 11.00 (s, 2H), 8.04 (d, J=8.8 Hz, 1H), 7.71 (d, 1H), 7.49-7.40 (m, 2H), 7.29-7.24 (m, 1H), 7.22-7.20 (m, 1H), 7.16-7.10 (m, 2H), 6.96 (d, J=7.5 Hz, 1H), 3.58-3.34 (m, 2H), 2.54-2.45 (m, 2H), 1.67 (s, 3H).

### Example 118: 3-(cyclopropyl(6-(N-(6-(o-tolyl)-5-(trifluoromethyl)pyridin-2-yl)sulfamoyl)pyridin-2-yl)amino)propanoic Acid (118)

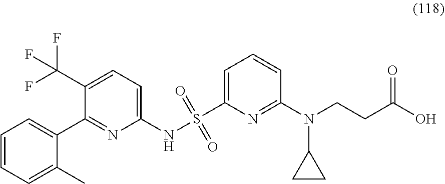 [SMILES: Cc1ccccc1-c1nc(NS(=O)(=O)c2cccc(N(CCC(=O)O)C3CC3)n2)ccc1C(F)(F)F]

3-(cyclopropyl(6-(N-(6-(o-tolyl)-5-(trifluoromethyl)pyridin-2-yl)sulfamoyl)pyridin-2-yl)amino)propanoic acid (118) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced with 6-fluoro-N-(6-(o-tolyl)-5-(trifluoromethyl)pyridin-2-yl)pyridine-2-sulfonamide (int-b1), 3-(ethylamino)propanoic acid hydrochloride was replaced with tert-butyl 3-(cyclopropylamino)propanoate, and D-α-tocopherol polyethylene glycol 1000 succinate (TPGS) and water (0.5 mL) were added to the reaction mixture. LCMS (Condition 1): m/z 520.9 [M+H]⁺, 1.70 min. ¹H NMR (400 MHz, Methanol-d₄) δ 8.05 (d, J=8.9 Hz, 1H), 7.66 (dd, J=8.6, 7.3 Hz, 1H), 7.55 (dd, J=8.9, 0.9 Hz, 1H), 7.34-7.13 (m, 5H), 7.04 (d, J=7.6 Hz, 1H), 3.80-3.74 (m, 2H), 2.60-2.55 (m, 1H), 2.40 (t, J=7.3 Hz, 2H), 1.87 (s, 3H), 1.02-0.87 (m, 2H), 0.74-0.60 (m, 2H). ¹⁹F NMR (376 MHz, Methanol-d₄) δ −59.78.

### Example 119: 3-((6-(N-(6-(2-ethyl-5-fluorophenyl)-5-(trifluoromethyl)pyridin-2-yl)sulfamoyl)pyridin-2-yl)(isopropyl)amino)propanoic Acid (119)

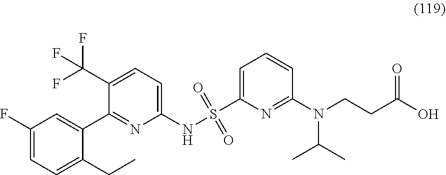 [SMILES: CCc1ccc(F)cc1-c1nc(NS(=O)(=O)c2cccc(N(CCC(=O)O)C(C)C)n2)ccc1C(F)(F)F]

3-((6-(N-(6-(2-ethyl-5-fluorophenyl)-5-(trifluoromethyl)pyridin-2-yl)sulfamoyl)pyridin-2-yl)(isopropyl)amino)propanoic acid (119) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(6-(2-ethyl-5-fluorophenyl)-5-(trifluoromethyl)pyridin-2-yl)-6-fluoropyridine-2-sulfonamide (int-b3), 4-(butylamino)butan-1-ol was replaced with 3-(isopropylamino)propan-1-ol, and heating in microwave at 200° C. for 1 hour was replaced with heating in an oil bath at 120° C. for 18 hours. LCMS (Condition 1): m/z 554.9 [M+H]⁺, 1.74 min. ¹H NMR (400 MHz, Methanol-d₄) δ 8.08-8.05 (m, 1H), 7.68-7.54 (m, 2H), 7.26 (dd, J=8.6, 5.6 Hz, 1H), 7.18 (dd, J=7.2, 1.2 Hz, 1H), 7.10-7.05 (m, 1H), 6.87-6.84 (m, 1H), 6.76 (dd, J=9.2, 2.8 Hz, 1H), 4.47-4.40 (m, 1H), 3.64-3.49 (m, 2H), 2.60-2.43 (m, 2H), 2.28-2.19 (m, 1H), 2.16-2.05 (m, 1H), 1.16-1.12 (m, 6H), 0.93 (t, J=7.6 Hz, 3H).

### Example 120: 4-((6-(N-(6-(2-ethylphenyl)-5-(trifluoromethyl)pyridin-2-yl)sulfamoyl)pyridin-2-yl)ₙxy)cyclohexane-1-carboxylic Acid (120)

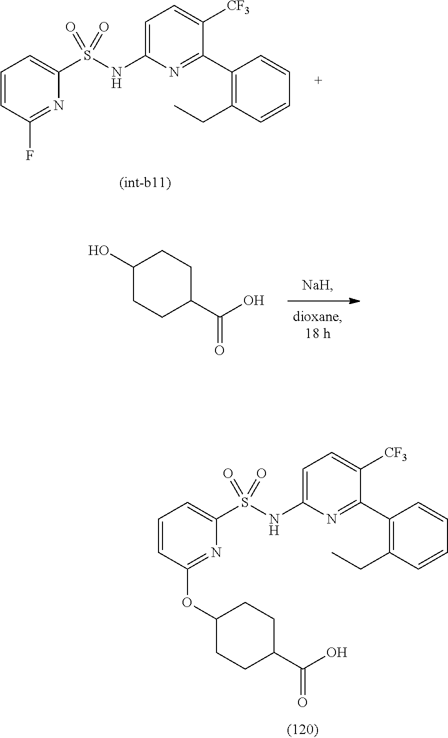 [SMILES: CCc1ccccc1-c1nc(NS(=O)(=O)c2cccc(F)n2)ccc1C(F)(F)F ; O=C(O)C1CCC(O)CC1 ; CCc1ccccc1-c1nc(NS(=O)(=O)c2cccc(OC3CCC(C(=O)O)CC3)n2)ccc1C(F)(F)F]

In a vial, 4-hydroxycyclohexanecarboxylic acid (42.4 mg, 0.294 mmol) was dissolved in dioxane (3 mL) and treated with sodium hydride (23.5 mg, 0.588 mmol). The resulting mixture was treated with N-(6-(2-ethylphenyl)-5-(trifluoromethyl)pyridin-2-yl)-6-fluoropyridine-2-sulfonamide (int-b11) (50.0 mg, 0.118 mmol) and stirred for 18 hr. The mixture was diluted with water and pH adjusted to 3-4 with 1 M aq. HCl and then extracted with EtOAc. The organic layer was isolated, dried over Na₂SO₄, filtered and concentrated. The crude product was purified by silica gel chromatography (4 g silica gel column, 0-40% EtOAc/EtOH (3:1) mixture in heptane) to give 4-((6-(N-(6-(2-ethylphenyl)-5-(trifluoromethyl)pyridin-2-yl)sulfamoyl)pyridin-2-yl)ₙxy)cyclohexane-1-carboxylic acid (120) (˜3:1 ratio of isomers). LCMS (Condition 1): m/z=550.1 [M+H]⁺, 1.63 min. Mixture of geometric isomers: ¹H NMR (400 MHz, Chloroform-d) δ 8.01 (d, J=8.9 Hz, 1H), 7.81-7.69 (m, 2H), 7.69-7.56 (m, 1H), 7.46-7.36 (m, 1H), 7.36-7.30 (m, 1H), 7.23 (d, J=7.4 Hz, 1H), 7.08 (d, J=7.6 Hz, 1H), 6.93 (d, J=8.3 Hz, 1H), 6.88 (d, J=8.3 Hz, 0H), 4.96 (s, 1H), 4.85 (m, 1H), 3.92 (s, 1H), 2.55-2.20 (m, 4H), 2.14-1.80 (m, 7H), 1.79-1.67 (m, 7H), 1.05 (m, 3H). ¹⁹F NMR (376 MHz, Chloroform-d) δ −58.31 (s, 3F), −58.45 (s, 1F).

### Example 121: 3-((6-(N-(6-(o-tolyl)-5-(trifluoromethyl)pyridin-2-yl)sulfamoyl)pyridin-2-yl)ₙxy)propanoic Acid (121)

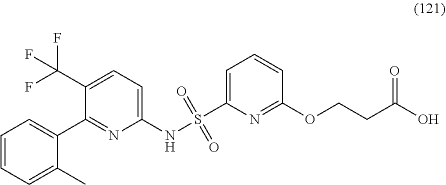 [SMILES: Cc1ccccc1-c1nc(NS(=O)(=O)c2cccc(OCCC(=O)O)n2)ccc1C(F)(F)F]

3-((6-(N-(6-(o-tolyl)-5-(trifluoromethyl)pyridin-2-yl)sulfamoyl)pyridin-2-yl)ₙxy)propanoic acid (121) was synthesized using the procedure described in Example 87, except in step 1, 4-(butylamino)butan-1-ol and NMP were replaced with 1,3-propanediol as solvent and N,N-diisopropylethylamine was replaced with K₂CO₃. LCMS (Condition 1): m/z 482.0 [M+H]⁺, 1.47 min. ¹H NMR (400 MHz, DMSO-d₆) δ 12.38 (s, 1H), 11.85 (s, 1H), 8.18 (d, J=8.7 Hz, 1H), 7.91-7.85 (m, 1H), 7.54 (d, J=7.3 Hz, 1H), 7.50-7.43 (m, 1H), 7.35-7.29 (m, 1H), 7.25-7.18 (m, 2H), 7.09-7.03 (m, 2H), 4.25 (t, J=6.2 Hz, 2H), 2.59 (t, J=6.2 Hz, 2H), 1.76 (s, 3H).

### Example 122: (S)-2-(3-methyl-1-(6-(N-(6-(o-tolyl)-5-(trifluoromethyl)pyridin-2-yl)sulfamoyl)pyridin-2-yl)piperidin-3-yl)acetic Acid (122)

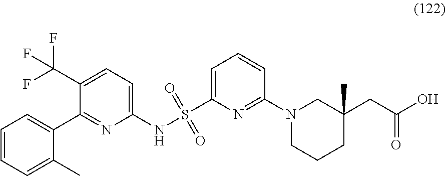 [SMILES: Cc1ccccc1-c1nc(NS(=O)(=O)c2cccc(N3CCC[C@@](C)(CC(=O)O)C3)n2)ccc1C(F)(F)F]

(S)-2-(3-methyl-1-(6-(N-(6-(o-tolyl)-5-(trifluoromethyl)pyridin-2-yl)sulfamoyl)pyridin-2-yl)piperidin-3-yl)acetic acid (122) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). Single enantiomer of unknown absolute stereochemistry obtained from chiral supercritical fluid chromatography. LCMS (Condition 1): m/z 549.0 [M+H]⁺, 1.55 min. ¹H NMR (400 MHz, DMSO-d₆) δ 12.04 (s, 1H), 11.53 (s, 1H), 8.19 (d, J=8.4 Hz, 1H), 7.67-7.61 (m, 1H), 7.53 (s, 1H), 7.31 (t, J=7.3 Hz, 1H), 7.21 (dd, J=14.9, 7.4 Hz, 2H), 7.08-7.00 (m, 3H), 3.44 (s, 2H), 3.38-3.34 (m, 1H), 3.29-3.17 (m, 1H), 2.56-2.52 (m, 1H), 2.08 (s, 1H), 2.02-1.93 (m, 1H), 1.78 (s, 3H), 1.64-1.57 (m, 1H), 1.44-1.34 (m, 2H), 0.88-0.81 (m, 3H).

### Example 123: (R)-2-(3-methyl-1-(6-(N-(6-(o-tolyl)-5-(trifluoromethyl)pyridin-2-yl)sulfamoyl)pyridin-2-yl)piperidin-3-yl)acetic Acid (123)

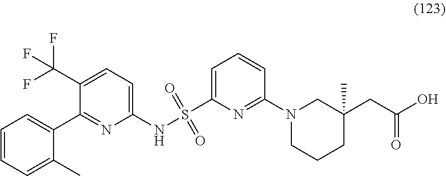 [SMILES: Cc1ccccc1-c1nc(NS(=O)(=O)c2cccc(N3CCC[C@](C)(CC(=O)O)C3)n2)ccc1C(F)(F)F]

(R)-2-(3-methyl-1-(6-(N-(6-(o-tolyl)-5-(trifluoromethyl)pyridin-2-yl)sulfamoyl)pyridin-2-yl)piperidin-3-yl)acetic acid (123) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with crude Boc-deprotected (using TFA) 2-(1-(tert-butoxycarbonyl)-3-methylpiperidin-3-yl)acetic acid (int-a5). Single enantiomer of unknown absolute stereochemistry obtained from chiral supercritical fluid chromatography. LCMS (Condition 1): m/z 549.2 [M+H]⁺, 1.55 min. ¹H NMR (400 MHz, DMSO-d₆) δ 12.06 (s, 1H), 11.54 (s, 1H), 8.19 (s, 1H), 7.68-7.60 (m, 1H), 7.54 (s, 1H), 7.32 (t, J=7.1 Hz, 1H), 7.21 (dd, J=14.8, 7.3 Hz, 2H), 7.11-6.97 (m, 3H), 3.51-3.39 (m, 2H), 3.38-3.25 (m, 1H), 3.24-3.12 (m, 1H), 2.57-2.52 (m, 1H), 2.14-2.05 (m, 1H), 2.03-1.93 (m, 1H), 1.79 (s, 3H), 1.66-1.57 (m, 1H), 1.45-1.35 (m, 2H), 0.91-0.79 (m, 3H).

### Example 124: (S)-6-(2-(1H-tetrazol-5-yl)pyrrolidin-1-yl)-N-(5-chloro-6-(2,6-dimethylphenyl)pyridin-2-yl)pyridine-2-sulfonamide (124)

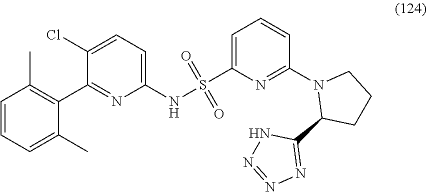 [SMILES: Cc1cccc(C)c1-c1nc(NS(=O)(=O)c2cccc(N3CCC[C@H]3c3nnn[nH]3)n2)ccc1Cl]

(S)-6-(2-(1H-tetrazol-5-yl)pyrrolidin-1-yl)-N-(5-chloro-6-(2,6-dimethylphenyl)pyridin-2-yl)pyridine-2-sulfonamide (124) was synthesized using the procedure described in Example 6, except 6-fluoro-N-(6-(5-fluoro-2-methylphenyl)-5-(trifluoromethyl)pyridin-2-yl)pyridine-2-sulfonamide (int-b10) was replaced with N-(5-chloro-6-(2,6-dimethylphenyl)pyridin-2-yl)-6-fluoropyridine-2-sulfonamide (int-b6), 2-(4-methylpiperidin-4-yl)acetic acid hydrochloride was replaced with (S)-5-(pyrrolidin-2-yl)-1H-tetrazole, and NMP was replaced with DMA. LCMS (Condition 1): m/z 511.0 [M+H]⁺, 1.67 min. ¹H NMR (400 MHz, DMSO-d₆) δ 11.12 (s, 1H), 7.89-7.88 (m, 1H), 7.71-7.70 (m, 1H), 7.31-7.14 (m, 3H), 7.08 (dd, J=7.9, 4.4 Hz, 2H), 6.78-6.63 (m, 1H), 5.31 (dd, J=8.3, 1.8 Hz, 1H), 3.97 (s, 1H), 3.66-3.64 (m, 1H), 3.35-3.33 (m, 1H), 2.38-2.23 (m, 1H), 2.13-1.90 (m, 3H), 1.79 (app d, J=6.6 Hz, 6H).

### Example 125: 4-(cyclopropyl(6-(N-(6-(o-tolyl)-5-(trifluoromethyl)pyridin-2-yl)sulfamoyl)pyridin-2-yl)amino)butanoic Acid (125)

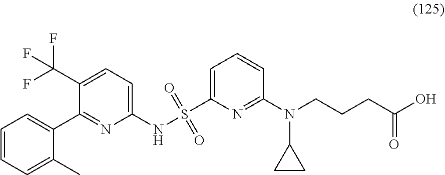 [SMILES: Cc1ccccc1-c1nc(NS(=O)(=O)c2cccc(N(CCCC(=O)O)C3CC3)n2)ccc1C(F)(F)F]

4-(cyclopropyl(6-(N-(6-(o-tolyl)-5-(trifluoromethyl)pyridin-2-yl)sulfamoyl)pyridin-2-yl)amino)butanoic acid (125) was synthesized using the procedure described in Example 87, except in step 1, 4-(butylamino)butan-1-ol was replaced with 4-(cyclopropylamino)butan-1-ol, and heating in microwave at 200° C. for 1 hour was replaced with heating in an oil bath at 120° C. for 5 days. LCMS (Condition 1): m/z 534.9 [M+H]⁺, 1.76 min. ¹H NMR (400 MHz, Methanol-d₄) δ 8.04 (d, J=8.9 Hz, 1H), 7.63 (dd, J=8.6, 7.3 Hz, 1H), 7.48 (dd, J=8.9, 0.9 Hz, 1H), 7.31-7.27 (m, 1H), 7.25-7.13 (m, 4H), 7.03 (d, J=7.6 Hz, 1H), 3.58-3.51 (m, 2H), 2.58-2.53 (m, 1H), 2.11 (t, J=7.4 Hz, 2H), 1.86 (s, 3H), 1.75-1.68 (m, 2H), 0.99-0.87 (m, 2H), 0.65-0.61 (m, 2H).

### Example 126: 3-((6-(N-(6-(5-fluoro-2-methylphenyl)-5-(trifluoromethyl)pyridin-2-yl)sulfamoyl)pyridin-2-yl)(isobutyl)amino)propanoic Acid (126)

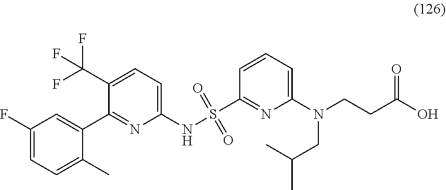 [SMILES: Cc1ccc(F)cc1-c1nc(NS(=O)(=O)c2cccc(N(CCC(=O)O)CC(C)C)n2)ccc1C(F)(F)F]

3-((6-(N-(6-(5-fluoro-2-methylphenyl)-5-(trifluoromethyl)pyridin-2-yl)sulfamoyl)pyridin-2-yl)(isobutyl)amino)propanoic acid (126) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 3-(isobutylamino)propan-1-ol, and heating in microwave at 200° C. for 1 hour was replaced with heating in an oil bath at 120° C. for 18 hours. LCMS (Condition 1): m/z 555.1 [M+H]⁺, 1.44 min. ¹H NMR (400 MHz, DMSO-d₆) δ 12.25 (s, 1H), 11.64 (s, 1H), 8.16 (d, J=8.8 Hz, 1H), 7.63 (dd, J=8.7, 7.2 Hz, 1H), 7.42 (d, J=8.7 Hz, 1H), 7.24 (dd, J=8.6, 5.7 Hz, 1H), 7.19-7.11 (m, 1H), 7.07 (d, J=7.2 Hz, 1H), 6.87 (t, J=7.2 Hz, 2H), 3.60-3.45 (m, 1H), 3.23-3.15 (dd, J=25.6, 7.5 Hz, 1H), 2.37-2.26 (m, 2H), 1.91-1.70 (m, 1H), 1.66 (s, 3H), 1.33-1.14 (m, 2H), 0.79-0.67 (m, 6H).

### Example 127: 4-((6-(N-(6-(5-fluoro-2-methylphenyl)-5-(trifluoromethyl)pyridin-2-yl)sulfamoyl)pyridin-2-yl)(isobutyl)amino)butanoic Acid (127)

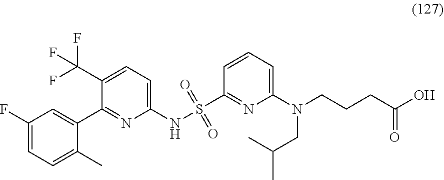 [SMILES: Cc1ccc(F)cc1-c1nc(NS(=O)(=O)c2cccc(N(CCCC(=O)O)CC(C)C)n2)ccc1C(F)(F)F]

4-((6-(N-(6-(5-fluoro-2-methylphenyl)-5-(trifluoromethyl)pyridin-2-yl)sulfamoyl)pyridin-2-yl)(isobutyl)amino)butanoic acid (127) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 4-(isobutylamino)butan-1-ol (int-a6), and heating in microwave at 200° C. for 1 hour was replaced with heating in an oil bath at 120° C. for 18 hours. LCMS (Condition 1): m/z 569.2 [M+H]⁺, 1.89 min. ¹H NMR (400 MHz, DMSO-d₆) δ 12.11 (s, 1H), 11.59 (s, 1H), 8.18 (d, J=8.9 Hz, 1H), 7.62 (dd, J=8.7, 7.2 Hz, 1H), 7.40 (d, J=8.8 Hz, 1H), 7.23 (dd, J=8.6, 5.7 Hz, 1H), 7.19-7.11 (m, 1H), 7.03 (d, J=7.3 Hz, 1H), 6.93-6.73 (m, 2H), 3.28-3.02 (m, 2H), 2.17 (t, J=7.2 Hz, 2H), 1.86-1.72 (m, 1H), 1.63 (s, 3H), 1.57 (s, 2H), 1.33-1.16 (m, 2H), 0.69 (d, J=5.0 Hz, 6H).

### Example 128: 3-(isobutyl(6-(N-(6-(o-tolyl)-5-(trifluoromethyl)pyridin-2-yl)sulfamoyl)pyridin-2-yl)amino)propanoic Acid (128)

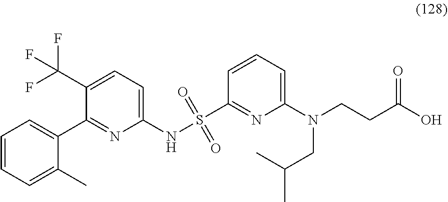 [SMILES: Cc1ccccc1-c1nc(NS(=O)(=O)c2cccc(N(CCC(=O)O)CC(C)C)n2)ccc1C(F)(F)F]

3-(isobutyl(6-(N-(6-(o-tolyl)-5-(trifluoromethyl)pyridin-2-yl)sulfamoyl)pyridin-2-yl)amino)propanoic acid (128) was synthesized using the procedure described in Example 87, except in step 1, 4-(butylamino)butan-1-ol was replaced with 3-(isobutylamino)propan-1-ol, and heating in microwave at 200° C. for 1 hour was replaced with heating in an oil bath at 120° C. for 18 hours. LCMS (Condition 1): m/z 536.9 [M+H]⁺, 1.73 min. ¹H NMR (400 MHz, Methanol-d₄) δ 8.05 (d, J=8.9 Hz, 1H), 7.63-7.55 (m, 1H), 7.52 (dd, J=8.9, 0.9 Hz, 1H), 7.32-7.24 (m, 1H), 7.22-7.11 (m, 3H), 7.04 (d, J=7.5 Hz, 1H), 6.80 (d, J=8.7 Hz, 1H), 3.77-3.57 (m, 2H), 3.26 (t, J=6.9 Hz, 2H), 2.46 (t, J=7.1 Hz, 2H), 2.02-1.90 (m, 1H), 1.87 (s, 3H), 0.85 (d, J=6.7 Hz, 6H).

### Example 129: 4-(isobutyl(6-(N-(6-(o-tolyl)-5-(trifluoromethyl)pyridin-2-yl)sulfamoyl)pyridin-2-yl)amino)butanoic Acid (129)

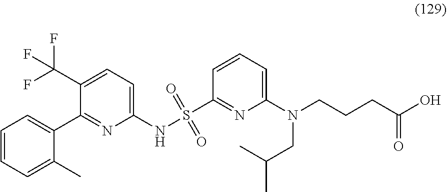 [SMILES: Cc1ccccc1-c1nc(NS(=O)(=O)c2cccc(N(CCCC(=O)O)CC(C)C)n2)ccc1C(F)(F)F]

4-(isobutyl(6-(N-(6-(o-tolyl)-5-(trifluoromethyl)pyridin-2-yl)sulfamoyl)pyridin-2-yl)amino)butanoic acid (129) was synthesized using the procedure described in Example 87, except in step 1, 4-(butylamino)butan-1-ol was replaced with 4-(isobutylamino)butan-1-ol (int-a6), and heating in microwave at 200° C. for 1 hour was replaced with heating in an oil bath at 120° C. for 18 hours. LCMS (Condition 1): m/z 551.0 [M+H]⁺, 1.82 min. ¹H NMR (400 MHz, DMSO-d₆) δ 12.12 (s, 1H), 11.53 (s, 1H), 8.16 (d, J=8.9 Hz, 1H), 7.63 (dd, J=8.8, 7.3 Hz, 1H), 7.41 (d, J=8.8 Hz, 1H), 7.32-7.25 (m, 1H), 7.23-7.11 (m, 2H), 7.02 (dd, J=11.4, 7.4 Hz, 2H), 6.87 (d, J=8.7 Hz, 1H), 3.27-3.02 (m, 2H), 2.18 (t, J=7.2 Hz, 2H), 1.77 (dd, J=13.9, 7.1 Hz, 1H), 1.69 (s, 3H), 1.64-1.49 (m, 2H), 1.33-1.18 (m, 2H), 0.69 (dd, J=6.4, 2.8 Hz, 6H).

### Example 130: 1-((methyl(6-(N-(6-(o-tolyl)-5-(trifluoromethyl)pyridin-2-yl)sulfamoyl)pyridin-2-yl)amino)methyl)cyclopentane-1-carboxylic Acid (130)

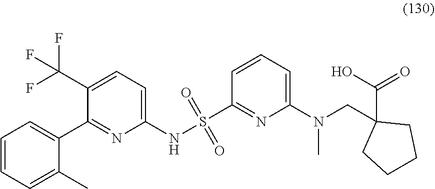 [SMILES: Cc1ccccc1-c1nc(NS(=O)(=O)c2cccc(N(C)CC3(C(=O)O)CCCC3)n2)ccc1C(F)(F)F]

1-((methyl(6-(N-(6-(o-tolyl)-5-(trifluoromethyl)pyridin-2-yl)sulfamoyl)pyridin-2-yl)amino)methyl)cyclopentane-1-carboxylic acid (130) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced 6-fluoro-N-(6-(o-tolyl)-5-(trifluoromethyl)pyridin-2-yl)pyridine-2-sulfonamide (int-b1) and 3-(ethylamino)propanoic acid hydrochloride was replaced with 1-((methylamino)methyl)cyclopentanecarboxylic acid hydrobromide (int-a7), and K₂CO₃ was replaced with N,N-diisopropylethylamine. LCMS (Condition 1): m/z 549.2 [M+H]⁺, 1.88 min. ¹H NMR (400 MHz, DMSO-d₆) δ 12.45 (s, 1H), 11.58 (s, 1H), 8.17 (d, J=8.9 Hz, 1H), 7.64 (dd, J=8.7, 7.3 Hz, 1H), 7.40 (d, J=8.7 Hz, 1H), 7.33-7.29 (m, 1H), 7.23-7.18 (m, 2H), 7.08 (d, J=7.2 Hz, 1H), 7.03 (d, J=7.4 Hz, 1H), 6.86 (d, J=8.7 Hz, 1H), 3.86 (d, J=13.9 Hz, 1H), 3.71 (d, J=14.2 Hz, 1H), 2.83 (s, 3H), 1.84-1.80 (m, 2H), 1.74 (s, 3H), 1.48-1.25 (m, 6H). ¹⁹F NMR (471 MHz, DMSO-d₆) δ −57.05 (s).

### Example 131: 4-((6-(N-(6-(5-fluoro-2-methylphenyl)-5-(trifluoromethyl)pyridin-2-yl)sulfamoyl)pyridin-2-yl)(propyl)amino)butanoic Acid (131)

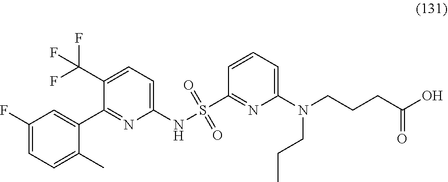 [SMILES: CCCN(CCCC(=O)O)c1cccc(S(=O)(=O)Nc2ccc(C(F)(F)F)c(-c3cc(F)ccc3C)n2)n1]

4-((6-(N-(6-(5-fluoro-2-methylphenyl)-5-(trifluoromethyl)pyridin-2-yl)sulfamoyl)pyridin-2-yl)(propyl)amino)butanoic acid (131) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 4-(propylamino)butan-1-ol (int-a8), and heating in microwave at 200° C. for 1 hour was replaced with heating in an oil bath at 130° C. for 3 days. LCMS (Condition 1): m/z 555.1 [M+H]⁺, 1.43 min. ¹H NMR (400 MHz, DMSO-d₆) δ 12.11 (s, 1H), 11.58 (s, 1H), 8.18 (d, J=8.9 Hz, 1H), 7.63 (dd, J=8.7, 7.2 Hz, 1H), 7.43 (d, J=8.8 Hz, 1H), 7.24 (dd, J=8.7, 5.7 Hz, 1H), 7.17-7.12 (m, 1H), 7.03 (d, J=7.3 Hz, 1H), 6.94-6.78 (m, 2H), 3.30-3.04 (m, 4H), 2.16 (t, J=7.3 Hz, 2H), 1.75-1.48 (m, 5H), 1.34-1.28 (m, 2H), 0.73 (t, J=7.3 Hz, 3H).

### Example 132: 4-(propyl(6-(N-(6-(o-tolyl)-5-(trifluoromethyl)pyridin-2-yl)sulfamoyl)pyridin-2-yl)amino)butanoic Acid (132)

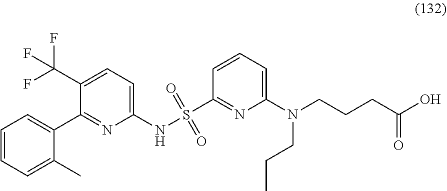 [SMILES: CCCN(CCCC(=O)O)c1cccc(S(=O)(=O)Nc2ccc(C(F)(F)F)c(-c3ccccc3C)n2)n1]

4-(propyl(6-(N-(6-(o-tolyl)-5-(trifluoromethyl)pyridin-2-yl)sulfamoyl)pyridin-2-yl)amino)butanoic acid (132) was synthesized using the procedure described in Example 87, except in step 1, 4-(butylamino)butan-1-ol was replaced with 4-(propylamino)butan-1-ol (int-a8) and heating in microwave at 200° C. for 1 hour was replaced with heating in an oil bath at 120° C. for 18 hours. LCMS (Condition 1): m/z 537.1 [M+H]⁺, 1.42 min. ¹H NMR (400 MHz, DMSO-d₆) δ 12.11 (s, 1H), 11.52 (s, 1H), 8.16 (d, J=8.9 Hz, 1H), 7.63 (dd, J=8.7, 7.2 Hz, 1H), 7.44 (d, J=8.9 Hz, 1H), 7.32-7.26 (m, 1H), 7.24-7.13 (m, 2H), 7.02 (dd, J=7.2, 5.1 Hz, 2H), 6.85 (d, J=8.6 Hz, 1H), 3.32-3.10 (m, 4H), 2.17 (t, J=7.3 Hz, 2H), 1.70 (s, 3H), 1.65-1.49 (m, 2H), 1.41-1.26 (m, 2H), 0.73 (t, J=7.4 Hz, 3H).

### Example 133: 4-((methyl(6-(N-(6-(o-tolyl)-5-(trifluoromethyl)pyridin-2-yl)sulfamoyl)pyridin-2-yl)amino)methyl)tetrahydro-2H-pyran-4-carboxylic Acid (133)

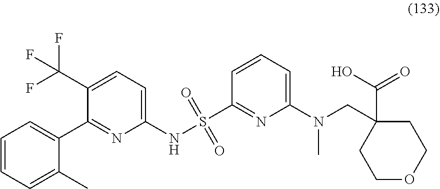 [SMILES: Cc1ccccc1-c1nc(NS(=O)(=O)c2cccc(N(C)CC3(C(=O)O)CCOCC3)n2)ccc1C(F)(F)F]

4-((methyl(6-(N-(6-(o-tolyl)-5-(trifluoromethyl)pyridin-2-yl)sulfamoyl)pyridin-2-yl)amino)methyl)tetrahydro-2H-pyran-4-carboxylic acid (133) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced 6-fluoro-N-(6-(o-tolyl)-5-(trifluoromethyl)pyridin-2-yl)pyridine-2-sulfonamide (int-b1), 3-(ethylamino)propanoic acid hydrochloride was replaced with 4-((methylamino)methyl)tetrahydro-2H-pyran-4-carboxylic acid hydrobromide (int-a9), and K₂CO₃ was replaced with N,N-diisopropylethylamine. LCMS (Condition 1): m/z 565.2 [M+H]⁺, 1.82 min. ¹H NMR (400 MHz, DMSO-d₆) δ 12.83 (s, 1H), 11.67 (s, 1H), 8.16 (d, J=8.8 Hz, 1H), 7.66 (dd, J=8.6, 7.4 Hz, 1H), 7.42 (d, J=8.8 Hz, 1H), 7.33-7.29 (m, 1H), 7.23-7.18 (m, 2H), 7.10 (d, J=7.2 Hz, 1H), 7.05 (d, J=7.5 Hz, 1H), 6.88 (d, J=8.7 Hz, 1H), 3.76 (d, J=14.2 Hz, 1H), 3.64-3.54 (m, 3H), 3.17 (t, J=11.4 Hz, 2H), 2.86 (s, 3H), 1.73-1.67 (m, 5H), 1.43-1.32 (m, 2H). ¹⁹F NMR (376 MHz, DMSO-d₆) δ −57.08 (s).

### Example 134: 4-((6-(N-(6-(o-tolyl)-5-(trifluoromethyl)pyridin-2-yl)sulfamoyl)pyridin-2-yl)(3,3,3-trifluoropropyl)amino)butanoic Acid (134)

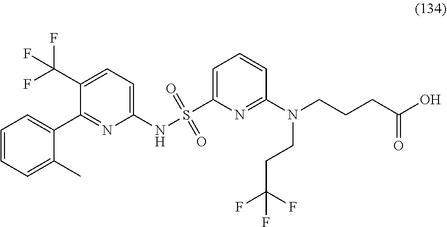 [SMILES: Cc1ccccc1-c1nc(NS(=O)(=O)c2cccc(N(CCCC(=O)O)CCC(F)(F)F)n2)ccc1C(F)(F)F]

4-((6-(N-(6-(o-tolyl)-5-(trifluoromethyl)pyridin-2-yl)sulfamoyl)pyridin-2-yl)(3,3,3-trifluoropropyl)amino)butanoic acid (134) was synthesized using the procedure described in Example 87, except in step 1, 4-(butylamino)butan-1-ol was replaced with crude 4-((3,3,3-trifluoropropyl)amino)butan-1-ol (int-a10) and heating in microwave at 200° C. for 1 hour was replaced with heating in an oil bath at 120° C. for 4 days. LCMS (Condition 1): m/z 590.9 [M+H]⁺, 1.91 min. ¹H NMR (400 MHz, Methanol-d₄) δ 8.03 (d, J=8.9 Hz, 1H), 7.62 (dd, J=8.7, 7.3 Hz, 1H), 7.49-7.40 (m, 1H), 7.31-7.27 (m, 1H), 7.23-7.12 (m, 3H), 7.01 (d, J=7.6 Hz, 1H), 6.95-6.85 (m, 1H), 3.69-3.64 (m, 2H), 3.47-3.42 (m, 2H), 2.44-2.24 (m, 4H), 1.85 (s, 3H), 1.83-1.77 (m, 2H).

### Example 135: 3-((6-(N-(6-(5-fluoro-2-methylphenyl)-5-(trifluoromethyl)pyridin-2-yl)sulfamoyl)pyridin-2-yl)(isopropyl)amino)propanoic Acid (135)

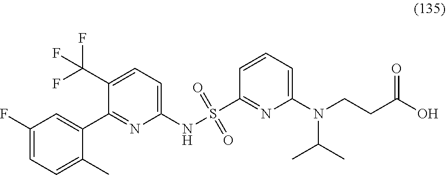 [SMILES: Cc1ccc(F)cc1-c1nc(NS(=O)(=O)c2cccc(N(CCC(=O)O)C(C)C)n2)ccc1C(F)(F)F]

3-((6-(N-(6-(5-fluoro-2-methylphenyl)-5-(trifluoromethyl)pyridin-2-yl)sulfamoyl)pyridin-2-yl)(isopropyl)amino)propanoic acid (135) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced 6-fluoro-N-(6-(5-fluoro-2-methylphenyl)-5-(trifluoromethyl)pyridin-2-yl)pyridine-2-sulfonamide (int-b10), 4-(butylamino)butan-1-ol was replaced with 3-(isopropylamino)propan-1-ol, and heating in microwave at 200° C. for 1 hour was replaced with heating in an oil bath at 120° C. for 6 days. LCMS (Condition 1): m/z 540.9 [M+H]⁺, 1.91 min. ¹H NMR (400 MHz, Methanol-d₄) δ 8.07 (d, J=8.9 Hz, 1H), 7.61 (dd, J=8.8, 7.3 Hz, 1H), 7.57-7.51 (m, 1H), 7.23-7.14 (m, 2H), 7.05-7.00 (m, 1H), 6.85 (d, J=8.7 Hz, 1H), 6.78 (dd, J=9.1, 2.8 Hz, 1H), 4.47-4.39 (m, 1H), 3.58-3.53 (m, 2H), 2.56-2.43 (m, 2H), 1.82 (s, 3H), 1.13 (d, J=6.7 Hz, 6H).

### Example 136: 2-(4-((6-(N-(5-chloro-6-(o-tolyl)pyridin-2-yl)sulfamoyl)pyridin-2-yl)amino)cyclohexane-1-carboxamido)butanoic Acid (136)

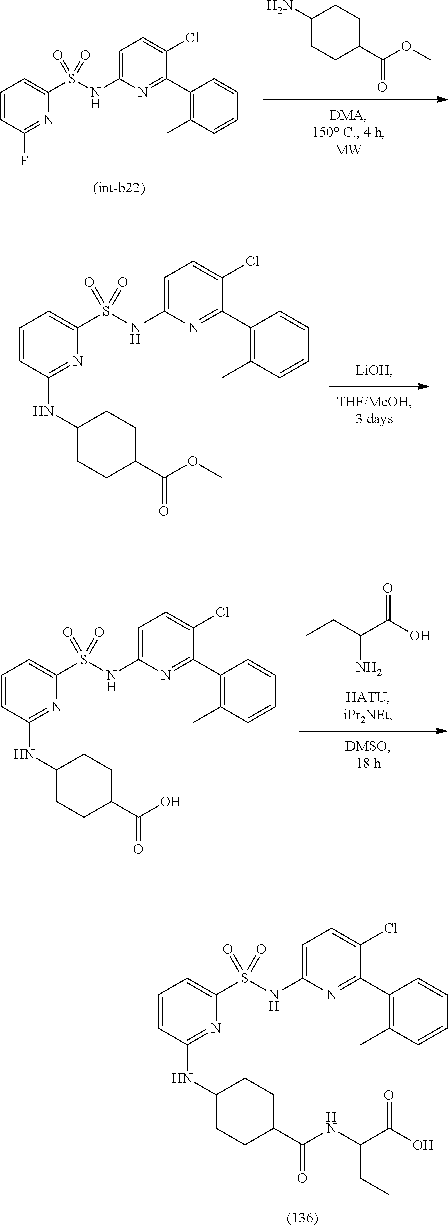

### Step 1. Synthesis of methyl 4-((6-(N-(5-chloro-6-(o-tolyl)pyridin-2-yl)sulfamoyl)pyridin-2-yl)amino)cyclohexanecarboxylate

In a microwave vial, N-(5-chloro-6-(o-tolyl)pyridin-2-yl)-6-fluoropyridine-2-sulfonamide (int-b22) (0.30 g 0.79 mmol) and methyl 4-aminocyclohexanecarboxylate hydrochloride (0.53 g, 2.7 mmol) were dissolved in DMA (11 mL), then N,N-diisopropylethylamine (2.1 mL, 12 mmol) was added, and the reaction was heated in the microwave at 150° C. for 2 hours. An additional portion of methyl 4-aminocyclohexanecarboxylate hydrochloride (0.53 g, 2.7 mmol) was added and the reaction was again heated in the microwave at 150° C. for 2 hours. The reaction was purified by preparatory HPLC to give the anticipated product methyl 4-((6-(N-(5-chloro-6-(o-tolyl)pyridin-2-yl)sulfamoyl)pyridin-2-yl)amino)cyclohexanecarboxylate. LCMS (Condition 1): m/z 515.2 [M+H]⁺, 1.92 min.

### Step 2. Synthesis of 4-((6-(N-(5-chloro-6-(o-tolyl)pyridin-2-yl)sulfamoyl)pyridin-2-yl)amino)cyclohexane-1-carboxylic Acid

In a flask, methyl 4-((6-(N-(5-chloro-6-(o-tolyl)pyridin-2-yl)sulfamoyl)pyridin-2-yl)amino)cyclohexanecarboxylate (240 mg, 0.47 mmol) was dissolved in a 3:2 mixture of THF:MeOH (2 mL), then LiOH (0.47 mL, 1.4 mmol, 3 M in water) was added, and the mixture was heated with a heat gun for 30 seconds (×2). A second and third portion of LiOH (0.47 mL, 1.4 mmol, 3 M in water) were added and the reaction was allowed to stir at room temperature for 3 days. The solvent was then removed and the crude product 4-((6-(N-(5-chloro-6-(o-tolyl)pyridin-2-yl)sulfamoyl)pyridin-2-yl)amino)cyclohexane-1-carboxylic acid was used directly in the next reaction without purification. LCMS (Condition 1): m/z 501.1 [M+H]⁺, 1.79 min.

### Step 3. Synthesis of 2-(4-((6-(N-(5-chloro-6-(o-tolyl)pyridin-2-yl)sulfamoyl)pyridin-2-yl)amino)cyclohexane-1-carboxamido)butanoic Acid (136)

In a vial crude 4-((6-(N-(5-chloro-6-(o-tolyl)pyridin-2-yl)sulfamoyl)pyridin-2-yl)amino)cyclohexane-1-carboxylic acid (26 mg, 0.052 mmol) was dissolved in DMSO (1 mL), then HATU (40 mg, 0.10 mmol), 2-aminobutanoic acid (21 mg, 0.21 mmol), and N,N-diisopropylethylamine (67 mg, 0.52 mmol) were subsequently added to the reaction mixture. After stirring overnight the reaction was purified by preparatory HPLC using ammonium acetate as a solvent modifier to provide the anticipated product 2-(4-((6-(N-(5-chloro-6-(o-tolyl)pyridin-2-yl)sulfamoyl)pyridin-2-yl)amino)cyclohexanecarboxamido)butanoic acid (136). LCMS (Condition 1): m/z 586.0 [M+H]⁺, 1.70 min. ¹H (400 MHz, DMSO-d₆) δ 11.01 (s, 1H), 7.95 (d, J=8.8 Hz, 1H), 7.52-7.51 (m, 1H), 7.44 (d, J=8.7 Hz, 1H), 7.35-7.15 (m, 4H), 7.03 (d, J=7.2 Hz, 2H), 6.98 (d, J=7.2 Hz, 1H), 6.76 (d, J=8.1 Hz, 1H), 6.69 (s, 1H), 3.62 (b s, 1H), 3.34 (s, 3H, obscured by solvent), 2.55-2.53 (m, 3H), 2.19-2.07 (m, 1H), 1.92 (s, 3H), 1.69 (q, J=9.8 Hz, 2H), 1.50 (s, 2H), 1.47-1.27 (m, 4H).

### Example 137: 1-((methyl(6-(N-(6-(o-tolyl)-5-(trifluoromethyl)pyridin-2-yl)sulfamoyl)pyridin-2-yl)amino)methyl)cyclobutane-1-carboxylic Acid (137)

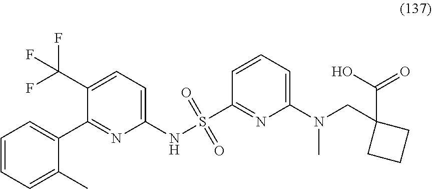 [SMILES: Cc1ccccc1-c1nc(NS(=O)(=O)c2cccc(N(C)CC3(C(=O)O)CCC3)n2)ccc1C(F)(F)F]

1-((methyl(6-(N-(6-(o-tolyl)-5-(trifluoromethyl)pyridin-2-yl)sulfamoyl)pyridin-2-yl)amino)methyl)cyclobutane-1-carboxylic acid (137) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced 6-fluoro-N-(6-(o-tolyl)-5-(trifluoromethyl)pyridin-2-yl)pyridine-2-sulfonamide (int-b1), 3-(ethylamino)propanoic acid hydrochloride was replaced with 1-((methylamino)methyl)cyclobutane-1-carboxylic acid hydrobromide (int-a2), and K₂CO₃ was replaced with N,N-diisopropylethylamine. LCMS (Condition 1): m/z 535.1 [M+H]⁺, 1.87 min. ¹H NMR (400 MHz, DMSO-d₆) δ 12.48 (s, 1H), 11.61 (s, 1H), 8.17 (d, J=8.9 Hz, 1H), 7.65 (dd, J=8.7, 7.3 Hz, 1H), 7.44 (d, J=8.8 Hz, 1H), 7.33-7.29 (m, 1H), 7.23-7.18 (m, 2H), 7.10 (d, J=7.2 Hz, 1H), 7.02 (d, J=7.5 Hz, 1H), 6.88 (d, J=8.7 Hz, 1H), 3.91 (d, J=14.3 Hz, 1H), 3.81 (d, J=14.1 Hz, 1H), 2.82 (s, 3H), 2.14-2.11 (m, 2H), 1.87-1.76 (m, 6H), 1.67-1.60 (m, 1H). ¹⁹F NMR (376 MHz, DMSO-d₆) δ −57.07 (s).

### Example 138: 4-(((6-(N-(6-(5-fluoro-2-methylphenyl)-5-(trifluoromethyl)pyridin-2-yl)sulfamoyl)pyridin-2-yl)(methyl)amino)methyl)tetrahydro-2H-pyran-4-carboxylic Acid (138)

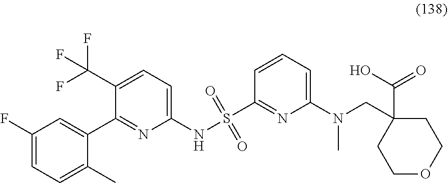 [SMILES: Cc1ccc(F)cc1-c1nc(NS(=O)(=O)c2cccc(N(C)CC3(C(=O)O)CCOCC3)n2)ccc1C(F)(F)F]

4-(((6-(N-(6-(5-fluoro-2-methylphenyl)-5-(trifluoromethyl)pyridin-2-yl)sulfamoyl)pyridin-2-yl)(methyl)amino)methyl)tetrahydro-2H-pyran-4-carboxylic acid (138) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced 6-fluoro-N-(6-(5-fluoro-2-methylphenyl)-5-(trifluoromethyl)pyridin-2-yl)pyridine-2-sulfonamide (int-b10), 3-(ethylamino)propanoic acid hydrochloride was replaced with 4-((methylamino)methyl)tetrahydro-2H-pyran-4-carboxylic acid hydrobromide (int-a9), and K₂CO₃ was replaced with N,N-diisopropylethylamine. LCMS (Condition 1): m/z 583.1 [M+H]⁺, 1.82 min. ¹H NMR (400 MHz, DMSO-d₆) δ 12.82 (s, 1H), 11.72 (s, 1H), 8.18 (d, J=8.8 Hz, 1H), 7.66 (dd, J=8.6, 7.4 Hz, 1H), 7.43 (d, J=8.7 Hz, 1H), 7.26 (dd, J=8.5, 5.8 Hz, 1H), 7.19-7.14 (m, 1H), 7.11 (d, J=7.2 Hz, 1H), 6.95 (dd, J=9.3, 2.4 Hz, 1H), 6.88 (d, J=8.7 Hz, 1H), 3.76 (d, J=14.4 Hz, 1H), 3.65-3.52 (m, 3H), 3.17 (t, J=11.5 Hz, 2H), 2.86 (s, 3H), 1.74-1.66 (m, 5H), 1.42-1.25 (m, 2H). ¹⁹F NMR (376 MHz, DMSO-d₆) δ −57.20 (s, 3F), −118.33 (s, 1F).

### Example 139: 1-(((6-(N-(6-(5-fluoro-2-methylphenyl)-5-(trifluoromethyl)pyridin-2-yl)sulfamoyl)pyridin-2-yl)(methyl)amino)methyl)cyclobutane-1-carboxylic Acid (139)

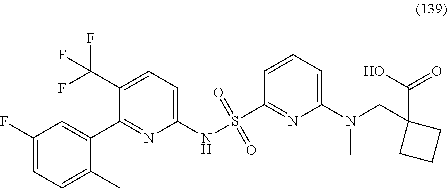 [SMILES: Cc1ccc(F)cc1-c1nc(NS(=O)(=O)c2cccc(N(C)CC3(C(=O)O)CCC3)n2)ccc1C(F)(F)F]

1-(((6-(N-(6-(5-fluoro-2-methylphenyl)-5-(trifluoromethyl)pyridin-2-yl)sulfamoyl)pyridin-2-yl)(methyl)amino)methyl)cyclobutane-1-carboxylic acid (139) was synthesized using the procedure described in Example 70, except 6-fluoro-N-(6-(2-isopropylphenyl)-5-(trifluoromethyl)pyridin-2-yl)pyridine-2-sulfonamide (int-b2) was replaced 6-fluoro-N-(6-(5-fluoro-2-methylphenyl)-5-(trifluoromethyl)pyridin-2-yl)pyridine-2-sulfonamide (int-b10), 3-(ethylamino)propanoic acid hydrochloride was replaced with 1-((methylamino)methyl)cyclobutane-1-carboxylic acid hydrobromide (int-a2), and K₂CO₃ was replaced with N,N-diisopropylethylamine. LCMS (Condition 1): m/z 553.2 [M+H]⁺, 1.86 min. ¹H NMR (400 MHz, DMSO-d₆) δ 12.44 (s, 1H), 11.65 (s, 1H), 8.18 (d, J=8.9 Hz, 1H), 7.65 (dd, J=8.7, 7.3 Hz, 1H), 7.44 (d, J=8.8 Hz, 1H), 7.26 (dd, J=8.5, 5.8 Hz, 1H), 7.19-7.14 (m, 1H), 7.10 (d, J=7.2 Hz, 1H), 6.91-6.87 (m, 2H), 3.91 (d, J=14.1 Hz, 1H), 3.81 (d, J=14.2 Hz, 1H), 2.82 (s, 3H), 2.14-2.08 (m, 2H), 1.83-1.80 (m, 3H), 1.71 (s, 3H), 1.68-1.63 (m, 1H). ¹⁹F NMR (376 MHz, DMSO-d₆) δ −57.18 (s, 3F), −118.34 (s, 1F).

### Example 140: 3-((4-(N-(5-chloro-6-(2,6-dimethylphenyl)pyridin-2-yl)sulfamoyl)pyridin-2-yl)(ethyl)amino)propanoic Acid (140)

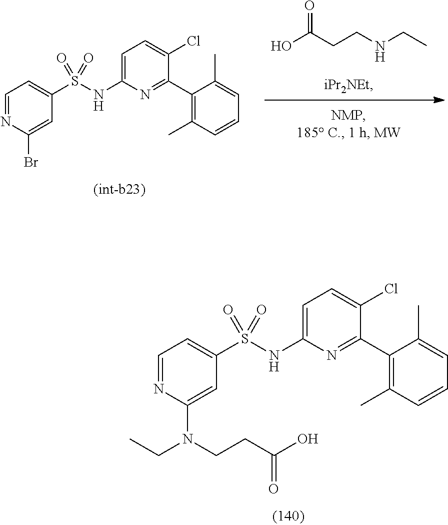

To a microwave vial was added 2-bromo-N-(5-chloro-6-(2,6-dimethylphenyl)pyridin-2-yl)pyridine-4-sulfonamide (int-b23) (60 mg, 0.13 mmol), 3-(ethylamino)propanoic acid (150 mg, 1.3 mmol), N,N-diisopropylethylamine (100 mg, 0.77 mmol), and NMP (1.5 mL). The mixture was heated to 185° C. for 1 h in the microwave. The reaction was cooled, diluted with MeOH, and directly purified by mass-triggered preparatory HPLC (25-70% MeCN/H2O+0.05% TFA, 100 mL/min). The fractions were lyophilized to dryness, resuspended in MeOH, and treated with 0.1 mL of 1 M aqueous HCl. The material was concentrated in vacuo to give 3-((4-(N-(5-chloro-6-(2,6-dimethylphenyl)pyridin-2-yl)sulfamoyl)pyridin-2-yl)(ethyl)amino)propanoic acid (140). LCMS (Condition 1): m/z 489.0 [M+H]⁺, 1.48 min. ¹H NMR (400 MHz, DMSO-d₆) δ 12.51 (br s, 1H), 11.42 (s, 1H), 8.20 (dd, J=5.1, 3.7 Hz, 1H), 7.97 (d, J=8.5 Hz, 1H), 7.31-6.99 (m, 4H), 6.80 (d, J=4.7 Hz, 2H), 3.56 (t, J=7.0 Hz, 2H), 3.29 (q, J=6.9 Hz, 2H), 2.44 (t, J=7.2 Hz, 2H), 1.72 (d, J=3.7 Hz, 6H), 0.96 (t, J=6.8 Hz, 3H).

### Example 141: (3-((4-(N-(5-chloro-6-(2,6-dimethylphenyl)pyridin-2-yl)sulfamoyl)pyridin-2-yl)amino)pentanoic Acid (141)

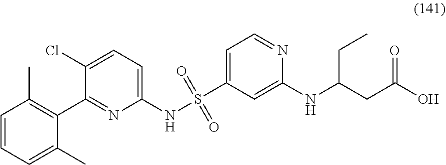 [SMILES: CCC(CC(=O)O)Nc1cc(S(=O)(=O)Nc2ccc(Cl)c(-c3c(C)cccc3C)n2)ccn1]

(3-((4-(N-(5-chloro-6-(2,6-dimethylphenyl)pyridin-2-yl)sulfamoyl)pyridin-2-yl)amino)pentanoic acid (141) was synthesized using the procedure described in Example 140, except 3-(ethylamino)propanoic acid was replaced with 3-aminopentanoic acid. LCMS (Condition 1): m/z 489.0 [M+H]⁺, 1.58 min. ¹H NMR NMR (400 MHz, DMSO-d₆) δ 12.30 (br s, 1H), 11.21 (br s, 1H), 8.30 (d, J=5.0 Hz, 1H), 7.78 (s, 1H), 7.58-7.42 (m, 2H), 7.16 (dd, J=8.3, 6.7 Hz, 1H), 7.06 (d, J=7.9 Hz, 2H), 6.61 (d, J=8.7 Hz, 1H), 3.41-3.03 (m, 2H), 2.55 (d, J=5.3 Hz, 1H), 2.43-2.39 (m, 1H), 1.77 (s, 6H), 1.66-1.47 (m, 2H), 0.90 (t, J=7.5 Hz, 3H).

### Example 142: 3-((4-(N-(5-chloro-6-(2,6-dimethylphenyl)pyridin-2-yl)sulfamoyl)pyridin-2-yl)amino)butanoic Acid (142)

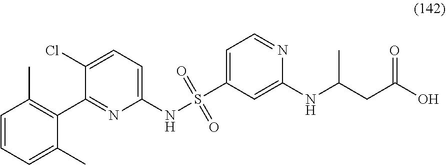 [SMILES: Cc1cccc(C)c1-c1nc(NS(=O)(=O)c2ccnc(NC(C)CC(=O)O)c2)ccc1Cl]

3-((4-(N-(5-chloro-6-(2,6-dimethylphenyl)pyridin-2-yl)sulfamoyl)pyridin-2-yl)amino)butanoic acid (142) was synthesized using the procedure described in Example 140, except 3-(ethylamino)propanoic acid was replaced with 3-aminobutanoic acid. LCMS (Condition 1): m/z 475.0 [M+H]⁺, 1.54 min. ¹H NMR (400 MHz, DMSO-d₆) δ 12.82 (br s, 1H), 11.00 (br s, 1H), 8.26 (d, J=5.0 Hz, 1H), 7.67 (s, 1H), 7.54 (d, J=4.9 Hz, 1H), 7.47 (d, J=8.7 Hz, 2H), 7.17-7.12 (m, 1H), 7.05 (d, J=7.5 Hz, 2H), 6.53 (d, J=8.7 Hz, 1H), 3.80 (br s, 1H), 3.56 (q, J=8.2 Hz, 1H), 2.41-2.39 (m, 2H), 1.78 (s, 6H), 1.18 (d, J=6.5 Hz, 2H).

### Example 143: 2-(adamantan-1-yl)-2-((6-(N-(6-(o-tolyl)-5-(trifluoromethyl)pyridin-2-yl)sulfamoyl)pyridin-2-yl)amino)acetic Acid (143)

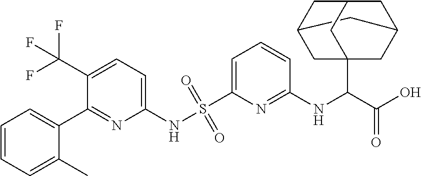 [SMILES: Cc1ccccc1-c1nc(NS(=O)(=O)c2cccc(NC(C(=O)O)C34CC5CC(CC(C5)C3)C4)n2)ccc1C(F)(F)F]

2-(adamantan-1-yl)-2-((6-(N-(6-(o-tolyl)-5-(trifluoromethyl)pyridin-2-yl)sulfamoyl)pyridin-2-yl)amino)acetic acid (143) was synthesized using the procedure described in Example 140, except 2-bromo-N-(5-chloro-6-(2,6-dimethylphenyl)pyridin-2-yl)pyridine-4-sulfonamide (int-b23) was replaced with 6-fluoro-N-(6-(o-tolyl)-5-(trifluoromethyl)pyridin-2-yl)pyridine-2-sulfonamide (int-b1) and 3-(ethylamino)propanoic acid was replaced with 2-(adamantan-1-yl)-2-aminoacetic acid. LCMS (Condition 1): m/z 601.0 [M+H]⁺, 1.81 min. ¹H NMR (400 MHz, DMSO-d₆) δ 12.63 (s, 1H), 11.60 (s, 1H), 8.14 (d, J=8.9 Hz, 1H), 7.54-7.51 (m, 2H), 7.24-6.79 (m, 6H), 4.33 (d, J=9.1 Hz, 1H), 4.17 (s, 1H), 2.05-1.30 (m, 18H).

### Example 144: 3-((4-(N-(5-chloro-6-(5-fluoro-2-methylphenyl)pyridin-2-yl)sulfamoyl)pyridin-2-yl)(ethyl)amino)propanoic Acid (144)

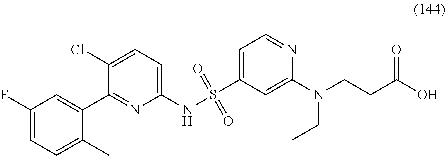 [SMILES: CCN(CCC(=O)O)c1cc(S(=O)(=O)Nc2ccc(Cl)c(-c3cc(F)ccc3C)n2)ccn1]

3-((4-(N-(5-chloro-6-(5-fluoro-2-methylphenyl)pyridin-2-yl)sulfamoyl)pyridin-2-yl)(ethyl)amino)propanoic acid (144) was synthesized using the procedure described in Example 140, except 2-bromo-N-(5-chloro-6-(2,6-dimethylphenyl)pyridin-2-yl)pyridine-4-sulfonamide (int-b23) was replaced with 2-bromo-N-(5-chloro-6-(5-fluoro-2-methylphenyl)pyridin-2-yl)pyridine-4-sulfonamide (int-b24). LCMS (Condition 1): m/z 493.1 [M+H]⁺, 1.59 min. ¹H NMR 400 MHz, DMSO-d₆) δ 12.90 (s, 1H), 11.50 (s, 1H), 8.22 (d, J=4.3 Hz, 1H), 7.99 (d, J=8.7 Hz, 1H), 7.39-7.05 (m, 3H), 7.00-6.95 (m, 1H), 6.83 (d, J=6.5 Hz, 2H), 3.58 (q, J=7.2 Hz, 2H), 3.31 (q, J=7.0 Hz, 2H), 2.46-2.41 (m, 2H), 1.81 (br s, 3H), 0.96 (t, J=7.0 Hz, 3H).

### Example 145: 3-((6-(N-(6-(2,6-dimethylphenyl)-5-(trifluoromethyl)pyridin-2-yl)sulfamoyl)pyridin-2-yl)(ethyl)amino)propanoic Acid (145)

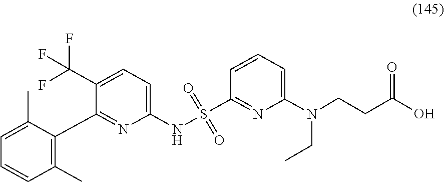 [SMILES: CCN(CCC(=O)O)c1cccc(S(=O)(=O)Nc2ccc(C(F)(F)F)c(-c3c(C)cccc3C)n2)n1]

3-((6-(N-(6-(2,6-dimethylphenyl)-5-(trifluoromethyl)pyridin-2-yl)sulfamoyl)pyridin-2-yl)(ethyl)amino)propanoic acid (145) was synthesized using the procedure described in Example 140, except 2-bromo-N-(5-chloro-6-(2,6-dimethylphenyl)pyridin-2-yl)pyridine-4-sulfonamide (int-b23) was replaced with N-(6-(2,6-dimethylphenyl)-5-(trifluoromethyl)pyridin-2-yl)-6-fluoropyridine-2-sulfonamide (int-b25). LCMS (Condition 1): m/z 523.1 [M+H]⁺, 1.76 min. ¹H NMR 400 MHz, DMSO-d₆) δ 12.90 (s, 1H), 11.50 (s, 1H), 8.22 (d, J=4.3 Hz, 1H), 7.99 (d, J=8.7 Hz, 1H), 7.39-7.05 (m, 3H), 7.00-6.95 (m, 1H), 6.83 (d, J=6.5 Hz, 2H), 3.58 (t, J=7.2 Hz, 2H), 3.31 (q, J=7.0 Hz, 2H), 2.46-2.41 (m, 2H), 1.81 (br s, 3H), 1.78 (s, 3H), 0.96 (t, J=7.0 Hz, 3H).

### Example 146: (6-(N-(6-(o-tolyl)-5-(trifluoromethyl)pyridin-2-yl)sulfamoyl)pyridin-2-yl)glycine (146)

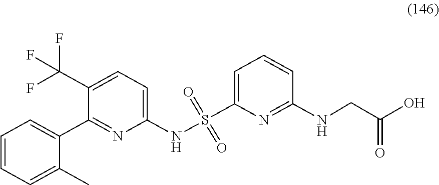 [SMILES: Cc1ccccc1-c1nc(NS(=O)(=O)c2cccc(NCC(=O)O)n2)ccc1C(F)(F)F]

(6-(N-(6-(o-tolyl)-5-(trifluoromethyl)pyridin-2-yl)sulfamoyl)pyridin-2-yl)glycine (146) was synthesized using the procedure described in Example 18, except 6-fluoro-N-(6-(2-(pyrimidin-2-yl)phenyl)-5-(trifluoromethyl)pyridin-2-yl)pyridine-2-sulfonamide (int-b15) was replaced with 6-fluoro-N-(6-(o-tolyl)-5-(trifluoromethyl)pyridin-2-yl)pyridine-2-sulfonamide (int-b1) and 4-methylpiperidine-4-carboxylic acid hydrochloride was replaced with glycine. LCMS (Condition 1): m/z 467.1 [M+H]⁺, 1.61 min. ¹H NMR (400 MHz, DMSO-d₆) δ 12.56 (brs, 1H), 7.60 (d, J=9.0 Hz, 1H), 7.43-7.34 (m, 1H), 7.29-7.09 (m, 5H), 7.01 (d, J=7.4 Hz, 1H), 6.94 (d, J=7.0 Hz, 1H), 6.52 (d, J=8.3 Hz, 1H), 6.42 (s, 1H), 3.59 (d, J=4.1 Hz, 2H), 1.92 (s, 3H).

### Example 147: 3-(cyclobutyl(6-(N-(6-(o-tolyl)-5-(trifluoromethyl)pyridin-2-yl)sulfamoyl)pyridin-2-yl)amino)propanoic acid (147)

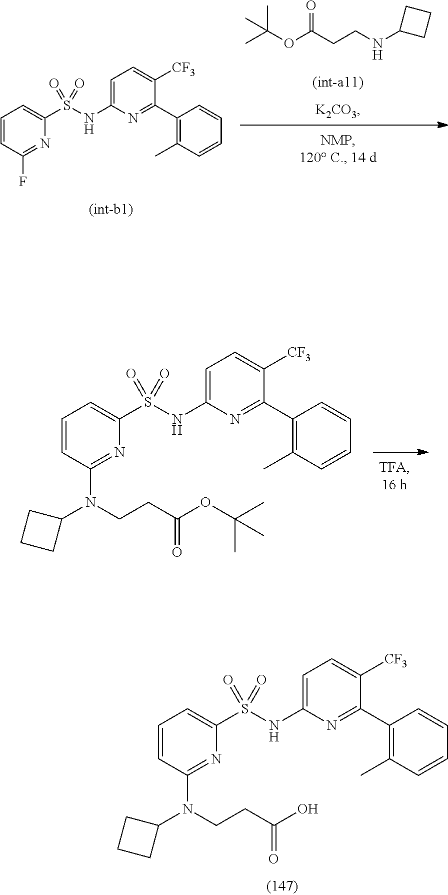 [SMILES: CC(C)(C)OC(=O)CCNC1CCC1 ; Cc1ccccc1-c1nc(NS(=O)(=O)c2cccc(F)n2)ccc1C(F)(F)F ; Cc1ccccc1-c1nc(NS(=O)(=O)c2cccc(N(CCC(=O)OC(C)(C)C)C3CCC3)n2)ccc1C(F)(F)F ; Cc1ccccc1-c1nc(NS(=O)(=O)c2cccc(N(CCC(=O)O)C3CCC3)n2)ccc1C(F)(F)F]

### Step 1. Synthesis of tert-butyl 3-(cyclobutyl(6-(N-(6-(o-tolyl)-5-(trifluoromethyl)pyridin-2-yl)sulfamoyl)pyridin-2-yl)amino)propanoate

In a vial, 6-fluoro-N-(6-(o-tolyl)-5-(trifluoromethyl)pyridin-2-yl)pyridine-2-sulfonamide (int-b1) (250 mg, 0.608 mmol) was taken up in NMP (3 mL), tert-butyl 3-(cyclobutylamino)propanoate (int-a11) (242 mg, 1.22 mmol) and K₂CO₃ (252 mg, 1.82 mmol) were added and the reaction was heated to 120° C. After 14 days, the reaction was poured into 1 M HCl and extracted with EtOAc (×3). The organics were then washed with water and brine, dried over MgSO₄, and concentrated. The crude material was purified by flash column chromatography (24 g RediSep Rf Gold silica gel column, 0-30% EtOAc/heptane) to give the product tert-butyl 3-(cyclobutyl(6-(N-(6-(o-tolyl)-5-(trifluoromethyl)pyridin-2-yl)sulfamoyl)pyridin-2-yl)amino)propanoate as a yellow oil. Condition 1, LCMS: m/z 590.9 [M+H]⁺, 1.93 min.

### Step 2. Synthesis of 3-(cyclobutyl(6-(N-(6-(o-tolyl)-5-(trifluoromethyl)pyridin-2-yl)sulfamoyl)pyridin-2-yl)amino)propanoic Acid (147)

In a vial, tert-butyl 3-(cyclobutyl(6-(N-(6-(o-tolyl)-5-(trifluoromethyl)pyridin-2-yl)sulfamoyl)pyridin-2-yl)amino)propanoate (26.8 mg, 0.045 mmol) was taken up in TFA (1 mL) and allowed to stand overnight. After 16 h, the material was concentrated in vacuo. The crude material was purified by flash column chromatography (4 g RediSep Rf Gold silica gel column, 0-40% EtOAc/heptane) to give 3-(cyclobutyl(6-(N-(6-(o-tolyl)-5-(trifluoromethyl)pyridin-2-yl)sulfamoyl)pyridin-2-yl)amino)propanoic acid (147) as a yellow oil. LCMS (Condition 1): m/z 534.9 [M+H]⁺, 1.71 min. ¹H NMR (400 MHz, Methanol-d₄) δ 8.07 (d, J=8.9 Hz, 1H), 7.66-7.54 (m, 2H), 7.31-7.27 (m, 1H), 7.23-7.18 (m, 2H), 7.18-7.15 (m, 1H), 7.05 (d, J=7.6 Hz, 1H), 6.80 (d, J=8.7 Hz, 1H), 4.37-4.23 (m, 1H), 3.76-3.71 (m, 2H), 2.41 (t, J=7.5 Hz, 2H), 2.31-2.20 (m, 2H), 2.17-2.03 (m, 2H), 1.88 (s, 3H), 1.80-1.58 (m, 2H).

### Example 148: 3-(propyl(6-(N-(6-(o-tolyl)-5-(trifluoromethyl)pyridin-2-yl)sulfamoyl)pyridin-2-yl)amino)propanoic Acid (148)

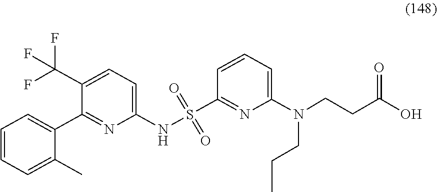 [SMILES: CCCN(CCC(=O)O)c1cccc(S(=O)(=O)Nc2ccc(C(F)(F)F)c(-c3ccccc3C)n2)n1]

3-(propyl(6-(N-(6-(o-tolyl)-5-(trifluoromethyl)pyridin-2-yl)sulfamoyl)pyridin-2-yl)amino)propanoic acid (148) was synthesized using the procedure described in Example 147, except in step 1, tert-butyl 3-(cyclobutylamino)propanoate (int-a11) was replaced with tert-butyl 3-(propylamino)propanoate and K₂CO₃ was replaced with N,N-diisopropylethylamine, and in step 2, TFA was replaced with HCl. LCMS (Condition 1): m/z 523.2 [M+H]⁺, 1.41 min. ¹H NMR (400 MHz, Methanol-d₄) δ 8.05 (d, J=8.9 Hz, 1H), 7.63-7.49 (m, 2H), 7.33-7.26 (m, 1H), 7.23-7.14 (m, 3H), 7.05 (d, J=7.5 Hz, 1H), 6.79 (d, J=8.6 Hz, 1H), 3.69-3.64 (m, 2H), 3.39 (dd, J=7.7, 4.0 Hz, 2H), 2.47 (t, J=7.1 Hz, 2H), 1.88 (s, 3H), 1.60-1.43 (m, 2H), 0.91-0.83 (m, 3H).

### Example 149: 3-((6-(N-(6-(5-fluoro-2-methylphenyl)-5-(trifluoromethyl)pyridin-2-yl)sulfamoyl)pyridin-2-yl)(propyl)amino)propanoic Acid (149)

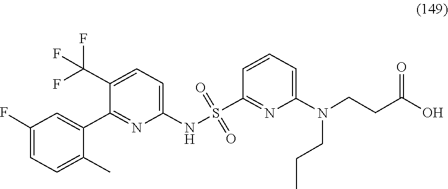 [SMILES: CCCN(CCC(=O)O)c1cccc(S(=O)(=O)Nc2ccc(C(F)(F)F)c(-c3cc(F)ccc3C)n2)n1]

3-((6-(N-(6-(5-fluoro-2-methylphenyl)-5-(trifluoromethyl)pyridin-2-yl)sulfamoyl)pyridin-2-yl)(propyl)amino)propanoic acid (149) was synthesized using the procedure described in Example 147, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), tert-butyl 3-(cyclobutylamino)propanoate (int-a11) was replaced with tert-butyl 3-(propylamino)propanoate and K₂CO₃ was replaced with N,N-diisopropylethylamine, and in step 2, TFA was replaced with HCl. LCMS (Condition 1): m/z 541.1 [M+H]⁺, 1.42 min. ¹H NMR (400 MHz, Methanol-d₄) δ 8.06 (d, J=8.9 Hz, 1H), 7.58 (dd, J=8.7, 7.3 Hz, 1H), 7.51 (dd, J=8.7, 0.8 Hz, 1H), 7.22-7.16 (m, 1H), 7.15 (d, J=7.2 Hz, 1H), 7.06-6.98 (m, 1H), 6.78 (dd, J=8.8, 2.4 Hz, 2H), 3.69-3.64 (m, 2H), 3.43-3.33 (m, 2H), 2.44 (t, J=7.2 Hz, 2H), 1.81 (s, 3H), 1.57-1.43 (m, 2H), 0.86 (t, J=7.4 Hz, 3H).

### Example 150: 3-((6-(N-(6-(o-tolyl)-5-(trifluoromethyl)pyridin-2-yl)sulfamoyl)pyridin-2-yl)(3,3,3-trifluoropropyl)amino)propanoic Acid (150)

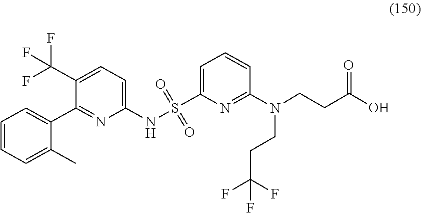 [SMILES: Cc1ccccc1-c1nc(NS(=O)(=O)c2cccc(N(CCC(=O)O)CCC(F)(F)F)n2)ccc1C(F)(F)F]

3-((6-(N-(6-(o-tolyl)-5-(trifluoromethyl)pyridin-2-yl)sulfamoyl)pyridin-2-yl)(3,3,3-trifluoropropyl)amino)propanoic acid (150) was synthesized using the procedure described in Example 147, except in step 1, tert-butyl 3-(cyclobutylamino)propanoate (int-a11) was replaced with tert-butyl 3-((3,3,3-trifluoropropylamino)propanoate (int-a12) and K₂CO₃ was replaced with N,N-diisopropylethylamine, and in step 2, TFA was replaced with HCl. LCMS (Condition 1): m/z 577.1 [M+H]⁺, 1.40 min. ¹H NMR (400 MHz, Methanol-d₄) δ 8.04 (d, J=8.8 Hz, 1H), 7.65 (dd, J=8.7, 7.3 Hz, 1H), 7.48 (dd, J=8.9, 0.9 Hz, 1H), 7.32-7.26 (m, 1H), 7.24 (d, J=7.3 Hz, 1H), 7.20 (d, J=8.0 Hz, 1H), 7.18-7.14 (m, 1H), 7.03 (d, J=7.6 Hz, 1H), 6.86 (d, J=8.7 Hz, 1H), 3.77-3.61 (m, 4H), 2.52 (t, J=6.8 Hz, 2H), 2.45-2.31 (m, 2H), 1.86 (s, 3H).

### Example 151: 3-((6-(N-(6-(5-fluoro-2-methylphenyl)-5-(trifluoromethyl)pyridin-2-yl)sulfamoyl)pyridin-2-yl)(3,3,3-trifluoropropyl)amino)propanoic Acid (151)

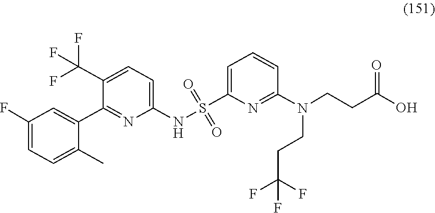 [SMILES: Cc1ccc(F)cc1-c1nc(NS(=O)(=O)c2cccc(N(CCC(=O)O)CCC(F)(F)F)n2)ccc1C(F)(F)F]

3-((6-(N-(6-(5-fluoro-2-methylphenyl)-5-(trifluoromethyl)pyridin-2-yl)sulfamoyl)pyridin-2-yl)(3,3,3-trifluoropropyl)amino)propanoic acid (151) was synthesized using the procedure described in Example 147, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), tert-butyl 3-(cyclobutylamino)propanoate (int-a11) was replaced with tert-butyl 3-((3,3,3-trifluoropropylamino)propanoate (int-a12), and K₂CO₃ was replaced with N,N-diisopropylethylamine, and in step 2, TFA was replaced with HCl. LCMS (Condition 1): m/z 595.1 [M+H]⁺, 1.41 min. ¹H NMR (400 MHz, Methanol-d₄) δ 8.05 (d, J=8.8 Hz, 1H), 7.69-7.61 (m, 1H), 7.45 (dd, J=8.8, 0.9 Hz, 1H), 7.24 (d, J=7.3 Hz, 1H), 7.20 (dd, J=8.5, 5.6 Hz, 1H), 7.06-6.98 (m, 1H), 6.86 (d, J=8.7 Hz, 1H), 6.77 (dd, J=9.2, 2.8 Hz, 1H), 3.79-3.64 (m, 4H), 2.51 (t, J=6.8 Hz, 2H), 2.45-2.30 (m, 2H), 1.80 (s, 3H).

### Example 152: 3-(butyl(6-(N-(6-(o-tolyl)-5-(trifluoromethyl)pyridin-2-yl)sulfamoyl)pyridin-2-yl)amino)propanoic Acid (152)

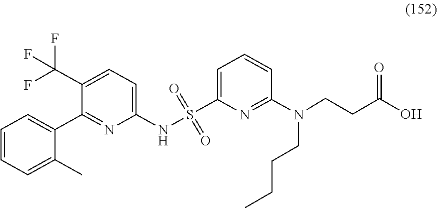 [SMILES: CCCCN(CCC(=O)O)c1cccc(S(=O)(=O)Nc2ccc(C(F)(F)F)c(-c3ccccc3C)n2)n1]

3-(butyl(6-(N-(6-(o-tolyl)-5-(trifluoromethyl)pyridin-2-yl)sulfamoyl)pyridin-2-yl)amino)propanoic acid (152) was synthesized using the procedure described in Example 147, except in step 1, tert-butyl 3-(cyclobutylamino)propanoate (int-a11) was replaced with tert-butyl 3-(butylamino)propanoate and K₂CO₃ was replaced with N,N-diisopropylethylamine, and in step 2, TFA was replaced with HCl. LCMS (Condition 1): m/z 536.9 [M+H]⁺, 1.72 min. ¹H NMR (400 MHz, Methanol-d₄) δ 8.05 (d, J=8.9 Hz, 1H), 7.64-7.49 (m, 2H), 7.32-7.25 (m, 1H), 7.23-7.13 (m, 3H), 7.05 (d, J=7.5 Hz, 1H), 6.78 (d, J=8.7 Hz, 1H), 3.74-3.59 (m, 2H), 3.47-3.37 (m, 2H), 2.46 (t, J=7.1 Hz, 2H), 1.87 (s, 3H), 1.56-1.42 (m, 2H), 1.34-1.28 (m, 2H), 0.92 (t, J=7.3 Hz, 3H).

### Example 153: 3-(butyl(6-(N-(6-(5-fluoro-2-methylphenyl)-5-(trifluoromethyl)pyridin-2-yl)sulfamoyl)pyridin-2-yl)amino)propanoic Acid (153)

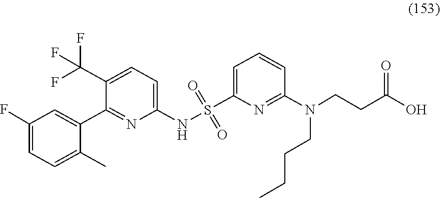 [SMILES: CCCCN(CCC(=O)O)c1cccc(S(=O)(=O)Nc2ccc(C(F)(F)F)c(-c3cc(F)ccc3C)n2)n1]

3-(butyl(6-(N-(6-(5-fluoro-2-methylphenyl)-5-(trifluoromethyl)pyridin-2-yl)sulfamoyl)pyridin-2-yl)amino)propanoic acid (153) was synthesized using the procedure described in Example 147, except in step 1, 6-fluoro-N-(6-(o-tolyl)-5-(trifluoromethyl)pyridin-2-yl)pyridine-2-sulfonamide (int-b1) was replaced with 6-fluoro-N-(6-(5-fluoro-2-methylphenyl)-5-(trifluoromethyl)pyridin-2-yl)pyridine-2-sulfonamide (int-b10), tert-butyl 3-(cyclobutylamino)propanoate (int-a11) was replaced with tert-butyl 3-(butylamino)propanoate, and K₂CO₃ was replaced with N,N-diisopropylethylamine, and in step 2, TFA was replaced with HCl. LCMS (Condition 1): m/z 554.9 [M+H]⁺, 1.74 min. ¹H NMR (400 MHz, Methanol-d₄) δ 8.07 (dd, J=8.9, 7.4 Hz, 1H), 7.62-7.55 (m, 1H), 7.51-7.43 (m, 1H), 7.24-7.16 (m, 1H), 7.15-7.11 (m, 1H), 7.07-6.98 (m, 1H), 6.83-6.63 (m, 1H), 6.78 (dd, J=8.9, 2.1 Hz, 1H), 3.71-3.51 (m, 2H), 3.49-3.36 (m, 2H), 2.47-2.34 (m, 2H), 1.98-1.77 (m, 3H), 1.63-1.42 (m, 2H), 1.38-1.17 (m, 2H), 1.03-0.82 (m, 3H).

### Example 154: 4-((6-(N-(5-chloro-6-(o-tolyl)pyridin-2-yl)sulfamoyl)pyridin-2-yl)(isobutyl)amino)butanoic Acid (154)

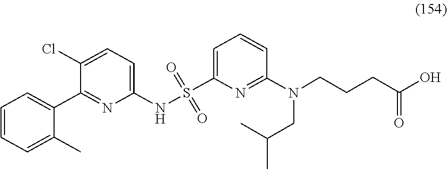 [SMILES: Cc1ccccc1-c1nc(NS(=O)(=O)c2cccc(N(CCCC(=O)O)CC(C)C)n2)ccc1Cl]

4-((6-(N-(5-chloro-6-(o-tolyl)pyridin-2-yl)sulfamoyl)pyridin-2-yl)(isobutyl)amino)butanoic acid (154) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(5-chloro-6-(o-tolyl)pyridin-2-yl)-6-fluoropyridine-2-sulfonamide (int-b22), 4-(butylamino)butan-1-ol was replaced with 4-(isobutylamino)butan-1-ol (int-a6), and heating in microwave at 200° C. for 1 hour was replaced with heating in an oil bath at 130° C. for 18 hours. LCMS (Condition 1): m/z 517.1 [M+H]⁺, 1.44 min. ¹H NMR (400 MHz, DMSO-d₆) δ 12.11 (s, 1H), 11.12 (s, 1H), 7.91 (d, J=8.7 Hz, 1H), 7.63 (dd, J=8.7, 7.2 Hz, 1H), 7.32-7.25 (m, 2H), 7.25-7.15 (m, 2H), 7.03 (d, J=7.2 Hz, 1H), 6.97 (dd, J=7.5, 1.5 Hz, 1H), 6.87 (d, J=8.7 Hz, 1H), 3.31-3.30 (m, 2H), 3.16 (d, J=7.5 Hz, 2H), 2.19 (t, J=7.2 Hz, 2H), 1.84-1.75 (m, 4H), 1.64-1.53 (m, 2H), 0.72 (d, J=6.6 Hz, 6H).

### Example 155: 4-((6-(N-(5-chloro-6-(2,6-dimethylphenyl)pyridin-2-yl)sulfamoyl)pyridin-2-yl)(isobutyl)amino)butanoic Acid (155)

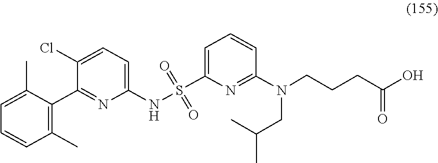 [SMILES: Cc1cccc(C)c1-c1nc(NS(=O)(=O)c2cccc(N(CCCC(=O)O)CC(C)C)n2)ccc1Cl]

4-((6-(N-(5-chloro-6-(2,6-dimethylphenyl)pyridin-2-yl)sulfamoyl)pyridin-2-yl)(isobutyl)amino)butanoic acid (155) was synthesized using the procedure described in Example 87, except in step 1, 6-fluoro-N-(6-(o-tolyl)-5-(trifluoromethyl)pyridin-2-yl)pyridine-2-sulfonamide (int-b1) was replaced with N-(5-chloro-6-(2,6-dimethylphenyl)pyridin-2-yl)-6-fluoropyridine-2-sulfonamide (int-b6), 4-(butylamino)butan-1-ol was replaced with 4-(isobutylamino)butan-1-ol (int-a6), and heating in microwave at 200° C. for 1 hour was replaced with heating in an oil bath at 130° C. for 18 hours. LCMS (Condition 1): m/z 531.1 [M+H]⁺, 1.47 min. ¹H NMR (400 MHz, DMSO-d₆) δ 12.12 (s, 1H), 11.14 (s, 1H), 7.92 (d, J=8.8 Hz, 1H), 7.60 (dd, J=8.7, 7.2 Hz, 1H), 7.26 (d, J=8.8 Hz, 1H), 7.16 (dd, J=8.1, 7.0 Hz, 1H), 7.02 (dd, J=14.0, 7.5 Hz, 3H), 6.85 (d, J=8.8 Hz, 1H), 3.30 (s, 2H), 3.16 (d, J=7.4 Hz, 2H), 2.20 (t, J=7.2 Hz, 2H), 1.92-1.77 (m, 1H), 1.71 (s, 6H), 1.59 (t, J=7.7 Hz, 2H), 0.74 (d, J=6.6 Hz, 6H).

### Example 156: 4-((6-(N-(5-chloro-6-(o-tolyl)pyridin-2-yl)sulfamoyl)pyridin-2-yl)(propyl)amino)butanoic Acid (156)

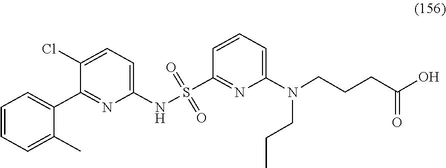 [SMILES: CCCN(CCCC(=O)O)c1cccc(S(=O)(=O)Nc2ccc(Cl)c(-c3ccccc3C)n2)n1]

4-((6-(N-(5-chloro-6-(o-tolyl)pyridin-2-yl)sulfamoyl)pyridin-2-yl)(propyl)amino)butanoic acid (156) was synthesized using the procedure described in Example 87, except 6-fluoro-N-(6-(o-tolyl)-5-(trifluoromethyl)pyridin-2-yl)pyridine-2-sulfonamide (int-b1) was replaced with N-(5-chloro-6-(o-tolyl)pyridin-2-yl)-6-fluoropyridine-2-sulfonamide (int-b22), 4-(butylamino)butan-1-ol was replaced with 4-(propylamino)butan-1-ol (int-a8), and heating in microwave at 200° C. for 1 hour was replaced with heating in an oil bath at 130° C. for 18 hours. LCMS (Condition 1): m/z 503.1 [M+H]⁺, 1.40 min. ¹H NMR (400 MHz, DMSO-d₆) δ 12.09 (s, 1H), 11.11 (s, 1H), 7.91 (d, J=8.8 Hz, 1H), 7.64 (dd, J=8.7, 7.3 Hz, 1H), 7.35-7.26 (m, 2H), 7.26-7.15 (m, 2H), 7.09-6.94 (m, 2H), 6.85 (d, J=8.7 Hz, 1H), 3.32-3.19 (m, 4H), 2.18 (t, J=7.3 Hz, 2H), 1.84 (s, 3H), 1.67-1.51 (m, 2H), 1.42-1.29 (m, 2H), 0.75 (t, J=7.3 Hz, 3H).

### Example 157: 4-((6-(N-(5-chloro-6-(2,6-dimethylphenyl)pyridin-2-yl)sulfamoyl)pyridin-2-yl)(propyl)amino)butanoic Acid (157)

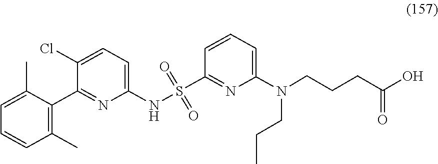 [SMILES: CCCN(CCCC(=O)O)c1cccc(S(=O)(=O)Nc2ccc(Cl)c(-c3c(C)cccc3C)n2)n1]

4-((6-(N-(5-chloro-6-(2,6-dimethylphenyl)pyridin-2-yl)sulfamoyl)pyridin-2-yl)(propyl)amino)butanoic acid (157) was synthesized using the procedure described in Example 87, except 6-fluoro-N-(6-(o-tolyl)-5-(trifluoromethyl)pyridin-2-yl)pyridine-2-sulfonamide (int-b1) was replaced with N-(5-chloro-6-(2,6-dimethylphenyl)pyridin-2-yl)-6-fluoropyridine-2-sulfonamide (int-b6), 4-(butylamino)butan-1-ol was replaced with 4-(propylamino)butan-1-ol (int-a8), and heating in microwave at 200° C. for 1 hour was replaced with heating in an oil bath at 130° C. for 18 hours. LCMS (Condition 1): m/z 517.1 [M+H]⁺, 1.44 min. ¹H NMR (400 MHz, DMSO-d₆) δ 12.12 (s, 1H), 11.13 (s, 1H), 7.92 (d, J=8.8 Hz, 1H), 7.61 (dd, J=8.7, 7.3 Hz, 1H), 7.29 (d, J=8.8 Hz, 1H), 7.16 (dd, J=8.1, 7.0 Hz, 1H), 7.02 (dd, J=14.7, 7.4 Hz, 3H), 6.82 (d, J=8.7 Hz, 1H), 3.27 (dd, J=15.4, 7.9 Hz, 4H), 2.20 (t, J=7.3 Hz, 2H), 1.71 (s, 6H), 1.65-1.53 (m, 2H), 1.42-1.29 (m, 2H), 0.77 (t, J=7.4 Hz, 3H).

### Example 158: 4-((2-methoxyethyl)(6-(N-(6-(o-tolyl)-5-(trifluoromethyl)pyridin-2-yl)sulfamoyl)pyridin-2-yl)amino)butanoic Acid (158)

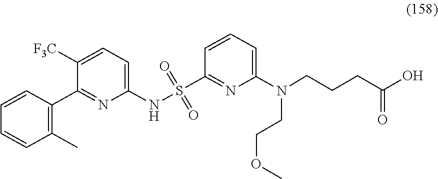 [SMILES: COCCN(CCCC(=O)O)c1cccc(S(=O)(=O)Nc2ccc(C(F)(F)F)c(-c3ccccc3C)n2)n1]

4-((2-methoxyethyl)(6-(N-(6-(o-tolyl)-5-(trifluoromethyl)pyridin-2-yl)sulfamoyl)pyridin-2-yl)amino)butanoic acid (158) was synthesized using the procedure described in Example 87, except in step 1, 4-(butylamino)butan-1-ol was replaced with 4-((2-methoxyethyl)amino)butan-1-ol, and heating in microwave at 200° C. for 1 hour was replaced with heating in an oil bath at 120° C. for 18 hours. LCMS (Condition 1): m/z 553.2 [M+H]⁺, 1.35 min. ¹H NMR (400 MHz, Methanol-d₄) δ 8.05 (d, J=8.9 Hz, 1H), 7.61-7.49 (m, 2H), 7.31-7.27 (m, 1H), 7.24-7.12 (m, 3H), 7.04 (d, J=7.6 Hz, 1H), 6.88 (d, J=8.7 Hz, 1H), 3.61-3.56 (m, 2H), 3.49-3.44 (m, 2H), 3.42-3.37 (m, 2H), 3.24 (s, 3H), 2.26 (t, J=7.1 Hz, 2H), 1.88 (s, 3H), 1.83-1.75 (m, 2H).

### Administration and Pharmaceutical Compositions

For the therapeutic uses of compounds of the present invention, such compounds are administered either alone or as part of a pharmaceutical composition. Accordingly, in another aspect of the present invention provides a pharmaceutical composition, which comprises a compound of the present invention, or pharmaceutically acceptable salt thereof, and one or more pharmaceutically acceptable carriers. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. In certain embodiments, the pharmaceutical composition comprising a compound of the present invention can be formulated for intramuscularly, intravenously, subcutaneously, orally, pulmonary, intrathecally, topically or intranasally administration.

The pharmaceutical compositions of the present invention can be made up in a solid form (including without limitation capsules, tablets, pills, granules, powders or suppositories), or in a liquid form (including without limitation solutions, suspensions or emulsions). Tablets may be either film coated or enteric coated according to methods known in the art.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Suitable compositions for oral administration include a compound of the present invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present invention or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an iv formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

Certain aspects and examples of the pharmaceutical compositions of the present invention are provided in the following listing of enumerated embodiments. It will be recognized that features specified in each embodiment may be combined with other specified features to provide further embodiments of the present invention.

### Embodiment 173

A pharmaceutical composition comprising a compound of any one of Embodiments 1 to 172, or a pharmaceutically acceptable salt thereof, and one or more pharmaceutically acceptable carriers.

### Embodiment 174

A pharmaceutical composition comprising a compound of Embodiment 172, or a pharmaceutically acceptable salt thereof, and one or more pharmaceutically acceptable carriers.

### Embodiment 175

The pharmaceutical composition of Embodiment 173 or 174 comprising one or more additional additional pharmaceutical agents.

### Embodiment 176

The pharmaceutical composition of Embodiment 175, wherein the additional pharmaceutical agent(s) is selected from a mucolytic agent, nebulized hypertonic saline, bronchodilator, an antibiotic, an anti-infective agent, a CFTR modulator, and an anti-inflammatory agent.

### Embodiment 177

The pharmaceutical composition of Embodiment 176, wherein the one or more additional pharmaceutical agents is a CFTR modulator.

### Embodiment 178

The pharmaceutical composition of Embodiment 176, wherein the one or more additional pharmaceutical agents a CFTR corrector.

### Embodiment 179

The pharmaceutical composition of Embodiment 176, wherein the one or more additional pharmaceutical agents is a CFTR potentiator.

### Embodiment 180

The pharmaceutical composition of Embodiment 176, wherein the one or more additional pharmaceutical agent is a CFTR modulator and a CFTR potentiator.

### Embodiment 181

The pharmaceutical composition of Embodiment 176, wherein the one or more additional pharmaceutical agent is a CFTR corrector and a CFTR potentiator.

### Pharmacology and Utility

Compounds of the present invention have been found to modulate CFTR activity and may be beneficial for the treatment of cystic fibrosis and additional diseases not directly caused by mutations in CFTR, such as secretory diseases and other protein folding diseases mediated by CFTR. These include, but are not limited to, chronic obstructive pulmonary disease (COPD), dry eye disease, and Sjogren's Syndrome.

COPD is characterized by airflow limitation that is progressive and not fully reversible. The airflow limitation is due to mucus hypersecretion, emphysema, and bronchiolitis. Activators of mutant or wild-type CFTR offer a potential treatment of mucus hypersecretion and impaired mucociliary clearance that is common in COPD. Specifically, increasing anion secretion across CFTR may facilitate fluid transport into the airway surface liquid to hydrate the mucus and optimized periciliary fluid viscosity. This would lead to enhanced mucociliary clearance and a reduction in the symptoms associated with COPD.

Dry eye disease is characterized by a decrease in tear aqueous production and abnormal tear film lipid, protein and mucin profiles. There are many causes of dry eye, some of which include age, Lasik eye surgery, arthritis, medications, chemical/thermal burns, allergies, and diseases, such as cystic fibrosis and Sjogrens's syndrome. Increasing anion secretion via CFTR would enhance fluid transport from the corneal endothelial cells and secretory glands surrounding the eye to increase corneal hydration. This would help to alleviate the symptoms associated with dry eye disease.

Sjogrens's syndrome is an autoimmune disease in which the immune system attacks moisture-producing glands throughout the body, including the eye, mouth, skin, respiratory tissue, liver, vagina, and gut. Symptoms, include, dry eye, mouth, and vagina, as well as lung disease. The disease is also associated with rheumatoid arthritis, systemic lupus, systemic sclerosis, and polymypositis/dermatomyositis. Defective protein trafficking is believed to cause the disease, for which treatment options are limited. Augmenters or inducers of CFTR activity may hydrate the various organs afflicted by the disease and help to elevate the associated symptoms.

The compounds of the present invention, in free form or in pharmaceutically acceptable salt form, exhibit valuable pharmacological properties, e.g. CFTR modulating properties, as indicated by the in vitro tests provided herein, and are therefore indicated for therapy or for use as research chemicals, e.g. as tool compounds.

Compounds of the present invention may be useful in the treatment of cystic fibrosis, chronic bronchitis, recurrent bronchitis, acute bronchitis, male infertility caused by congenital bilateral absence of the vas deferens (CBAVD), female infertility caused by congenital absence of the uterus and vagina (CAUV), chronic rhinosinusitis, primary sclerosing cholangitis, allergic bronchopulmonary aspergillosis, diabetes, dry eye, constipation, allergic bronchopulmonary aspergillosis (ABPA), bone diseases (e.g., osteoporosis), asthma, chronic obstructive pulmonary disease (COPD), chronic bronchitis or dyspnea associated therewith, recurrent bronchitis, acute bronchitis, rhinosinusitis, constipation, pancreatitis including idiopathic chronic pancreatitis (ICP), idiopathic recurrent pancreatitis, idiopathic acute pancreatitis, chronic pancreatitis, recurrent pancreatitis, and acute pancreatitis, pancreatic insufficiency, male infertility caused by congenital bilateral absence of the vas deferens (CBAVD), mild pulmonary disease, idiopathic pancreatitis, liver disease, emphysema, hereditary emphysema, gallstones, gastro-esophageal reflux disease, gastrointestinal malignancies, inflammatory bowel disease, constipation, diabetes, arthritis, osteoporosis, osteopenia, dry eye disease, and Sjogren's Syndrome.

Compounds of the present invention may be useful in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

Thus, as a further aspect, the present invention provides the use of a compound of the present invention, or pharmaceutically acceptable salt thereof, in therapy. In a further embodiment, the therapy is selected from a disease which may be treated by modulation of CFTR activity. In another embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

Thus, as a further aspect, the present invention provides a compound of the present invention or pharmaceutically acceptable salt thereof, for use in therapy. In a further embodiment, the therapy is selected from a disease which may be treated by modulation of CFTR activity. In another embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

Thus, as a further aspect, the present invention provides the use of a compound of the present invention, or pharmaceutically acceptable salt thereof, in therapy for the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In another aspect, the invention provides a method of treating a disease which is treated by modulation of CFTR comprising administration of a therapeutically acceptable amount of a compound of the present invention, or pharmaceutically acceptable salt thereof. In further embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

In another aspect, the invention provides a method of treating pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, wheren the method compises administration of a therapeutically acceptable amount of a compound of the present invention, or pharmaceutically acceptable salt thereof.

Thus, as a further aspect, the present invention provides the use of a compound of the present invention, or pharmaceutically acceptable salt thereof, for the manufacture of a medicament. In a further embodiment, the medicament is for treatment of a disease which may be treated by modulation of CFTR. In another embodiment, the disease is selected from the aforementioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

Thus, as a further aspect, the present invention provides the use of a compound of the present invention, or pharmaceutically acceptable salt thereof, for the manufacture of a medicament for the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(isobutyl(6-(N-(6-(o-tolyl)-5-(trifluoromethyl)pyridin-2-yl)sulfamoyl)pyridin-2-yl)amino)butanoic acid for use in the treatment of cystic fibrosis, asthma, COPD, chronic bronchitis, pancreatitis and emphysema.

In one embodiment of the present invention, there is provided 4-((6-(N-(6-(5-fluoro-2-methylphenyl)-5-(trifluoromethyl)pyridin-2-yl)sulfamoyl)pyridin-2-yl)(propyl)amino)butanoic acid for use in the treatment of cystic fibrosis, asthma, COPD, chronic bronchitis, pancreatitis and emphysema.

In one embodiment of the present invention, there is provided 3-((cyclopropylmethyl)(6-(N-(6-(o-tolyl)-5-(trifluoromethyl)pyridin-2-yl)sulfamoyl)pyridin-2-yl)amino)propanoic acid for use in the treatment of cystic fibrosis, asthma, COPD, chronic bronchitis, pancreatitis and emphysema.

In one embodiment of the present invention, there is provided 4-((6-(N-(6-(5-fluoro-2-methylphenyl)-5-(trifluoromethyl)pyridin-2-yl)sulfamoyl)pyridin-2-yl)(isopropyl)amino)butanoic acid for use in the treatment of cystic fibrosis, asthma, COPD, chronic bronchitis, pancreatitis and emphysema.

In one embodiment of the present invention, there is provided 4-(isobutyl(6-(N-(6-(o-tolyl)-5-(trifluoromethyl)pyridin-2-yl)sulfamoyl)pyridin-2-yl)amino)butanoic acid for use in the treatment of cystic fibrosis, asthma, COPD, chronic bronchitis, and emphysema.

In one embodiment of the present invention, there is provided 4-((6-(N-(6-(5-fluoro-2-methylphenyl)-5-(trifluoromethyl)pyridin-2-yl)sulfamoyl)pyridin-2-yl)(propyl)amino)butanoic acid for use in the treatment of cystic fibrosis.

In one embodiment of the present invention, there is provided 3-((cyclopropylmethyl)(6-(N-(6-(o-tolyl)-5-(trifluoromethyl)pyridin-2-yl)sulfamoyl)pyridin-2-yl)amino)propanoic acid for use in the treatment of cystic fibrosis.

In one embodiment of the present invention, there is provided 4-((6-(N-(6-(5-fluoro-2-methylphenyl)-5-(trifluoromethyl)pyridin-2-yl)sulfamoyl)pyridin-2-yl)(isopropyl)amino)butanoic acid for use in the treatment of cystic fibrosis.

In one embodiment of the present invention, there is provided 4-(isobutyl(6-(N-(6-(o-tolyl)-5-(trifluoromethyl)pyridin-2-yl)sulfamoyl)pyridin-2-yl)amino)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-((6-(N-(6-(5-fluoro-2-methylphenyl)-5-(trifluoromethyl)pyridin-2-yl)sulfamoyl)pyridin-2-yl)(propyl)amino)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 3-((cyclopropylmethyl)(6-(N-(6-(o-tolyl)-5-(trifluoromethyl)pyridin-2-yl)sulfamoyl)pyridin-2-yl)amino)propanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-((6-(N-(6-(5-fluoro-2-methylphenyl)-5-(trifluoromethyl)pyridin-2-yl)sulfamoyl)pyridin-2-yl)(isopropyl)amino)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

Another aspect of the present invention provides a method for treating or lessening the severity of a disease, disorder, or condition associated with the modulation of CFTR in a subject, which comprises administering to the subject a compound of the present invention, or a pharmaceutically acceptable salt thereof.

In certain embodiments, the present invention provides a method of treating a condition, disease, or disorder implicated by a deficiency of the CFTR activity, the method comprising administering a compound of the present invention to a subject in need of treatment thereof. In certain embodiments, the present invention provides a method of treating a condition, disease, or disorder implicated by a deficiency of the CFTR activity, the method comprising administering a composition comprising a compound of the present invention to a subject in need of treatment thereof.

In certain embodiments, the present invention provides a method of treating diseases associated with reduced CFTR function due to mutations in the gene encoding CFTR or environmental factors (e.g., smoke), the method comprising administering a compound of the present invention to a subject in need of treatment thereof. In certain embodiments, the present invention provides a method of treating diseases associated with reduced CFTR function due to mutations in the gene encoding CFTR or environmental factors (e.g., smoke), the method comprising administering a composition comprising a compound of the present invention to a subject in need of treatment thereof. The diseases associated with reduced CFTR function due to mutations in the gene encoding CFTR or environmental factors (e.g., smoke) include, cystic fibrosis, chronic bronchitis, recurrent bronchitis, acute bronchitis, male infertility caused by congenital bilateral absence of the vas deferens (CBAVD), female infertility caused by congenital absence of the uterus and vagina (CAUV), idiopathic chronic pancreatitis (ICP), idiopathic recurrent pancreatitis, idiopathic acute pancreatitis, chronic rhinosinusitis, primary sclerosing cholangitis, allergic bronchopulmonary aspergillosis, diabetes, dry eye, constipation, allergic bronchopulmonary aspergillosis (ABPA), bone diseases (e.g., osteoporosis), and asthma.

In certain embodiments, the present invention provides a method for treating diseases associated with normal CFTR function, the method comprising administering a compound of the present invention to a subject in need of treatment thereof. In certain embodiments, the present invention provides a method for treating diseases associated with normal CFTR function), the method comprising administering a composition comprising a compound of the present invention to a subject in need of treatment thereof. The diseases associated with normal CFTR function include, chronic obstructive pulmonary disease (COPD), chronic bronchitis or dyspnea associated therewith, recurrent bronchitis, acute bronchitis, rhinosinusitis, constipation, pancreatitis including chronic pancreatitis, recurrent pancreatitis, and acute pancreatitis, pancreatic insufficiency, male infertility caused by congenital bilateral absence of the vas deferens (CBAVD), mild pulmonary disease, idiopathic pancreatitis, liver disease, emphysema, hereditary emphysema, gallstones, gastro-esophageal reflux disease, gastrointestinal malignancies, inflammatory bowel disease, constipation, diabetes, arthritis, osteoporosis, and osteopenia.

According to an alternative preferred embodiment, the present invention provides a method of treating cystic fibrosis comprising administering to a subject in need of such treatment a compound of the present invention, or a pharmaceutically acceptable salt thereof. According to an alternative preferred embodiment, the present invention provides a method of treating cystic fibrosis comprising administering to a subject in need of such treatment a composition comprising a compound of the present invention, or a pharmaceutically acceptable salt thereof.

According to an alternative preferred embodiment, the present invention provides a method of treating chronic obstructive pulmonary disease (COPD) comprising administering to a subject in need of such treatment a compound of the present invention, or a pharmaceutically acceptable salt thereof. According to an alternative preferred embodiment, the present invention provides a method of treating chronic obstructive pulmonary disease (COPD) comprising administering to a subject in need of such treatment a composition comprising a compound of the present invention, or a pharmaceutically acceptable salt thereof.

According to an alternative preferred embodiment, the present invention provides a method of treating pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, comprising administering to a subject in need of such treatment a compound of the present invention, or a pharmaceutically acceptable salt thereof. According to an alternative preferred embodiment, the present invention provides a method of treating pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, comprising administering to a subject in need of such treatment a composition comprising a compound of the present invention, or a pharmaceutically acceptable salt thereof.

According to the invention an “effective dose” or an “effective amount” of the compound or pharmaceutical composition is that amount effective for treating or lessening the severity of one or more of the diseases, disorders or conditions as recited above.

The compounds and compositions, according to the methods of the present invention, may be administered using any amount and any route of administration effective for treating or lessening the severity of one or more of the diseases, disorders or conditions recited above.

In certain embodiments, the present invention relates to the aforementioned methods, wherein said compound is administered parenterally.

In certain embodiments, the present invention relates to the aforementioned methods, wherein said compound is administered intramuscularly, intravenously, subcutaneously, orally, pulmonary, intrathecally, topically or intranasally.

In certain embodiments, the present invention relates to the aforementioned methods, wherein said compound is administered systemically.

In certain embodiments, the present invention relates to the aforementioned methods, wherein said subject is a mammal.

In certain embodiments, the present invention relates to the aforementioned methods, wherein said subject is a primate.

In certain embodiments, the present invention relates to the aforementioned methods, wherein said subject is a human.

Certain aspects and examples of the use of compounds of the present invention and pharmaceutical compositions of the present invention are provided in the following listing of enumerated embodiments. It will be recognized that features specified in each embodiment may be combined with other specified features to provide further embodiments of the present invention.

### Embodiment 182

A compound of any one of Embodiments 1 to 172, or a pharmaceutically acceptable salt thereof, for use in the treatment of a CFTR mediated disease which is selected cystic fibrosis, asthma, COPD, chronic bronchitis and emphysema.

### Embodiment 183

A compound of any one of Embodiments 1 to 172, or a pharmaceutically acceptable salt thereof, for use in the treatment of a CFTR mediated disease which is selected from cystic fibrosis, asthma, COPD and chronic bronchitis.

### Embodiment 184

A compound of any one of Embodiments 1 to 172, or a pharmaceutically acceptable salt thereof, for use in the treatment of a CFTR mediated disease which is selected from cystic fibrosis, COPD and emphysema.

### Embodiment 185

A compound of any one of Embodiments 1 to 172, or a pharmaceutically acceptable salt thereof, for use in the treatment of a CFTR mediated disease which is cystic fibrosis.

### Embodiment 186

A compound of any one of Embodiments 1 to 172, or a pharmaceutically acceptable salt thereof, for use in the treatment of pancreatitis.

### Embodiment 187

A method of treating a CFTR mediated disease in a subject comprising administering to the subject a compound of any one of Embodiments 1 to 172, or a pharmaceutically acceptable salt thereof, or administering a pharmaceutical composition of any one of Embodiments 173 to 181.

### Embodiment 188

A method of treating a CFTR mediated disease in a subject comprising administering to the subject a therapeutically effective amount of a compound of any one of Embodiments 1 to 172, or a pharmaceutically acceptable salt thereof, or administering a pharmaceutical composition of any one of Embodiments 173 to 181.

### Embodiment 189

The method of Embodiments 187 or 188, wherein the CFTR mediated disease is selected from cystic fibrosis, asthma, COPD and chronic bronchitis.

### Embodiment 190

The method of Embodiments 187 or 188, wherein the CFTR mediated disease is selected from cystic fibrosis, COPD and emphysema.

### Embodiment 191

The method of Embodiments 187 or 188, wherein the CFTR mediated disease is cystic fibrosis.

### Embodiment 192

A method of treating pancreatitis in a subject comprising administering to the subject a therapeutically effective amount of a compound of any one of Embodiments 1 to 172, or a pharmaceutically acceptable salt thereof, or administering a pharmaceutical composition of any one of Embodiments 173 to 181.

### Embodiment 193

The method of any one of Embodiments 187 to 192, further comprising administering to the subject one or more additional pharmaceutical agent(s) prior to, concurrent with, or subsequent to the administration of a compound of any one of Embodiments 1 to 172 or the pharmaceutical composition of any one of Embodiments 173 to 181.

### Embodiment 194

The method of Embodiment 193, wherein additional pharmaceutical agent(s) is selected from a mucolytic agent, nebulized hypertonic saline, bronchodilator, an antibiotic, an anti-infective agent, a CFTR modulator, and an anti-inflammatory agent.

### Embodiment 195

The method of Embodiment 191, wherein the additional pharmaceutical agent(s) is selected from a CFTR modulator.

### Embodiment 196

The method of Embodiment 193, wherein the additional pharmaceutical agent(s) is selected from a CFTR potentiator.

### Embodiment 197

The method of Embodiment 193, wherein the additional pharmaceutical agent(s) is selected from a CFTR modulator and a CFTR potentiator.

### Embodiment 198

The method of Embodiment 193, wherein the additional pharmaceutical agent(s) is selected from a CFTR corrector.

### Embodiment 199

The method of Embodiment 193, wherein the additional pharmaceutical agent(s) is selected from a CFTR corrector and a CFTR potentiator.

### Embodiment 200

The use of a compound of any one of Embodiments 1 to 172, or pharmaceutically acceptable salt thereof, in the manufacture of a medicament for treating a disease in which CFTR modulation contributes to the pathology and/or symptomology of a disease.

### Embodiment 201

The use of a compound of any one of Embodiments 1 to 172, or pharmaceutically acceptable salt thereof, in the manufacture of a medicament for treating pancreatitis.

### Combination Therapy

In certain instances, it may be advantageous to administer a compound of the present invention in combination with, before, or after, one or more additional therapeutic agent(s). The compound of the present invention may be administered separately, by the same or different route of administration, or together in the same pharmaceutical composition as the other therapeutic agents. A therapeutic agent is, for example, a chemical compound, peptide, antibody, antibody fragment or nucleic acid, which is therapeutically active or enhances the therapeutic activity when administered to a patient in combination with a compound of the present invention.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by CFTR. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by CFTR, wherein the medicament is prepared for administration with another therapeutic agent. The invention also provides the use of another therapeutic agent for treating a disease or condition mediated by CFTR, wherein the medicament is administered with a compound of the present invention.

The invention also provides a compound of the present invention for use in a method of treating a disease or condition mediated by CFTR, wherein the compound of the present invention is prepared for administration with another therapeutic agent. The invention also provides another therapeutic agent for use in a method of treating a disease or condition mediated by CFTR, wherein the other therapeutic agent is prepared for administration with a compound of the present invention. The invention also provides a compound of the present invention for use in a method of treating a disease or condition mediated by CFTR, wherein the compound of the present invention is administered with another therapeutic agent. The invention also provides another therapeutic agent for use in a method of treating a disease or condition mediated by CFTR, wherein the other therapeutic agent is administered with a compound of the present invention.

The invention also provides the use of a compound of the present invention for treating a disease or condition mediated by CFTR, wherein the patient has previously (e.g. within 24 hours) been treated with another therapeutic agent. The invention also provides the use of another therapeutic agent for treating a disease or condition mediated by CFTR, wherein the patient has previously (e.g. within 24 hours) been treated with a compound of the present invention.

In one embodiment, the other therapeutic agent is selected from osmotic agents, ion channel modulating agents, mucolytic agents, bronchodilators, antihistamines, antibiotics, anti-inflammatory agents and CFTR modulators.

In another embodiment the other therapeutic agent is an osmotic agent, for example, nebulized hypertonic saline, dextran, mannitol or Xylitol.

In another embodiment the other therapeutic agent is a mucolytic agent, for example, Pulmozyme™.

In another embodiment, the other therapeutic agent is a bronchodilator, for example, albuterol, metaprotenerol sulfate, pirbuterol acetate, salmeterol, indacaterol or tetrabuline sulfate; suitable bronchodilatory agents also include anticholinergic and antimuscarinic agents, in particular, ipratropium bromide, oxitropium bromide, glycopyrronium saltsor tiotropium salts.

In another embodiment, the other therapeutic agent is an antihistamine, for example, cetirizine hydrochloride, clemastine fumarate, promethazine, loratidine, desloratidine, diphenhydramine fexofenadine hydrochloride, activastine, astemizole, azelastine, ebastine, epinastine, mizolastine or tefenadine

In another embodiment the other therapeutic agent is an antibiotic, for example tobramycin, including tobramycin inhaled powder, azithromycin, cayston, aztreonam, including the aerosolized for of aztreonam, amikacin, including liposomal formulations thereof, ciprofloxacin, including formulations thereof suitable of administration by inhalation, levofloxacin, including aerosolized formulations thereof and combinations of two antibiotics, for example, fosfomycin and tobramycin.

In another embodiment the other therapeutic agent is an anti-inflammatory agent, for example ibuprofen, docosahexanoic acid, sildenafil, inhaled glutathione, pioglitazone, hydroxychloroquine or simavastatin; a steroid, for example, glucocorticosteroids, such as budesonide, beclamethasone dipropionate, fluticasone propionate, ciclesonide or mometasone furoate; an LTD4 antagonist, such as montelukast or zafirlukast; a PDE4 inhibitor, such as Enprofylline, Theophylline, Roflumilaste, Ariflo (Cilomilaste), Tofimilaste, Pumafentrine, Lirimilaste, Apremilaste, Arofylline, Atizorame, Oglemilasturn, or Tetomilaste.

In another embodiment the other therapeutic agent is a CFTR modulator. In another embodiment the other therapeutic agent is a CFTR potentiator. In another embodiment the other therapeutic agent is a CFTR corrector. Exemplary CFTR modulators include N-(2-(5-chloro-2-methoxy-phenylamino)-4′-methyl-[4, 5′]bithiazolyl-2′-yl)-benzamide (Corr-4a), N-(2,4-di-tert-butyl-5-hydroxyphenyl)-1,4-dihydro-4-oxoquinoline-3-carboxamide (Ivacaftor), N-[2-(1,1-Dimethylethyl)-4-[1,1-di(methyl-da)ethyl-2,2,2-d]-5-hydroxyphenyl]-1,4-dihydro-4-oxo-3-quinolinecarboxamide (CTP-656), (((3-((3-carbamoyl-5,5,7,7-tetramethyl-4,7-dihydro-5H-thieno[2,3-c]pyran-2-yl)carbamoyl)-1H-pyrazol-1-yl)methoxy)methyl)phosphonic acid (GLPG1833), 3-[6-({[1-(2,2-difluoro-1,3-benzodioxol-5-yl)cyclopropyl]carbonyl}amino)-3-methylpyridin-2-yl]benzoic acid (Lumacaftor), N-(3-carbamoyl-5,5,7,7-tetramethyl-4,7-dihydro-5H-thieno[2,3-c]pyran-2-yl)-1H-pyrazole-3-carboxamide, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide (VX-661, Tezacaftor), 4-((2R,4R)-4-(1-(2,2-difluorobenzo[d][1,3]dioxol-5-yl)cyclopropane-1-carboxamido)-7-(difluoromethoxy)chroman-2-yl)benzoic acid (GLPG2222), 4-(3-(1-(2,2-difluorobenzo[d][1,3]dioxol-5-yl)cyclopropane-1-carboxamido)isoquinolin-1-yl)benzoic acid, N-(4-(7-azabicyclo[2.2.1]heptan-7-yl)-2-(trifluoromethyl)phenyl)-4-oxo-5-(trifluromethyl)-1,4-dihydroquinoline-3-carboxamide, 3-[5-(2-fluorophenyl)-1,2,4-oxadiazol-3-yl]benzoic acid (Ataluren), 5,7-Dihydroxy-3-(4-hydroxyphenyl)chromen-4-one (Genistein), N-(2-(tert-butyl)-5-hydroxy-4-(2-(methyl-d₃)propan-2-yl-1,1,1,3,3,3-d₆)phenyl)-4-oxo-1,4-dihydroquinoline-3-carboxamide (CTP-656), N-(3-carbamoyl-5,5,7,7-tetramethyl-4,7-dihydro-5H-thieno[2,3-c]pyran-2-yl)-1H-pyrazole-5-carboxamide (GLPG1837), 3-Chloro-4-(6-hydroxyquinolin-2-yl)benzoic acid (N-91115) and (S)-3-amino-6-methoxy-N-(3,3,3-trifluoro-2-hydroxy-2-methylpropyl)-5-(trifluoromethyl)picolinamide.

In one embodiment of the invention, there is provided a product comprising a compound of the present invention or a pharmaceutically acceptable salt thereof and a CFTR modulator as a combined preparation for simultaneous, separate or sequential use in therapy. In another embodiment, there is provided a product comprising a compound of the present invention and a CFTR potentiator as a combined preparation for simultaneous, separate or sequential use in therapy. In another embodiment there is provided a product comprising a compound of the present invention, a CFTR potentiator and a CFTR corrector as a combined preparation for simultaneous, separate or sequential use in therapy.

Certain aspects and examples of the combinations and combination therapy of the present invention are provided in the following listing of additional, enumerated embodiments. It will be recognized that features specified in each embodiment may be combined with other specified features to provide further embodiments of the present invention.

### Embodiment 202

A product comprising a compound of any one of Embodiments 1 to 172, or pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 203

A product comprising 4-(isobutyl(6-(N-(6-(o-tolyl)-5-(trifluoromethyl)pyridin-2-yl)sulfamoyl)pyridin-2-yl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 204

A product comprising 4-((6-(N-(6-(5-fluoro-2-methylphenyl)-5-(trifluoromethyl)pyridin-2-yl)sulfamoyl)pyridin-2-yl)(propyl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 205

A product comprising 3-((cyclopropylmethyl)(6-(N-(6-(o-tolyl)-5-(trifluoromethyl)pyridin-2-yl)sulfamoyl)pyridin-2-yl)amino)propanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 206

A product comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 207

A product comprising a compound of any one of Embodiments 1 to 172, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 208

A product comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 209

A product comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 210

A product comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 211

A product comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 212

A product comprising a compound of any one of Embodiments 1 to 172, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

### Embodiment 213

A product comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

### Embodiment 214

A product comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

### Embodiment 215

A product comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

### Embodiment 216

A product comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

### Embodiment 217

A product comprising a compound of any one of Embodiments 1 to 172, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

### Embodiment 218

A product comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

### Embodiment 219

A product comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

### Embodiment 220

A product comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

### Embodiment 221

A product comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

### Embodiment 222

A pharmaceutical composition comprising a compound of any one of Embodiments 1 to 172, or a pharmaceutically acceptable salt thereof, a CFTR modulator and a pharmaceutically acceptable carrier.

### Embodiment 223

A pharmaceutical composition comprising a compound of any one of Embodiments 1 to 172, or a pharmaceutically acceptable salt thereof, a CFTR potentiator and a pharmaceutically acceptable carrier.

### Embodiment 224

A pharmaceutical composition comprising a compound of any one of Embodiments 1 to 172, or a pharmaceutically acceptable salt thereof, a CFTR corrector and a pharmaceutically acceptable carrier.

### Embodiment 225

A pharmaceutical composition comprising a compound of any one of Embodiments 1 to 172, or pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 226

A pharmaceutical composition comprising 4-(isobutyl(6-(N-(6-(o-tolyl)-5-(trifluoromethyl)pyridin-2-yl)sulfamoyl)pyridin-2-yl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 227

A pharmaceutical composition comprising 4-((6-(N-(6-(5-fluoro-2-methylphenyl)-5-(trifluoromethyl)pyridin-2-yl)sulfamoyl)pyridin-2-yl)(propyl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 228

A pharmaceutical composition comprising 3-((cyclopropylmethyl)(6-(N-(6-(o-tolyl)-5-(trifluoromethyl)pyridin-2-yl)sulfamoyl)pyridin-2-yl)amino)propanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 229

A pharmaceutical composition comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 230

A pharmaceutical composition comprising a compound of any one of Embodiments 1 to 172, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 231

A pharmaceutical composition comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 232

A pharmaceutical composition comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 233

A pharmaceutical composition comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 234

A pharmaceutical composition comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 235

A pharmaceutical composition comprising a compound of any one of Embodiments 1 to 172, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

### Embodiment 236

A pharmaceutical composition comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

### Embodiment 237

A pharmaceutical composition comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

### Embodiment 238

A pharmaceutical composition comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

### Embodiment 239

A pharmaceutical composition comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

### Embodiment 240

A pharmaceutical composition comprising a compound of any one of Embodiments 1 to 172, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

### Embodiment 241

A pharmaceutical composition comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

### Embodiment 242

A pharmaceutical composition comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

### Embodiment 243

A pharmaceutical composition comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

### Embodiment 244

A pharmaceutical composition comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

### Embodiment 245

A combination comprising a compound of any one of Embodiments 1 to 172, or pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 246

A combination comprising 4-(isobutyl(6-(N-(6-(o-tolyl)-5-(trifluoromethyl)pyridin-2-yl)sulfamoyl)pyridin-2-yl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 247

A combination comprising 4-((6-(N-(6-(5-fluoro-2-methylphenyl)-5-(trifluoromethyl)pyridin-2-yl)sulfamoyl)pyridin-2-yl)(propyl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 248

A combination comprising 3-((cyclopropylmethyl)(6-(N-(6-(o-tolyl)-5-(trifluoromethyl)pyridin-2-yl)sulfamoyl)pyridin-2-yl)amino)propanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 249

A combination comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 250

A combination comprising a compound of any one of Embodiments 1 to 172, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 251

A combination comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 252

A combination comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 253

A combination comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 254

A combination comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 255

A combination comprising a compound of any one of Embodiments 1 to 172, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier, wherein the combination is a fixed combination or a non-fixed combination.

### Embodiment 256

A combination comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier, wherein the combination is a fixed combination or a non-fixed combination.

### Embodiment 257

A combination comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier, wherein the combination is a fixed combination or a non-fixed combination.

### Embodiment 258

A combination comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier, wherein the combination is a fixed combination or a non-fixed combination.

### Embodiment 259

A combination comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier, wherein the combination is a fixed combination or a non-fixed combination.

### Embodiment 260

A combination comprising a compound of any one of Embodiments 1 to 172, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier, wherein the combination is a fixed combination or a non-fixed combination.

### Embodiment 261

A combination comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier, wherein the combination is a fixed combination or a non-fixed combination.

### Embodiment 262

A combination comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier, wherein the combination is a fixed combination or a non-fixed combination.

### Embodiment 263

A combination comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier, wherein the combination is a fixed combination or a non-fixed combination.

### Embodiment 264

A combination comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier, wherein the combination is a fixed combination or a non-fixed combination.

### Embodiment 265

A product comprising a compound of any one of Embodiments 1 to 172, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 266

A product comprising 4-(isobutyl(6-(N-(6-(o-tolyl)-5-(trifluoromethyl)pyridin-2-yl)sulfamoyl)pyridin-2-yl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 267

A product comprising 4-((6-(N-(6-(5-fluoro-2-methylphenyl)-5-(trifluoromethyl)pyridin-2-yl)sulfamoyl)pyridin-2-yl)(propyl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 268

A product comprising 3-((cyclopropylmethyl)(6-(N-(6-(o-tolyl)-5-(trifluoromethyl)pyridin-2-yl)sulfamoyl)pyridin-2-yl)amino)propanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 269

A product comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

### Embodiment 270

A product comprising a compound of any one of Embodiments 1 to 172, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 271

A product comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 272

A product comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 273

A product comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 274

A product comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

### Embodiment 275

A pharmaceutical composition comprising a compound of any one of Embodiments 1 to 172, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 276

A pharmaceutical composition comprising 4-(isobutyl(6-(N-(6-(o-tolyl)-5-(trifluoromethyl)pyridin-2-yl)sulfamoyl)pyridin-2-yl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 277

A pharmaceutical composition comprising 4-((6-(N-(6-(5-fluoro-2-methylphenyl)-5-(trifluoromethyl)pyridin-2-yl)sulfamoyl)pyridin-2-yl)(propyl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 278

A pharmaceutical composition comprising 3-((cyclopropylmethyl)(6-(N-(6-(o-tolyl)-5-(trifluoromethyl)pyridin-2-yl)sulfamoyl)pyridin-2-yl)amino)propanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 279

A pharmaceutical composition comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

### Embodiment 280

A pharmaceutical composition comprising a compound of any one of Embodiments 1 to 172, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 281

A pharmaceutical composition comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 282

A pharmaceutical composition comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 283

A pharmaceutical composition comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 284

A pharmaceutical composition comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

### Embodiment 285

A combination comprising a compound of any one of Embodiments 1 to 172, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 286

A combination comprising 4-(isobutyl(6-(N-(6-(o-tolyl)-5-(trifluoromethyl)pyridin-2-yl)sulfamoyl)pyridin-2-yl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 287

A combination comprising 4-((6-(N-(6-(5-fluoro-2-methylphenyl)-5-(trifluoromethyl)pyridin-2-yl)sulfamoyl)pyridin-2-yl)(propyl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 288

A combination comprising 3-((cyclopropylmethyl)(6-(N-(6-(o-tolyl)-5-(trifluoromethyl)pyridin-2-yl)sulfamoyl)pyridin-2-yl)amino)propanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 289

A combination comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier, wherein the combination is a fixed combination or a non-fixed combination.

### Embodiment 290

A combination comprising a compound of any one of Embodiments 1 to 172, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 291

A combination comprising 4-(isobutyl(6-(N-(6-(o-tolyl)-5-(trifluoromethyl)pyridin-2-yl)sulfamoyl)pyridin-2-yl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 292

A combination comprising 4-((6-(N-(6-(5-fluoro-2-methylphenyl)-5-(trifluoromethyl)pyridin-2-yl)sulfamoyl)pyridin-2-yl)(propyl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 293

A combination comprising 3-((cyclopropylmethyl)(6-(N-(6-(o-tolyl)-5-(trifluoromethyl)pyridin-2-yl)sulfamoyl)pyridin-2-yl)amino)propanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 294

A combination comprising 4-((6-(N-(6-(5-fluoro-2-methylphenyl)-5-(trifluoromethyl)pyridin-2-yl)sulfamoyl)pyridin-2-yl)(isopropyl)amino)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier, wherein the combination is a fixed combination or a non-fixed combination.

### Embodiment 296

The product of any one of Embodiments 202-221 or Embodiments 265-274 for use in treating a disease, disorder, or condition associated with the modulation of CFTR.

### Embodiment 297

The product of any one of Embodiments 202-221 or Embodiments 265-274 for use in the treatment of cystic fibrosis, asthma, COPD, chronic bronchitis, pancreatitis or emphysema.

### Embodiment 298

The product of any one of Embodiments 202-221 or Embodiments 265-274 for use in the treatment of pancreatitis.

### Embodiment 299

The pharmaceutical composition of any one of Embodiments 222-264 or Embodiments 275-284 for use in treating a disease, disorder, or condition associated with the modulation of CFTR.

### Embodiment 300

The pharmaceutical composition of any one of Embodiments 222-264 or Embodiments 275-284 for use in the treatment of cystic fibrosis, asthma, COPD, chronic bronchitis, pancreatitis or emphysema.

### Embodiment 301

The pharmaceutical composition of any one of Embodiments 222-264 or Embodiments 275-284 for use in the treatment of pancreatitis.

### Embodiment 302

The combination of any one of Embodiments 245-264 or Embodiments 285-294 for use in treating a disease, disorder, or condition associated with the modulation of CFTR.

### Embodiment 303

The combination of any one of Embodiments 245-264 or Embodiments 285-294 for use in the treatment of cystic fibrosis, asthma, COPD, chronic bronchitis, pancreatitis or emphysema.

### Embodiment 304

The combination of any one of Embodiments 245-264 or Embodiments 285-294 for use in the treatment of pancreatitis.

### Biological Assays

Measurement of delF508-CFTR-HRP Surface Expression in CFBE41o-Cells

This assay quantifies the cell surface expressions of the mutant CFTR channel using an extracellular HRP tag.

A cellular assay was developed to measure surface expression of horseradish peroxidase (HRP) tagged delF508-CFTR in the human bronchial epithelial immortalized CFBE41o-cell line (Phuan, P. W., et al, (2014) Molecular Pharmacology 86:42-51). Specifically, the HRP sequence was inserted into the fourth extracellular loop of delF508-CFTR and stably expressed in CFBE41o-cells. Cells were seeded in 384 well plate at a density of 5000 cells/well and incubated at 37° C. for 12 to 24 hours in medium (Gibco MEM #11095, 10% FBS, 10 mM HEPES, 200 mM L-Glutamine, 200 μg/mL G418, 3 μg/mL Puromycin). The delF508-CFTR-HRP expression was induced with 500 ng/mL doxycycline (Sigma D-9891, dissolved in H₂O and sterile filtered) in medium and the cells were incubated at 37° C. for 48 h. Old medium was removed and fresh medium was added containing 500 ng/mL doxycycline and unknown test compound at required test concentration in DMSO, not exceeding 0.5% final DMSO concentration. The highest concentration tested was 10 μM with a 10-point concentration response curve using a 3-fold dilution. After addition of compounds, the cells were incubated for 24 h at 37° C. On the final day, cells were washed four times in PBS containing 1 mM MgCl₂ and 0.1 mM CaCl₂. HRP-Substrate (SuperSignal ELISA Pico, Fisher #37069) 20 μl/well was added and the luminescence signal was determined (Viewlux, Perkin Elmer). Light was emitted upon addition of exogenous HRP-Substrate only when delF508-CFTR-HRP reached the cell surface and the HRP tag was accessible to the HRP-Substrate (note: HRP-Substrate cannot cross the lipid bilayer to reach delF508-CFTR-HRP misfolded within the cell).

The median activity for the lowest concentration of the compounds on each assay plate was calculated and this value was used to normalize the signal for each well on the respective plate. Three replicates at each concentrations for every compound were run to determine one EC₅₀. The median value was determined and used to calculate compound activities as described below. Effective half maximal values (EC₅₀) were calculated for each compound by performing logistic regression on measured dose-response data points using the equation:

$$Y = Bottom + \frac{Top - Bottom}{1 + {( \frac{X}{{EC}_{50}} )}^{Hillcoefficient}}$$

where “Y” is the observed activity, “Bottom” is the lowest observed value, “Top” is the highest observed value, and the “Hill coefficient” gives the largest absolute value of the slope. The curve fitting is carried out by a curve fitting program implemented at GNF using Matlab (Mathworks).

The dose response curves also were used to calculate Fold Change (FC) using the equation:

$$Fold change = \frac{Top - Bottom}{Bottom}$$

Compound efficacy relative to the reference compound 3-(6-(1-(2,2-difluorobenzo[d][1,3]dioxol-5-yl)cyclopropane-1-carboxamido)-3-methylpyridin-2-yl)benzoic acid was determined using the following formula:

$$\% Amax = \frac{FC of test compound}{FC of reference compound} * 100$$

Measurement of delF508-CFTR Functional Activity in Primary Human Bronchial Epithelial Cells (HBECs) Using Multi-Transepithelial Clamp Circuit (MTECC-24) Assay

This assay measures the functional activity of the CFTR channel (Chloride ion transport) in patient derived primary human bronchial epithelial cells with forskolin activation and in the presence of the CFTR corrector/potentiator combination.

Primary human delF508-CFTR bronchial epithelial cells were and cultured according to previously established methods (Neuberger, T. et, al., Methods in Molecular Biology, 2011, 741, pp 39-54). Briefly, before thawing the HBEC cells, T25 flasks were coated with 2 mL of 3T3 conditioned media (MTI-GlobalStem, Cat #GSM9100) for at least 12 hours in 37° C. CO₂ incubator. 1.7×10⁵ cells were seeded into one T25 flask with 5 mL of HBE growth media (BEBM with supplements (Lonza, Cat #CC-3170) with 10 nM of retinoic acid (Sigma, Cat #R-2625) and 1% of PSA (Hyclone, Cat #SV30079.01). Media was changed every day till the cells were 100% confluent. Cells were seeded into T75 flasks (pre-coated with 5 mL of 3T3 conditioned media for more than 12 hrs) at 4.95×10⁵ cells/T75 flask in 15 mL of HBE growth media. Flasks were fed everyday with fresh HBE growth media until the cells were confluent. 24-well transwell plates (Corning, Cat #3378) were coated with 3T3 conditioned media (70 μL on top of the filter, 350 μL to the bottom of the well) overnight in the 37° C. CO₂ incubator. Cells were seeded in the pre-coated 24-well transwell plates at 1.7×10⁵ cell/well with 700 μL of growth media at the bottom of the well and 200 μL of growth media at the top of the filter. After 24 hrs, cells were switched into HBE differentiation media ((DMEM/F12 (Gibco, Cat #11330-032) supplement with 2% Ultroser G (Pall, Cat #15950-017), 2% of Fetal Clone II (Hyclone, Cat #SH30066.03), 0.25% Bovine Brain Extract (Lonza, Cat #CC-4098), 1% of PSA, 2.5 μg/mL of Insulin (Sigma, Cat #19278), 20 nM of Hydrocortisone (Sigma, Cat #H0888), 500 nM of Triiodothyronine (Sigma, Cat #T6397), 2.5 μg/mL of Transferrin (Sigma, Cat #T8158), 250 nM of Ethanolamine (Sigma, Cat #E0135), 1.5 μM of Epinephrine (Sigma, Cat #E4250), 250 nM of Phosphoethanolamine (Sigma, Cat #P0503), 10 nM of retinoic acid)) with 200 μL on top of the filter (apical side) and 700 μL at the bottom of the well (basolateral side). Cells were fed every other day with the differentiation media. After 4 days, cells were exposed to air/liquid interface. Then cells were fed every other day for 4 weeks before fully differentiated.

Cells were washed with 3 mM DTT in PBS at 70 ul/well for 30 min at 37° C. 6 days before the assay. 3 days before the assay, cells were washed again with PBS at 70 ul/well for 30 min at 37° C. Then cells were treated with compound for 24 hrs before the assay.

Following 24 hr treatment with compound, cells were transferred into plates containing 750 ul of assay media on basolateral side and 250 ul on apical side (Assay medium: F-12 Coon's modified, 20 mM HEPES pH7.4 with TRIS Base, No FCS or bicarbonate). The plates wee then transferred to the heated plate compartments (basolateral buffer temperature ˜36.5° C. on the heating block) of the TECC24 system for 45 mins prior to measurements.

Modulators were added sequentially as follows while the TECC24 instrument recorded the equivalent short circuit current (Ieq):

| | | Stock | Approx. |
|---|---|---|---|
| | | (in | incuba- |
| | | Assay | tion |
| Final | Added to plate | medium) | time |
| 3 μM Benzamil | 25 μL Apical | 33 μM | 15 min |
| 10 μM Forskolin | 25 μL Apical | 120 μM | 15 min |
| | 75 μL Basolateral | | |
| 0.5 μM N-(2,4-di-tert-butyl-5- | 25 μL Apical/ | 6.5 μM | 15 min |
| hydroxyphenyl)-1,4-dihydro-4- | 75 μL Basolateral | | |
| oxoquinoline-3-carboxamide | | | |
| or | | | |
| 0.5 μM (S)-3-amino-6-methoxy- | | | |
| N-(3,3,3-trifluoro-2-hydroxy-2- | | | |
| methylpropyl)-5- | | | |
| (trifluoromethyl)picolinamide | | | |
| 20 μM Bumetanide | 25 μL Apical/ | 280 μM | 30 min |
| | 75 μL Basolateral | | |

Prior to dilution into assay medium the stocks were as follows:
Benzamil stock: 10 mM in DMSO
Forskolin Stock: 50 mM in DMSO
N-(2,4-di-tert-butyl-5-hydroxyphenyl)-1,4-dihydro-4-oxoquinoline-3-carboxamide: 50 mM in DMSO
(S)-3-amino-6-methoxy-N-(3,3,3-trifluoro-2-hydroxy-2-methylpropyl)-5-(trifluoromethyl)picolinamide: 50 mM stock 100% DMSO
Bumetanide stock: 20 mM in EtOH

The data was normalized using the median signal from wells treated with 0.1% DMSO as a baseline. Curve fitting and EC₅₀ calculations were performed using the following equation:

$$Y = Bottom + \frac{Top - Bottom}{1 + {( \frac{X}{{EC}_{50}} )}^{Hillcoefficient}}$$

where “Y” is the observed activity, “Bottom” is the lowest observed value, “Top” is the highest observed value, and the “Hill coefficient” gives the largest absolute value of the slope. The curve fitting is carried out by a curve fitting program implemented at GNF using Matlab (Mathworks). At least two replicates for every compound were run and EC₅₀ reported in the table are mean values.

The dose response curves also were used to calculate Fold Change (FC) using the equation:

$$Fold change = \frac{Top - Bottom}{Bottom}$$

% Amax calculations were performed using the equation:

$$\% Amax = \frac{FC of test compound}{FC of reference compound} * 100 \%$$

where the test compound (added 24 h before assay) was in the presence of the potentiator (S)-3-amino-6-methoxy-N-(3,3,3-trifluoro-2-hydroxy-2-methylpropyl)-5-(trifluoromethyl)picolinamide at the time of assay. The reference compound was a combination of 2 μM 3-(6-(1-(2,2-difluorobenzo[d][1,3]dioxol-5-yl)cyclopropane-1-carboxamido)-3-methylpyridin-2-yl)benzoic acid added 24 h prior to assay and 0.5 μM N-(2,4-di-tert-butyl-5-hydroxyphenyl)-4-oxo-1,4-dihydroquinoline-3-carboxamide added at the time of assay.

TABLE 1
Activity Table
CFTR-Linear sub series-shortened list

| | DelF508- | DelF508- | MTECC24- | MTECC24- |
|---|---|---|---|---|
| Compound | CFTR-HRP | CFTR-HRP | CFHBEC | CFHBEC |
| No. | EC₅₀ (μM) | Amax % | EC₅₀ (μM) | Amax % |
| 1 | 2.15 | 291 | n.d. | n.d. |
| 2 | 2.72 | 124 | n.d. | n.d. |
| 3 | 2.58 | 722 | n.d. | n.d. |
| 4 | 2.19 | 1151 | n.d. | n.d. |
| 5 | 2.58 | 596 | n.d. | n.d. |
| 6 | 1.52 | 529 | n.d. | n.d. |
| 7 | 1.88 | 363 | n.d. | n.d. |
| 8 | 1.85 | 271 | n.d. | n.d. |
| 9 | 2.02 | 102 | n.d. | n.d. |
| 10 | 0.91 | 266 | n.d. | n.d. |
| 11 | 2.25 | 415 | n.d. | n.d. |
| 12 | 2.30 | 608 | n.d. | n.d. |
| 13 | 2.40 | 370 | n.d. | n.d. |
| 14 | 2.55 | 283 | n.d. | n.d. |
| 15 | 2.14 | 397 | n.d. | n.d. |
| 16 | 2.01 | 495 | n.d. | n.d. |
| 17 | 1.46 | 369 | n.d. | n.d. |
| 18 | 2.39 | 97 | n.d. | n.d. |
| 19 | 2.24 | 378 | n.d. | n.d. |
| 20 | 0.80 | 973 | n.d. | n.d. |
| 21 | 2.93 | 361 | n.d. | n.d. |
| 22 | 1.06 | 485 | n.d. | n.d. |
| 23 | 1.26 | 1072 | n.d. | n.d. |
| 24 | 1.31 | 909 | n.d. | n.d. |
| 25 | 1.00 | 780 | n.d. | n.d. |
| 26 | 0.83 | 829 | n.d. | n.d. |
| 27 | 2.07 | 189 | n.d. | n.d. |
| 28 | 1.45 | 442 | n.d. | n.d. |
| 29 | 1.30 | 969 | n.d. | n.d. |
| 30 | 2.22 | 303 | n.d. | n.d. |
| 31 | 2.14 | 443 | n.d. | n.d. |
| 32 | 1.80 | 372 | n.d. | n.d. |
| 33 | 1.28 | 320 | n.d. | n.d. |
| 34 | 2.58 | 785 | n.d. | n.d. |
| 35 | 2.60 | 209 | n.d. | n.d. |
| 36 | 2.96 | 222 | n.d. | n.d. |
| 37 | 2.05 | 982 | n.d. | n.d. |
| 38 | 1.70 | 742 | n.d. | n.d. |
| 39 | 3.26 | 257 | n.d. | n.d. |
| 40 | 2.53 | 314 | n.d. | n.d. |
| 41 | 1.48 | 1307 | n.d. | n.d. |
| 42 | 2.67 | 507 | n.d. | n.d. |
| 43 | 2.37 | 407 | n.d. | n.d. |
| 44 | 1.52 | 1025 | n.d. | n.d. |
| 45 | 1.57 | 598 | n.d. | n.d. |
| 46 | 1.81 | 493 | n.d. | n.d. |
| 47 | 1.82 | 496 | n.d. | n.d. |
| 48 | 2.04 | 513 | n.d. | n.d. |
| 49 | 2.03 | 557 | n.d. | n.d. |
| 50 | 2.10 | 867 | n.d. | n.d. |
| 51 | 1.60 | 1115 | n.d. | n.d. |
| 52 | 1.49 | 1270 | n.d. | n.d. |
| 53 | 1.78 | 411 | n.d. | n.d. |
| 54 | 2.00 | 369 | n.d. | n.d. |
| 55 | 1.68 | 457 | n.d. | n.d. |
| 56 | 2.17 | 906 | 0.373 | 131 |
| 57 | 1.97 | 1039 | n.d. | n.d. |
| 58 | 2.37 | 1312 | n.d. | n.d. |
| 59 | 2.07 | 920 | n.d. | n.d. |
| 60 | 1.91 | 997 | 0.065 | 128 |
| 61 | 3.56 | 736 | n.d. | n.d. |
| 62 | 1.22 | 2003 | 0.009 | 150 |
| 63 | 1.46 | 1996 | 0.017 | 149 |
| 64 | 1.33 | 1070 | 0.002 | 107 |
| 65 | 1.73 | 1565 | 0.031 | 155 |
| 66 | 1.60 | 1046 | 0.185 | 133 |
| 67 | 1.63 | 1023 | 0.161 | 168 |
| 68 | 1.35 | 996 | 0.423 | 116 |
| 69 | 1.76 | 1953 | n.d. | n.d. |
| 70 | 1.57 | 1987 | 0.068 | 126 |
| 71 | 2.36 | 1491 | 0.365 | 137 |
| 72 | 1.88 | 1522 | 0.116 | 118 |
| 73 | 1.39 | 2276 | n.d. | n.d. |
| 74 | 1.09 | 2132 | n.d. | n.d. |
| 75 | 2.00 | 1572 | n.d. | n.d. |
| 76 | 1.46 | 1216 | n.d. | n.d. |
| 77 | 1.45 | 945 | n.d. | n.d. |
| 78 | 1.41 | 1056 | n.d. | n.d. |
| 79 | 2.91 | 330 | n.d. | n.d. |
| 80 | 1.93 | 1453 | 0.105 | 95 |
| 81 | 1.77 | 1722 | 0.035 | 145 |
| 82 | 1.04 | 2647 | n.d. | n.d. |
| 83 | 1.45 | 1873 | n.d. | n.d. |
| 84 | 1.54 | 1296 | n.d. | n.d. |
| 85 | 1.41 | 1317 | 0.030 | 152 |
| 86 | 1.38 | 3273 | 0.061 | 253 |
| 87 | 1.38 | 1604 | 0.104 | 269 |
| 88 | 2.04 | 1189 | n.d. | n.d. |
| 89 | 0.97 | 2235 | n.d. | n.d. |
| 90 | 1.35 | 1560 | 0.005 | 135 |
| 91 | 1.67 | 1691 | n.d. | n.d. |
| 92 | 1.82 | 1151 | 0.394 | 123 |
| 93 | 1.43 | 1419 | 0.040 | 235 |
| 94 | 1.37 | 3343 | 0.026 | 208 |
| 95 | 1.68 | 4050 | 0.024 | 148 |
| 96 | 0.72 | 3640 | 0.002 | 202 |
| 97 | 1.36 | 3205 | 0.004 | 190 |
| 98 | 1.54 | 2117 | n.d. | n.d. |
| 99 | 1.33 | 2844 | n.d. | n.d. |
| 100 | 2.49 | 1191 | n.d. | n.d. |
| 101 | 1.59 | 1951 | n.d. | n.d. |
| 102 | 1.26 | 1889 | 0.131 | 298 |
| 103 | 1.43 | 2369 | 0.073 | 142 |
| 104 | 1.28 | 1908 | n.d. | n.d. |
| 105 | 1.81 | 361 | n.d. | n.d. |
| 106a | | | | |
| 106b | 1.95 | 392 | n.d. | n.d. |
| (racemic | | | | |
| mixture) | | | | |
| 107a | 2.07 | 763 | n.d. | n.d. |
| 107b | | | | |
| (racemic | | | | |
| mixture) | | | | |
| 108a | 2.07 | 569 | n.d. | n.d. |
| 108b | | | | |
| (racemic | | | | |
| mixture) | | | | |
| 109a | 2.32 | 700 | n.d. | n.d. |
| 109b | | | | |
| (racemic | | | | |
| mixture) | | | | |
| 110 | 1.03 | 2193 | 0.006 | 182 |
| 111 | 1.78 | 1535 | n.d. | n.d. |
| 112 | 1.57 | 1199 | n.d. | n.d. |
| 113 | 1.77 | 715 | n.d. | n.d. |
| 114 | 1.68 | 1576 | 0.101 | 211 |
| 115 | 1.51 | 2171 | 0.070 | 146 |
| 116 | 1.21 | 3373 | n.d. | n.d. |
| 117 | 2.43 | 196 | n.d. | n.d. |
| 118 | 1.85 | 563 | 0.270 | 151 |
| 119 | 1.41 | 4609 | n.d. | n.d. |
| 120 | 1.39 | 330 | n.d. | n.d. |
| 121 | 3.28 | 167 | n.d. | n.d. |
| 122 | 1.62 | 785 | n.d. | n.d. |
| 123 | 1.61 | 1284 | n.d. | n.d. |
| 124 | 1.73 | 297 | n.d. | n.d. |
| 125 | 1.36 | 643 | 0.422 | 114 |
| 126 | 0.97 | 1855 | 0.014 | 207 |
| 127 | 0.72 | 1859 | 0.021 | 247 |
| 128 | 1.11 | 1419 | 0.031 | 277 |
| 129 | 1.08 | 1568 | 0.154 | 83 |
| 130 | 2.06 | 403 | n.d. | n.d. |
| 131 | 1.38 | 2015 | 0.327 | 95 |
| 132 | 1.58 | 1914 | 0.095 | 232 |
| 133 | 1.92 | 547 | 0.901 | 108 |
| 134 | 1.37 | 2720 | 0.114 | 189 |
| 135 | 1.57 | 3052 | 0.065 | 292 |
| 136 | 2.31 | 952 | n.d. | n.d. |
| 137 | 2.03 | 1087 | n.d. | n.d. |
| 138 | 1.98 | 463 | n.d. | n.d. |
| 139 | 1.12 | 1033 | n.d. | n.d. |
| 140 | 2.92 | 209 | n.d. | n.d. |
| 141 | 3.94 | 291 | n.d. | n.d. |
| 142 | 4.46 | 84 | n.d. | n.d. |
| 143 | 2.39 | 2721 | n.d. | n.d. |
| 144 | 4.55 | 52 | n.d. | n.d. |
| 145 | 1.83 | 3054 | n.d. | n.d. |
| 146 | 2.50 | 60 | n.d. | n.d. |
| 147 | 1.15 | 802 | n.d. | n.d. |
| 148 | 1.45 | 1268 | 0.179 | 247 |
| 149 | 1.49 | 1650 | 0.088 | 133 |
| 150 | 1.67 | 2125 | n.d. | n.d. |
| 151 | 1.90 | 2679 | n.d. | n.d. |
| 152 | 0.78 | 641 | n.d. | n.d. |
| 153 | 0.82 | 770 | n.d. | n.d. |
| 154 | 0.93 | 686 | n.d. | n.d. |
| 155 | 0.67 | 699 | n.d. | n.d. |
| 156 | 0.92 | 549 | 0.230 | 158 |
| 157 | 0.77 | 589 | 0.586 | 59 |
| 158 | 1.81 | 839 | n.d. | n.d. |

n.d. indicates not determined

1. A compound, or pharmaceutically acceptable salt thereof, having the structure of Formula (I), 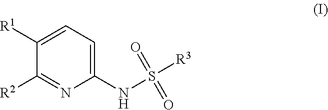
wherein:
R¹ is H, C₁₋C₆alkyl, halo, halo-substituted C₁₋C₆alkyl, deuterium substituted C₁₋C₆alkyl, C₁₋C₆alkoxy or halo-substituted C₁₋C₆alkoxy;
R² is selected from:
a) a phenyl substituted with 1 to 2 R⁴ groups;
b) an unsubstituted 5 to 6 membered heteroaryl having 1 to 3 heteroatoms independently selected from N, O and S,
and
c) a 5 to 6 membered heteroaryl having 1 to 3 heteroatoms independently selected from N, O and S, wherein the heteroaryl is substituted with 1 to 3 R⁴ groups;
R³ is a pyridin-2-yl or a pyridin-4-yl, wherein the pyridin-2-yl or a pyridin-4-yl is substituted with an R⁵ group;
each R⁴ is independently selected from D, C₁₋C₆alkyl, phenyl, phenoxy, halo, C₁₋C₆alkoxy, C₃₋C₈cycloalkyl, C₂₋C₆alkenyl, halo-substituted C₁₋C₆alkyl, deuterium substituted C₁₋C₆alkyl, halo-substituted C₁₋C₆alkoxy and a 5 to 6 membered heteroaryl having 1 to 3 heteroatoms independently selected from N, O and S;
R⁵ is selected from
a) —NR⁶R⁷;
b) —OR¹¹;
c) —S(CR⁸R⁹)ₙC(═O)OR¹⁰,
d) an unsubstituted 6 membered heterocycloalkyl having 1 to 2 ring members independently selected from N, O and S;
and
e) a 5 to 6 membered monocyclic heterocycloalkyl having 1 to 2 N heteroatoms as ring members, wherein the heterocycloalkyl is substituted with 1 to 2 R¹² groups;
R⁶ is H, —C₁₋C₆alkyl, halo-substituted C₁₋C₆alkyl, C₃₋C₈cycloalkyl, —(CR⁸R⁹)ₙOR¹⁴ or —(CR⁸R⁹)ₘR¹⁶;
R⁷ is H, —C₁₋C₆alkyl, —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, (CR¹⁴R¹⁵)ₘC(═O)OR¹⁰, —CHR¹²R¹⁸, a monocyclic C₃₋C₈cycloalkyl substituted with 1 to 2 R¹² groups, a bicyclic C₇₋C₁₀cycloalkenyl substituted with 1 to 2 R¹² groups or a bicyclic C₇₋C₁₀cycloalkyl substituted with 1 to 2 R¹² groups;
each R⁸ is independently selected from H, D, deuterium substituted C₁₋C₆alkyl and C₁₋C₆alkyl;
each R⁹ is independently selected from H, D, deuterium substituted C₁₋C₆alkyl and C₁₋C₆alkyl;
each R¹⁰ is independently selected from H, and C₁₋C₆alkyl;
R¹¹ is —(CR⁸R⁹)ₙC(═O)OR¹³ or a C₃₋C₈cycloalkyl substituted with 1 to 2 R¹² groups,
each R¹² is independently selected from C₁₋C₆alkyl, —OH, —(CR⁸R⁹)ₙC(═O)OR¹³, —C(═O)NH(CR¹⁴R¹⁵)ₘC(═O)OR¹³, —C(═O)OR¹³, (CR¹⁴R¹⁵)ₘC(═O)OR¹³, —O(CR⁸R⁹)˜C(═O)OR¹³, benzoic acid and tetrazolyl;
each R¹³ is independently selected from H, and C₁₋C₆alkyl;
each R¹⁴ is independently selected from H, D, deuterium substituted C₁₋₆alkyl and C₁₋₆alkyl;
each R¹⁵ is independently selected from H, D, deuterium substituted C₁₋C₆alkyl and C₁₋C₆alkyl;
or R¹⁴ and R¹⁵ together with the carbon in CR¹⁴R¹⁵ form a C₃₋C₈cycloalkyl;
R¹⁶ is a C₃₋C₈cycloalkyl;
R¹⁷ is
a) a C₃₋C₈cycloalkyl substituted with 1 to 2 R¹² groups,
or
b) a 5 to 6 membered monocyclic heterocycloalkyl having 1 to 2 ring members independently selected from N, O and S substituted with 1 to 2 R¹² groups;
R¹⁸ is adamantanyl;
each m is independently selected from 1, 2, 3, 4, 5, 6, 7, 8, 9 and 10;
and
each n is independently selected from 1, 2, 3, 4, 5, 6, 7, 8, 9 and 10.

2. The compound of claim 1, or pharmaceutically acceptable salt thereof, wherein R² is a phenyl substituted with 1 to 2 R⁴ groups, or R² is a 5 to 6 membered heteroaryl having 1 to 3 heteroatoms independently selected from N, O and S substituted with 1 to 3 R⁴ groups.

3. The compound of claim 1, or pharmaceutically acceptable salt thereof, wherein each R⁴ is independently selected from D, methyl, ethyl, propyl, isopropyl, butyl, tert-butyl, CD₃, phenyl, phenoxy, Cl, F, methoxy, cyclopropyl, cyclobutyl, ethenyl, pyrimidinyl and pyridyl.

4. The compound of claim 1, or pharmaceutically acceptable salt thereof, wherein each R⁴ is independently selected from D, C₁₋₆alkyl, halo, C₁₋₆alkoxy and C₂₋₆alkenyl.

5. The compound of claim 1, or a pharmaceutically acceptable salt thereof, wherein each R⁴ is independently selected from methyl, ethyl, isopropyl, tert-butyl, F and ethenyl.

6. The compound of claim 1, or pharmaceutically acceptable salt thereof, wherein:
R⁵ is selected from
a) —NR⁶R⁷;
b) —OR¹¹;
c) —S(CR⁸R⁹)ₙC(═O)OR¹⁰,
and
d) a 5 to 6 membered monocyclic heterocycloalkyl having 1 to 2 N heteroatoms as ring members, wherein the heterocycloalkyl is substituted with 1 to 2 R¹² groups;
R⁶ is H, —C₁₋₆alkyl, halo-substituted C₁₋₆alkyl, C₃₋₈cycloalkyl, —(CR⁸R⁹)ₙOR¹⁴, or —(CR⁸R⁹)ₘR¹⁶;
R⁷ is —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, —(CR¹⁴R¹⁵)ₘC(═O)OR¹⁰, —CHR¹²R¹⁸, a monocyclic C₃₋₈cycloalkyl substituted with 1 to 2 R¹² groups, a bicyclic C₇₋₁₀cycloalkenyl substituted with 1 to 2 R¹² groups or a bicyclic C₇₋₁₀cycloalkyl substituted with 1 to 2 R¹² groups;
each R⁸ is H;
each R⁹ is H;
each R¹⁰ is H or C₁₋₆alkyl;
R¹¹ is —(CR⁸R⁹)ₙC(═O)OR¹³;
each R¹² is independently selected from C₁₋₆alkyl, —OH, —(CR⁸R⁹)ₙC(═O)OR¹³, —C(═O)NH(CR¹⁴R¹⁵)ₘC(═O)OR¹³, —C(═O)OR¹³, —(CR¹⁴R¹⁵)ₘC(═O)OR¹³, —O(CR⁸R⁹)ₙC(═O)OR¹³, benzoic acid and tetrazolyl;
each R¹³ is independently selected from H, and C₁₋₆alkyl;
each R¹⁴ is independently selected from H and C₁₋₆alkyl;
each R¹⁵ is independently selected from H and C₁₋₆alkyl;
or R¹⁴ and R¹⁵ together with the carbon in CR¹⁴R¹⁵ form a C₃₋₈cycloalkyl;
R¹⁶ is a C₃₋₈cycloalkyl;
R¹⁷ is
a) C₃₋₈cycloalkyl substituted with 1 to 2 R¹² groups,
or
b) a 5 to 6 membered monocyclic heterocycloalkyl having 1 to 2 ring members independently selected from N, O and S substituted with 1 to 2 R¹² groups;
R¹⁸ is adamantanyl;
each m is independently selected from 1, 2, 3 or 4;
and
each n is independently selected from 1, 2, 3 or 4.

7. The compound of claim 6, or pharmaceutically acceptable salt thereof, wherein:
R⁵ is selected from
a) —NR⁶R⁷;
b) —OR¹¹;
c) —S(CH₂)₂C(═O)OH,
d) a piperidinyl substituted with 1 to 2 R¹² groups;
e) a piperazinyl is substituted with 1 to 2 R¹² groups;
and
f) a pyrrolidinyl substituted with 1 to 2 R¹² groups;
R⁶ is H, methyl, ethyl, propyl, isopropyl, butyl, isobutyl, 3,3,3-trifluoropropyl, cyclopropyl, cyclobutyl, —(CH₂)ₙOR¹⁰, or —CH₂R¹⁶;
R⁷ is —CH₂C(═O)OH, —(CH₂)₂C(═O)OH, —(CH₂)₃C(═O)OH, —(CH₂)₄C(═O)OH, —CH₂R¹⁷, —CH₂(CR¹⁴R¹⁵)C(═O)OH, —(CR¹⁴R¹⁵)₂C(═O)OH, —CHR¹²R¹⁸, a cyclohexyl substituted with 1 to 2 R¹² groups, a bicyclo[2.2.1]heptenyl substituted with 1 to 2 R¹² groups, or a bicyclo[2.2.1]heptanyl substituted with 1 to 2 R¹² groups;
R¹⁰ is H or methyl;
R¹¹ is —(CH₂)₂C(═O)OR¹³;
each R¹² is independently selected from methyl, —OH, —C(═O)OR¹³, —CH₂C(═O)OR¹³, —(CH₂)₂C(═O)OR¹³, —(CH₂)₃C(═O)OR¹³, —(CR¹⁴R¹⁵)C(═O)OR¹³, —(CR¹⁴R¹⁵)₂C(═O)OR¹³, —OCH₂C(═O)OR¹³, —CH₂C(═O)OR¹³, —C(═O)NH(CR¹⁴R¹⁵)C(═O)OR¹³, benzoic acid and tetrazolyl;
each R¹³ is independently selected from H, methyl and ethyl;
each R¹⁴ is independently selected from H, methyl and ethyl;
each R¹⁵ is independently selected from H, methyl and ethyl;
or R¹⁴ and R¹⁵ together with the carbon in CR¹⁴R¹⁵ form a cyclopropyl, a cyclobutyl or a cyclopentyl;
R¹⁶ is a cyclopropyl;
R¹⁷ is
a) a cyclobutyl substituted with 1 to 2 R¹² groups;
b) a cyclopentyl substituted with 1 to 2 R¹² groups;
or
c) a tetrahydro-2H-pyranyl substituted with 1 to 2 R¹² groups;
R¹⁸ is adamantanyl.

8. The compound of claim 1, or pharmaceutically acceptable salt thereof, wherein R¹ is halo or halo-substituted C₁₋₆alkyl.

9. The compound of claim 8, or pharmaceutically acceptable salt thereof, wherein R¹ is Cl, F or CF₃.

10. The compound of claim 9, or pharmaceutically acceptable salt thereof, wherein R¹ is Cl or CF₃.

11. The compound of claim 10, or pharmaceutically acceptable salt thereof, wherein R¹ is CF₃.

12. The compound of claim 1 selected from:
3-((6-(N-(5-chloro-6-(2,6-dimethylphenyl)pyridin-2-yl)sulfamoyl)pyridin-2-yl)amino)propanoic acid;
3-((4-(N-(5-chloro-6-(2,6-dimethylphenyl)pyridin-2-yl)sulfamoyl)pyridin-2-yl)amino)propanoic acid;
4-((6-(N-(5-chloro-6-(2,6-dimethylphenyl)pyridin-2-yl)sulfamoyl)pyridin-2-yl)amino)butanoic acid;
5-((6-(N-(5-chloro-6-(2,6-dimethylphenyl)pyridin-2-yl)sulfamoyl)pyridin-2-yl)amino)pentanoic acid;
2-(1-(6-(N-(5-chloro-6-(2,6-dimethylphenyl)pyridin-2-yl)sulfamoyl)pyridin-2-yl)piperidin-4-yl)acetic acid;
1-(6-(N-(6-([1,1′-biphenyl]-2-yl)-5-chloropyridin-2-yl)sulfamoyl)pyridin-2-yl)-4-methylpiperidine-4-carboxylic acid;
1-(6-(N-(6-([1,1′-biphenyl]-2-yl)-5-(trifluoromethyl)pyridin-2-yl)sulfamoyl)pyridin-2-yl)-4-methylpiperidine-4-carboxylic acid;
4-(4-(6-(N-(6-(2-chloro-5-methoxyphenyl)-5-(trifluoromethyl)pyridin-2-yl)sulfamoyl)pyridin-2-yl)piperazin-1-yl)benzoic acid;
2-(1-(6-(N-(6-(5-fluoro-2-methylphenyl)-5-(trifluoromethyl)pyridin-2-yl)sulfamoyl)pyridin-2-yl)-4-methylpiperidin-4-yl)acetic acid;
2-(4-methyl-1-(6-(N-(6-(o-tolyl)-5-(trifluoromethyl)pyridin-2-yl)sulfamoyl)pyridin-2-yl)piperidin-4-yl)acetic acid;
2,2-dimethyl-3-((6-(N-(6-(o-tolyl)-5-(trifluoromethyl)pyridin-2-yl)sulfamoyl)pyridin-2-yl)amino)propanoic acid;
2-(1-(6-(N-(6-(2-ethylphenyl)-5-(trifluoromethyl)pyridin-2-yl)sulfamoyl)pyridin-2-yl)-4-methylpiperidin-4-yl)acetic acid;
2-(1-(6-(N-(6-([1,1′-biphenyl]-2-yl)-5-(trifluoromethyl)pyridin-2-yl)sulfamoyl)pyridin-2-yl)-4-methylpiperidin-4-yl)acetic acid;
2-methyl-2-(1-(6-(N-(6-(o-tolyl)-5-(trifluoromethyl)pyridin-2-yl)sulfamoyl)pyridin-2-yl)piperidin-4-yl)propanoic acid;
2-(4-hydroxy-1-(6-(N-(6-(o-tolyl)-5-(trifluoromethyl)pyridin-2-yl)sulfamoyl)pyridin-2-yl)piperidin-4-yl)-2-methylpropanoic acid;
1-(((6-(N-(6-(o-tolyl)-5-(trifluoromethyl)pyridin-2-yl)sulfamoyl)pyridin-2-yl)amino)methyl)cyclopropane-1-carboxylic acid;
1-(((6-(N-(6-(o-tolyl)-5-(trifluoromethyl)pyridin-2-yl)sulfamoyl)pyridin-2-yl)amino)methyl)cyclobutane-1-carboxylic acid;
2-(1-(6-(N-(6-(3-fluoro-2-methylphenyl)-5-(trifluoromethyl)pyridin-2-yl)sulfamoyl)pyridin-2-yl)-4-methylpiperidin-4-yl)acetic acid;
2-(1-(6-(N-(6-(o-tolyl)-5-(trifluoromethyl)pyridin-2-yl)sulfamoyl)pyridin-2-yl)piperidin-4-yl)acetic acid;
2-(1-(6-(N-(6-(2-ethylphenyl)-5-(trifluoromethyl)pyridin-2-yl)sulfamoyl)pyridin-2-yl)piperidin-4-yl)acetic acid;
2-(1-(6-(N-(6-([1,1′-biphenyl]-2-yl)-5-(trifluoromethyl)pyridin-2-yl)sulfamoyl)pyridin-2-yl)piperidin-4-yl)acetic acid;
2-(1-(6-(N-(6-(2-cyclobutylphenyl)-5-(trifluoromethyl)pyridin-2-yl)sulfamoyl)pyridin-2-yl)-4-methylpiperidin-4-yl)acetic acid;
2-(1-(6-(N-(6-(2-isopropylphenyl)-5-(trifluoromethyl)pyridin-2-yl)sulfamoyl)pyridin-2-yl)-4-methylpiperidin-4-yl)acetic acid;
2-(1-(6-(N-(6-(2-(tert-butyl)phenyl)-5-(trifluoromethyl)pyridin-2-yl)sulfamoyl)pyridin-2-yl)-4-methylpiperidin-4-yl)acetic acid;
2-(1-(6-(N-(6-(2-cyclobutylphenyl)-5-(trifluoromethyl)pyridin-2-yl)sulfamoyl)pyridin-2-yl)piperidin-4-yl)acetic acid;
4-methyl-1-(6-(N-(6-(2-(pyrimidin-2-yl)phenyl)-5-(trifluoromethyl)pyridin-2-yl)sulfamoyl)pyridin-2-yl)piperidine-4-carboxylic acid;
4-methyl-1-(6-(N-(6-(2-(pyridin-4-yl)phenyl)-5-(trifluoromethyl)pyridin-2-yl)sulfamoyl)pyridin-2-yl)piperidine-4-carboxylic acid;
4-methyl-1-(6-(N-(6-(2-phenoxyphenyl)-5-(trifluoromethyl)pyridin-2-yl)sulfamoyl)pyridin-2-yl)piperidine-4-carboxylic acid;
1-(6-(N-(6-([1,1′-biphenyl]-2-yl)-5-chloropyridin-2-yl)sulfamoyl)pyridin-2-yl)-3-methylpiperidine-3-carboxylic acid;
1-(6-(N-(4′-isopropyl-3-(trifluoromethyl)-[2,3′-bipyridin]-6-yl)sulfamoyl)pyridin-2-yl)-3-methylpiperidine-3-carboxylic acid;
2-((1-(6-(N-(6-(2-isopropylphenyl)-5-(trifluoromethyl)pyridin-2-yl)sulfamoyl)pyridin-2-yl)piperidin-4-yl)ₙxy)acetic acid;
2-(1-(6-(N-(6-(o-tolyl)-5-(trifluoromethyl)pyridin-2-yl)sulfamoyl)pyridin-2-yl)pyrrolidin-3-yl)acetic acid;
2-(1-(6-(N-(6-(2-cyclopropylphenyl)-5-(trifluoromethyl)pyridin-2-yl)sulfamoyl)pyridin-2-yl)pyrrolidin-3-yl)acetic acid;
3-(methyl(6-(N-(6-(o-tolyl)-5-(trifluoromethyl)pyridin-2-yl)sulfamoyl)pyridin-2-yl)amino)propanoic acid;
3-((6-(N-(6-(o-tolyl)-5-(trifluoromethyl)pyridin-2-yl)sulfamoyl)pyridin-2-yl)amino)propanoic acid;
4-((6-(N-(6-(o-tolyl)-5-(trifluoromethyl)pyridin-2-yl)sulfamoyl)pyridin-2-yl)amino)butanoic acid;
4-(methyl(6-(N-(6-(o-tolyl)-5-(trifluoromethyl)pyridin-2-yl)sulfamoyl)pyridin-2-yl)amino)butanoic acid;
3-((6-(N-(6-(2-cyclopropylphenyl)-5-(trifluoromethyl)pyridin-2-yl)sulfamoyl)pyridin-2-yl)(methyl)amino)propanoic acid;
3-((6-(N-(6-(2-cyclopropylphenyl)-5-(trifluoromethyl)pyridin-2-yl)sulfamoyl)pyridin-2-yl)amino)propanoic acid;
4-((6-(N-(6-(2-cyclopropylphenyl)-5-(trifluoromethyl)pyridin-2-yl)sulfamoyl)pyridin-2-yl)amino)butanoic acid;
4-((6-(N-(6-(2-cyclopropylphenyl)-5-(trifluoromethyl)pyridin-2-yl)sulfamoyl)pyridin-2-yl)(methyl)amino)butanoic acid;
5-((6-(N-(6-(2-cyclopropylphenyl)-5-(trifluoromethyl)pyridin-2-yl)sulfamoyl)pyridin-2-yl)amino)pentanoic acid;
5-((6-(N-(6-(o-tolyl)-5-(trifluoromethyl)pyridin-2-yl)sulfamoyl)pyridin-2-yl)amino)pentanoic acid;
2-(1-(6-(N-(6-(o-tolyl)-5-(trifluoromethyl)pyridin-2-yl)sulfamoyl)pyridin-2-yl)piperidin-3-yl)acetic acid;
3-((6-(N-(6-(2-isopropylphenyl)-5-(trifluoromethyl)pyridin-2-yl)sulfamoyl)pyridin-2-yl)(methyl)amino)propanoic acid;
3-((6-(N-(6-(5-fluoro-2-methylphenyl)-5-(trifluoromethyl)pyridin-2-yl)sulfamoyl)pyridin-2-yl)(methyl)amino)-2,2-dimethylpropanoic acid;
2,2-dimethyl-3-(methyl(6-(N-(6-(o-tolyl)-5-(trifluoromethyl)pyridin-2-yl)sulfamoyl)pyridin-2-yl)amino)propanoic acid;
3-((6-(N-(6-(3-fluoro-2-methylphenyl)-5-(trifluoromethyl)pyridin-2-yl)sulfamoyl)pyridin-2-yl)(methyl)amino)-2,2-dimethylpropanoic acid;
3-((6-(N-(6-(5-fluoro-2-methylphenyl)-5-(trifluoromethyl)pyridin-2-yl)sulfamoyl)pyridin-2-yl)(methyl)amino)propanoic acid;
3-((6-(N-(6-(3-fluoro-2-methylphenyl)-5-(trifluoromethyl)pyridin-2-yl)sulfamoyl)pyridin-2-yl)(methyl)amino)propanoic acid;
2-(1-(6-(N-(6-(3-fluoro-2-methylphenyl)-5-(trifluoromethyl)pyridin-2-yl)sulfamoyl)pyridin-2-yl)-3-methylpiperidin-3-yl)acetic acid;
2-(3-methyl-1-(6-(N-(6-(o-tolyl)-5-(trifluoromethyl)pyridin-2-yl)sulfamoyl)pyridin-2-yl)piperidin-3-yl)acetic acid;
2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid;
4-(ethyl(6-(N-(6-(2-isopropylphenyl)-5-(trifluoromethyl)pyridin-2-yl)sulfamoyl)pyridin-2-yl)amino)butanoic acid;
3-(ethyl(6-(N-(6-(o-tolyl)-5-(trifluoromethyl)pyridin-2-yl)sulfamoyl)pyridin-2-yl)amino)propanoic acid;
3-(ethyl(6-(N-(6-(2-ethylphenyl)-5-(trifluoromethyl)pyridin-2-yl)sulfamoyl)pyridin-2-yl)amino)propanoic acid;
4-((6-(N-(6-(2-isopropylphenyl)-5-(trifluoromethyl)pyridin-2-yl)sulfamoyl)pyridin-2-yl)(methyl)amino)butanoic acid;
3-(1-(6-(N-(6-(o-tolyl)-5-(trifluoromethyl)pyridin-2-yl)sulfamoyl)pyridin-2-yl)piperidin-3-yl)propanoic acid;
3-(1-(6-(N-(5-(trifluoromethyl)-6-(2-vinylphenyl)pyridin-2-yl)sulfamoyl)pyridin-2-yl)piperidin-3-yl)propanoic acid;
4-(1-(6-(N-(6-(o-tolyl)-5-(trifluoromethyl)pyridin-2-yl)sulfamoyl)pyridin-2-yl)piperidin-3-yl)butanoic acid;
4-(1-(6-(N-(5-(trifluoromethyl)-6-(2-vinylphenyl)pyridin-2-yl)sulfamoyl)pyridin-2-yl)piperidin-3-yl)butanoic acid;
ethyl (R)-2-(1-(6-(N-(5-(trifluoromethyl)-6-(2-vinylphenyl)pyridin-2-yl)sulfamoyl)pyridin-2-yl)piperidin-3-yl)acetate;
methyl (S)-2-(1-(6-(N-(5-(trifluoromethyl)-6-(2-vinylphenyl)pyridin-2-yl)sulfamoyl)pyridin-2-yl)piperidin-3-yl)acetate;
2-(3-methyl-1-(6-(N-(6-(o-tolyl)-5-(trifluoromethyl)pyridin-2-yl)sulfamoyl)pyridin-2-yl)pyrrolidin-3-yl)acetic acid;
2-(1-(6-(N-(6-(3-fluoro-2-methylphenyl)-5-(trifluoromethyl)pyridin-2-yl)sulfamoyl)pyridin-2-yl)-3-methylpyrrolidin-3-yl)acetic acid;
2-(1-(6-(N-(6-(5-fluoro-2-methylphenyl)-5-(trifluoromethyl)pyridin-2-yl)sulfamoyl)pyridin-2-yl)-3-methylpyrrolidin-3-yl)acetic acid;
3-(ethyl(6-(N-(6-(3-fluoro-2-methylphenyl)-5-(trifluoromethyl)pyridin-2-yl)sulfamoyl)pyridin-2-yl)amino)propanoic acid;
3-(1-(6-(N-(6-(2-ethylphenyl)-5-(trifluoromethyl)pyridin-2-yl)sulfamoyl)pyridin-2-yl)piperidin-3-yl)propanoic acid;
4-(1-(6-(N-(6-(2-ethylphenyl)-5-(trifluoromethyl)pyridin-2-yl)sulfamoyl)pyridin-2-yl)piperidin-3-yl)butanoic acid;
(R)-2-(1-(6-(N-(5-(trifluoromethyl)-6-(2-vinylphenyl)pyridin-2-yl)sulfamoyl)pyridin-2-yl)piperidin-3-yl)acetic acid;
(S)-2-(1-(6-(N-(5-(trifluoromethyl)-6-(2-vinylphenyl)pyridin-2-yl)sulfamoyl)pyridin-2-yl)piperidin-3-yl)acetic acid;
(R)-2-(1-(6-(N-(6-(o-tolyl)-5-(trifluoromethyl)pyridin-2-yl)sulfamoyl)pyridin-2-yl)piperidin-3-yl)acetic acid;
(S)-2-(1-(6-(N-(6-(o-tolyl)-5-(trifluoromethyl)pyridin-2-yl)sulfamoyl)pyridin-2-yl)piperidin-3-yl)acetic acid;
3-(ethyl(6-(N-(6-(5-fluoro-2-methylphenyl)-5-(trifluoromethyl)pyridin-2-yl)sulfamoyl)pyridin-2-yl)amino)propanoic acid;
3-(1-(6-(N-(6-(o-tolyl)-5-(trifluoromethyl)pyridin-2-yl)sulfamoyl)pyridin-2-yl)piperidin-4-yl)propanoic acid;
(R)-3-(1-(6-(N-(6-(o-tolyl)-5-(trifluoromethyl)pyridin-2-yl)sulfamoyl)pyridin-2-yl)piperidin-3-yl)propanoic acid;
(S)-3-(1-(6-(N-(6-(o-tolyl)-5-(trifluoromethyl)pyridin-2-yl)sulfamoyl)pyridin-2-yl)piperidin-3-yl)propanoic acid;
3-(isopropyl(6-(N-(6-(o-tolyl)-5-(trifluoromethyl)pyridin-2-yl)sulfamoyl)pyridin-2-yl)amino)propanoic acid;
(R)-2-(1-(6-(N-(6-(2-ethylphenyl)-5-(trifluoromethyl)pyridin-2-yl)sulfamoyl)pyridin-2-yl)piperidin-3-yl)acetic acid;
(S)-2-(1-(6-(N-(6-(2-ethylphenyl)-5-(trifluoromethyl)pyridin-2-yl)sulfamoyl)pyridin-2-yl)piperidin-3-yl)acetic acid;
(S)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid;
(R)-2-(1-(6-(N-(6-(5-fluoro-2-methylphenyl)-5-(trifluoromethyl)pyridin-2-yl)sulfamoyl)pyridin-2-yl)-3-methylpiperidin-3-yl)acetic acid;
5-(methyl(6-(N-(6-(o-tolyl)-5-(trifluoromethyl)pyridin-2-yl)sulfamoyl)pyridin-2-yl)amino)pentanoic acid;
5-((6-(N-(6-(3-fluoro-2-methylphenyl)-5-(trifluoromethyl)pyridin-2-yl)sulfamoyl)pyridin-2-yl)(methyl)amino)pentanoic acid;
5-((6-(N-(6-(5-fluoro-2-methylphenyl)-5-(trifluoromethyl)pyridin-2-yl)sulfamoyl)pyridin-2-yl)(methyl)amino)pentanoic acid;
4-((6-(N-(6-(5-fluoro-2-methylphenyl)-5-(trifluoromethyl)pyridin-2-yl)sulfamoyl)pyridin-2-yl)(methyl)amino)butanoic acid;
4-((6-(N-(6-(2-ethyl-5-fluorophenyl)-5-(trifluoromethyl)pyridin-2-yl)sulfamoyl)pyridin-2-yl)(methyl)amino)butanoic acid;
4-(isopropyl(6-(N-(6-(o-tolyl)-5-(trifluoromethyl)pyridin-2-yl)sulfamoyl)pyridin-2-yl)amino)butanoic acid;
3-((cyclopropylmethyl)(6-(N-(6-(5-fluoro-2-methylphenyl)-5-(trifluoromethyl)pyridin-2-yl)sulfamoyl)pyridin-2-yl)amino)propanoic acid;
3-(ethyl(6-(N-(6-(2-isopropylphenyl)-5-(trifluoromethyl)pyridin-2-yl)sulfamoyl)pyridin-2-yl)amino)propanoic acid;
4-((6-(N-(6-(5-fluoro-2-methylphenyl)-5-(trifluoromethyl)pyridin-2-yl)sulfamoyl)pyridin-2-yl)(isopropyl)amino)butanoic acid;
4-((6-(N-(6-(2-ethylphenyl)-5-(trifluoromethyl)pyridin-2-yl)sulfamoyl)pyridin-2-yl)(isopropyl)amino)butanoic acid;
3-((6-(N-(6-(2-cyclopropylphenyl)-5-(trifluoromethyl)pyridin-2-yl)sulfamoyl)pyridin-2-yl)(ethyl)amino)propanoic acid;
4-(ethyl(6-(N-(6-(2-ethylphenyl)-5-(trifluoromethyl)pyridin-2-yl)sulfamoyl)pyridin-2-yl)amino)butanoic acid;
3-(1-(6-(N-(6-(o-tolyl)-5-(trifluoromethyl)pyridin-2-yl)sulfamoyl)pyridin-2-yl)piperidin-2-yl)propanoic acid;
3-(ethyl(6-(N-(6-(2-ethyl-5-fluorophenyl)-5-(trifluoromethyl)pyridin-2-yl)sulfamoyl)pyridin-2-yl)amino)propanoic acid;
5-((6-(N-(6-(2-ethyl-5-fluorophenyl)-5-(trifluoromethyl)pyridin-2-yl)sulfamoyl)pyridin-2-yl)(isopropyl)amino)pentanoic acid;
5-((6-(N-(6-(5-fluoro-2-methylphenyl)-5-(trifluoromethyl)pyridin-2-yl)sulfamoyl)pyridin-2-yl)(isopropyl)amino)pentanoic acid;
5-(isopropyl(6-(N-(6-(o-tolyl)-5-(trifluoromethyl)pyridin-2-yl)sulfamoyl)pyridin-2-yl)amino)pentanoic acid;
3-((cyclopropylmethyl)(6-(N-(6-(o-tolyl)-5-(trifluoromethyl)pyridin-2-yl)sulfamoyl)pyridin-2-yl)amino)propanoic acid;
4-(butyl(6-(N-(6-(o-tolyl)-5-(trifluoromethyl)pyridin-2-yl)sulfamoyl)pyridin-2-yl)amino)butanoic acid;
4-(butyl(6-(N-(6-(5-fluoro-2-methylphenyl)-5-(trifluoromethyl)pyridin-2-yl)sulfamoyl)pyridin-2-yl)amino)butanoic acid;
4-((cyclopropylmethyl)(6-(N-(6-(o-tolyl)-5-(trifluoromethyl)pyridin-2-yl)sulfamoyl)pyridin-2-yl)amino)butanoic acid;
4-((cyclopropylmethyl)(6-(N-(6-(5-fluoro-2-methylphenyl)-5-(trifluoromethyl)pyridin-2-yl)sulfamoyl)pyridin-2-yl)amino)butanoic acid;
3-((6-(N-(6-(o-tolyl)-5-(trifluoromethyl)pyridin-2-yl)sulfamoyl)pyridin-2-yl)amino)cyclohexane-1-carboxylic acid;
(1S,3R)-3-((6-(N-(6-(o-tolyl)-5-(trifluoromethyl)pyridin-2-yl)sulfamoyl)pyridin-2-yl)amino)cyclohexane-1-carboxylic acid;
(1R,3S)-3-((6-(N-(6-(o-tolyl)-5-(trifluoromethyl)pyridin-2-yl)sulfamoyl)pyridin-2-yl)amino)cyclohexane-1-carboxylic acid;
(1R,2S,3R,4S)-3-((6-(N-(6-(o-tolyl)-5-(trifluoromethyl)pyridin-2-yl)sulfamoyl)pyridin-2-yl)amino)bicyclo[2.2.1]hept-5-ene-2-carboxylic acid;
(1S,2R,3S,4R)-3-((6-(N-(6-(o-tolyl)-5-(trifluoromethyl)pyridin-2-yl)sulfamoyl)pyridin-2-yl)amino)bicyclo[2.2.1]hept-5-ene-2-carboxylic acid;
(1R,2S,3R,4S)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
(1S,2S,3R,4R)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
(1R,2R,3S,4S)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
4-(ethyl(6-(N-(6-(2-ethyl-5-fluorophenyl)-5-(trifluoromethyl)pyridin-2-yl)sulfamoyl)pyridin-2-yl)amino)butanoic acid;
3-((6-(N-(6-(2-cyclopropyl-5-fluorophenyl)-5-(trifluoromethyl)pyridin-2-yl)sulfamoyl)pyridin-2-yl)(ethyl)amino)propanoic acid;
4-(butyl(6-(N-(3-chloro-2′-isopropyl-[2,3′-bipyridin]-6-yl)sulfamoyl)pyridin-2-yl)amino)butanoic acid;
3-(cyclopropyl(6-(N-(6-(2-ethyl-5-fluorophenyl)-5-(trifluoromethyl)pyridin-2-yl)sulfamoyl)pyridin-2-yl)amino)propanoic acid;
4-(ethyl(6-(N-(6-(o-tolyl)-5-(trifluoromethyl)pyridin-2-yl)sulfamoyl)pyridin-2-yl)amino)butanoic acid;
4-(ethyl(6-(N-(6-(5-fluoro-2-methylphenyl)-5-(trifluoromethyl)pyridin-2-yl)sulfamoyl)pyridin-2-yl)amino)butanoic acid;
3-((cyclopropylmethyl)(6-(N-(6-(2-ethyl-5-fluorophenyl)-5-(trifluoromethyl)pyridin-2-yl)sulfamoyl)pyridin-2-yl)amino)propanoic acid;
3-((6-(N-(6-(o-tolyl)-5-(trifluoromethyl)pyridin-2-yl)sulfamoyl)pyridin-2-yl)thio)propanoic acid;
3-(cyclopropyl(6-(N-(6-(o-tolyl)-5-(trifluoromethyl)pyridin-2-yl)sulfamoyl)pyridin-2-yl)amino)propanoic acid;
3-((6-(N-(6-(2-ethyl-5-fluorophenyl)-5-(trifluoromethyl)pyridin-2-yl)sulfamoyl)pyridin-2-yl)(isopropyl)amino)propanoic acid;
4-((6-(N-(6-(2-ethylphenyl)-5-(trifluoromethyl)pyridin-2-yl)sulfamoyl)pyridin-2-yl)ₙxy)cyclohexane-1-carboxylic acid;
3-((6-(N-(6-(o-tolyl)-5-(trifluoromethyl)pyridin-2-yl)sulfamoyl)pyridin-2-yl)ₙxy)propanoic acid;
(1S,2R,3S,4R)-3-((6-(N-(6-(o-tolyl)-5-(trifluoromethyl)pyridin-2-yl)sulfamoyl)pyridin-2-yl)amino)bicyclo[2.2.1]heptane-2-carboxylic acid;
3-(propyl(6-(N-(6-(o-tolyl)-5-(trifluoromethyl)pyridin-2-yl)sulfamoyl)pyridin-2-yl)amino)propanoic acid;
3-((6-(N-(6-(5-fluoro-2-methylphenyl)-5-(trifluoromethyl)pyridin-2-yl)sulfamoyl)pyridin-2-yl)(propyl)amino)propanoic acid;
(S)-2-(3-methyl-1-(6-(N-(6-(o-tolyl)-5-(trifluoromethyl)pyridin-2-yl)sulfamoyl)pyridin-2-yl)piperidin-3-yl)acetic acid;
(R)-2-(3-methyl-1-(6-(N-(6-(o-tolyl)-5-(trifluoromethyl)pyridin-2-yl)sulfamoyl)pyridin-2-yl)piperidin-3-yl)acetic acid;
(S)-6-(2-(1H-tetrazol-5-yl)pyrrolidin-1-yl)-N-(5-chloro-6-(2,6-dimethylphenyl)pyridin-2-yl)pyridine-2-sulfonamide;
4-(cyclopropyl(6-(N-(6-(o-tolyl)-5-(trifluoromethyl)pyridin-2-yl)sulfamoyl)pyridin-2-yl)amino)butanoic acid;
3-((6-(N-(6-(5-fluoro-2-methylphenyl)-5-(trifluoromethyl)pyridin-2-yl)sulfamoyl)pyridin-2-yl)(isobutyl)amino)propanoic acid;
4-((6-(N-(6-(5-fluoro-2-methylphenyl)-5-(trifluoromethyl)pyridin-2-yl)sulfamoyl)pyridin-2-yl)(isobutyl)amino)butanoic acid;
3-(isobutyl(6-(N-(6-(o-tolyl)-5-(trifluoromethyl)pyridin-2-yl)sulfamoyl)pyridin-2-yl)amino)propanoic acid;
4-(isobutyl(6-(N-(6-(o-tolyl)-5-(trifluoromethyl)pyridin-2-yl)sulfamoyl)pyridin-2-yl)amino)butanoic acid;
1-((methyl(6-(N-(6-(o-tolyl)-5-(trifluoromethyl)pyridin-2-yl)sulfamoyl)pyridin-2-yl)amino)methyl)cyclopentane-1-carboxylic acid;
4-((6-(N-(6-(5-fluoro-2-methylphenyl)-5-(trifluoromethyl)pyridin-2-yl)sulfamoyl)pyridin-2-yl)(propyl)amino)butanoic acid;
4-(propyl(6-(N-(6-(o-tolyl)-5-(trifluoromethyl)pyridin-2-yl)sulfamoyl)pyridin-2-yl)amino)butanoic acid;
4-((methyl(6-(N-(6-(o-tolyl)-5-(trifluoromethyl)pyridin-2-yl)sulfamoyl)pyridin-2-yl)amino)methyl)tetrahydro-2H-pyran-4-carboxylic acid;
4-((6-(N-(6-(o-tolyl)-5-(trifluoromethyl)pyridin-2-yl)sulfamoyl)pyridin-2-yl)(3,3,3-trifluoropropyl)amino)butanoic acid;
3-((6-(N-(6-(5-fluoro-2-methylphenyl)-5-(trifluoromethyl)pyridin-2-yl)sulfamoyl)pyridin-2-yl)(isopropyl)amino)propanoic acid;
2-(4-((6-(N-(5-chloro-6-(o-tolyl)pyridin-2-yl)sulfamoyl)pyridin-2-yl)amino)cyclohexane-1-carboxamido)butanoic acid;
3-((6-(N-(6-(o-tolyl)-5-(trifluoromethyl)pyridin-2-yl)sulfamoyl)pyridin-2-yl)(3,3,3-trifluoropropyl)amino)propanoic acid;
3-((6-(N-(6-(5-fluoro-2-methylphenyl)-5-(trifluoromethyl)pyridin-2-yl)sulfamoyl)pyridin-2-yl)(3,3,3-trifluoropropyl)amino)propanoic acid;
1-((methyl(6-(N-(6-(o-tolyl)-5-(trifluoromethyl)pyridin-2-yl)sulfamoyl)pyridin-2-yl)amino)methyl)cyclobutane-1-carboxylic acid;
4-(((6-(N-(6-(5-fluoro-2-methylphenyl)-5-(trifluoromethyl)pyridin-2-yl)sulfamoyl)pyridin-2-yl)(methyl)amino)methyl)tetrahydro-2H-pyran-4-carboxylic acid;
1-(((6-(N-(6-(5-fluoro-2-methylphenyl)-5-(trifluoromethyl)pyridin-2-yl)sulfamoyl)pyridin-2-yl)(methyl)amino)methyl)cyclobutane-1-carboxylic acid;
3-((4-(N-(5-chloro-6-(2,6-dimethylphenyl)pyridin-2-yl)sulfamoyl)pyridin-2-yl)(ethyl)amino)propanoic acid;
3-((4-(N-(5-chloro-6-(2,6-dimethylphenyl)pyridin-2-yl)sulfamoyl)pyridin-2-yl)amino)pentanoic acid;
3-((4-(N-(5-chloro-6-(2,6-dimethylphenyl)pyridin-2-yl)sulfamoyl)pyridin-2-yl)amino)butanoic acid;
2-(adamantan-1-yl)-2-((6-(N-(6-(o-tolyl)-5-(trifluoromethyl)pyridin-2-yl)sulfamoyl)pyridin-2-yl)amino)acetic acid;
3-((4-(N-(5-chloro-6-(5-fluoro-2-methylphenyl)pyridin-2-yl)sulfamoyl)pyridin-2-yl)(ethyl)amino)propanoic acid;
3-((6-(N-(6-(2,6-dimethylphenyl)-5-(trifluoromethyl)pyridin-2-yl)sulfamoyl)pyridin-2-yl)(ethyl)amino)propanoic acid;
(6-(N-(6-(o-tolyl)-5-(trifluoromethyl)pyridin-2-yl)sulfamoyl)pyridin-2-yl)glycine;
3-(butyl(6-(N-(6-(o-tolyl)-5-(trifluoromethyl)pyridin-2-yl)sulfamoyl)pyridin-2-yl)amino)propanoic acid;
3-(butyl(6-(N-(6-(5-fluoro-2-methylphenyl)-5-(trifluoromethyl)pyridin-2-yl)sulfamoyl)pyridin-2-yl)amino)propanoic acid;
3-(cyclobutyl(6-(N-(6-(o-tolyl)-5-(trifluoromethyl)pyridin-2-yl)sulfamoyl)pyridin-2-yl)amino)propanoic acid;
4-((6-(N-(5-chloro-6-(o-tolyl)pyridin-2-yl)sulfamoyl)pyridin-2-yl)(isobutyl)amino)butanoic acid;
4-((6-(N-(5-chloro-6-(2,6-dimethylphenyl)pyridin-2-yl)sulfamoyl)pyridin-2-yl)(isobutyl)amino)butanoic acid;
4-((6-(N-(5-chloro-6-(o-tolyl)pyridin-2-yl)sulfamoyl)pyridin-2-yl)(propyl)amino)butanoic acid;
4-((6-(N-(5-chloro-6-(2,6-dimethylphenyl)pyridin-2-yl)sulfamoyl)pyridin-2-yl)(propyl)amino)butanoic acid, and
4-((2-methoxyethyl)(6-(N-(6-(o-tolyl)-5-(trifluoromethyl)pyridin-2-yl)sulfamoyl)pyridin-2-yl)amino)butanoic acid.

13. The compound of claim 1 selected from:
4-(isobutyl(6-(N-(6-(o-tolyl)-5-(trifluoromethyl)pyridin-2-yl)sulfamoyl)pyridin-2-yl)amino)butanoic acid;
4-((6-(N-(6-(5-fluoro-2-methylphenyl)-5-(trifluoromethyl)pyridin-2-yl)sulfamoyl)pyridin-2-yl)(propyl)amino)butanoic acid;
3-((cyclopropylmethyl)(6-(N-(6-(o-tolyl)-5-(trifluoromethyl)pyridin-2-yl)sulfamoyl)pyridin-2-yl)amino)propanoic acid;
4-((6-(N-(6-(5-fluoro-2-methylphenyl)-5-(trifluoromethyl)pyridin-2-yl)sulfamoyl)pyridin-2-yl)(isopropyl)amino)butanoic acid;
5-(isopropyl(6-(N-(6-(o-tolyl)-5-(trifluoromethyl)pyridin-2-yl)sulfamoyl)pyridin-2-yl)amino)pentanoic acid, and
4-(butyl(6-(N-(6-(5-fluoro-2-methylphenyl)-5-(trifluoromethyl)pyridin-2-yl)sulfamoyl)pyridin-2-yl)amino)butanoic acid.

14. A pharmaceutical composition comprising a compound of claim 1, or a pharmaceutically acceptable salt thereof, and a pharmaceutically acceptable carrier, or diluent.

15. The pharmaceutical composition of claim 14, further comprising one or more additional pharmaceutical agent(s).

16. The pharmaceutical composition of claim 15, wherein the additional pharmaceutical agent(s) is selected from a mucolytic agent, nebulized hypertonic saline, bronchodilator, an antibiotic, an anti-infective agent, a CFTR modulator, and an anti-inflammatory agent.

17. (canceled)

18. The pharmaceutical composition of claim 15, wherein the additional pharmaceutical agent is a CFTR corrector or a CFTR potentiator.

19. (canceled)

20. The pharmaceutical composition of claim 15, wherein the additional pharmaceutical agents are a CFTR modulator and a CFTR potentiator.

21. A method for treating a CFTR mediated disease in a subject comprising administering to the subject a compound, or a pharmaceutically acceptable salt thereof, of having the structure of Formula (I), 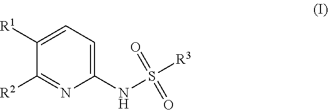
wherein:
R¹ is H, C₁₋C₆alkyl, halo, halo-substituted C₁₋C₆alkyl, deuterium substituted C₁₋C₆alkyl, C₁₋C₆alkoxy or halo-substituted C₁₋C₆alkoxy;
R² is selected from:
a) a phenyl substituted with 1 to 2 R⁴ groups;
b) an unsubstituted 5 to 6 membered heteroaryl having 1 to 3 heteroatoms independently selected from N, O and S,
and
c) a 5 to 6 membered heteroaryl having 1 to 3 heteroatoms independently selected from N, O and S, wherein the heteroaryl is substituted with 1 to 3 R⁴ groups;
R³ is a pyridin-2-yl or a pyridin-4-yl, wherein the pyridin-2-yl or a pyridin-4-yl is substituted with an R⁵ group;
each R⁴ is independently selected from D, C₁₋C₆alkyl, phenyl, phenoxy, halo, C₁₋C₆alkoxy, C₃₋C₈cycloalkyl, C₂-C₆alkenyl, halo-substituted C₁₋C₆alkyl, deuterium substituted C₁₋C₆alkyl, halo-substituted C₁₋C₆alkoxy and a 5 to 6 membered heteroaryl having 1 to 3 heteroatoms independently selected from N, O and S;
R⁵ is selected from
a) —NR⁶R⁷;
b) —OR¹¹;
c) —S(CR⁸R⁹)ₙC(═O)OR¹⁰,
d) an unsubstituted 6 membered heterocycloalkyl having 1 to 2 ring members independently selected from N, O and S;
and
e) a 5 to 6 membered monocyclic heterocycloalkyl having 1 to 2 N heteroatoms as ring members, wherein the heterocycloalkyl is substituted with 1 to 2 R¹² groups;
R⁶ is H, —C₁-C₆alkyl, halo-substituted C₁-C₆alkyl, C₃-C₈cycloalkyl, —(CR⁸R⁹)˜OR¹⁴ or —(CR⁸R⁹)ₘR¹⁶;
R⁷ is H, —C₁₋C₆alkyl, —(CR⁸R⁹)ₙC(═O)OR¹⁰, —(CR⁸R⁹)ₙ(CR¹⁴R¹⁵)ₘC(═O)OR¹⁰, —(CR¹⁴R¹⁵)ₙR¹⁷, (CR¹⁴R¹⁵)ₘC(═O)OR¹⁰, —CHR¹²R¹⁸, a monocyclic C₃₋C₈cycloalkyl substituted with 1 to 2 R¹² groups, a bicyclic C₇₋C₁₀cycloalkenyl substituted with 1 to 2 R¹² groups or a bicyclic C₇₋C₁₀cycloalkyl substituted with 1 to 2 R¹² groups;
each R⁸ is independently selected from H, D, deuterium substituted C₁₋C₆alkyl and C₁₋C₆alkyl;
each R⁹ is independently selected from H, D, deuterium substituted C₁₋C₆alkyl and C₁₋C₆alkyl;
each R¹⁰ is independently selected from H, and C₁₋C₆alkyl;
R¹¹ is —(CR⁸R⁹)ₙC(═O)OR¹³ or a C₃₋C₈cycloalkyl substituted with 1 to 2 R¹² groups,
each R¹² is independently selected from C₁₋C₆alkyl, —OH, —(CR⁸R⁹)ₙC(═O)OR¹³, —C(═O)NH(CR¹⁴R¹⁵)ₘC(═O)OR¹³, —C(═O)OR¹³, —(CR¹⁴R¹⁵)ₘC(═O)OR¹³, —O(CR⁸R⁹)ₙC(═O)OR¹³, benzoic acid and tetrazolyl;
each R¹³ is independently selected from H, and C₁-C₆alkyl;
each R¹⁴ is independently selected from H, D, deuterium substituted C₁₋₆alkyl and C₁₋₆alkyl;
each R¹⁵ is independently selected from H, D, deuterium substituted C₁₋C₆alkyl and C₁₋C₆alkyl;
or R¹⁴ and R¹⁵ together with the carbon in CR¹⁴R¹⁵ form a C₃₋C₈cycloalkyl;
R¹⁶ is a C₃₋C₈cycloalkyl;
R¹⁷ is
a) a C₃₋C₈cycloalkyl substituted with 1 to 2 R¹² groups,
or
b) a 5 to 6 membered monocyclic heterocycloalkyl having 1 to 2 ring members independently selected from N, O and S substituted with 1 to 2 R¹² groups;
R¹⁸ is adamantanyl;
each m is independently selected from 1, 2, 3, 4, 5, 6, 7, 8, 9 and 10; and
each n is independently selected from 1, 2, 3, 4, 5, 6, 7, 8, 9 and 10.

22. The method of claim 21, wherein the CFTR mediated disease is selected from cystic fibrosis, asthma, COPD, emphysema and chronic bronchitis.

23. (canceled)

24. The method of claim 21, wherein the CFTR mediated disease is cystic fibrosis.

25. The method of claim 21, further comprising administering to the subject one or more additional pharmaceutical agent(s) prior to, concurrent with, or subsequent to the administration of the compound of Formula (I), or a pharmaceutically acceptable salt thereof.

26. The method of claim 25, wherein the additional pharmaceutical agent(s) is selected from a mucolytic agent, nebulized hypertonic saline, bronchodilator, an antibiotic, an anti-infective agent, a CFTR modulator, and an anti-inflammatory agent.

27. The method of claim 25, wherein the additional pharmaceutical agent is a CFTR modulator or a CFTR potentiator.

28. (canceled)

29. The method of claim 25, wherein the additional pharmaceutical agents are a CFTR modulator and a CFTR potentiator.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)
